(19) United States
(12) Patent Application Publication
(10) Pub. No.: US 2025/0064968 A1
(43) Pub. Date: Feb. 27, 2025

(54) ANTIBODY-CONJUGATED CHEMICAL INDUCERS OF DEGRADATION WITH HYDOLYSABLE MALEIMIDE LINKERS AND METHODS THEREOF

(71) Applicant: Genentech, Inc., South San Francisco, CA (US)

(72) Inventors: Daniel P. SUTHERLIN, Burlingame, CA (US); Donglu ZHANG, San Mateo, CA (US); Summer A. BAKER DOCKERY, San Francisco, CA (US); Peter Scott DRAGOVICH, San Mateo, CA (US)

(73) Assignee: Genentech, Inc., South San Francisco, CA (US)

(21) Appl. No.: 18/784,386

(22) Filed: Jul. 25, 2024

Related U.S. Application Data
(63) Continuation of application No. PCT/US2023/061216, filed on Jan. 25, 2023.
(60) Provisional application No. 63/303,447, filed on Jan. 26, 2022.

(51) Int. Cl. A61K 47/68

(52) U.S. Cl. CPC A61K 47/6889; A61K 47/6803

(57) ABSTRACT

The subject matter described herein relates generally to hydrolysable maleimide-containing molecules that are useful as linkers to covalently bind chemical inducers of degradation to antibodies and to the conjugates produced therefrom and their uses to treat conditions, and to the uses of the conjugates in treating diseases and conditions where targeted protein degradation is beneficial.

### CROSS-REFERENCE TO RELATED APPLICATIONS

This application is a Continuation of International Application No. PCT/US2023/061216, filed Jan. 25, 2023, which claims the benefit of U.S. Provisional Application No. 63/303,447, filed Jan. 26, 2022, the content of each of which is incorporated by reference herein in its entirety.

### FIELD

The subject matter described herein relates generally to hydrolysable maleimide-containing molecules that are useful as linkers to covalently bind chemical inducers of degradation to antibodies and to the conjugates produced therefrom.

### BACKGROUND

Cell maintenance and normal function requires controlled degradation of cellular proteins. For example, degradation of regulatory proteins triggers events in the cell cycle, such as DNA replication, chromosome segregation, etc. Accordingly, such degradation of proteins has implications for the cell's proliferation, differentiation, and death.

While inhibitors of proteins can block or reduce protein activity in a cell, protein degradation in a cell can also reduce activity or remove altogether the target protein. Utilizing a cell's protein degradation pathway can, therefore, provide a means for reducing or removing protein activity. One of the cell's major degradation pathways is known as the ubiquitin-proteasome system. In this system, a protein is marked for degradation by the proteasome by ubiquitinating the protein. The ubiqitinization of the protein is accomplished by an E3 ubiquitin ligase that binds to a protein and adds ubiquitin molecules to the protein. The E3 ubiquitin ligase is part of a pathway that includes E1 and E2 ubiquitin ligases, which make ubiquitin available to the E3 ubiquitin ligase to add to the protein.

To harness this degradation pathway, molecular constructs known as chemical inducers of degradation (CIDEs) bring together an E3 ubiquitin ligase with a protein that is to be targeted for degradation. To facilitate a protein for degradation by the proteasome, the CIDE is comprised of a group that binds to an E3 ubiquitin ligase and a group that binds to the protein target for degradation. These groups are typically connected with a linker. This CIDE can bring the E3 ubiquitin ligase in proximity with the protein so that it is ubiquitinated and marked for degradation. However, the relatively large size of the CIDE can be problematic for targeted delivery, as well as contribute to undesirable properties, such as fast metabolism/clearance, short half-life, and low bioavailability.

There is an ongoing need in the art for improving CIDEs, including enhancing targeted delivery of CIDEs to cells that contain the protein target. The subject matter described herein addresses this and other shortcomings in the art.

### BRIEF SUMMARY

In certain embodiments, the present disclosure is directed to hydrolysable maleimide-containing linkers useful for covalently binding a CIDE to an antibody in an Ab-conjugated CIDE to form an Ab-L1-CIDE.

In certain embodiments, the present disclosure is directed to a CIDE covalently bound to a hydrolysable maleimide-containing linker.

In certain embodiments, the present disclosure is directed to an Ab-L1-CIDE, wherein L1 is a hydrolysable maleimide-containing linker covalently bound to Ab and to CIDE.

In certain embodiments, the hydrolysable maleimide-containing linker has a structure of Formula I, I-A, I-B, I-C or I-G.

In certain embodiments, the subject matter described herein is directed to a pharmaceutical composition comprising an Ab-L1-CIDE, as described herein, and one or more pharmaceutically acceptable excipients.

In certain embodiments, the subject matter described herein is directed to the use of an Ab-L1-CIDE, as described herein, in methods of treating conditions and diseases by administering to a subject a pharmaceutical composition comprising an Ab-L1-CIDE.

In certain embodiments, of the subject matter described herein is a method of making an Ab-L1-CIDE.

In certain embodiments, the subject matter described herein is directed to an article of manufacture comprising a pharmaceutical composition comprising an Ab-L1-CIDE, a container, and a package insert or label indicating that the pharmaceutical composition can be used to treat a disease or condition.

Yet other embodiments are also fully described herein.

### BRIEF DESCRIPTION OF THE FIGURES

FIG. 1 is a mechanistic representation of a possible route of conjugation, hydrolysis and cleavage of an Ab-L1-CIDE.

### DETAILED DESCRIPTION

Disclosed herein are hydrolysable maleimide-containing linkers (referred to in embodiments as L1, L1, L_{X} or L_{Z}). The linkers are useful for covalently binding a degrader, also referred to as a PROTAC or CIDE (“Chemical Inducers of Degradation), to an antibody. The hydrolysable maleimide-containing linkers are a component in an Ab-conjugated CIDE (“antibody-conjugated CIDE,” “Ab-L1-CIDE” or “Ab-CIDE”), wherein a hydrolysable maleimide-containing linker is covalently bound to the CIDE and to an antibody. These conjugates are useful in targeted protein degradation.

The subject matter described herein utilizes hydrolysable maleimide-containing linkers that are able to bind covalently in a stable manner to provide increased number of bound linkers to the antibody. Thus, the number of CIDEs per antibody can be increased in a stable manner. It is advantageous to have a relatively higher number of stably bound linkers on the antibody, which linkers in turn can be used to conjugate to a CIDE. Higher DAR enables lower potency, targeted payloads vs traditional highly cyctotoxic payloads. The use of these less-potent payloads increases the therapeutic window. Dosing can also be lowered for a high DAR vs low DAR conjugate.

The subject matter described herein utilizes an antibody to target or direct a CIDE to a target cell or tissue. As described herein, delivery of a CIDE to a target cell or tissue is improved by connecting an antibody to the CIDE to form an Ab-CIDE. As shown herein, e.g. in the Examples, a cell that expresses an antigen can be targeted by an antigen-specific antibody of an Ab-CIDE, whereby the Ab-CIDE is delivered to the target cell expressing such antigen, and the CIDE portion of the Ab-CIDE is delivered intracellularly to the target cell.

Accordingly, the subject matter described herein is directed to Ab-CIDE compositions that result in the ubiquitination of a target protein and subsequent degradation of the protein. The compositions comprise an antibody covalently linked to a hydrolysable Linker 1 (L1), which is covalently linked at any available point of attachment to a CIDE. The CIDE comprises an E3 ubiquitin ligase binding (E3LB) moiety, wherein the E3LB moiety recognizes a E3 ubiquitin ligase protein that is VHL, a Linker 2 (L2) covalently connecting the E3LB moeity to the protein binding moiety (PB), which is the moeity that recognizes a target protein. The subject matter described herein is useful for degrading, and thus regulating protein activity, and treating diseases and conditions related to protein activity.

The presently disclosed subject matter will now be described more fully hereinafter. However, modifications and other embodiments of the presently disclosed subject matter set forth herein will come to mind to one skilled in the art to which the presently disclosed subject matter pertains having the benefit of the teachings presented herein. Therefore, it is to be understood that the presently disclosed subject matter is not to be limited to the specific embodiments disclosed and that modifications and other embodiments are intended to be included within the scope of the appended claims. In other words, the subject matter described herein covers all alternatives, modifications, and equivalents. In the event that one or more of the incorporated literature, patents, and similar materials differs from or contradicts this application, including but not limited to defined terms, term usage, described techniques, or the like, this application controls. Unless otherwise defined, all technical and scientific terms used herein have the same meaning as commonly understood by one of ordinary skill in this field. All publications, patent applications, patents, and other references mentioned herein are incorporated by reference in their entirety.

### I. Definitions

The term “hydrolysable” refers to a linker that contains a moiety that, under physiological conditions, can induce the hydrolysis of the thio-substituted succinimide, formed when the maleimide is conjugated to an antibody through a thioether bond.

The term “CIDE” refers to Chemical Inducers of Degradation that are proteolysis-targeting chimera molecules having generally three components, an E3 ubiquitin ligase-binding group (E3LB), a Linker 2 (L2), and a protein-binding group (PB).

The terms “residue,” “moiety,” “portion,” or “group” refers to a component that is covalently bound or linked to another component. The term “component” is also used herein to describe such a residue, moiety, portion or group. By way of example, a residue of a compound will have an atom or atoms of the compound, such as a hydrogen or hydroxy, replaced with a covalent bond, thereby binding the residue to another component of the CIDE, L1-CIDE or Ab-CIDE. For example a “residue of a CIDE” refers to a CIDE that is covalently linked to one or more groups such as a Linker L2, which itself can be optionally further linked to an antibody.

The term “covalently bound” or “covalently linked” refers to a chemical bond formed by sharing of one or more pairs of electrons.

The term “protein binding group” or “PB” refers to a residue of a small molecule or other compound which is capable of binding to a target protein or other polypeptide target of interest. In the conjugate molecules described herein, the PB binds to the target, which places the target in proximity to a ubiquitin ligase such that degradation of the protein or polypeptide by ubiquitin ligase may occur. As such, the conjugates described herein can include any PB so long as it is covalently bound to L2 and interacts or binds to a target of interest. Non-limiting examples of small molecule target protein binding moieties include compounds that bind BRM (BRAHMA), Hsp90 inhibitors, Tau and Androgen Receptors (AR), kinase inhibitors, such as BRG1, AKT, HPK1 and IRE1, MDM2 inhibitors, compounds targeting Human BET Bromodomain-containing proteins, HDAC inhibitors, human lysine methyltransferase inhibitors, such as KDM5, angiogenesis inhibitors, immunosuppressive compounds, and compounds targeting the aryl hydrocarbon receptor (AHR), among numerous others. In certain embodiments, the CIDES and conjugated CIDEs described herein are not limited to the type of PB. In certain embodiments, the CIDES and conjugated CIDEs described herein are not limited to the type of PB, wherein the PB is covalently bound to L2; and, the conjugated CIDE comprises a hydrolysable linker.

The term “E3 ligase binding (E3LB) ligand” refers to a molecule that is capable of binding Von Hippel-Lindau (VHL) E3 Ubiquitin Ligase. The terms “VCB E3 Ubiquitin Ligase,” “Von Hippel-Lindau (or VHL) E3 Ubiquitin Ligase,” “VHL,” and “Ubiquitin Ligase,” all generally describe a target enzyme(s) binding site for the E3LB portion of the conjugates described herein. VCB E3 is a protein that in combination with an E2 ubiquitin-conjugating enzyme causes the attachment of ubiquitin to a lysine on a target protein; the E3 ubiquitin ligase targets specific protein substrates for degradation by the proteasome. Thus, E3 ubiquitin ligase alone or in complex with an E2 ubiquitin conjugating enzyme is responsible for the transfer of ubiquitin to targeted proteins. In certain embodiments, the CIDES and conjugated CIDEs described herein are not limited to the type of E3LB. In certain embodiments, the CIDES and conjugated CIDEs described herein are not limited to the type of E3LB, wherein the E3LB is covalently bound to L2; and, the conjugated CIDE is further covalently attached to a hydrolysable linker.

The term “Linker”, “Linker Unit”, or “link” as used herein means a chemical moiety comprising a chain of one or more atoms that covalently attaches a CIDE moiety to an antibody, or a residue, portion, moiety, group or component of a CIDE to another residue, portion, moiety, group or component of the CIDE. In various embodiments, a linker is a divalent radical, specified as Linker 1, Linker 2, L1, L₁, L2, or L₂, and the like.

The term “peptidomimetic” as used herein means a non-peptide chemical moiety. Peptides are short chains of amino acid monomers linked by peptide (amide) bonds, the covalent chemical bonds formed when the carboxyl group of one amino acid reacts with the amino group of another. The shortest peptides are dipeptides, consisting of 2 amino acids joined by a single peptide bond, followed by tripeptides, tetrapeptides, etc. A peptidomimetic chemical moiety includes non-amino acid chemical moieties. A peptidomimetic chemical moiety may also include one or more amino acids that are separated by one or more non-amino acid chemical units. A peptidomimetic chemical moiety does not contain in any portion of its chemical structure, two or more adjacent amino acids that are linked by peptide bonds. A “peptidomimetic linker” is the portion of the molecule that is bound to the CIDE and to the antibody. Useful petpidomimetic linkers are known in the art and others are disclosed herein. The peptidomimetic linker may be a linker such as those described in WO2015/095227, WO2015/095124 or WO2015/095223, each of which is hereby incorporated by reference in its entirety.

The term “antibody” herein is used in the broadest sense and specifically covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York). A target antigen generally has numerous binding sites, also called epitopes, recognized by CDRs (complementary determining regions) on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin disclosed herein can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species. In one aspect, however, the immunoglobulin is of human, murine, or rabbit origin.

The term “antibody fragment(s)” as used herein comprises a portion of a full-length antibody, generally the antigen binding or variable region thereof. Examples of antibody fragments include Fab, Fab′, F(ab′)₂, and Fv fragments; diabodies; linear antibodies; minibodies (Olafsen et al (2004) Protein Eng. Design & Sel. 17(4):315-323), fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the subject matter described herein may be made by the hybridoma method first described by Kohler et al (1975) Nature, 256:495, or may be made by recombinant DNA methods (see for example: U.S. Pat. Nos. 4,816,567; 5,807,715). The monoclonal antibodies may also be isolated from phage antibody libraries using the techniques described in Clackson et al (1991) Nature, 352:624-628; Marks et al (1991) J. Mol. Biol., 222:581-597; for example.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al (1984) Proc. Natl. Acad. Sci. USA, 81:6851-6855). Chimeric antibodies of interest herein include “primatized” antibodies comprising variable domain antigen-binding sequences derived from a non-human primate (e.g., Old World Monkey, Ape, etc.) and human constant region sequences.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are designated α, δ, ε, γ and μ, respectively.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following. In certain embodiments, an antibody as described herein has dissociation constant (Kd) of ≤1 μM, ≤100 nM, ≤10 nM, ≤5 nm, ≤4 nM, ≤3 nM, ≤2 nM, ≤1 nM, ≤0.1 nM, ≤0.01 nM, or ≤0.001 nM (e.g., 10⁻⁸M or less, e.g. from 10⁻⁸ M to 10⁻¹³ M, e.g., from 10⁻⁹ M to 10⁻¹³ M).

The term “free cysteine amino acid” as used herein refers to a cysteine amino acid residue which has been engineered into a parent antibody, has a thiol functional group (—SH), and is not paired as an intramolecular or intermolecular disulfide bridge. The term “amino acid” as used herein means glycine, alanine, valine, leucine, isoleucine, phenylalanine, proline, serine, threonine, tyrosine, cysteine, methionine, lysine, arginine, histidine, tryptophan, aspartic acid, glutamic acid, asparagine, glutamine or citrulline.

A “patient” or “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the patient, individual, or subject is a human. In some embodiments, the patient may be a “cancer patient,” i.e. one who is suffering or at risk for suffering from one or more symptoms of cancer.

A “patient population” refers to a group of cancer patients. Such populations can be used to demonstrate statistically significant efficacy and/or safety of a drug.

The terms “cancer” and “cancerous” refer to or describe the physiological condition in mammals that is typically characterized by unregulated cell growth/proliferation. A “tumor” comprises one or more cancerous cells. Examples of cancer are provided elsewhere herein.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the subject matter described herein are used to delay development of a disease or to slow the progression of a disease.

An “effective amount” of an agent, e.g., a pharmaceutical formulation, refers to an amount effective, at dosages and for periods of time necessary, to achieve the desired therapeutic or prophylactic result. For example, an effective amount of the drug for treating cancer may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. The effective amount may extend progression free survival (e.g. as measured by Response Evaluation Criteria for Solid Tumors, RECIST, or CA-125 changes), result in an objective response (including a partial response, PR, or complete response, CR), increase overall survival time, and/or improve one or more symptoms of cancer (e.g. as assessed by FOSI).

As used herein, the term “therapeutically effective amount” means any amount which, as compared to a corresponding subject who has not received such amount, results in treatment of a disease, disorder, or side effect, or a decrease in the rate of advancement of a disease or disorder. The term also includes within its scope amounts effective to enhance normal physiological function. For use in therapy, therapeutically effective amounts of an Ab-CIDE, as well as salts thereof, may be administered as the raw chemical. Additionally, the active ingredient may be presented as a pharmaceutical composition.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical formulation, other than an active ingredient, which is nontoxic to a subject. A pharmaceutically acceptable excipient includes, but is not limited to, a buffer, carrier, stabilizer, or preservative.

The phrase “pharmaceutically acceptable salt,” as used herein, refers to pharmaceutically acceptable organic or inorganic salts of a molecule. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

Other salts, which are not pharmaceutically acceptable, may be useful in the preparation of compounds of described herein and these should be considered to form a further aspect of the subject matter. These salts, such as oxalic or trifluoroacetate, while not in themselves pharmaceutically acceptable, may be useful in the preparation of salts useful as intermediates in obtaining the compounds described herein and their pharmaceutically acceptable salts.

The term “alkyl” as used herein refers to a saturated linear or branched-chain monovalent hydrocarbon radical of any length from one to five carbon atoms (C₁-C₅), wherein the alkyl radical may be optionally substituted independently with one or more substituents. In another embodiment, an alkyl radical is one, two, three, four or five carbon atoms. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), and the like. It is understood that an alkyl chain that is covalently bound at each end can form an alkylene chain as described below.

“Halogen” or “halo” refers to atoms occupying group VIIA of the periodic table, such as fluoro, chloro, bromo or iodo.

“Haloalkyl” refers to an unbranched or branched alkyl group as defined above, wherein one or more (e.g., 1 to 6, or 1 to 3) hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl and the like. Furthermore, “halo-C₁-C₃ alkyl” refers to an alkyl group of 1 to 3 carbons wherein at least one hydrogen atom is replaced by a halogen. Additionally, “halo-C₁-C₆ alkyl” refers to an alkyl group of 1 to 6 carbons wherein at least one hydrogen atom is replaced by a halogen.

The term “alkylene” as used herein refers to a saturated linear or branched-chain divalent hydrocarbon radical of any length from one to twelve carbon atoms (C₁-C₁₂), wherein the alkylene radical may be optionally substituted independently with one or more substituents described below. In another embodiment, an alkylene radical is one to eight carbon atoms (C₁-C₈), or one to six carbon atoms (C₁-C₆). Examples of alkylene groups include, but are not limited to, methylene (—CH₂—), ethylene (—CH₂CH₂—), propylene (—CH₂CH₂CH₂—), and the like.

The terms “carbocycle”, “carbocyclyl”, “carbocyclic ring” and “cycloalkyl” refer to a monovalent non-aromatic, saturated or partially unsaturated ring having 3 to 5 carbon atoms (C₃-C₅) as a monocyclic ring. Examples of monocyclic carbocycles include, but are not limited to, cyclopropyl, cyclobutyl, cyclopentyl, 1-cyclopent-1-enyl, 1-cyclopent-2-enyl, 1-cyclopent-3-enyl, and the like. Carbocyclyl groups can be optionally substituted independently with one or more alkyl groups.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆-C₂₀ aryl), 6 to 12 carbon ring atoms (i.e., C₆-C₁₂ aryl), or 6 to 10 carbon ring atoms (i.e., C₆-C₁₀ aryl). Examples of aryl groups include, e.g., phenyl, naphthyl, fluorenyl and anthryl.

“Heteroaryl” refers to an aromatic group having a single ring, multiple rings or multiple fused rings, with one or more ring heteroatoms independently selected from nitrogen, oxygen, and sulfur. As used herein, heteroaryl includes 1 to 20 ring carbon atoms (i.e., C₁-C₂₀ heteroaryl), 3 to 12 ring carbon atoms (i.e., C₃-C₁₂ heteroaryl), or 3 to 8 carbon ring atoms (i.e., C₃-C₈ heteroaryl), and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 9-10 membered ring systems, 6-10 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Examples of heteroaryl groups include, e.g., acridinyl, benzimidazolyl, benzothiazolyl, benzindolyl, benzofuranyl, benzothiazolyl, benzothiadiazolyl, benzonaphthofuranyl, benzoxazolyl, benzothienyl (benzothiophenyl), benzotriazolyl, benzo[4,6]imidazo[1,2-a]pyridyl, carbazolyl, cinnolinyl, dibenzofuranyl, dibenzothiophenyl, furanyl, isothiazolyl, imidazolyl, indazolyl, indolyl, indazolyl, isoindolyl, isoquinolyl, isoxazolyl, naphthyridinyl, oxadiazolyl, oxazolyl, 1-oxidopyridinyl, 1-oxidopyrimidinyl, 1-oxidopyrazinyl, 1-oxidopyridazinyl, phenazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, thiazolyl, thiadiazolyl, triazolyl, tetrazolyl and triazinyl. Examples of the fused-heteroaryl rings include, but are not limited to, benzo[d]thiazolyl, quinolinyl, isoquinolinyl, benzo[b]thiophenyl, indazolyl, benzo[d]imidazolyl, pyrazolo[1,5-a]pyridinyl and imidazo[1,5-a]pyridinyl, where the heteroaryl can be bound via either ring of the fused system. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above.

“Heterocycle,” “heterocyclic,” “heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂— moieties. Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like. A heterocyclyl group can be substituted as described in WO2014/100762.

The term “chiral” refers to molecules which have the property of non-superimposability of the mirror image partner, while the term “achiral” refers to molecules which are superimposable on their mirror image partner.

The term “stereoisomers” refers to compounds which have identical chemical constitution, but differ with regard to the arrangement of the atoms or groups in space.

“Diastereomer” refers to a stereoisomer with two or more centers of chirality and whose molecules are not mirror images of one another. Diastereomers have different physical properties, e.g. melting points, boiling points, spectral properties, and reactivities. Mixtures of diastereomers may separate under high resolution analytical procedures such as electrophoresis and chromatography.

“Enantiomers” refer to two stereoisomers of a compound which are non-superimposable mirror images of one another.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

Other terms, definitions and abbreviations herein include: Wild-type (“WT”); Cysteine engineered mutant antibody (“thio”); light chain (“LC”); heavy chain (“HC”); 6-maleimidocaproyl (“MC”); maleimidopropanoyl (“MP”); valine-citrulline (“val-cit” or “ve”), alanine-phenylalanine (“ala-phe”), p-aminobenzyl (“PAB”), and p-aminobenzyloxycarbonyl (“PABC”); A118C (EU numbering)=A121C (Sequential numbering)=A114C (Kabat numbering) of heavy chain K149C (Kabat numbering) of light chain.

Additional definitions and abbreviations are provided elsewhere herein.

### II. Hydrolysable Linkers

In embodiments, the subject matter described herein is directed to a peptidomimetic linker comprising a hydrolysable moiety, wherein the linker is covalently bound to a CIDE or an antibody and a CIDE. In embodiments, the hydrolysable moiety of the maleimide portion of the peptidomimetic linker comprises a structure having formula I:

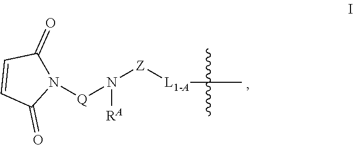

wherein,
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, phenyl or benzyl;
Q is selected from the group consisting of:
i)

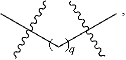

wherein q is 1, 2, 3 or 4; and
ii)

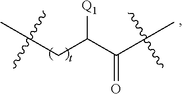

wherein t is 0, 1, 2, 3 or 4; and
wherein Q¹ is hydrogen,

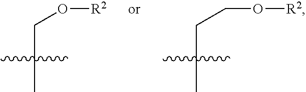

wherein R² is hydrogen, halo(C₁₋₆)alkyl or C₁₋₆ alkyl; and
L_{1-A} is the remainder of the peptidomimetic linker.

In certain embodiments, the subject matter described herein is directed to compounds having the structure:

L₁-D
wherein,
D is a CIDE;
L₁ is a linker covalently bound to D and having a structure of Formula I:

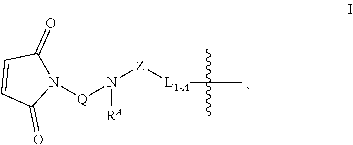

wherein,
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, phenyl, benzyl;
Q is selected from the group consisting of:
i)

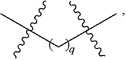

wherein q is 1, 2, 3 or 4; and
ii)

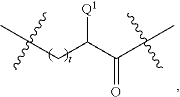

wherein,
t is 0, 1, 2, 3 or 4; and
Q¹ is hydrogen,

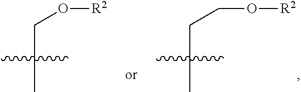

wherein R² is hydrogen, halo(C₁₋₆)alkyl or C₁₋₆ alkyl;
and
   L_{1-A} is:

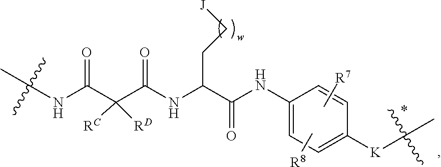

wherein

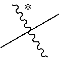

indicates the attachment point to D;
w is 0, 1, 2, 3, 4 or 5;
J is selected from the group consisting of C₁₋₅ alkyl, —N(R^{x})(R^{y}), —C(O)NH₂, —NH—C(O)—NH₂, and —NH—C(NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein Rₓ and R_{y} are each independently selected from hydrogen and C₁₋₃alkyl, or Rₓ and R_{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

In embodiments, L₁-D is selected from the group consisting of

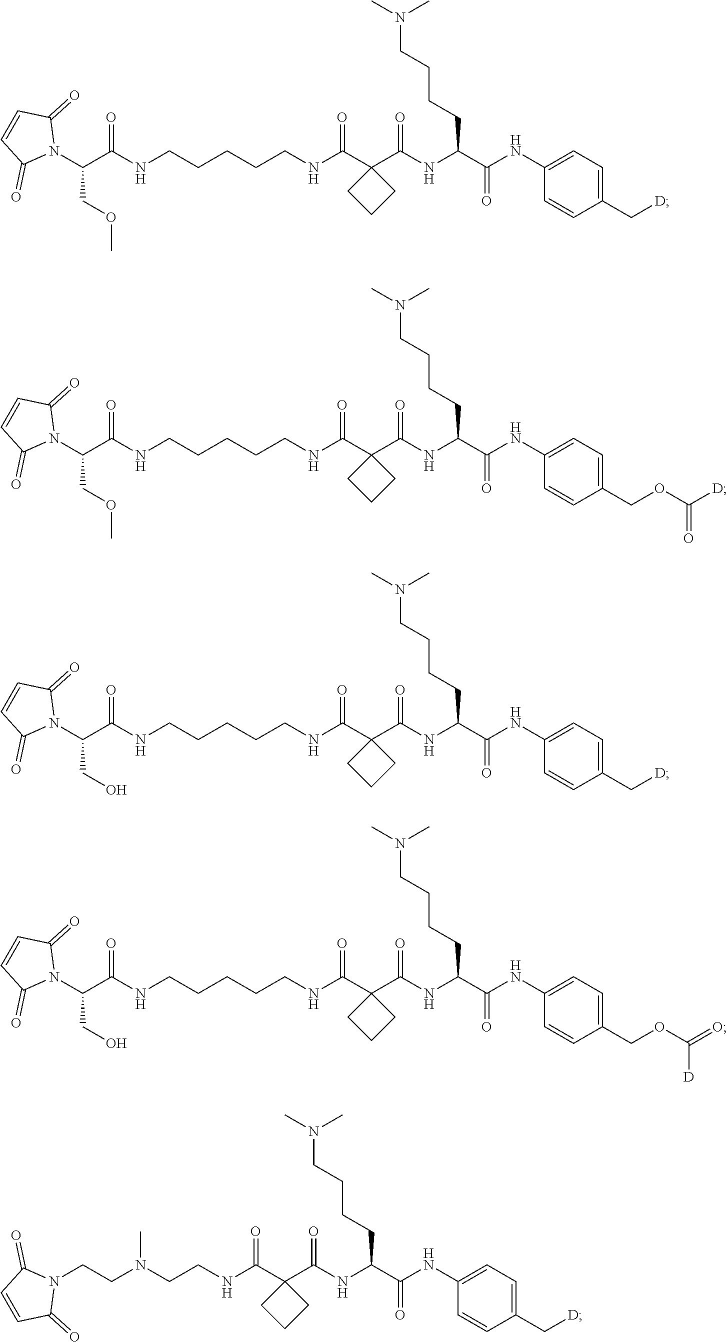

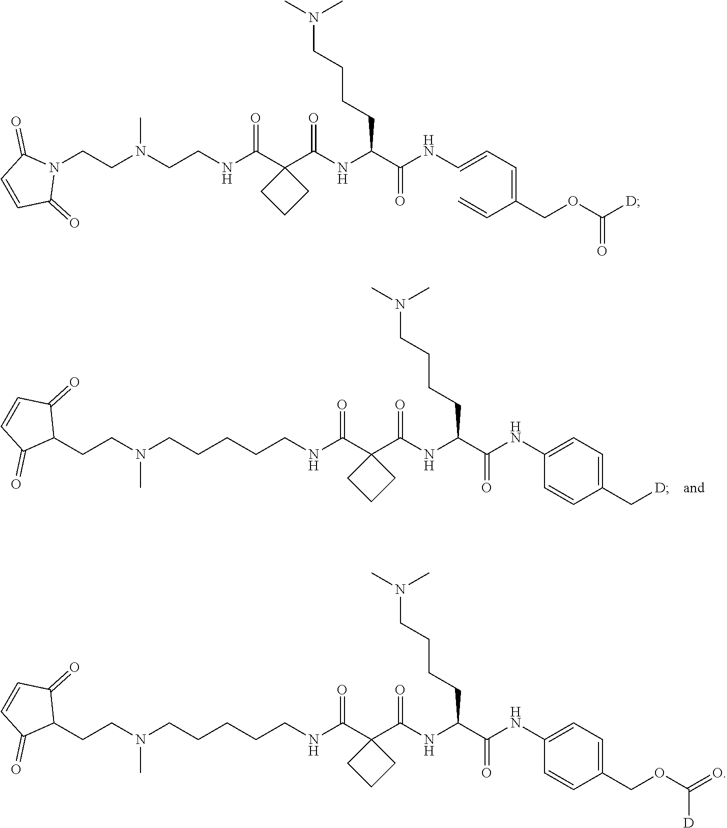

In certain embodiments, the subject matter described herein is directed to antibody-CIDE conjugates having the structure:

Ab-(L₁-D)j
wherein,
Ab is an antibody;
j is 1 to 16;
D is a CIDE;
L₁ is a linker covalently bound to Ab and to D and having a structure of Formula I-A:

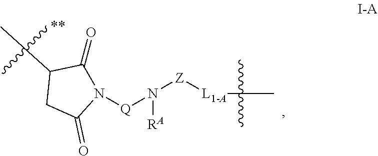

wherein,
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, phenyl, benzyl;
Q is selected from the group consisting of:
i)

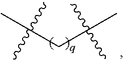

wherein q is 1, 2, 3 or 4; and
ii)

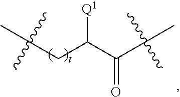

wherein t is 0, 1, 2, 3 or 4;
Q¹ is hydrogen,

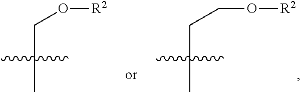

wherein R² is hydrogen, halo(C₁₋₆)alkyl or C₁₋₆ alkyl; and
L_{1-A} is:

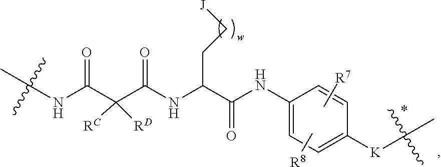

wherein

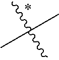

indicates the attachment point to D;
w is 0, 1, 2, 3, 4 or 5;
J is selected from the group consisting of C₁₋₅ alkyl, —N(R^{x})(R^{y}), —C(O)NH₂, —NH—C(O)—NH₂, and —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein Rₓ and R_{y} are each independently selected from hydrogen and C₁₋₃alkyl, or Rₓ and R_{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

In certain embodiments, the subject matter described herein is directed to antibody-CIDE conjugates having the structure:

Ab-((L_{X})-D)ⱼ
wherein
Ab is an antibody;
j is 1 to 16;
D is a CIDE;
L_{X} is selected from the group consisting of L₁ and L₁ₕ, wherein L₁ₕ is present in at least one instance of L_{X}:
L₁ is a linker-1 covalently bound to Ab and to D and having a structure of Formula I-A:

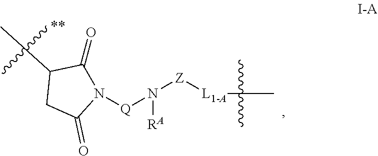

and
L₁ₕ is a linker covalently bound to D and having a structure of Formula I-B:

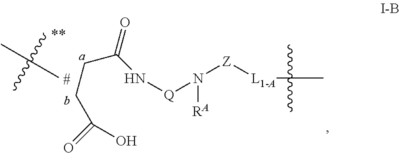

wherein # indicates the point of attachment at position a or b;
in L₁ and L₁ₕ,

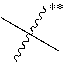

indicates the point of attachment to the antibody;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, phenyl, benzyl;
Q is selected from the group consisting of:
i)

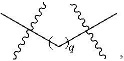

wherein q is 1, 2, 3 or 4; and
ii)

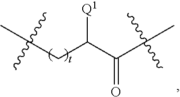

wherein t is 0, 1, 2, 3 or 4;
Q¹ is hydrogen,

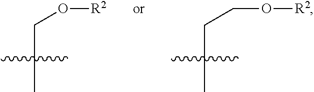

wherein R² is hydrogen, halo(C₁₋₆)alkyl or C₁₋₆ alkyl;
and,
   L_{1-A} is:

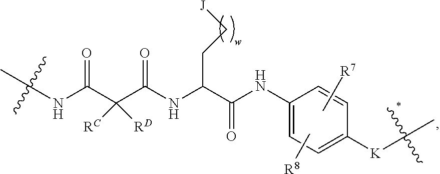

wherein

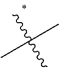

indicates the attachment point to D;
w is 0, 1, 2, 3, 4 or 5;
J is selected from the group consisting of C₁₋₅ alkyl, —N(R^{x})(R^{y}), —C(O)NH₂, —NH—C(O)—NH₂, and —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

In certain embodiments, Ab-((L_{X})-D)ⱼ is an Ab-CIDE as described above that comprises at least one linker covalently bound to the antibody and having a structure of Formula I-B.

In certain embodiments, the Ab-((L_{X})-D)ⱼ conjugate is a product of complete or partial hydrolysis of Ab-(L₁-D)ⱼ.

In certain embodiments, the subject matter described herein is directed to antibody-linker conjugates having the structure:

Ab-(L_{Z})ⱼ
wherein,
Ab is an antibody;
j is 1 to 16;
L_{Z} is selected from the group consisting of L_{X1} and Lx-D, wherein L_{X1} is present in at least one instance of L_{Z}:
L_{X1} is covalently bound to Ab and has a structure of Formula I-G:

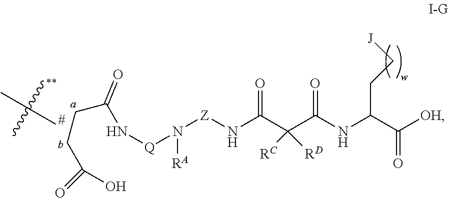

and,
Lx-D, wherein L_{X} is selected from the group consisting of L₁ and L₁ₕ:
L₁ is a linker-1 covalently bound to Ab and to D and having a structure of Formula I-A or Formula I-C:

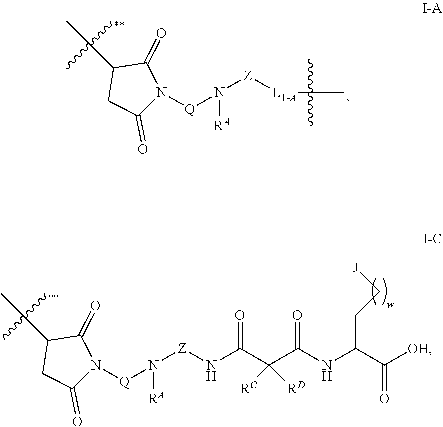

and,
L₁ₕ is a linker-1 covalently bound to Ab and to D and having a structure of Formula I-B:

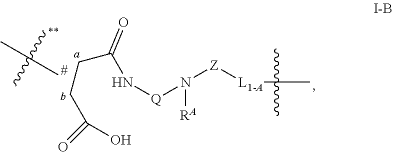

wherein, # indicates the point of attachment at position a or b;

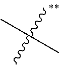

indicates the point of attachment to the antibody;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, phenyl, benzyl;
Q is selected from the group consisting of:
i)

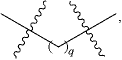

wherein q is 1, 2, 3 or 4; and
ii)

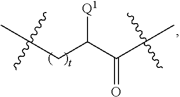

wherein t is 0, 1, 2, 3 or 4;

Q¹ is hydrogen,

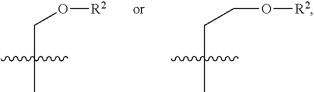

wherein R² is hydrogen, halo C₁₋₆ alkyl or C₁₋₆ alkyl; and
L_{1-A} is:

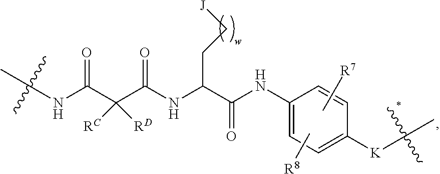

wherein

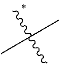

indicates the attachment point to D;
w is 0, 1, 2, 3, 4 or 5;
J is selected from the group consisting of C₁₋₅ alkyl, —N(R^{x})(R^{y}), —C(O)NH₂, —NH—C(O)—NH₂, and —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}) or C(O)—N(R^{x})(R^{y}), wherein u is 0, 1, 2, or 3, and wherein Rₓ and R_{y} are each independently selected from hydrogen and C₁₋₃alkyl, or Rₓ and R_{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

In certain embodiments, Ab-((L_{z})ⱼ is an Ab-CIDE as described above that comprises at least one linker covalently bound to the antibody and having a structure of Formula I-G.

In certain embodiments, the Ab-((L_{z})ⱼ conjugate is a product of partial or complete cleavage and/or partial or complete hydrolysis of Ab-(L₁-D)ⱼ.

In certain embodiments of Formulae I, I-A, I-B, I-C and I-G, Q is:

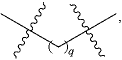

wherein, q is 1, 2, 3 or 4. In certain embodiments, q is 1, 2 or 3. In certain embodiments, q is 2.

In certain embodiments of Formulae I, I-A, I-B, I-C and I-G, Q is:

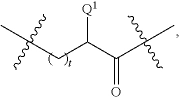

wherein t is 0, 1, 2, 3 or 4;
Q¹ is hydrogen,

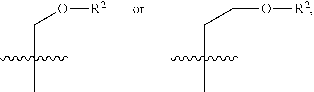

wherein R² is hydrogen, halo C₁₋₆ alkyl or C₁₋₆ alkyl.

In certain embodiments of Formulae I, I-A, I-B, I-C and I-G, R² is hydrogen or C₁₋₆ alkyl. In certain embodiments, R² is methyl, ethyl, propyl or butyl. In certain embodiments, R² is methyl.

In certain embodiments of Formulae I, I-A, I-B, I-C and I-G, Q¹ is:

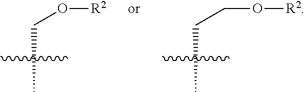

In certain aspects of these embodiments, R² is hydrogen or methyl.

In certain embodiments of Formulae I, I-A, I-B, I-C and I-G, Q¹ is:

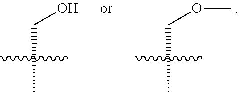

In certain embodiments of Formulae I, I-A, I-B, I-C and I-G, t is 0.

In certain embodiments of Formulae I, I-A, I-B, I-C and I-G, the compound of claim 1, wherein Z is —(CH₂)ₚ—, wherein p is 1, 2, 3, 4, 5 or 6. In aspects of these embodiments, p is 4, 5 or 6. In an aspect of these embodiments, p is 5.

In certain embodiments of Formulae I, I-A, I-B, I-C and I-G, Z is —CH₂—(CH₂—O—CH₂)ₚ—CH₂—, wherein p is 1, 2, 3, 4, 5 or 6. In aspects of these embodiments, p is 1, 2 or 3. In an aspect of these embodiments, p is 1.

In certain embodiments of Formulae I, I-A, I-B, I-C and I-G, R^{A} is hydrogen or C₁₋₆ alkyl. In aspects of these embodiments, R^{A} is hydrogen or methyl.

In certain embodiments of Formulae I, I-A, I-B, I-C and I-G, R^{A} is —(CH₂)ᵥ—Ar, wherein Ar is an optionally substituted aryl. In certain embodiments, Ar is a C₆₋₁₀ aryl. In certain embodiments, Ar is a C₆ aryl. In certain embodiments, v is 0 or 1. In certain embodiments, the aryl is optionally substituted once or twice with a hydroxy or C₁₋₃ alkyl. In certain embodiments, R^{A} is phenyl or benzyl.

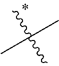

In certain embodiments of L_{1-A}, indicates the attachment point to the PB of D.

In certain embodiments of Formulae I, I-A, I-B, I-C and I-G, R⁷ and R⁸ are each hydrogen.

In certain embodiments of Formulae I, I-A, I-B, I-C and I-G, R^{C} and R^{D} together with the carbon to which each is attached, form an optionally substituted C₃₋₆ cycloalkyl. In an aspect of these embodiments, the C₃₋₆ cycloalkyl is cyclobutane.

In certain embodiments when w is 0, J is C₁₋₅ alkyl, such as methyl, and L₁, L1, L_{X} or L_{Z} comprises:

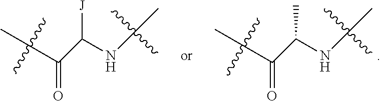

In certain embodiments, K is —CH(R)—, wherein R is hydrogen, C₁₋₃ alkylene, —CH₂O(CO)—, N(R^{x})(R^{y}), —O—N(R^{x})(R^{y}) or C(O)—N(R^{x})(R^{y}), wherein Rₓ and R_{y} are each independently selected from hydrogen and C₁₋₃ alkyl, or Rₓ and R_{y} together with the nitrogen to which each is attached form an optionally substituted 5- to 7-member heterocyclyl. In aspects of these embodiments, R is hydrogen or C₁₋₃ alkyl. In an aspect of these embodiments, R is hydrogen. In certain embodiments, K is selected from the group consisting of

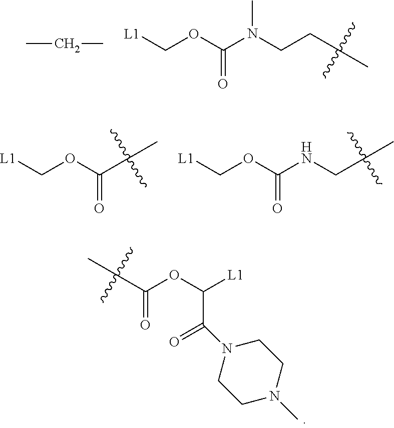

In an embodiment,
Z is —(CH₂)ₚ—, wherein p is 2 or 5;
R^{A} is hydrogen or C₁₋₆ alkyl;
Q is

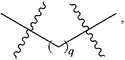

wherein, q is 2;
w is 2 or 3;
J is selected from the group consisting of C₁₋₅ alkyl, —N(R^{x})(R^{y}), wherein R^{x} and R^{y} are each C₁₋₃ alkyl, and —NH—C(O)—NH₂;
K is C₁₋₃alkylene or —CH₂—O—C(O)—;
R^{C} and R^{D}, together with the carbon to which each is attached, form an optionally substituted C₃₋₆ cycloalkyl; and
R⁷ and R⁸ are each hydrogen.

In an embodiment,
Z is —(CH₂)ₚ—, wherein p is 2 or 5;
R^{A} is hydrogen or C₁₋₆ alkyl;

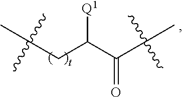

Q is

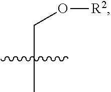

wherein t is 0, and Q¹ is r, wherein R² is hydrogen or C₁₋₆ alkyl;
w is 3;
J is selected from the group consisting of C₁₋₅ alkyl, —N(R^{x})(R^{y}), wherein R^{x} and R^{y} are each C₁₋₃ alkyl;
K is C₁₋₃alkylene or —CH₂—O—C(O)—;
R^{C} and R^{D}, together with the carbon to which each is attached, form an optionally substituted C₃₋₆ cycloalkyl; and
R⁷ and R⁸ are each hydrogen.

In certain embodiments, the subject matter described herein is directed to a compound of Formula II having the structure:

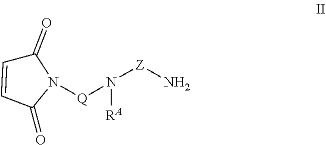

wherein
Z is —(CH₂)ₚ— or —CH₂—(CH₂—O—CH₂)ₚ—CH₂—, wherein p is an integer from 1 to 24;
R^{A} is hydrogen, C₁₋₆ alkyl, or —(CH₂)ᵥ-aryl, wherein v is 0 or 1;
Q is selected from the group consisting of:
i)

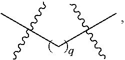

wherein q is 1, 2, 3 or 4; and
ii)

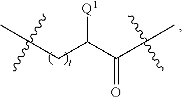

wherein t is 0, 1, 2, 3 or 4;
Q¹ is hydrogen,

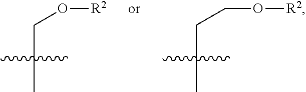

wherein R² is hydrogen, halo(C₁₋₆)alkyl or C₁₋₆ alkyl.

In certain embodiments of Formula II, Q is:

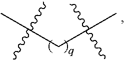

wherein, q is 1, 2, 3 or 4. In certain embodiments of Formula II, q is 2.

In certain embodiments of Formula II, The compound of claim 26, wherein Q is:

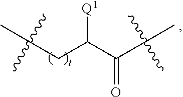

wherein t is 0, 1, 2, 3 or 4; Q¹ is hydrogen,

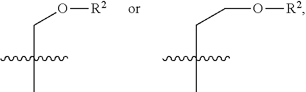

wherein, R² is hydrogen, halo C₁₋₆ alkyl or C₁₋₆ alkyl. In certain embodiments of Formula II, R² is hydrogen or C₁₋₆ alkyl. In certain aspects of these embodiments, the C₁₋₆ alkyl is methyl.

In certain embodiments of Formula II, Q¹ is:

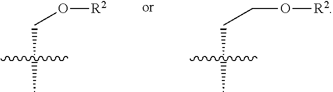

In certain embodiments of Formula II, R² is hydrogen or methyl.

In certain embodiments of Formula II, Q¹ is:

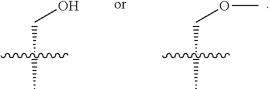

In certain embodiments of Formula II, t is 0.

In certain embodiments of Formula II, Z is —(CH₂)ₚ—, wherein p is 1, 2, 3, 4, 5 or 6. In certain aspects of these embodiments, p is 4, 5 or 6. In certain aspects of these embodiments, p is 5.

In certain embodiments of Formula II, Z is —CH₂—(CH₂—O—CH₂)ₚ—CH₂—, wherein p is 1, 2, 3, 4, 5 or 6. In certain aspects of these embodiments, p is 1, 2 or 3; or p is 1.

In certain embodiments of Formula II, R^{A} is hydrogen or C₁₋₆ alkyl. In certain aspects of these embodiments, R^{A} is hydrogen or methyl.

In certain embodiments of Formula II, R^{A} is phenyl or benzyl.

In certain embodiments, Formula II does not include a compound where: Q is —CH₂CH₂—; R^{A} is hydrogen and Z is —CH₂CH₂—; and a compound where Q is —(CH₂)₃—; R^{A} is hydrogen and Z is —(CH₂)₃—; and a compound where Q is —CH₂CH₂—; R^{A} is methyl and Z is —CH₂CH₂—.

### III. CIDEs Covalently Bound to Hydrolysable Linkers

Chemical Inducers of Degradation (CIDE) molecules are covalently bound to the hydrolysable maleimide-containing linkers described herein. In certain embodiments, a CIDE includes degraders that are bifunctional molecules, having a ubiquitin binding portion linked to a protein targeting portion, such as those described in WO2017/201449; WO 2020/086858; U.S. Pat. No. 7,208,157; US 2014/0356322; US 2015/0291562; WO2017/030814; US 2017/0008904; U.S. Pat. No. 9,938,264; US 2019/300521, US 2020/0038378, WO2021/067606; and WO2021/207291. In view of the subject matter disclosed herein, those of skill in the art would understand that the attachment point of the hydrolysable linker to the CIDE can vary and can be any available attachment point on the CIDE.

In certain embodiments, CIDEs include those having the following components:

a. E3 Ubiquitin Ligases Binding Groups (E3LB)

E3 ubiquitin ligases (of which over 600 are known in humans) confer substrate specificity for ubiquitination. There are known ligands which bind to these ligases. As described herein, an E3 ubiquitin ligase binding group is a peptide or small molecule that can bind an E3 ubiquitin ligase that is von Hippel-Lindau (VHL).

A particular E3 ubiquitin ligase is von Hippel-Lindau (VHL) tumor suppressor, the substrate recognition subunit of the E3 ligase complex VCB, which also consists of elongins B and C, Cul2 and Rbxl. The primary substrate of VHL is Hypoxia Inducible Factor 1α (HIF-1α), a transcription factor that upregulates genes such as the pro-angiogenic growth factor VEGF and the red blood cell inducing cytokine erythropoietin in response to low oxygen levels.

In certain embodiments, the E3LB portion of the CIDE can be any known E3LB ligand. In certain embodiments, the CIDES and conjugated CIDEs described herein are not limited to the type of E3LB, wherein the CIDE is covalently linked to a hydrolysable linker as described herein.

The E3LB portion has at least one terminus with a moeity that is or can be covalently linked to the L2 portion, and at least one terminus with a moeity that is or can be covalently linked to the L1 portion. For example, the E3LB portion terminates in a —NHCOOH moeity that can be covalently linked to the L2 portion through an amide bond.

In any of the aspects or embodiments described herein, the E3LB as described herein may be a pharmaceutically acceptable salt, enantiomer, diastereomer, solvate or polymorph thereof. In addition, in any of the aspects or embodiments described herein, the E3LB as described herein may be coupled to a PB directly via a bond or by a chemical linker.

In certain embodiments, the E3LB include compounds comprising a hydroxyproline moeity as described in WO 2020/086858, WO2013/106643 and WO2013/106646, each of which is incorporated herein by reference in its entirety.

In certain embodiments, the subject matter herein is directed to an E3LB portion that comprises a residue of a hydroxyproline:

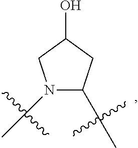

wherein, in all embodiments and substructures, the hydroxyl can be optionally replaced with another group, such as a phosphate moiety.

In certain embodiments, the E3LB comprises:

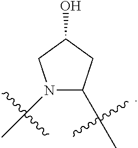 [SMILES: *C1C[C@@H](O)CN1*]

In certain embodiments, the E3LB comprises:

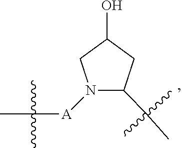

wherein, A is a group covalently bound to L2.

In certain embodiments, the E3LB comprises:

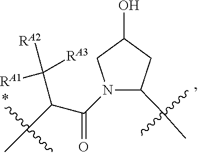

wherein,

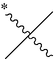

is the attachment point to L2, and
R^{A1}, R^{A2} and R^{A3} are each independently hydrogen, or C₁₋₅ alkyl; or two of R^{A1}, R^{A2} and R^{A3} together with the carbon to which each is attached form a C₁₋₅ cycloalkyl.

In certain embodiments, the E3LB comprises:

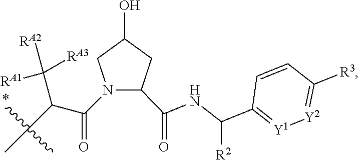

wherein,
   R² is hydrogen or C₁₋₅ alkyl;
Y¹ and Y² are each —CH or one of Y¹ and Y² is —CH and the other is N; and
R³ is cyano,

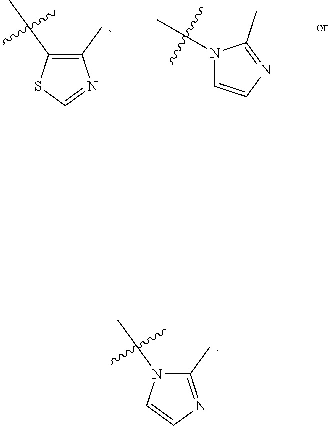

In certain embodiments, the E3LB comprises:

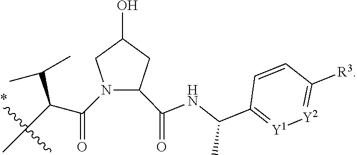

In certain embodiments, the E3LB comprises:

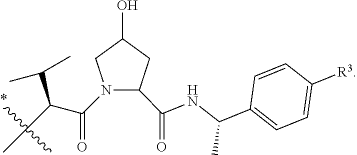

In certain embodiments, E3LB has the structure wherein R₃ is cyano.

In certain embodiments, E3LB has the structure wherein R₃ is

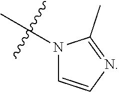 [SMILES: *n1ccnc1C]

In certain embodiments, E3LB has the structure wherein R₃ is

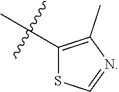 [SMILES: *c1scnc1C]

In certain embodiments, E3LB has the structure wherein R₃ is

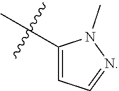 [SMILES: *c1ccnn1C]

In certain embodiments, E3LB has the structure wherein R₂ is hydrogen, methyl, ethyl or propyl.

In certain embodiments, E3LB has the structure wherein R₂ is methyl.

In certain embodiments, E3LB has the structure wherein R₂ is

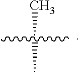

In certain embodiments, the hydroxyproline portion of E3LB has the structure:

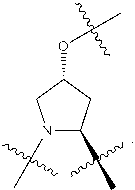 [SMILES: *O[C@@H]1C[C@@H](*)N(*)C1]

The E3LB portion has at least one terminus with a moiety that is or can be covalently linked to the L2 portion, and at least one terminus with a moiety that is or can be covalently linked to the L1 portion. For example, the E3LB portion terminates in a —NHCOOH moiety that can be covalently linked to the L2 portion through an amide bond.

In any of the aspects or embodiments described herein, the E3LB as described herein may be a pharmaceutically acceptable salt, enantiomer, diastereomer, solvate or polymorph thereof.

b. Protein Binding Group (PB)

The “protein binding group” or “PB” refers to a residue of a small molecule or other compound which is capable of binding to a target protein or other polypeptide target of interest. The PB binds to or otherwise interacts with the target, which places the target in proximity to a ubiquitin ligase such that degradation of the protein or polypeptide by ubiquitin ligase may occur. The PB can be any molecule so long as it is covalently bound to L2 and interacts or binds to a target of interest. Non-limiting examples of small molecule target protein binding moieties include compounds that bind BRM (BRAHMA), Hsp90 inhibitors, Tau and Androgen Receptors (AR), kinase inhibitors, such as BRG1, AKT, HPK1 and IRE1, MDM2 inhibitors, compounds targeting Human BET Bromodomain-containing proteins, HDAC inhibitors, human lysine methyltransferase inhibitors, such as KDM5, angiogenesis inhibitors, immunosuppressive compounds, and compounds targeting the aryl hydrocarbon receptor (AHR), among numerous others. In certain embodiments, the CIDES and conjugated CIDEs described herein are not limited to the type of PB. In certain embodiments, the CIDES and conjugated CIDEs described herein are not limited to the type of PB, wherein the CIDE is covalently linked to a hydrolysable linker as described herein.

In certain embodiments, the PB portion of the CIDE is a small molecule moeity that binds to BRM, including all variants, mutations, splice variants, indels and fusions of BRM. BRM is also known as Subfamily A, Member 2, SMARCA2 and BRAHMA. Such small molecule target protein binding moieties also include pharmaceutically acceptable salts, enantiomers, solvates and polymorphs of these compositions, as well as other small molecules that may target a protein of interest. The CIDEs described herein can comprise any residue of a known BRM binding compound, binding compounds including those disclosed in WO2019/195201, herein incorporated by reference in its entirety.

In certain embodiments, the BRM binding compound is a compound of Formula I:

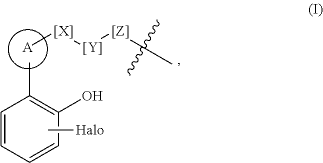

or a stereoisomer or tautomer thereof, or a pharmaceutically acceptable salt of any of the foregoing, wherein:

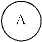

is selected from the group consisting of:
   (a)

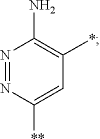

(b)

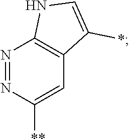

(c)

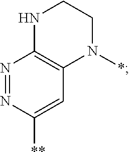

(d)

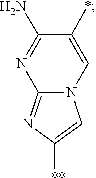

and
(e)

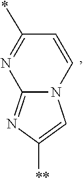

wherein, for (a)-(e), * denotes the point of attachment to [X], or, if [X] is absent, * denotes the point of attachment to [Y], and ** denotes the point of attachment to the phenyl ring; and wherein:
(i) [X] is 3-15 membered heterocyclyl or 5-20 membered heteroaryl,
provided that, when

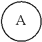

is (a), then [X] is not

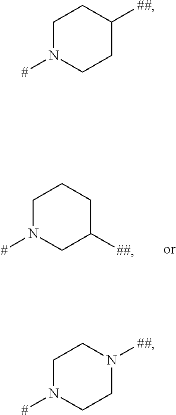

wherein # denotes the point of attachment to

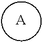

and ## denotes the point of attachment to L2,
[Y] is absent, and
[Z] is absent; or
(ii) [X] is 3-15 membered heterocyclyl or 5-20 membered heteroaryl, wherein the 3-15 membered heterocyclyl of [X] is optionally substituted with one or more —OH or C₁₋₆alkyl,
   [Y] is absent, and
[Z] is 3-15 membered heterocyclyl or 5-20 membered heteroaryl,
provided that, when

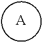

is (a) and [X] is

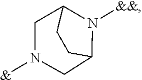

wherein & denotes the point of attachment to

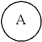

and && denotes the point of attachment to [Z], then [Z] is not

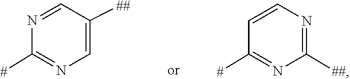

wherein # denotes the point of attachment to [X] and ## denotes the point of attachment to L2; or
(iii) [X] is 3-15 membered heterocyclyl or 5-20 membered heteroaryl,
[Y] is methylene, wherein the methylene of [Y] is optionally substituted with one or more methyl group, and
   [Z] is 3-15 membered heterocyclyl; or
(iv) [X] is absent,
[Y] is ethenylene, wherein the ethenylene of [Y] is optionally substituted with one or more halo, and
   [Z] is 5-20 membered heteroaryl,
provided that

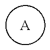

is (a), (b), (d), or (e); or
(v) [X] is absent,
   [Y] is ethynylene, and
   [Z] is 5-20 membered heteroaryl,
provided that

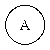

is (a), (b), (d), or (e); or
(vi) [X] is absent,
   [Y] is cyclopropyl or cyclobutyl, and
   [Z] is 5-20 membered heteroaryl,
provided that

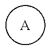

is (a), (b), (d), or (e).

In certain embodiments, the BRM binding compound is a compound of formula (I-A):

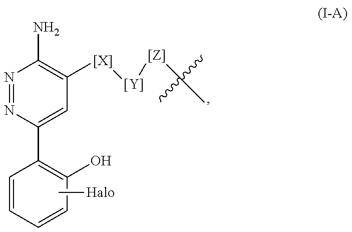

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-B):

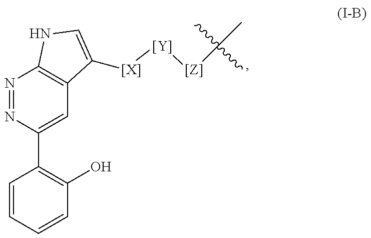

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-C):

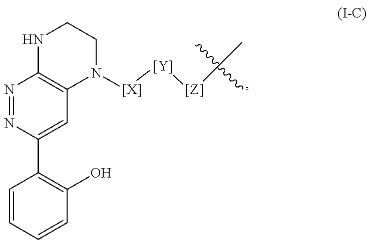

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-M):

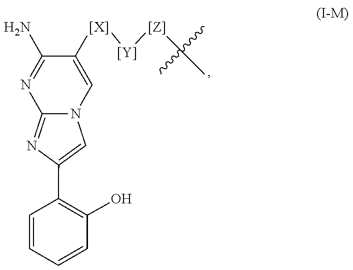

or a stereoisomer or tautomer thereof, or a pharmaceutically acceptable salt of any of the foregoing, wherein [X], [Y] and [Z] are as defined above for a compound of formula (I).

In certain embodiments, the BRM binding compound is a compound of formula (I-E):

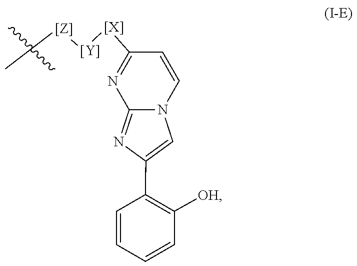

or a stereoisomer or tautomer thereof, or a pharmaceutically acceptable salt of any of the foregoing, wherein [X], [Y] and [Z] are as defined above for a compound of formula (I).

In certain embodiments, the PB (BRM) portion of the CIDE has the structure:

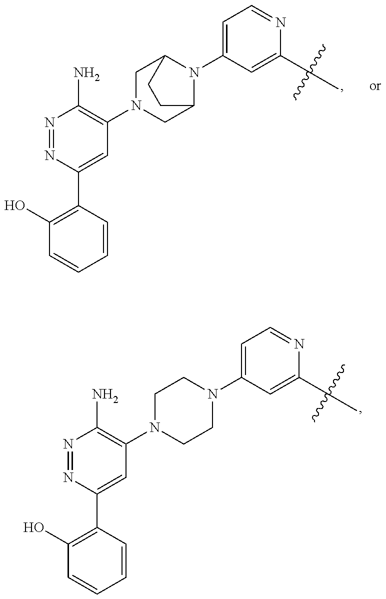

wherein,

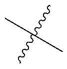

is the point of covalent attachment to L2.
   c. Linker L2

The E3LB and PB portions of CIDEs as described herein can be connected with linker (L2, Linker L2, Linker-2). In certain embodiments, the Linker L2 is covalently bound to the E3LB portion and covalently bound to the PB portion, thus making up the CIDE.

In certain embodiments, the L2 portion can be selected from linkers disclosed in WO2019/195201, herein incorporated by reference in its entirety.

Although the E3LB group and PB group may be covalently linked to the linker group through any group which is appropriate and stable to the chemistry of the linker, in certain aspects, the L2 is independently covalently bonded to the E3LB group and the PB group through an amide, ester, thioester, keto group, carbamate (urethane) or ether, each of which groups may be inserted anywhere on the E3LB group and PB group to allow binding of the E3LB group to the ubiquitin ligase and the PB group to the BRM target protein to be degraded. In other words, as shown herein, the linker can be designed and connected to E3LB and PB to modulate the binding of E3LB and PB to their respective binding partners.

In certain embodiments, L2 is a linker covalently bound to E3LB and PB, the L2 having the formula:

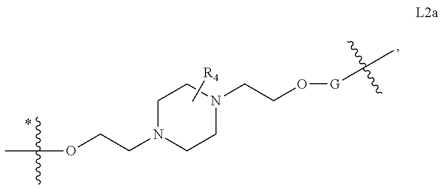

wherein,
R₄ is hydrogen or methyl,

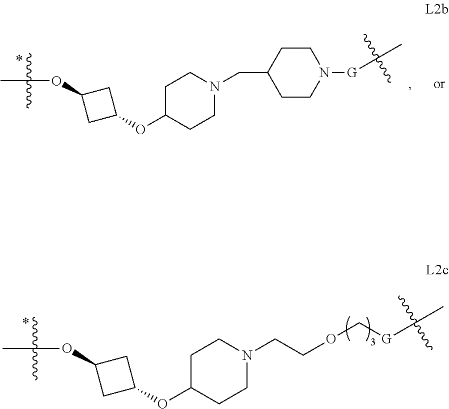

wherein,
z is one or zero,
G is

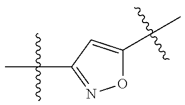 [SMILES: *c1cc(*)on1]

or C(O)NH—; and

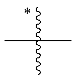

is the point of attachment to PB.

In certain embodiments of L2a, R₄ is hydrogen.

In certain embodiments of L2a, R₄ is methyl.

In certain embodiments of L2a, R₄ is a methyl, such that the methyl is oriented relative to the piperazine to which it is attached as follows:

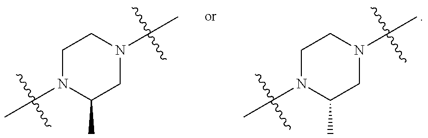

In certain embodiments of L2c, z is zero.

In certain embodiments of L2c, z is one.

### IV. Antibodies Covalently Bound to Hydrolysable Linkers

Referring now to antibody-hydrolysable linker conjugates and antibody-hydrolysable linker-CIDE conjugates, such conjugates can comprise a single antibody where the single antibody can have more than one hydrolysable linker and/or hydrolysable linker-CIDE. The antibody is covalently bound to the hydrolysable linker (L₁, L_{X} or L_{Z}). The conjugates are selected from the following formulae as described elsewhere herein:

Ab-(L₁-D)ⱼ;

Ab-((Lₓ)-D)ⱼ; and,

Ab-(L_{z})ⱼ.

As described herein, antibodies, e.g., a monoclonal antibodies (mABs) are used to deliver a CIDE to target cells, e.g., cells that express the specific protein that is targeted by the antibody. The antibody portion of an Ab-CIDE can target a cell that expresses an antigen whereby the antigen specific Ab-CIDE is delivered intracellularly to the target cell, typically through endocytosis. In some instances, pinocytocis or similar non-specific routes of uptake may result in general cellular uptake of the Ab-CIDE within antigen expressing or non-expressing cells. The Ab-CIDEs and method of their use described herein advantageously utilize antibody recognition of the cellular surface and/or endocytosis of the Ab-CIDE to deliver the CIDE portion inside cells. Antibodies are described in WO2020/086858, which is herein incorporated by reference in its entirety.

In particular embodiments, the antibody may be mutated to reduce effector function. Examples of mutations that modulate the Fc effector function include LALAPG mutations and NG2LH mutations.

In particular embodiments, the antibody is a THIOMAB™ as previously described in WO2016/04856. Further, combinations are contemplated, such that any antibody target can be combined with any suitable combination of THIOMAB™ mutations with or without any Fc effector modulation including LALAPG or NG2LH mutations.

The antibody can be a human antibody, for example, as described generally in van Dijk and van de Winkel, Curr. Opin. Pharmacol. 5: 368-74 (2001) and Lonberg, Curr. Opin. Immunol. 20:450-459 (2008). The antibody can be a library-derived antibody. A variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, NJ, 2001) and further described, e.g., in the McCafferty et al., Nature 348:552-554; Clackson et al., Nature 352: 624-628 (1991); Marks et al., J Mol. Biol. 222: 581-597 (1992); Marks and Bradbury, in Methods in Molecular Biology 248:161-175 (Lo, ed., Human Press, Totowa, NJ, 2003); Sidhu et al., J. Mol. Biol. 338(2): 299-310 (2004); Lee et al., J Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

The antibody can be a chimeric and humanized antibody. Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing SDR (a-CDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

The antibody can be a multispecific antibody, e.g. a bispecific antibody. The term “multispecific antibody” as used herein refers to an antibody comprising an antigen-binding domain that has polyepitopic specificity (i.e., is capable of binding to two, or more, different epitopes on one molecule or is capable of binding to epitopes on two, or more, different molecules). The term “bispecific antibody” as used herein refers to a multispecific antibody comprising an antigen-binding domain that is capable of binding to two different epitopes on one molecule or is capable of binding to epitopes on two different molecules. A bispecific antibody may also be referred to herein as having “dual specificity” or as being “dual specific.” Exemplary bispecific antibodies may bind both protein and any other antigen. Techniques for making multispecific antibodies include, but are not limited to, recombinant co-expression of two immunoglobulin heavy chain-light chain pairs having different specificities (see Milstein and Cuello, Nature 305: 537 (1983), WO 93/08829, and Traunecker et al., EMBO J 10: 3655 (1991)), and “knob-in-hole” engineering (see, e.g., U.S. Pat. No. 5,731,168, WO2009/089004, US2009/0182127, US2011/0287009, Marvin and Zhu, Acta Pharmacol. Sin. (2005) 26(6):649-658, and Kontermann (2005) Acta Pharmacol. Sin., 26:1-9). Multispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991). Engineered antibodies with three or more functional antigen binding sites, including “Octopus antibodies” or “dual-variable domain immunoglobulins” (DVDs) are also included herein (see, e.g., US 2006/0025576A1, and Wu et al. Nature Biotechnology (2007)).). The antibody or fragment herein also includes a “Dual Acting FAb” or “DAF” comprising an antigen binding site that binds to a target protein as well as another, different antigen (see, US 2008/0069820, for example).

The antibody can be an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat. Med 9:129-134 (2003); and Hollinger et al., Proc. Natl. Acad Sci. USA 90: 6444-6448 (1993). Triabodies and tetrabodies are also described in Hudson et al., Nat. Med 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see, e.g., U.S. Pat. No. 6,248,516 B1). Antibody fragments can be made by various techniques, including but not limited to proteolytic digestion of an intact antibody as well as production by recombinant host cells.

The antibody can be an antibody variant. In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

The antibody can be produced using recombinant methods and compositions, e.g., as described in U.S. Pat. No. 4,816,567. Referring now to antibody affinity, in embodiments, the antibody binds to one or more tumor-associated antigens or cell-surface receptors.

In certain embodiments, the tumor-associated antigen or cell surface receptor is selected from CD71, Trop2, MSLN, NaPi2b, Ly6E, EpCAM, and CD22. As such, in certain embodiments, an Ab-CIDE may comprise an antibody or fragment selected from: Anti-Ly6E Antibodies, Anti-NaPi2b Antibodies, Anti-CD22 Antibodies, Anti-CD71 Antibodies, Anti-Trop2 Antibodies, Anti-MSLN Antibodies, Anti-EpCAM Antibodies, Anti-Steap1 Antibodies, Anti-CD33 Antibodies, and Anti-iER2 Antibodies.

Particular antibodies include but are not limited to:

i. Anti-Ly6E Antibodies

In certain embodiments, an Ab-CIDE can comprise anti-Ly6E antibodies: Ly6E (lymphocyte antigen 6 complex, locus E; Ly67, RIG-E, SCA-2, TSA-1); NP_002337.1; NM_002346.2; de Nooij-van Dalen, A. G. et al (2003) Int. J. Cancer 103 (6), 768-774; Zammit, D. J. et al (2002) Mol. Cell. Biol. 22 (3):946-952; WO 2013/17705.

Ly6E is a GPI linked, 131 amino acid length, ˜8.4 kDa protein of unknown function with no known binding partners. It was initially identified as a transcript expressed in immature thymocyte, thymic medullary epithelial cells in mice (Mao, et al. (1996) Proc. Natl. Acad. Sci. U.S.A. 93:5910-5914). In some embodiments, the subject matter described herein provides an Ab-CIDE comprising an anti-Ly6E antibody described in PCT Publication No. WO 2013/177055.

ii. Anti-NaPi2b Antibodies

In certain embodiments, an Ab-CIDE comprises anti-NaPi2b antibodies: Napi2b (Napi3b, NAPI-3B, NPTIIb, SLC34A2, solute carrier family 34 (sodium phosphate), member 2, type II sodium-dependent phosphate transporter 3b, Genbank accession no. NM_006424) J. Biol. Chem. 277 (22):19665-19672 (2002), Genomics 62 (2):281-284 (1999), Feild, J. A., et al (1999) Biochem. Biophys. Res. Commun. 258 (3):578-582); WO2004022778 (Claim 2); EP1394274 (Example 11); WO2002102235 (Claim 13; Page 326); EP875569 (Claim 1; Page 17-19); WO200157188 (Claim 20; Page 329); WO2004032842 (Example IV); WO200175177 (Claim 24; Page 139-140); Cross-references: MIM:604217; NP_006415.1; NM_006424_1.

iii. Anti-CD22 Antibodies

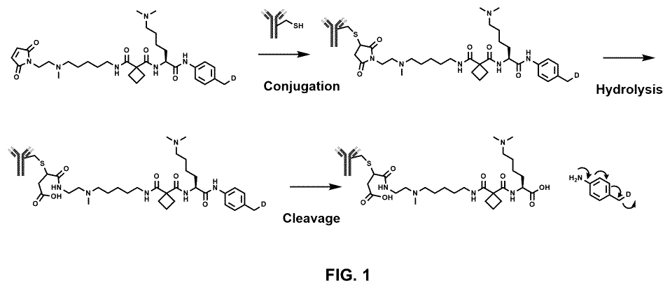

In certain embodiments, an Ab-CIDE can comprise anti-CD22 antibodies: CD22 (B-cell receptor CD22-B isoform, BL-CAM, Lyb-8, Lyb8, SIGLEC-2, FLJ22814, Genbank accession No. AK026467); Wilson et al (1991) J. Exp. Med. 173:137-146; WO2003072036 (Claim 1; FIG. 1); Cross-references: MIM:107266; NP_001762.1; NM_001771_1.

iv. Anti-CD71 Antibodies

In certain embodiments, an Ab-CIDE can comprise anti-CD71 antibodies. CD71 (transferrin receptor) is an integral membrane glycoprotein that plays an important role in cellular uptake of iron. It is well known as a marker for cell proliferation and activation. Although all proliferating cells in hematopoietic system express CD71, however, CD71 has been considered as a useful erythroid-associated antigen. In one embodiment, an anti-CD71 antibody is described in: WO2016081643 which is incorporated by reference in its entirety.

v. Anti-Trop2 Antibodies

In certain embodiments, an Ab-CIDE can comprise anti-Trop2 antibodies. Trop2 (trophoblast antigen 2) is a transmembrane glycoprotein that is an intracellular calcium signal transducer that is differentially expressed in many cancers. It signals cells for self-renewal, proliferation, invasion, and survival. Trop 2 is also known as cell surface glycoprotein Trop-2/Trop2, gastrointestinal tumor-associated antigen GA7331, pancreatic carcinoma marker protein GA733-1/GA733, membrane component chromosome 1 surface marker 1 MIS1, epithelial glycoprotein-1, EGP-1, CAA1, Gelatinous Drop-Like Corneal Dystrophy GDLD, and TTD2. In any of the above embodiments, an anti-Trop2 antibody of an Ab-CIDE is humanized. In one embodiment, the anti-Trop2 antibodies are described in US-2014/0377287 and US-2015/0366988, each of which is incorporated by reference in its entirety.

vi. Anti-MSLN Antibodies

In certain embodiments, an Ab-CIDE can comprise anti-MSLN antibodies. MSLN (mesothelin) is a glycosylphosphatidylinositol-anchored cell-surface protein that may function as a cell adhesion protein. MSLN is also known as CAK1 and MPF. This protein is overexpressed in epithelial mesotheliomas, ovarian cancers and in specific squamous cell carcinomas. In any of the above embodiments, an anti-MSLN antibody of an Ab-CIDE is humanized. In one embodiment, the anti-MSLN antibody is h7D9.v3 described in Scales, S. J. et al., Mol. Cancer Ther. 2014, 13(11), 2630-2640, which is incorporated by reference in its entirety.

vii. Anti-EpCAM Antibodies

In certain embodiments, an Ab-CIDE can comprise anti-EpCAM antibodies. In an aspect, the antibody of the Ab-CIDE may be an antibody that is directed to a protein that is found on numerous cells or tissue types. Examples of such antibodies include EpCAM. Epithelial cell adhesion molecule (EpCAM) is a transmembrane glycoprotein mediating Ca2+-independent homotypic cell-cell adhesion in epithelia (Litvinov, S. et al. (1994) Journal of Cell Biology 125(2):437-46). Also known as DIAR5, EGP-2, EGP314, EGP40, ESA, HNPCC8, KS1/4, KSA, M4S1, MIC18, MK-1, TACSTD1, TROP1, EpCAM is also involved in cell signaling, (Maetzel, D. et al. (2009) Nature Cell Biology 11(2):162-71), migration (Osta, W A; et al. (2004) Cancer Res. 64(16):5818-24), proliferation, and differentiation (Litvinov, S. et al. (1996) Am J Pathol. 148(3):865-75). Additionally, EpCAM has oncogenic potential via its capacity to upregulate c-myc, e-fabp, and cyclins A & E (Munz, M. et al. (2004) Oncogene 23(34):5748-58). Since EpCAM is expressed exclusively in epithelia and epithelial-derived neoplasms, EpCAM can be used as a diagnostic marker for various cancers. In other words, an Ab-CIDE can be used to deliver a CIDE to many cells or tissues rather than specific cell types or tissue types as when using a using a targeted antibody.

viii. Anti-Steap1 Antibodies

In certain embodiments, Ab-CIDEs comprise anti-STEAP1 antibodies. STEAP1 (six transmembrane epithelial antigen of prostate, Genbank accession no. NM_0 12449) Cancer Res. 6 1 (15), 5857-5860 (2001), Hubert, R. S., et al (1999) Proc. Natl. Acad. Sci. U.S.A. 96 (25): 14523-14528); WO2004065577 (Claim 6); WO2004027049 (FIG. 1L); EP1394274 (Example 11); WO2004016225 (Claim 2); WO2003042661 (Claim 12); US2003 157089 (Example 5); US2003 185830 (Example 5); US2003064397 (FIG. 2); WO200289747 (Example 5; Page 618-619); WO2003022995 (Example 9; FIG. 13A, Example 53; Page 173, Example 2; FIG. 2A).

viii. Anti-Steap2 Antibodies

In certain embodiments, Ab-CIDEs comprise anti-STEAP2 antibodies. STEAP2 (HGNC 8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, Genbank accession no. AF455138) Lab. Invest. 82 (11): 1573 1582 (2002)); WO2003087306; US2003064397 (Claim 1; FIG. 1); WO200272596 (Claim 13; Page 54-55); WO200172962 (Claim 1; FIG. 4B); WO2003 104270 (Claim 11); WO2003 104270 (Claim 16); US2004005598 (Claim 22); WO2003042661 (Claim 12); US2003060612 (Claim 12; FIG. 10); WO200226822 (Claim 23; FIG. 2); WO200216429 (Claim 12; FIG. 10); Cross-references: GF22655488; AAN04080.1; AF455138_I.

x. Anti-Her2 Antibodies

In certain embodiments, Ab-CIDEs comprise anti-HER2 antibodies. In one embodiment an anti-HER2 antibody of the Ab-CIDE comprises a humanized anti-HER2 antibody. In some embodiments, the Ab-CIDE comprises a humanized HER2 antibody also referred to as trastuzumab, commercially available under the tradename HERCEPTIN®. In another embodiment, an anti-HER2 antibody of a Ab-CIDE comprises a humanized anti-HER2 antibody, e.g., humanized 2C4, as described in U.S. Pat. No. 7,862,817. An exemplary humanized 2C4 antibody is pertuzumab, commercially available under the tradename PERJETA®.

x. Anti CD33 Antibodies

In certain embodiments, Ab-CIDEs comprise anti-CD33 antibodies. CD33, a member of the sialic acid binding, immunoglobulin-like lectin family, is a 67 kDa glycosylated transmembrane protein. CD33 is expressed on most myeloid and monocytic leukemia cells in addition to committed myelomonocytic and erythroid progenitor cells. It is not seen on the earliest pluripotent stem cells, mature granulocytes, lymphoid cells, or nonhematopoietic cells (Sabbath et al., (1985). Clin. Invest. 75:756-56; Andrews et al., (1986) Blood 68:1030-5). CD33 contains two tyrosine residues on its cytoplasmic tail, each of which is followed by hydrophobic residues similar to the immunoreceptor tyrosine-based inhibitory motif (ITIM) seen in many inhibitory receptors.

Referring specifically to the antibody-hydrolysable linker-CIDE (D) conjugates, the “CIDE loading” (CIDE/antibody ratio, “DAR” or j as described above) is the average number of CIDE moieties per antibody. CIDE loading may range from 1 to 20 CIDE (D) per antibody (Ab). That is, in the Ab-CIDE formula, Ab-(L1-D)ⱼ, j represents the number of CIDEs linked to the antibody and has a value from about 1 to about 20, from about 1 to about 16, from about 1 to about 10, from about 2 to about 8, or from about 4 to about 7. In embodiments, j is about 6. In embodiments, j is about 13 or 14. Each CIDE covalently linked to the antibody through linker L1 can be the same or different CIDE and can have a linker of the same type or different type as any other L1 covalently linked to the antibody. In certain embodiments, Ab is a cysteine engineered antibody.

The average number of CIDEs per antibody in preparations of Ab-CIDEs from conjugation reactions may be characterized by conventional means such as mass spectrometry, ELISA assay, electrophoresis, and HPLC. The quantitative distribution of Ab-CIDEs in terms of j may also be determined. By ELISA, the averaged value of j in a particular preparation of Ab-CIDE may be determined (Hamblett et al (2004) Clin. Cancer Res. 10:7063-7070; Sanderson et al (2005) Clin. Cancer Res. 11:843-852). However, the distribution of the value of j is not discernible by the antibody-antigen binding and detection limitation of ELISA. Also, ELISA assay for detection of Ab-CIDEs does not determine where the CIDE moieties are attached to the antibody, such as the heavy chain or light chain fragments, or the particular amino acid residues. In some instances, separation, purification, and characterization of homogeneous Ab-CIDEs where j is a certain value from Ab-CIDEs with other CIDE loadings may be achieved by means such as reverse phase HPLC or electrophoresis.

For some Ab-CIDEs, j may be limited by the number of attachment sites on the antibody. For example, an antibody may have only one or several cysteine thiol groups, or may have only one or several sufficiently reactive thiol groups through which a linker may be attached. Another reactive site on an Ab to connect L1-Ds are the amine functional group of lysine residues.

Generally, fewer than the theoretical maximum of CIDE moieties is conjugated to an antibody during a conjugation reaction. An antibody may contain, for example, many lysine residues that do not react with the linker L1-CIDE group (L1-D) or linker reagent. Only the most reactive lysine groups may react with an amine-reactive linker reagent. Also, only the most reactive cysteine thiol groups may react with a thiol-reactive linker reagent or linker L1-CIDE group. Generally, antibodies do not contain many, if any, free and reactive cysteine thiol groups which may be linked to a CIDE moiety. Most cysteine thiol residues in the antibodies of the compounds exist as disulfide bridges and must be reduced with a reducing agent such as dithiothreitol (DTT) or TCEP, under partial or total reducing conditions. However, the CIDE loading may be controlled in several different manners including: (i) limiting the molar excess of linker L1-CIDE group or linker reagent relative to antibody, (ii) limiting the conjugation reaction time or temperature, and (iii) partial or limiting reductive conditions for cysteine thiol modification.

Referring now to an Ab-CIDE and a L1-CIDE compound, as described herein, these can exist in solid or liquid form. In the solid state, it may exist in crystalline or noncrystalline form, or as a mixture thereof. The skilled artisan will appreciate that pharmaceutically acceptable solvates may be formed for crystalline or non-crystalline compounds. In crystalline solvates, solvent molecules are incorporated into the crystalline lattice during crystallization. Solvates may involve non-aqueous solvents such as, but not limited to, ethanol, isopropanol, DMSO, acetic acid, ethanolamine, or ethyl acetate, or they may involve water as the solvent that is incorporated into the crystalline lattice. Solvates wherein water is the solvent incorporated into the crystalline lattice are typically referred to as “hydrates.” Hydrates include stoichiometric hydrates as well as compositions containing variable amounts of water. The subject matter described herein includes all such solvates.

The skilled artisan will further appreciate that certain compounds and Ab-CIDEs described herein that exist in crystalline form, including the various solvates thereof, may exhibit polymorphism (i.e. the capacity to occur in different crystalline structures). These different crystalline forms are typically known as “polymorphs.” The subject matter disclosed herein includes all such polymorphs. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

Compounds and Ab-CIDEs described herein or a salt thereof may exist in stereoisomeric forms (e.g., it contains one or more asymmetric carbon atoms). The individual stereoisomers (enantiomers and diastereomers) and mixtures of these are included within the scope of the subject matter disclosed herein. Likewise, it is understood that a compound or salt of Formula (I) may exist in tautomeric forms other than that shown in the formula and these are also included within the scope of the subject matter disclosed herein. It is to be understood that the subject matter disclosed herein includes all combinations and subsets of the particular groups described herein. The scope of the subject matter disclosed herein includes mixtures of stereoisomers as well as purified enantiomers or enantiomerically/diastereomerically enriched mixtures. It is to be understood that the subject matter disclosed herein includes all combinations and subsets of the particular groups defined hereinabove.

The subject matter disclosed herein also includes isotopically-labelled forms of the compounds described herein, but for the fact that one or more atoms are replaced by an atom having an atomic mass or mass number different from the atomic mass or mass number usually found in nature. Examples of isotopes that can be incorporated into compounds described herein and pharmaceutically acceptable salts thereof include isotopes of hydrogen, carbon, nitrogen, oxygen, phosphorous, sulphur, fluorine, iodine, and chlorine, such as ²H, ³H, ¹¹C, ¹³C ¹⁴C, ¹⁵N, ¹⁷O, ¹⁸O, ³¹P, ³²P, ³⁵S, ¹⁸F, ³⁶Cl, ¹²³I and ¹²⁵I.

Compounds and Ab-CIDEs as disclosed herein and pharmaceutically acceptable salts thereof that contain the aforementioned isotopes and/or other isotopes of other atoms are within the scope of the subject matter disclosed herein. Isotopically-labelled compounds are disclosed herein, for example those into which radioactive isotopes such as ³H, ¹⁴C are incorporated, are useful in drug and/or substrate tissue distribution assays. Tritiated, i.e., ³H, and carbon-14, i.e., ¹⁴C, isotopes are commonly used for their ease of preparation and detectability. ¹¹C and ¹⁸F isotopes are useful in PET (positron emission tomography), and ¹²⁵I isotopes are useful in SPECT (single photon emission computerized tomography), all useful in brain imaging. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds can generally be prepared by carrying out the procedures disclosed in the Schemes and/or in the Examples below, by substituting a readily available isotopically labelled reagent for a non-isotopically labelled reagent.

The subject matter disclosed herein includes the following non-limiting embodiments:
1. A compound having the structure:

L₁-D
wherein,
D is a CIDE;
L₁ is a linker-1 covalently bound to D and having a structure of Formula I:

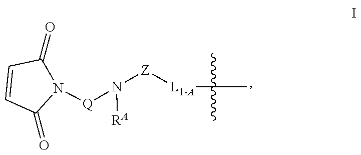

wherein,
Z is —(CH₂)ₚ— or —CH₂—(CH₂—O—CH₂)ₚ—CH₂—, wherein p is an integer from 1 to 24;
R^{A} is hydrogen, C₁₋₆ alkyl, or —(CH₂)ᵥ-aryl, wherein, v is 0 or 1;
Q is selected from the group consisting of:
i)

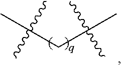

wherein, q is 1, 2, 3 or 4; and
ii)

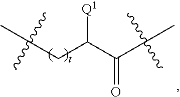

wherein t is 0, 1, 2, 3 or 4;
Q¹ is hydrogen,

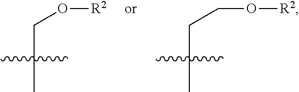

wherein R² is hydrogen, halo C₁₋₆ alkyl or C₁₋₆ alkyl; and
L_{1-A} is:

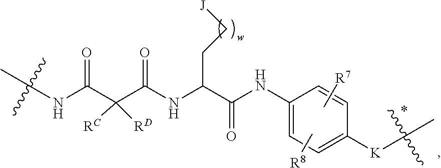

wherein

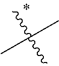

indicates the attachment point to D;
w is 0, 1, 2, 3, 4 or 5;
J is selected from the group consisting of —C₁₋₅ alkyl, —N(R^{x})(R^{y}), —C(O)NH₂, —NH—C(O)—NH₂, and —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.
2. The compound of embodiment 1, wherein Q is:

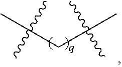

wherein q is 1, 2, 3 or 4.
3. The compound of embodiment 1 or 2, wherein q is 2.
4. The compound of embodiment 1, wherein Q is:

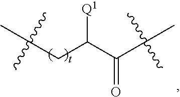

wherein t is 0, 1, 2, 3 or 4;
Q¹ is hydrogen,

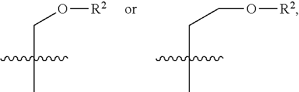

wherein R² is hydrogen, halo C₁₋₆ alkyl or C₁₋₆ alkyl.
5. The compound of embodiment 4, wherein R² is hydrogen or C₁₋₆ alkyl.
6. The compound of embodiment 5, wherein the C₁₋₆ alkyl is methyl.
7. The compound of embodiment 4, wherein Q¹ is:

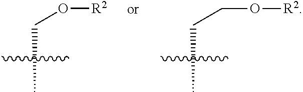

8. The compound of embodiment 4, wherein R² is hydrogen or methyl.
9. The compound of embodiment claim 8, wherein Q¹ is:

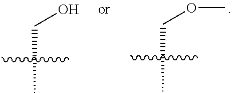

10. The compound of any one of embodiments 4-9, wherein t is 0.
11. The compound of any one of embodiments 1-10, wherein Z is —(CH₂)ₚ—, wherein p is 1, 2, 3, 4, 5 or 6.
12. The compound of embodiment 11, wherein p is 4, 5 or 6.
13. The compound of embodiment 11, wherein p is 5.
14. The compound of any one of embodiments 1-10, wherein Z is —CH₂—(CH₂—O—CH₂)ₚ—CH₂—, wherein p is 1, 2, 3, 4, 5 or 6.
15. The compound of embodiment 14, wherein p is 1, 2 or 3.
16. The compound of embodiment 14, wherein p is 1.
17. The compound of any one of embodiments 1-16, wherein R^{A} is hydrogen or C₁₋₆ alkyl.
18. The compound of embodiment 17, wherein R^{A} is hydrogen or methyl.
19. The compound of any one of embodiments 1-16, wherein R^{A} is phenyl or benzyl.
20. The compound of any one of embodiments 1-19, wherein R⁷ and R⁸ are each hydrogen.
21. The compound of any one of embodiments 1-20, wherein R^{C} and R^{D} together with the carbon to which each is attached form an optionally substituted C₃₋₆ cycloalkyl.
22. The compound of claim 21, wherein the C₃₋₆ cycloalkyl is cyclobutane.
23. The compound of any one of embodiments 1-22, wherein K is —CH(R)—, wherein R is hydrogen, C₁₋₃ alkyl, N(R^{x})(R^{y}) or C(O)—N(R^{x})(R^{y}), wherein R^{x} and R^{y} are each independently selected from hydrogen and C₁₋₃ alkyl, or R^{x} and R^{y} together with the nitrogen to which each is attached form an optionally substituted 5- to 7-member heterocyclyl.
24. The compound of embodiment 23, wherein R is hydrogen or C₁₋₃ alkyl.
25. The compound of embodiment 24, wherein R is hydrogen.
26. An antibody-CIDE conjugate having the structure:

Ab-(L₁-D)ⱼ
wherein,
Ab is an antibody;
j is 1 to 16;
D is a CIDE;
L₁ is a linker-1 covalently bound to Ab and to D and having a structure of Formula I-A:

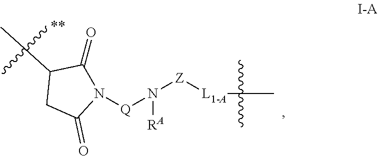

wherein,
Z is —(CH₂)ₚ— or —CH₂—(CH₂—O—CH₂)ₚ—CH₂—, wherein p is an integer from 1 to 24;
R^{A} is hydrogen, C₁₋₆ alkyl, or —(CH₂)ᵥ-aryl, wherein v is 0 or 1;
Q is selected from the group consisting of:
i)

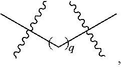

wherein q is 1, 2, 3 or 4; and
ii)

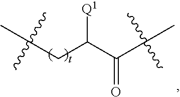

wherein t is 0, 1, 2, 3 or 4;
Q¹ is hydrogen,

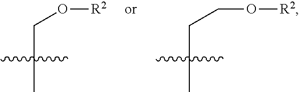

wherein R² is hydrogen, halo C₁₋₆ alkyl or C₁₋₆ alkyl; and
L_{1-A} is:

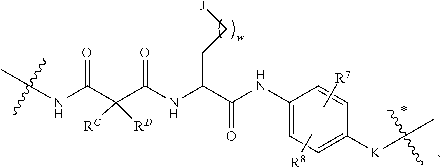

wherein

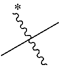

indicates the attachment point to D;
w is 0, 1, 2, 3, 4 or 5;
J is selected from the group consisting of —N(R^{x})(R^{y}), —C(O)NH₂, —NH—C(O)—NH₂, and —NH—C(═NH)(NH₂), wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.
27. The antibody-CIDE conjugate of embodiment 26, wherein Q is:

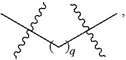

wherein q is 1, 2, 3 or 4.
28. The antibody-CIDE conjugate of embodiment 26 or 27, wherein q is 2.
29. The antibody-CIDE conjugate of claim 26, wherein Q is:

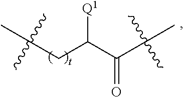

wherein t is 0, 1, 2, 3 or 4;
Q¹ is hydrogen

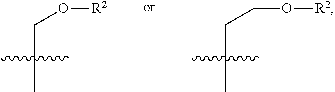

wherein R² is hydrogen, halo C₁₋₆ alkyl or C₁₋₆ alkyl.
30. The antibody-CIDE conjugate of embodiment 29, wherein R² is hydrogen or C₁₋₆ alkyl.
31. The antibody-CIDE conjugate of embodiment 30, wherein the C₁₋₆ alkyl is methyl.
32. The antibody-CIDE conjugate of embodiment 29, wherein Q¹ is:

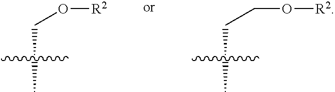

33. The antibody-CIDE conjugate of embodiment 29, wherein R² is hydrogen or methyl.
34. The antibody-CIDE conjugate of embodiment 33, wherein Q¹ is:

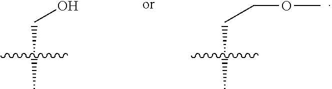

35. The antibody-CIDE conjugate of any one of embodiments 29-34, wherein t is 0.
36. The antibody-CIDE conjugate of any one of embodiments 26-35, wherein Z is —(CH₂)ₚ—, wherein p is 1, 2, 3, 4, 5 or 6.
37. The antibody-CIDE conjugate of embodiment 36, wherein p is 4, 5 or 6.
38. The antibody-CIDE conjugate of embodiment 36, wherein p is 5.
39. The antibody-CIDE conjugate of any one of embodiments 26-35, wherein Z is —CH₂—(CH₂—O—CH₂)ₚ—CH₂—, wherein p is 1, 2, 3, 4, 5 or 6.
40. The antibody-CIDE conjugate of embodiment 39, wherein p is 1, 2 or 3.
41. The antibody-CIDE conjugate of embodiment 39, wherein p is 1.
42. The antibody-CIDE conjugate of any one of embodiments 26-41, wherein R^{A} is hydrogen or C₁₋₆ alkyl.
43. The antibody-CIDE conjugate of embodiment 42, wherein R^{A} is hydrogen or methyl.
44. The antibody-CIDE conjugate of any one of embodiments 26-41, wherein R^{A} is phenyl or benzyl.
45. The antibody-CIDE conjugate of any one of embodiments 26-44, wherein R⁷ and R⁸ are each hydrogen.
46. The antibody-CIDE conjugate of any one of embodiments 26-45, wherein R^{C} and R^{D} together with the carbon to which each is attached form an optionally substituted C₃₋₆ cycloalkyl.
47. The antibody-CIDE conjugate of embodiment 46, wherein the C₃₋₆ cycloalkyl is cyclobutane.
48. The antibody-CIDE conjugate of any one of embodiments 26-47, wherein K is —CH(R)—O—, wherein R is hydrogen, C₁₋₃ alkyl, N(R^{x})(R^{y}) or C(O)—N(R^{x})(R^{y}), wherein R^{x} and R^{y} are each independently selected from hydrogen and C₁₋₃ alkyl, or R^{x} and R^{y} together with the nitrogen to which each is attached form an optionally substituted 5- to 7-member heterocyclyl.
49. The antibody-CIDE conjugate of embodiment 48, wherein R is hydrogen or C₁₋₃ alkyl.
50. The antibody-CIDE conjugate of embodiment 49, wherein R is hydrogen.
51. A pharmaceutical composition comprising a conjugate of any one of embodiments 26-50 and one or more pharmaceutically acceptable excipients.
52. A method of treating a disease in a human in need thereof, comprising administering to said human an effective amount of a conjugate any one of embodiments 26-50 or 57-108 (or a composition thereof) or a composition of embodiment 51; or, A conjugate of any one of embodiments 26-50 or 57-108 (or a composition thereof) or a composition of embodiment 51 for use in treating a disease in a human in need thereof; or, Use of a conjugate of any one of embodiments 26-50 or 57-108 (or a composition thereof) or a composition of embodiment 51 for the manufacture of a medicament for the treatment of a disease in a human in need thereof.
53. The method of embodiment 52, wherein said disease is cancer.
54. The method of embodiment 53, wherein said cancer is BRM-dependent.
55. The method of embodiment 54, wherein said cancer is non-small cell lung cancer.
56. A method of reducing the level of a target protein in a subject comprising, administering a conjugate of any one of embodiments 26-50 or 58-108 (or a composition thereof) or composition of embodiment 51 to said subject, wherein said D binds said target protein, wherein ubiquitin ligase effects degradation of said bound target protein, wherein the level of said target protein is reduced.
58. An antibody-CIDE conjugate having the structure:

Ab-((L_{X})-D)ⱼ
wherein,
Ab is an antibody;
j is 1 to 16;
D is a CIDE;
L_{X} is selected from the group consisting of L₁ and L₁ₕ, wherein L₁ₕ is present in at least one instance of L_{X}:
L₁ is a linker-1 covalently bound to Ab and to D and having a structure of Formula I-A:

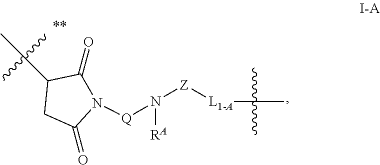

and,
L₁ₕ is a linker-1 covalently bound to D and having a structure of Formula I-B:

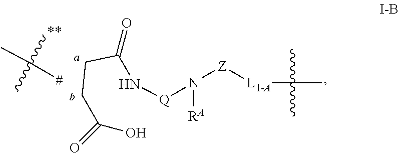

wherein, # indicates the point of attachment at position a or b
in L₁ and L₁ₕ,

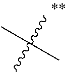

indicates the point of attachment to the antibody;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂)ᵥ-aryl, wherein v is 0 or 1;
Q is selected from the group consisting of:
i)

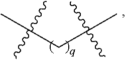

wherein q is 1, 2, 3 or 4; and
ii)

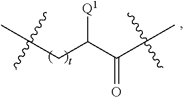

wherein t is 0, 1, 2, 3 or 4;
Q¹ is hydrogen,

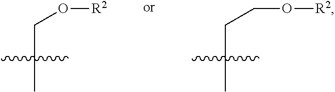

wherein R² is hydrogen, halo C₁₋₆ alkyl or C₁₋₆ alkyl; and
L_{1-A} is:

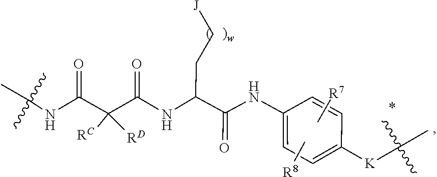

wherein

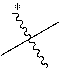

indicates the attachment point to D;
w is 0, 1, 2, 3, 4 or 5;
J is selected from the group consisting of —C₁₋₅ alkyl, —N(R^{x})(R^{y}), —C(O)NH₂, —NH—C(O)—NH₂, —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.
59. The antibody-CIDE conjugate of embodiment 58, wherein Q is:

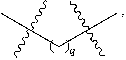

wherein q is 1, 2, 3 or 4.
60. The antibody-CIDE conjugate of embodiment 58 or 59, wherein q is 2.
61. The antibody-CIDE conjugate of embodiment 58, wherein Q is:

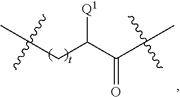

wherein t is 0, 1, 2, 3 or 4;
Q¹ is hydrogen,

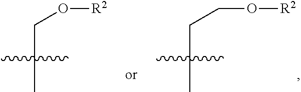

wherein R² is hydrogen, halo C₁₋₆ alkyl or C₁₋₆ alkyl.
62. The antibody-CIDE conjugate of embodiment 61, wherein R² is hydrogen or C₁₋₆ alkyl.
63. The antibody-CIDE conjugate of embodiment 62, wherein the C₁₋₆ alkyl is methyl.
64. The antibody-CIDE conjugate of embodiment 61, wherein Q¹ is:

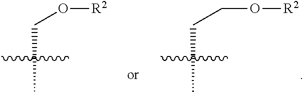

65. The antibody-CIDE conjugate of embodiment 61, wherein R² is hydrogen or methyl.
66. The antibody-CIDE conjugate of embodiment claim 65, wherein Q¹ is:

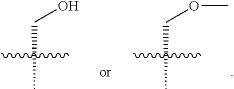

67. The antibody-CIDE conjugate of any one of embodiments 61-66, wherein t is 0.
68. The antibody-CIDE conjugate of any one of embodiments 58-67, wherein Z is —(CH₂)ₚ—, wherein p is 1, 2, 3, 4, 5 or 6.
69. The antibody-CIDE conjugate of embodiment 68, wherein p is 4, 5 or 6.
70. The antibody-CIDE conjugate of embodiment 68, wherein p is 5.
71. The antibody-CIDE conjugate of any one of embodiments 58-67, wherein Z is —CH₂—(CH₂—O—CH₂)ₚ—CH₂—, wherein p is 1, 2, 3, 4, 5 or 6.
72. The antibody-CIDE conjugate of embodiment 71, wherein p is 1, 2 or 3.
73. The antibody-CIDE conjugate of embodiment 71, wherein p is 1.
74. The antibody-CIDE conjugate of any one of embodiments 58-73, wherein R^{A} is hydrogen or C₁₋₆ alkyl.
75. The antibody-CIDE conjugate of embodiment 74, wherein R^{A} is hydrogen or methyl.
76. The antibody-CIDE conjugate of any one of embodiments 58-73, wherein R^{A} is phenyl or benzyl.
77. The antibody-CIDE conjugate of any one of embodiments 58-76, wherein R⁷ and R⁸ are each hydrogen.
78. The antibody-CIDE conjugate of any one of embodiments 58-77, wherein R^{C} and R^{D} together with the carbon to which each is attached form an optionally substituted C₃₋₆ cycloalkyl.
79. The antibody-CIDE conjugate of claim 78, wherein the C₃₋₆ cycloalkyl is cyclobutane.
80. The antibody-CIDE conjugate of any one of embodiments 58-79, wherein K is —CH(R)—, wherein R is hydrogen, C₁₋₃ alkyl, N(R^{x})(R^{y}) or C(O)—N(R^{x})(R^{y}), wherein R^{x} and R^{y} are each independently selected from hydrogen and C₁₋₃ alkyl, or R^{x} and R^{y} together with the nitrogen to which each is attached form an optionally substituted 5- to 7-member heterocyclyl.
81. The antibody-CIDE conjugate of embodiment 80, wherein R is hydrogen or C₁₋₃ alkyl.
82. The antibody-CIDE conjugate of embodiment 81, wherein R is hydrogen.
83. An antibody-conjugate having the structure:

Ab-(L_{Z})ⱼ
wherein,
Ab is an antibody;
j is 1 to 16;
L_{Z} is selected from the group consisting of L_{X1} and Lx-D, wherein L_{X1} is present in at least one instance of L_{Z}:
L_{X1} is covalently bound to Ab and has a structure of Formula I-G:

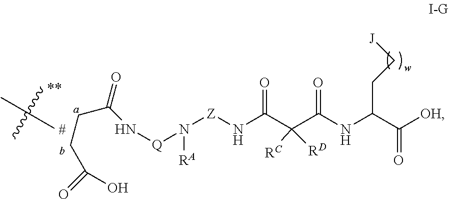

and,
Lx-D, wherein L_{X} is selected from the group consisting of L₁ and L₁ₕ:
L₁ is a linker-1 covalently bound to Ab and to D and having a structure of Formula I-A of Formula I-C:

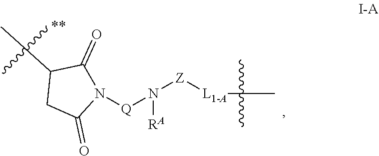

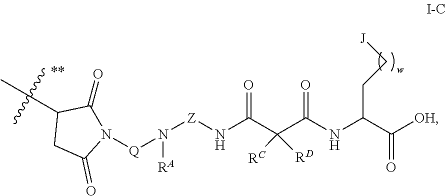

and
L₁ₕ is a linker-1 covalently bound to D and having a structure of Formula I-B:

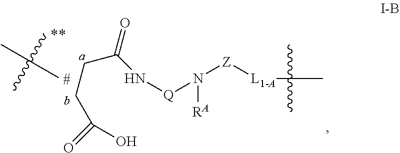

wherein, # indicates the point of attachment at position a or b

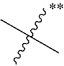

indicates the point of attachment to the antibody;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂),-aryl, wherein, v is 0 or 1;
Q is selected from the group consisting of:
i)

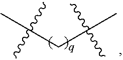

wherein q is 1, 2, 3 or 4; and

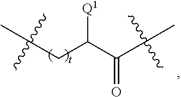

wherein t is 0, 1, 2, 3 or 4;
Q¹ is hydrogen,

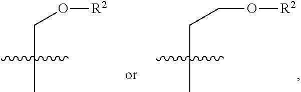

wherein R² is hydrogen, halo C₁₋₆ alkyl or C₁₋₆ alkyl; and
L_{1-A} is:

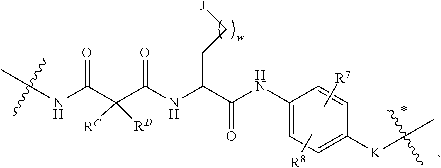

wherein

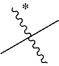

indicates the attachment point to D;
w is 0, 1, 2, 3, 4 or 5;
J is selected from the group consisting of —C₁₋₅ alkyl, —N(R^{x})(R^{y}), —C(O)NH₂, —NH—C(O)—NH₂, —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.
85. The antibody-conjugate of embodiment 84, wherein Q is:

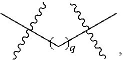

wherein q is 1, 2, 3 or 4.
86. The antibody-conjugate of embodiment 84 or 85, wherein q is 2.
87. The antibody-conjugate of embodiment 84, wherein Q is:

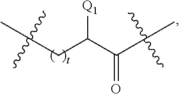

wherein t is 0, 1, 2, 3 or 4;
Q¹ is hydrogen,

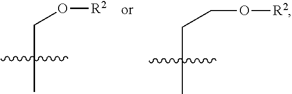

wherein R² is hydrogen, halo C₁₋₆ alkyl or C₁₋₆ alkyl.
88. The antibody-conjugate of embodiment 87, wherein R² is hydrogen or C₁₋₆ alkyl.
89. The antibody-conjugate of embodiment 88, wherein the C₁₋₆ alkyl is methyl.
90. The antibody-conjugate of embodiment 87, wherein Q¹ is:

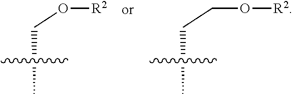

91. The antibody-conjugate of embodiment 87, wherein R² is hydrogen or methyl.
92. The antibody-conjugate of embodiment claim 91, wherein Q¹ is:

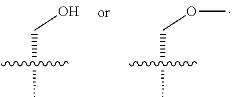

93. The antibody-conjugate of any one of embodiments 87-92, wherein t is 0.
94. The antibody-conjugate of any one of embodiments 84-93, wherein Z is —(CH₂)ₚ—, wherein p is 1, 2, 3, 4, 5 or 6.
95. The antibody-conjugate of embodiment 94, wherein p is 4, 5 or 6.
96. The antibody-conjugate of embodiment 94, wherein p is 5.
97. The antibody-conjugate of any one of embodiments 84-93, wherein Z is —CH₂—(CH₂—O—CH₂)ₚ—CH₂—, wherein p is 1, 2, 3, 4, 5 or 6.
98. The antibody-conjugate of embodiment 87, wherein p is 1, 2 or 3.
99. The antibody-conjugate of embodiment 87, wherein p is 1.
100. The antibody-conjugate of any one of embodiments 84-99, wherein R^{A} is hydrogen or C₁₋₆ alkyl.
101. The antibody-conjugate of embodiment 100, wherein R^{A} is hydrogen or methyl.
102. The antibody-conjugate of any one of embodiments 84-99, wherein R^{A} is phenyl or benzyl.
103. The antibody-conjugate of any one of embodiments 84-102, wherein R⁷ and R⁸ are each hydrogen.
104. The antibody-conjugate of any one of embodiments 84-103, wherein R^{C} and R^{D}, together with the carbon to which each is attached, form an optionally substituted C₃₋₆ cycloalkyl.
105. The antibody-conjugate of claim 104, wherein the C₃₋₆ cycloalkyl is cyclobutane.
106. The antibody-conjugate of any one of embodiments 84-105, wherein K is —CH(R)—, wherein R is hydrogen, C₁₋₃ alkyl, N(R^{x})(R^{y}) or C(O)—N(R^{x})(R^{y}), wherein R^{x} and R^{y} are each independently selected from hydrogen and C₁₋₃ alkyl, or R^{x} and R^{y} together with the nitrogen to which each is attached form an optionally substituted 5- to 7-member heterocyclyl.
107. The antibody-conjugate of embodiment 106, wherein R is hydrogen or C₁₋₃ alkyl.
108. The antibody-conjugate of embodiment 107, wherein R is hydrogen.
109. A method of making an Ab-conjugate comprising a DAR of at least 6, said method comprising:
   contacting a compound of Formula IA, Formula IC or Formula II with an antibody to prepare an Ab-conjugate comprising a DAR of at least 6.
110. The method of embodiment 109, wherein the DAR is from about 6 to about 16, or from about 8 to about 14, or 6, 7, 8, 9, 10, 11, 12 13, 14, 15 or 16.

A non-limiting, exemplary compound having the structure L₁-D is:

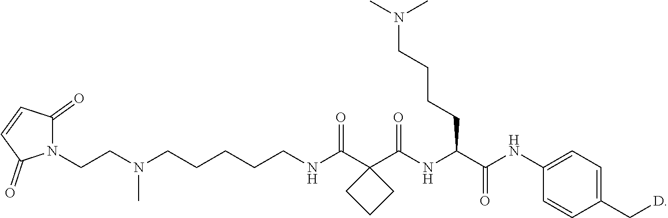

A non-limiting, exemplary compound having the structure Ab-((L_{X})-D)ⱼ is:

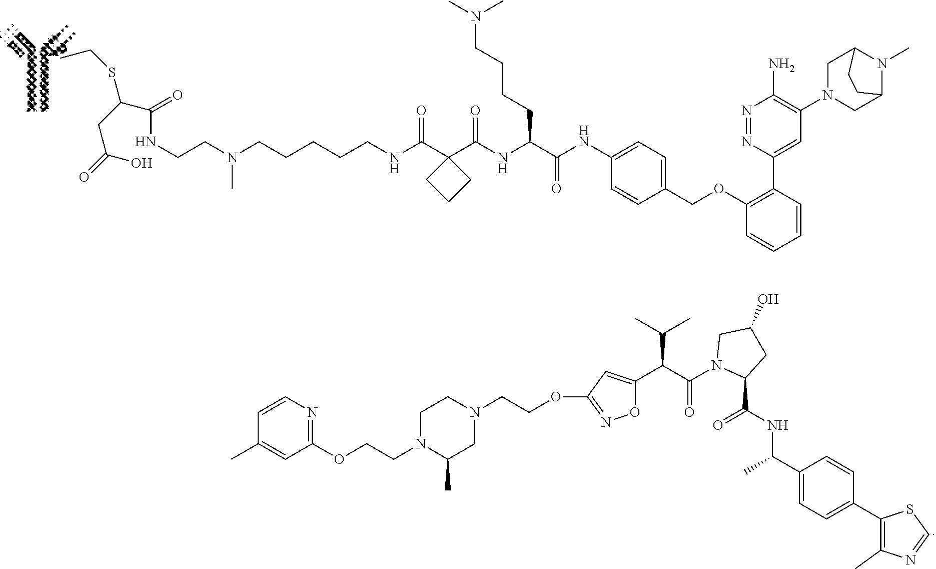

A non-limiting, exemplary compound having the structure Ab-(L_{Z})ⱼ is:

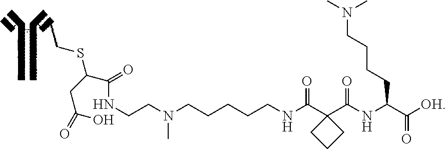 [SMILES: CSC(CC(=O)O)C(=O)NCCN(C)CCCCCNC(=O)C1(C(=O)N[C@@H](CCCCN(C)C)C(=O)O)CCC1]

A non-limiting example of a compound of Formula II is

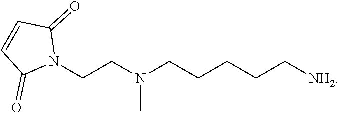 [SMILES: CN(CCCCCN)CCN1C(=O)C=CC1=O]

In certain embodiments, the subject matter described herein includes the following L1-CIDES, residues of which can be included in certain Ab-Hy-B-CIDE:

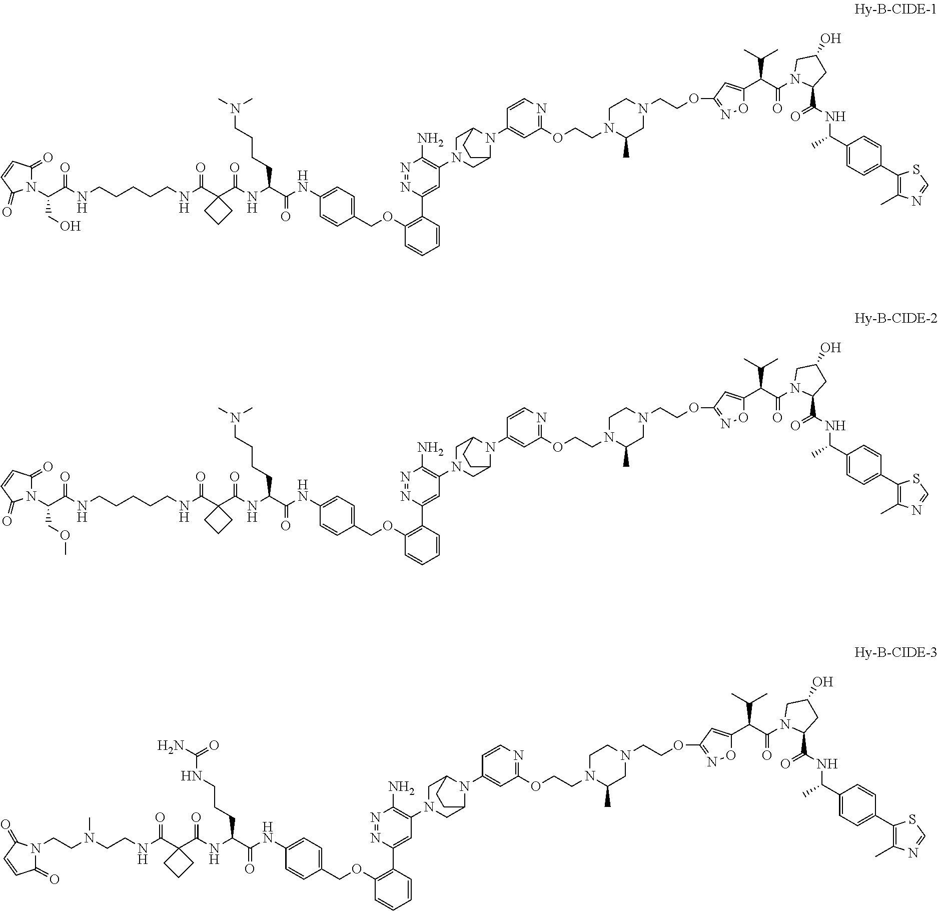

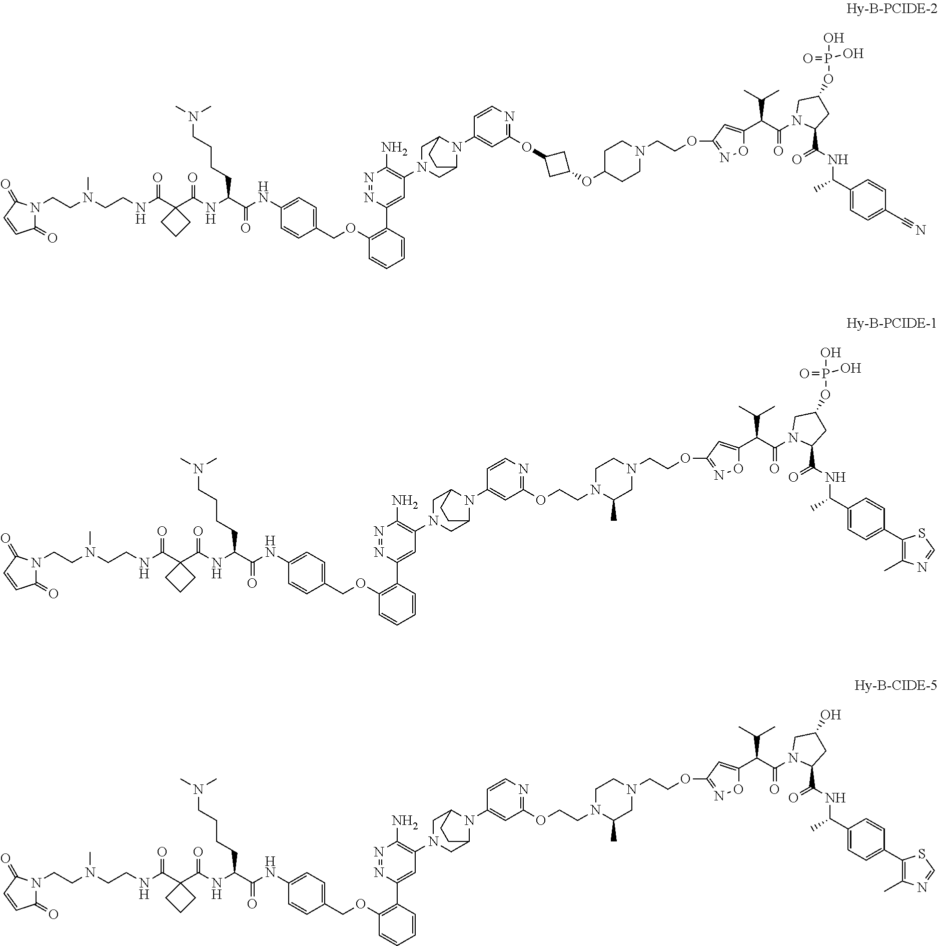

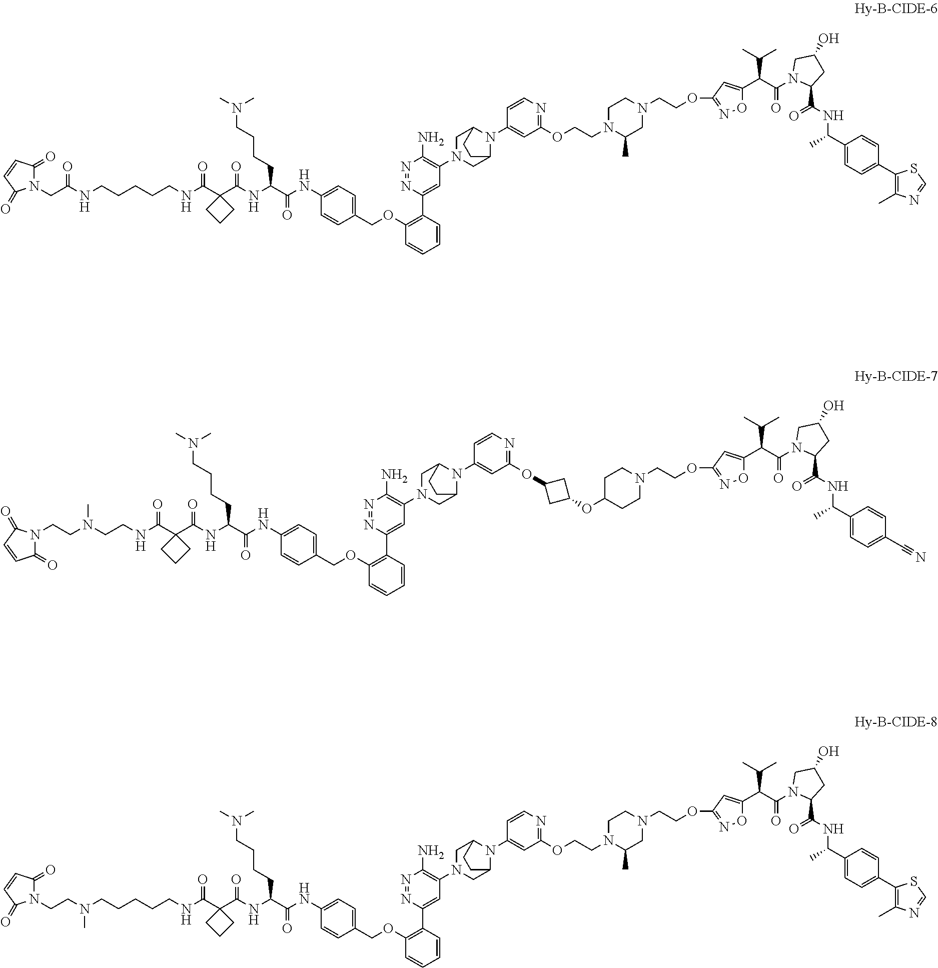

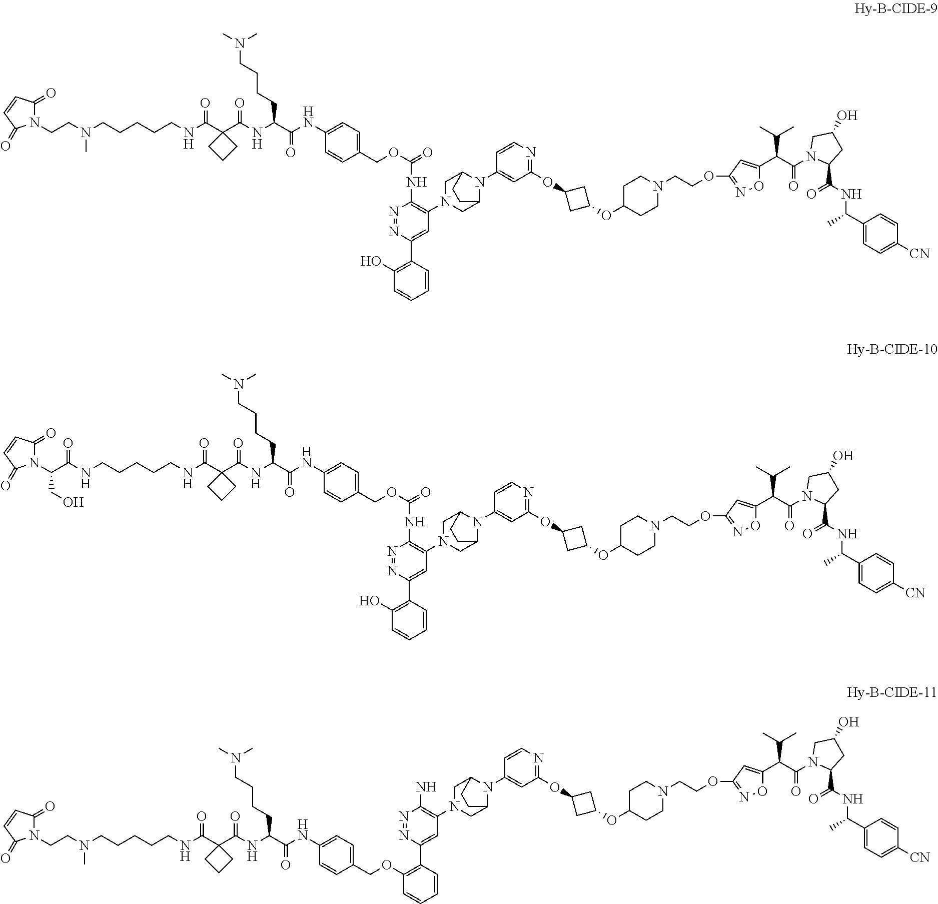

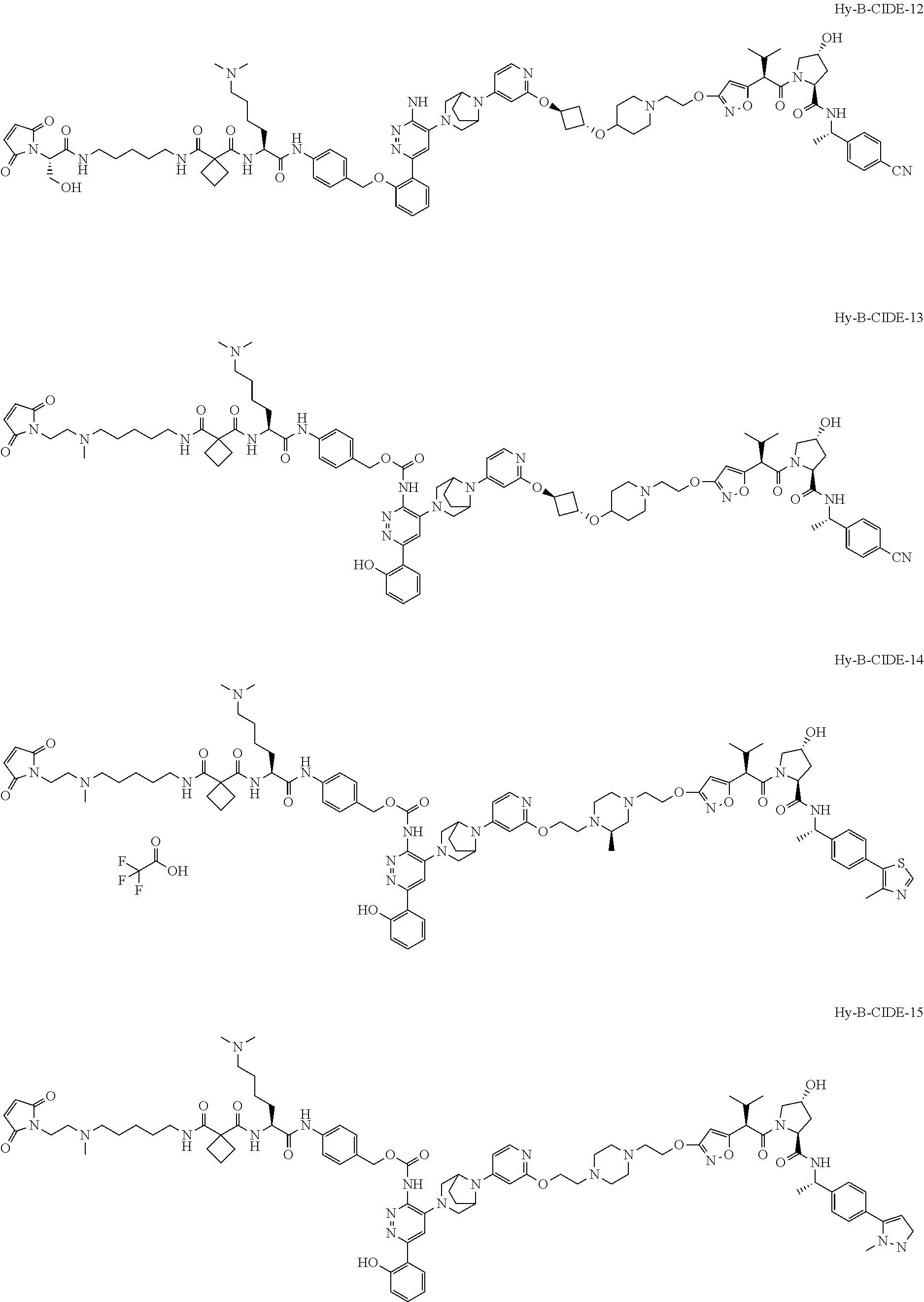

### V. Formulations

Pharmaceutical formulations of therapeutic Ab-CIDEs as described herein can be prepared for parenteral administration, e.g., bolus, intravenous, intratumor injection with a pharmaceutically acceptable parenteral vehicle and in a unit dosage injectable form. An Ab-CIDE having the desired degree of purity is optionally mixed with one or more pharmaceutically acceptable excipients (Remington's Pharmaceutical Sciences (1980) 16th edition, Osol, A. Ed.), in the form of a lyophilized formulation for reconstitution or an aqueous solution.

An Ab-CIDE can be formulated in accordance with standard pharmaceutical practice as a pharmaceutical composition. According to this aspect, there is provided a pharmaceutical composition comprising an Ab-CIDE in association with one or more pharmaceutically acceptable excipients.

A typical formulation is prepared by mixing an Ab-CIDE with excipients, such as carriers and/or diluents. Suitable carriers, diluents and other excipients are well known to those skilled in the art and include materials such as carbohydrates, waxes, water soluble and/or swellable polymers, hydrophilic or hydrophobic materials, gelatin, oils, solvents, water and the like. The particular carrier, diluent or other excipient used will depend upon the means and purpose for which the Ab-CIDE is being applied. Solvents are generally selected based on solvents recognized by persons skilled in the art as safe (GRAS) to be administered to a mammal.

In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. Acceptable diluents, carriers, excipients and stabilizers are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

The formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the Ab-CIDE or aid in the manufacturing of the pharmaceutical product. The formulations may be prepared using conventional dissolution and mixing procedures.

Formulation may be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but may range from about 3 to about 8. Formulation in an acetate buffer at pH 5 is a suitable embodiment.

The Ab-CIDE formulations can be sterile. In particular, formulations to be used for in vivo administration must be sterile. Such sterilization is readily accomplished by filtration through sterile filtration membranes.

The Ab-CIDE ordinarily can be stored as a solid composition, a lyophilized formulation or as an aqueous solution.

The pharmaceutical compositions comprising an Ab-CIDE can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The “therapeutically effective amount” of the compound to be administered will be governed by such considerations, and is the minimum amount necessary to prevent, ameliorate, or treat the coagulation factor mediated disorder. Such amount is preferably below the amount that is toxic to the host or renders the host significantly more susceptible to bleeding.

The Ab-CIDE can be formulated into pharmaceutical dosage forms to provide an easily controllable dosage of the drug and to enable patient compliance with the prescribed regimen. The pharmaceutical composition (or formulation) for application may be packaged in a variety of ways depending upon the method used for administering the drug. Generally, an article for distribution includes a container having deposited therein the pharmaceutical formulation in an appropriate form. Suitable containers are well known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The pharmaceutical compositions may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such 1,3-butanediol. The sterile injectable preparation may also be prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose any bland fixed oil may be employed including synthetic mono- or diglycerides. In addition, fatty acids such as oleic acid may likewise be used in the preparation of injectables.

The amount of Ab-CIDE that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 g of the active ingredient per milliliter of solution in order that infusion of a suitable volume at a rate of about 30 mL/hr can occur.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

The subject matter further provides veterinary compositions comprising at least one active ingredient as above defined together with a veterinary carrier therefore. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally or by any other desired route.

### VI. Indications and Methods of Treatment

It is contemplated that the Ab-CIDEs disclosed herein may be used to treat various diseases or disorders that are related to or involve the target protein such as BRM. Also provided herein is an Ab-CIDE or a composition comprising an Ab-CIDE for use in therapy. In some embodiments, provided herein is an Ab-CIDE or a composition comprising an Ab-CIDE for the treatment or prevention of diseases and disorders as disclosed herein. Also provided herein is the use of an Ab-CIDE or a composition comprising an Ab-CIDE in therapy. In some embodiments, provided herein is the use of an Ab-CIDE for the treatment or prevention of diseases and disorders as disclosed herein. Also provided herein is the use of an Ab-CIDE or a composition comprising an Ab-CIDE in the manufacture of a medicament for the treatment or prevention of diseases and disorders as disclosed herein.

Generally, the disease or disorder to be treated is a target protein-dependent disease or disorder, such as a BRM-dependent disease or disorder. For example, a target protein-dependent disease or disorder may be a hyperproliferative disease such as cancer. Examples of cancer to be treated herein include BRM-dependent cancers. In certain embodiments, the cancer is non-small cell lung cancer.

In certain embodiments, the subject matter described herein is directed to a method of reducing the level of a target protein in a subject comprising, administering an Ab-CIDE as described herein or composition comprising an Ab-CIDE as described herein to a subject, wherein the PB portion binds a target protein, wherein ubiquitin ligase effects degradation of a bound target protein, wherein the level of a target protein is reduced.

In certain embodiments, an Ab-CIDE comprising an antibody, such as those described above, is used in a method of treating cancer, such as a solid tumor.

An Ab-CIDE may be administered by any route appropriate to the condition to be treated. The Ab-CIDE will typically be administered parenterally, i.e. infusion, subcutaneous, intramuscular, intravenous, intradermal, intrathecal and epidural.

An Ab-CIDE can be used either alone or in combination with other agents in a therapy. For instance, an Ab-CIDE may be co-administered with at least one additional therapeutic agent. Such combination therapies noted above encompass combined administration (where two or more therapeutic agents are included in the same or separate formulations), and separate administration, in which case, administration of the Ab-CIDE can occur prior to, simultaneously, and/or following, administration of the additional therapeutic agent and/or adjuvant. An Ab-CIDE can also be used in combination with radiation therapy.

An Ab-CIDE (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

For the prevention or treatment of disease, the appropriate dosage of an Ab-CIDE (when used alone or in combination with one or more other additional therapeutic agents) will depend on the type of disease to be treated, the type of Ab-CIDE, the severity and course of the disease, whether the Ab-CIDE is administered for preventive or therapeutic purposes, previous therapy, the patient's clinical history and response to the Ab-CIDE, and the discretion of the attending physician. The Ab-CIDE is suitably administered to the patient at one time or over a series of treatments. Depending on the type and severity of the disease, about 1 μg/kg to 15 mg/kg (e.g. 0.1 mg/kg-10 mg/kg) of an Ab-CIDE can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of an Ab-CIDE would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses). An initial higher loading dose, followed by one or more lower doses may be administered. However, other dosage regimens may be useful. The progress of this therapy is easily monitored by conventional techniques and assays.

The methods described herein include methods of degrading target proteins. In certain embodiments, the methods comprise administering an Ab-CIDE to a subject, wherein the target protein is degraded. The level of degradation of the protein can be from about 1% to about 5%; or from about 1% to about 10%; or from about 1% to about 15%; or from about 1% to about 20%; from about 1% to about 30%; or from about 1% to about 40%; from about 1% to about 50%; or from about 10% to about 20%; or from about 10% to about 30%; or from about 10% to about 40%; or from about 10% to about 50%; or at least about 1%; or at least about 10%; or at least about 20%; or at least about 30%; or at least about 40%; or at least about 50%; or at least about 60%; or at least about 70%; or at least about 80%; or at least about 90%; or at least about 95%; or at least about 99%.

The methods described herein include methods of reducing proliferation of a neoplastic tissue, such as non-small cell lung cancer. In certain embodiments, the methods comprise administering an Ab-CIDE to a subject, wherein the proliferation of a neoplastic tissue is reduced. The level of reduction can be from about 1% to about 5%; or from about 1% to about 10%; or from about 1% to about 15%; or from about 1% to about 20%; from about 1% to about 30%; or from about 1% to about 40%; from about 1% to about 50%; or from about 10% to about 20%; or from about 10% to about 30%; or from about 10% to about 40%; or from about 10% to about 50%; or at least about 1%; or at least about 10%; or at least about 20%; or at least about 30%; or at least about 40%; or at least about 50%; or at least about 60%; or at least about 70%; or at least about 80%; or at least about 90%; or at least about 95%; or at least about 99%.

### VI. Articles of Manufacture

In another aspect, described herein are articles of manufacture, for example, a “kit,” containing materials useful for the treatment of the diseases and disorders described above is provided. The kit comprises a container comprising an Ab-CIDE. The kit may further comprise a label or package insert, on or associated with the container. The term “package insert” is used to refer to instructions customarily included in commercial packages of therapeutic products, that contain information about the indications, usage, dosage, administration, contraindications and/or warnings concerning the use of such therapeutic products.

Suitable containers include, for example, bottles, vials, syringes, blister pack, etc. A “vial” is a container suitable for holding a liquid or lyophilized preparation. In one embodiment, the vial is a single-use vial, e.g. a 20-cc single-use vial with a stopper. The container may be formed from a variety of materials such as glass or plastic. The container may hold an Ab-CIDE or a formulation thereof which is effective for treating the condition and may have a sterile access port (for example, the container may be an intravenous solution bag or a vial having a stopper pierceable by a hypodermic injection needle).

At least one active agent in the composition is an Ab-CIDE. The label or package insert indicates that the composition is used for treating the condition of choice, such as cancer. In addition, the label or package insert may indicate that the patient to be treated is one having a disorder such as a hyperproliferative disorder, neurodegeneration, cardiac hypertrophy, pain, migraine or a neurotraumatic disease or event. In one embodiment, the label or package inserts indicates that the composition comprising an Ab-CIDE can be used to treat a disorder resulting from abnormal cell growth. The label or package insert may also indicate that the composition can be used to treat other disorders. Alternatively, or additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

The kit may further comprise directions for the administration of the Ab-CIDE and, if present, the second pharmaceutical formulation. For example, if the kit comprises a first composition comprising an Ab-CIDE, and a second pharmaceutical formulation, the kit may further comprise directions for the simultaneous, sequential or separate administration of the first and second pharmaceutical compositions to a patient in need thereof.

In another embodiment, the kits are suitable for the delivery of solid oral forms of an Ab-CIDE, such as tablets or capsules. Such a kit preferably includes a number of unit dosages. Such kits can include a card having the dosages oriented in the order of their intended use. An example of such a kit is a “blister pack”. Blister packs are well known in the packaging industry and are widely used for packaging pharmaceutical unit dosage forms. If desired, a memory aid can be provided, for example in the form of numbers, letters, or other markings or with a calendar insert, designating the days in the treatment schedule in which the dosages can be administered.

According to one embodiment, a kit may comprise (a) a first container with an Ab-CIDE contained therein; and optionally (b) a second container with a second pharmaceutical formulation contained therein, wherein the second pharmaceutical formulation comprises a second compound with anti-hyperproliferative activity. Alternatively, or additionally, the kit may further comprise a third container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In certain other embodiments wherein the kit comprises an Ab-CIDE and a second therapeutic agent, the kit may comprise a container for containing the separate compositions such as a divided bottle or a divided foil packet; however, the separate compositions may also be contained within a single, undivided container. Typically, the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

### VII. Methods of Making Conjugates

### General Synthesis Routes

The subject matter described herein is also directed to methods of preparing a CIDE, a L1-CIDE, and an Ab-L1-CIDE from a L1-CIDE. Generally, the method comprises contacting an antibody, or variants, mutations, splice variants, indels and fusions thereof, with a L1-CIDE under conditions where the antibody is covalently bound to any available point of attachment on a L1-CIDE, wherein an Ab-L1-CIDE is prepared. The subject matter described herein is also directed to methods of preparing an Ab-L1-CIDE from an Ab-L1 portion, i.e., an antibody, or variants, mutations, splice variants, indels and fusions thereof, covalently attached to a L1, the methods comprising contacting a CIDE with an Ab-L1 under conditions where the CIDE is covalently bound to any available point of attachment on the Ab-L1, wherein an Ab-L1-CIDE is prepared. The methods can further comprise routine isolation and purification of the Ab-L1-CIDEs.

CIDEs, L1-CIDEs and Ab-L1-CIDEs and other compounds described herein can be synthesized by synthetic routes that include processes analogous to those well-known in the chemical arts, particularly in light of the description contained herein, and those for other heterocycles described in: Comprehensive Heterocyclic Chemistry II, Editors Katritzky and Rees, Elsevier, 1997, e.g. Volume 3; Liebigs Annalen der Chemie, (9):1910-16, (1985); Helvetica Chimica Acta, 41:1052-60, (1958); Arzneimittel-Forschung, 40(12):1328-31, (1990). Starting materials are generally available from commercial sources such as Aldrich Chemicals (Milwaukee, WI) or are readily prepared using methods well known to those skilled in the art (e.g., prepared by methods generally described in Louis F. Fieser and Mary Fieser, Reagents for Organic Synthesis, v. 1-23, Wiley, N.Y. (1967-2006 ed.), or Beilsteins Handbuch der organischen Chemie, 4, Aufl. ed. Springer-Verlag, Berlin, including supplements (also available via the Beilstein online database).

Synthetic chemistry transformations and protecting group methodologies (protection and deprotection) useful in synthesizing the CIDEs, L1-CIDEs and Ab-L1-CIDEs and other compounds as described herein and necessary reagents and intermediates are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); and L. Paquette, ed., Encyclopedia of Reagents for Organic Synthesis, John Wiley and Sons (1995) and subsequent editions thereof. In preparing CIDEs, L1-CIDEs and Ab-CIDEs and other compounds, protection of remote functionality (e.g., primary or secondary amine) of intermediates may be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz or CBZ) and 9-fluorenylmethyleneoxycarbonyl (Fmoc). The need for such protection is readily determined by one skilled in the art. For a general description of protecting groups and their use, see T. W. Greene, Protective Groups in Organic Synthesis, John Wiley & Sons, New York, 1991.

The General Procedures and Examples provide exemplary methods for preparing CIDEs, L1-CIDEs and Ab-L1-CIDEs and other compounds described herein. Those skilled in the art will appreciate that other synthetic routes may be used to synthesize the Ab-L1-CIDEs and compounds. Although specific starting materials and reagents are depicted and discussed in the Schemes, General Procedures, and Examples, other starting materials and reagents can be easily substituted to provide a variety of derivatives and/or reaction conditions. In addition, many of the exemplary compounds prepared by the described methods can be further modified in light of this disclosure using conventional chemistry well known to those skilled in the art.

Generally, an Ab-L1-CIDE can be prepared by connecting a CIDE with a L1 linker reagent according to the procedures of WO 2013/055987; WO 2015/023355; WO 2010/009124; WO 2015/095227, to prepare a L1-CIDE, and conjugating the L1-CIDE with any of the antibodies or variants, mutations, splice variants, indels and fusions thereof, including cysteine engineered antibodies, described herein. Alternatively, an Ab-CIDE can be prepared by first connecting an antibody or variant, mutation, splice variant, indel and fusion thereof, including a cysteine engineered antibody, described herein with a L1 linker reagent, and conjugating it with any CIDE.

The following synthetic routes describe exemplary methods of preparing CIDEs, L1-CIDEs and Ab-L1-CIDEs and other compounds and components thereof. Other synthetic routes for preparing CIDEs, L1-CIDEs and Ab-L1-CIDEs and other compounds and components thereof are disclosed elsewhere herein.

A non-limiting general synthetic route for preparing an Ab-CIDE is depicted in Scheme 1.

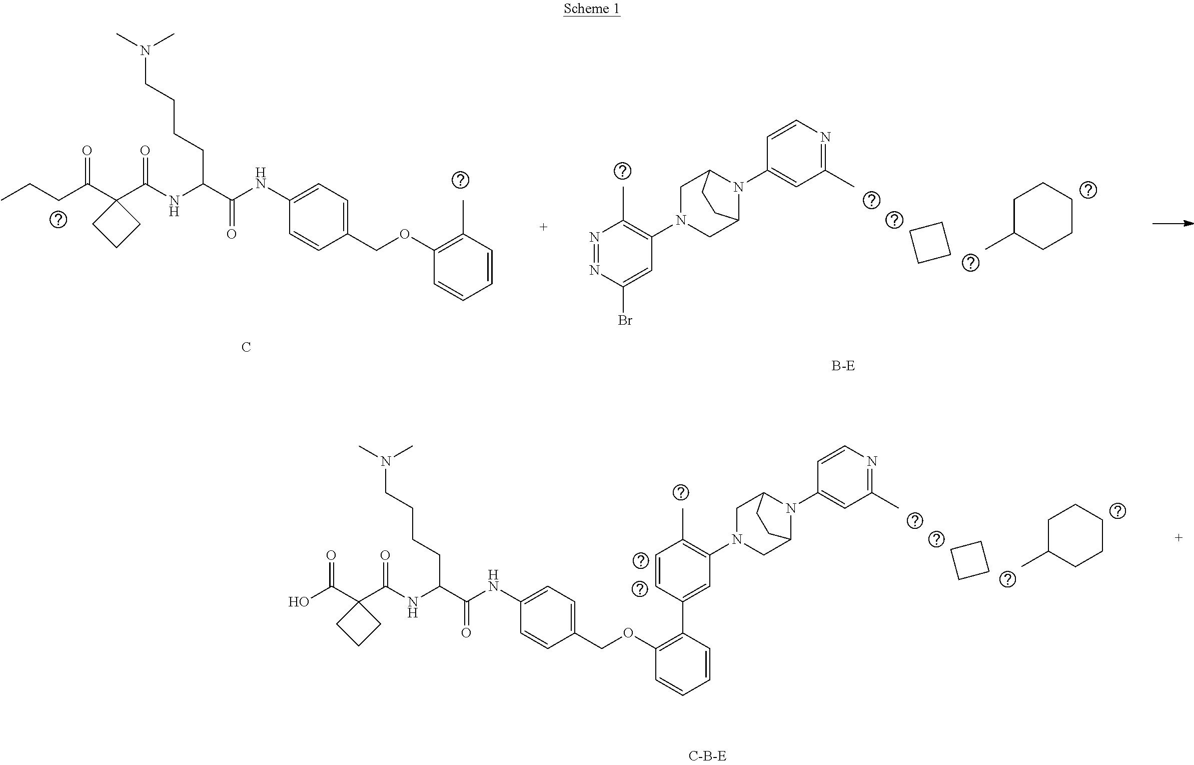

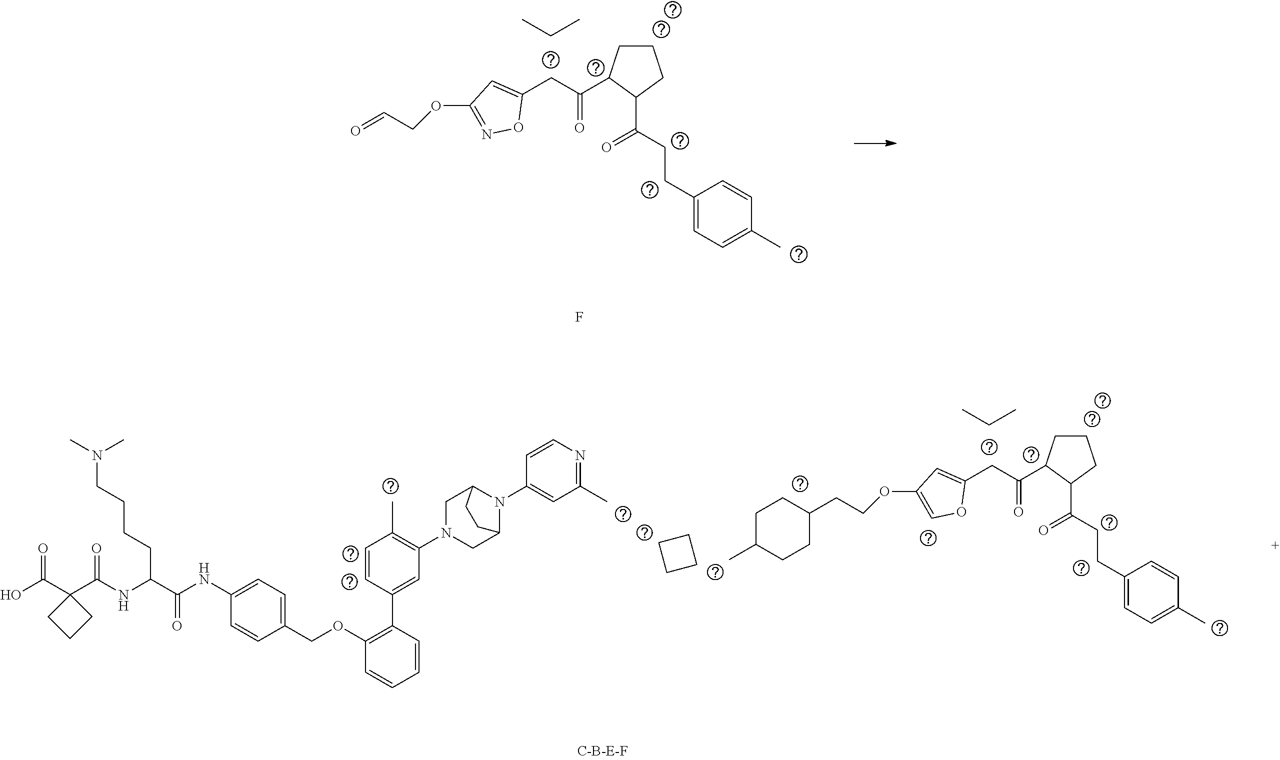

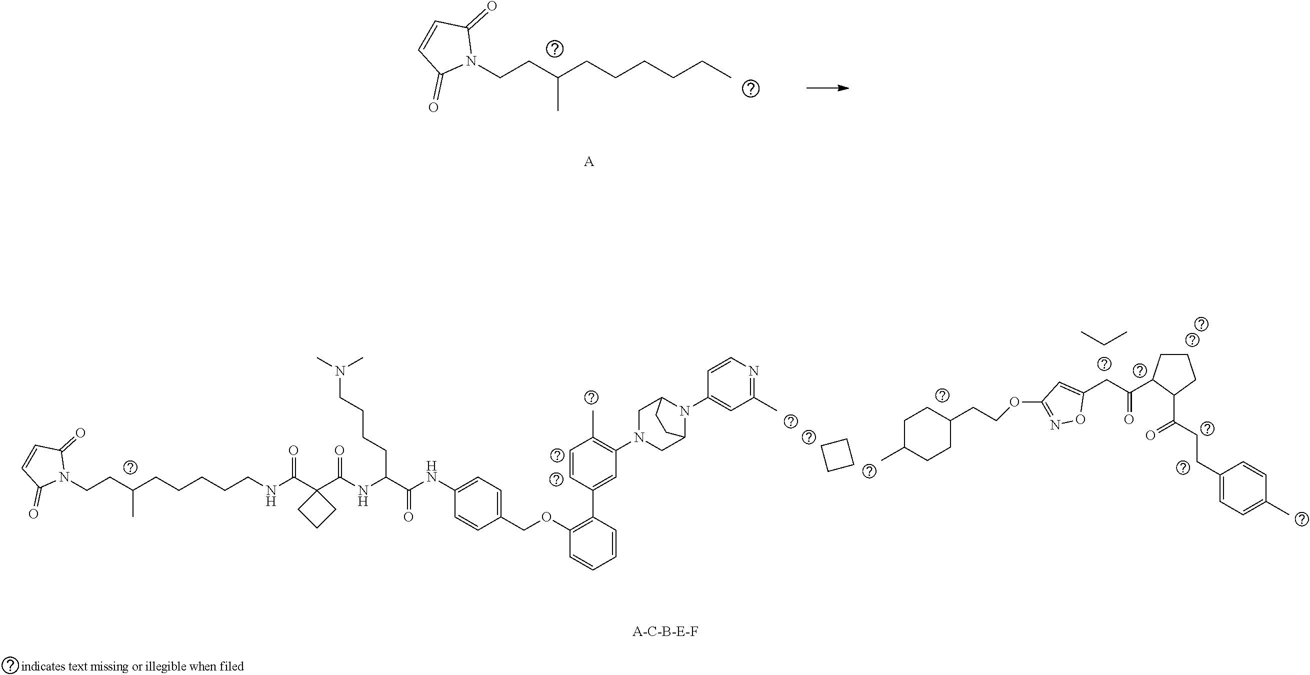

### 1. Cysteine Engineered Antibodies

Cysteine engineered antibodies (THIOMABs™) can be expressed and purified recombinantly using standard methods, and can generally be prepared for conjugation by reduction and reoxidation as follows.

Full length, cysteine engineered monoclonal antibodies (THIOMAB™ antibodies) expressed recombinantly bear cysteine adducts (cystines) or are glutathionylated on the engineered cysteines due to cell culture conditions. As is, THIOMAB™ antibodies purified from standard mammalian cell lines cannot be conjugated to Cys-reactive linker L1-CIDE intermediates. Cysteine engineered antibodies may be made reactive for conjugation with L1-CIDE intermediates described herein, by treatment with a reducing agent such as DTT (Cleland's reagent, dithiothreitol) or TCEP (tris(2-carboxyethyl)phosphine hydrochloride; Getz et al (1999) Anal. Biochem. Vol 273:73-80; Soltec Ventures, Beverly, MA) followed by re-formation of the inter-chain disulfide bonds (re-oxidation) with a mild oxidant such as dehydroascorbic acid. Full length, cysteine engineered monoclonal antibodies (THIOMAB™ antibodies) expressed in CHO cells (Gomez et al (2010) Biotechnology and Bioeng. 105(4):748-760; Gomez et al (2010) Biotechnol. Prog. 26:1438-1445) were reduced, for example, with about a 50 fold excess of DTT overnight in 50 mM Tris, pH 8.0 with 2 mM EDTA at room temperature, which removes Cys and glutathione adducts as well as reduces interchain disulfide bonds in the antibody. Removal of the adducts was monitored by reverse-phase LCMS using a PLRP-S column.

After the removal of Cys and glutathione adducts, THIOMAB™ antibodies can be purified by methods known commonly in the art, including cation exchange chromatography which is elaborated here. Reduced THIOMABs™ can be diluted and acidified by adding to at least four volumes of 10 mM succinate, pH 5 and/or titration with 10% acetic acid until the pH is approximately five. The pH-lowered and diluted THIOMAB™ antibody can be subsequently loaded onto a HiTrap S cation exchange column, washed with several column volumes of 10 mM sodium acetate, pH 5 and eluted with 50 mM Tris, pH 8.0, 150 mM sodium chloride. Disulfide bonds can be reestablished between cysteine residues present in the parent Mab by carrying out reoxidation. The eluted reduced THIOMAB™ antibody described above can be treated with 15×dehydroascorbic acid (DHAA) for about 3 hours or, alternatively, with 200 nM to 2 mM aqueous copper sulfate (CuSO₄) at room temperature overnight. Other oxidants, i.e. oxidizing agents, and oxidizing conditions, which are known in the art may be used. Ambient air oxidation may also be effective. This mild, partial reoxidation step forms intrachain disulfides efficiently with high fidelity. Reoxidation can be monitored by reverse-phase LCMS using a PLRP-S column. The reoxidized THIOMAB™ antibody can then be diluted with succinate buffer as described above to reach pH approximately 5, followed by purification on an S column as described above with the exception that elution was performed with a gradient of 10 mM succinate, pH 5, 300 mM sodium chloride (buffer B) and 10 mM succinate, pH 5 (buffer A). To the eluted THIOMAB™ antibody, EDTA can be added to a final concentration of 2 mM. The THIOMAB™ can be concentrated, if necessary, to reach a final concentration of more than 5 mg/mL. The resulting THIOMAB™ antibody, ready for conjugation, can be stored at −20° C. or −80° C. Liquid chromatography/Mass Spectrometric Analysis can be performed on a 6200 series TOF or QTOF Agilent LC/MS. Samples are chromatographed on a PRLP-S®, 1000 A, microbore column (50 mm×2.1 mm, Polymer Laboratories, Shropshire, UK) heated to 80° C. A linear gradient from 30-40% B (solvent A: 0.05% TFA in water, solvent B: 0.04% TFA in acetonitrile) can be used and the eluent is directly ionized using the electrospray source. Data were collected and deconvoluted by the MassHunter software (Agilent). Prior to LC/MS analysis, antibodies (50 micrograms) can be treated with PNGase F (2 units/ml; PROzyme, San Leandro, CA) for 2 hours at 37° C. to remove N-linked carbohydrates.

Alternatively, antibodies can be partially digested with LysC (0.25 μg per 50 μg (microgram) antibody) for 15 minutes at 37° C. to give a Fab and Fc fragment for analysis by LCMS 2. Conjugation of Linker L1-CIDE group to antibodies

In one method of conjugating Linker L1-CIDE compounds to antibodies, after the reduction and reoxidation procedures above, the cysteine-engineered antibody (THIOMAB™ antibody), in 10 mM succinate, pH 5, 150 mM NaCl, 2 mM EDTA, is pH-adjusted to pH 7.5-8.5 with 1M Tris. An excess, from about 3 molar to 20 equivalents of a linker-CIDE intermediate with a thiol-reactive group (e.g., maleimide or 4-nitropyridy disulfide, or methanethiosulfonyl (MTS) disulfide), is dissolved in DMF or DMA, with or without or propylene glycol before addition to the reduced, reoxidized, and pH-adjusted antibody. The reaction is incubated at room temperature or 37 C and monitored until completion (1 to about 24 hours), as determined by LC-MS analysis of the reaction mixture. When the reaction is complete, the conjugate is purified by one or any combination of several methods, the goal being to remove remaining unreacted L1-CIDE intermediate and aggregated protein (if present at significant levels). For example, the conjugate may be diluted with 10 mM histidine-acetate, pH 5.5 until final pH is approximately 5.5 and purified by S cation exchange chromatography using either HiTrap S columns connected to an Akta purification system (GE Healthcare) or S maxi spin columns (Pierce). Alternatively, the conjugate may be purified by gel filtration chromatography using an S200 column connected to an Akta purification system or Zeba spin columns. Alternatively, dialysis may be used to remove unreacted or excess linker drug. The THIOMAB™ antibody CIDE conjugates can be formulated into 20 mM His/acetate, pH 5, with 240 mM sucrose using either gel filtration or dialysis. The purified conjugate can be concentrated by centrifugal ultrafiltration and/or filtered through a 0.2-μm filter under sterile conditions and frozen for storage. The Ab-L1-CIDEs were characterized by BCA assay to determine protein concentration, analytical SEC (size-exclusion chromatography) for aggregation analysis and LC-MS after treatment with Lysine C endopeptidase (LysC) or reduction using standard procedures to calculate DAR.

Size exclusion chromatography is performed on conjugates using a Shodex KW802.5 column in 0.2M potassium phosphate pH 6.2 with 0.25 mM potassium chloride and 15% IPA at a flow rate of 0.75 ml/min. Aggregation state of the conjugate was determined by integration of eluted peak area absorbance at 280 nm.

LC-MS analysis may be performed on Ab-L1-CIDE using an Agilent QTOF 6520 ESI instrument. As an example, the Ab-L1-CIDE is treated with 1:500 w/w Endoproteinase Lys C (Promega) in Tris, pH 7.5, for 30 min at 37° C. The resulting cleavage fragments are loaded onto a 1000 (Angstrom), 8 m (micron) PLRP-S (highly cross-linked polystyrene) column heated to 80° C. and eluted with a gradient of 30% B to 40% B in 5 minutes. Mobile phase A was H₂O with 0.05% TFA and mobile phase B was acetonitrile with 0.04% TFA. The flow rate was 0.5 ml/min. Protein elution was monitored by UV absorbance detection at 280 nm prior to electrospray ionization and MS analysis. Chromatographic resolution of the unconjugated Fc fragment, residual unconjugated Fab and drugged Fab was usually achieved. The obtained m/z spectra were deconvoluted using Mass Hunter™ software (Agilent Technologies) to calculate the mass of the antibody fragments. Peaks in the deconvoluted LCMS spectra can be assigned and quantitated. CIDE-to-antibody ratios (DAR) are calculated by calculating the ratio of intensities of the peak or peaks corresponding to CIDE-conjugated antibody relative to all peaks observed.

### 3. General Synthetic Coupling of L2 to E3LB to Prepare a E3LB-L2 Intermediate

In certain embodiments, L2 is first contacted with a first suitable solvent, a first base and a first coupling reagent to prepare a first solution. In certain embodiments, the contacting of L2 with a first suitable solvent, a first base, and a first coupling reagent proceeds for about 15 minutes at room temperature (about 25° C.). The E3LB is then contacted with said first solution.

In certain embodiments, the contacting of E3LB with the first solution proceeds for about one hour at room temperature (about 25° C.). The solution is then concentrated and optionally purified.

In certain embodiments, the molar ratio of L2 to first base to first coupling reagent is about 1:4:1.19. In certain embodiments, the molar ratio of L2 to first base to first coupling reagent is about 1:2:0.5, about 1:3:1, about 1:4:2, about 1:5:3, or about 1:6:4.

In certain embodiments, the molar ratio of L2 to E3LB is about 1:1. In certain embodiments, the molar ratio of L2 to E3LB is about 1:0.5, about 1:0.75, about 1:2, or about 0.5:1.

### 4. General Synthetic Method for Coupling E3LB-L2 Intermediate to PB

In certain embodiments, the E3LB-L2 intermediate is coupled to a PB to prepare a CIDE. In certain embodiments, the PB is first contacted with a second suitable solvent, a second base, and second coupling reagent. In certain embodiments, the contacting proceeds for about 10 minutes at room temperature (about 25° C.). The solution is then contacted with the E3LB-L2 intermediate. In certain embodiments, the contacting of the second solution with the E3LB-L2 intermediate proceeds for about 1 hour at room temperature (about 25° C.). The solution is then concentrated and optionally purified to prepare a CIDE.

In certain embodiments, the molar ratio of PB to second base to second coupling reagent is about 1:4:1.2. In certain embodiments, the molar ratio of PB to second base to second coupling reagent is about 1:3:0.75, about 1:5:1, about 1:3:2, or about 1:5:3.

In certain embodiments, the molar ratio of PB to E3LB-L2 intermediate is about 1:1. In certain embodiments, the molar ratio of PB to E3LB-L2 intermediate is about 1:0.5, about 1:0.75, about 1:2, or about 0.5:1.

### 5. General Synthetic Method for Coupling CIDE to L1 to Prepare L1-CIDE

In certain embodiments, the CIDE is contacted with L1 and a third base in a third suitable solvent to prepare a solution. In certain embodiments, the contacting proceeds for about 2 hours at about (about 25° C.). The solution can then be optionally purified to prepare L1-CIDE.

In certain embodiments, the molar ratio of CIDE to L1 is about 1:4. In certain embodiments, the molar ratio of CIDE to L1 is about 1:1, 1:2, 1:3, 1:5, 1:6, 1:7, or about 1:8.

### 6. General Synthetic Method for Coupling L1-CIDE to Antibody

In certain embodiments, the L1-CIDE is contacted with a thiol and a fourth suitable solvent to form a fourth solution. This solution is then contacted with an antibody to prepare the conjugate. In certain embodiments, the

In certain embodiments, the thiol is maleimide or 4-nitropyridy disulfide. In certain embodiments, the suitable solvent is selected from the group consisting of dimethylformamide, dimethylacetamide, and propylene glycol.

In certain embodiments, the molar ratio of L1-CIDE to thiol-reactive group is about 3:1 to about 20:1.

In certain embodiments, contacting the solution comprising the L1-CIDE, the thiol-reactive group and the suitable solvent with the antibody proceeds for about 1 to about 24 hours. In certain embodiments, contacting the solution comprising the L1-CIDE, the thiol-reactive group and the suitable solvent with the antibody proceeds at about room temperature (about 25° C.) to about 37° C.

In certain embodiments of the general methods above, the suitable solvent is a polar aprotic solvent, selected from the group consisting of dimethylformamide, tetrahydrofuran, ethyl acetate, acetone, acetonitrile, dimethyl sulfoxide, and propylene carbonate.

In certain embodiments of the general methods above, the base is selected from the group consisting of N,N-Diisopropylethylamine (DIEA), triethylamine, and 2,2,2,6,6-tetramethylpiperidine. In certain embodiments, the coupling reagent is selected from the group consisting of 1-[Bis(dimethylamino)methylene]-1H-1,2,3-triazolo[4,5-b]pyridinium 3-oxide hexafluorophosphate (HATU), (Benzotriazol-1-yloxy)tris(dimethylamino)phosphonium hexafluorophosphate (BOP), (7-Azabenzotriazol-1-yloxy)tripyrrolidinophosphonium hexafluorophosphate (PyAOP), O-(Benzotriazol-1-yl)-N,N,N′,N′-tetramethyluronium hexafluorophosphate (HBTU), O-(Benzotriazol-1-yl)-N,N,N′,N′-tetramethyluronium tetrafluoroborate (TBTU), O-(6-Chlorobenzotriazol-1-yl)-N,N,N′,N′-tetramethyluronium hexafluorophosphate (HCTU), O—(N-Suc-cinimidyl)-1,1,3,3-tetramethyl-uronium tetrafluoroborate (TSTU), O-(5-Norbornene-2,3-dicarboximido)-N,N,N′,N′-tetramethyluronium tetrafluoroborate (TNTU), O-(1,2-Dihydro-2-oxo-1-pyridyl-N,N,N′,N′-tetramethyluronium tetrafluoroborate (TPTU), and Carbonyldiimnidazole (CDI).

In certain embodiments of the general methods above, contacting proceeds for about 30 seconds, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 16 minutes, 17 minutes, 18 minutes, 19 minutes, 20 minutes, 30 minutes, 60 minutes, 90 minutes, 120 minutes, 180 minutes, 4 hours, 5 hours, 6 hours, 7 hours, 8 hours, 9 hours, 10 hours, 20 hours, 40 hours, 60 hours, or 72 hours.

In certain embodiments of the general methods above, contacting proceeds at about 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 60° C., 70° C., 80° C., 90° C., or 100° C.

The following examples are offered by way of illustration and not by way of limitation.

### EXAMPLES

### Example 1

### Syntheses of a Portion of a Hydrolysable Linker and a PB

### Intermediate 1: Ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate

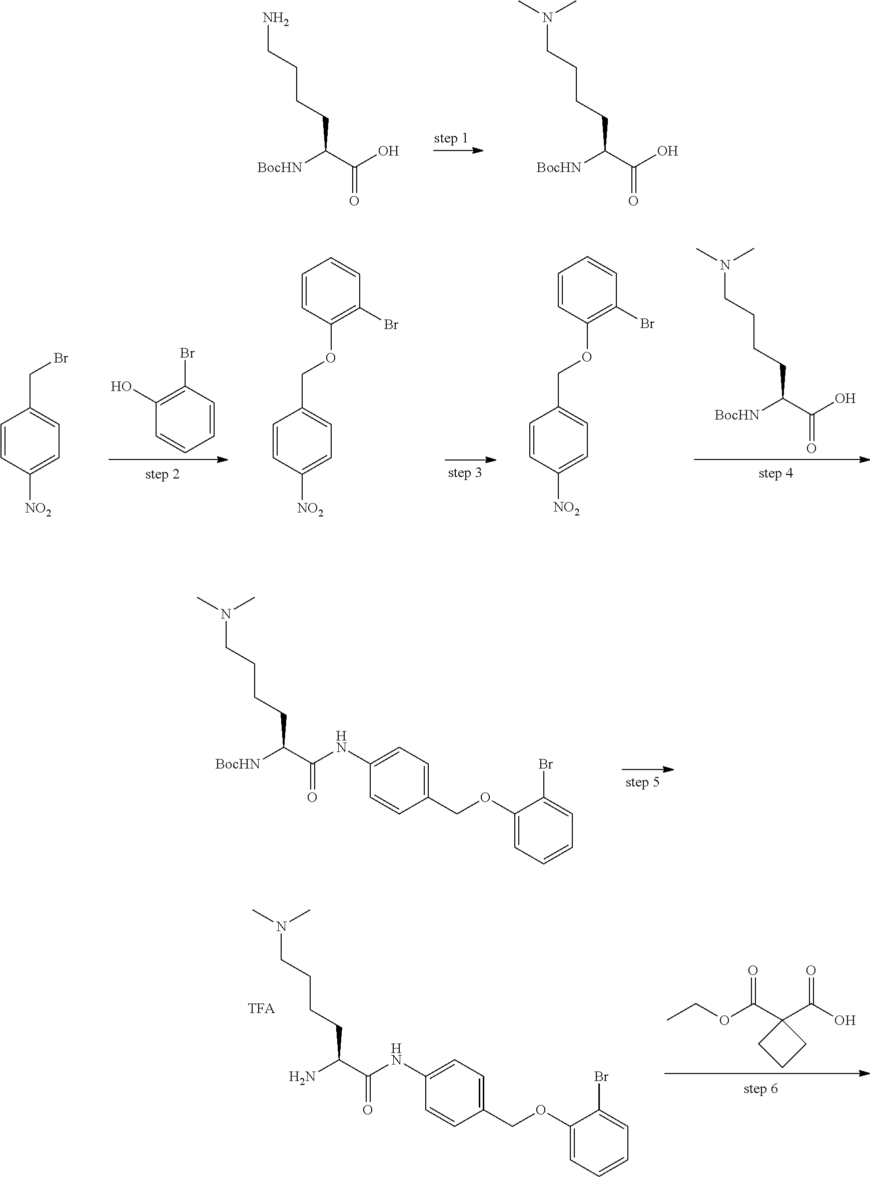

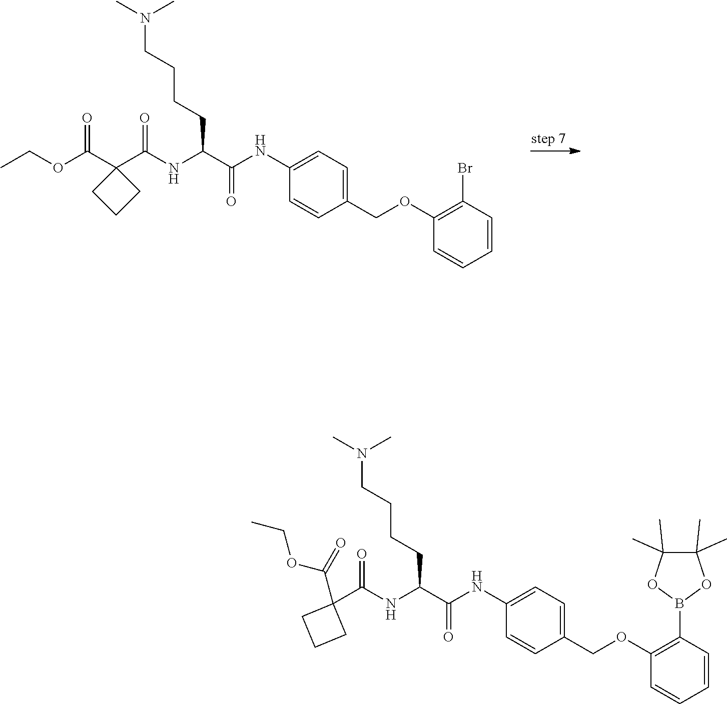 [SMILES: CCOC(=O)C1(C(=O)N[C@@H](CCCCN(C)C)C(=O)Nc2ccc(COc3ccccc3Br)cc2)CCC1 ; CCOC(=O)C1(C(=O)N[C@@H](CCCCN(C)C)C(=O)Nc2ccc(COc3ccccc3B3OC(C)(C)C(C)(C)O3)cc2)CCC1]

### Step 1: N²-(tert-Butoxycarbonyl)-N⁶,N⁶-dimethyl-L-lysine

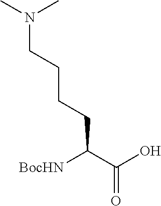 [SMILES: CN(C)CCCC[C@H](NC(=O)OC(C)(C)C)C(=O)O]

Under hydrogen (3 atm), a solution of (tert-butoxycarbonyl)-L-lysine (20.0 g, 81.2 mmol), 37% aqueous CH₂O (12.2 g, 150 mmol) and 10% Pd/C (2.00 g) in methyl alcohol (100 mL) was stirred at room temperature for 4 h. After filtration, the filtrate was concentrated under reduced pressure. The residue was washed with Et₂O. The solids were collected by filtration to afford 20.6 g (92% yield) of the title compound as a white solid.

LCMS (ESI) [M+H]+=275.

### Step 2: 1-Bromo-2-((4-nitrobenzyl)oxy)benzene

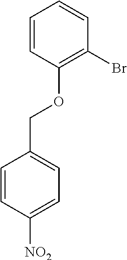 [SMILES: O=[N+]([O-])c1ccc(COc2ccccc2Br)cc1]

A solution of 2-bromophenol (52.6 g, 304 mmol), 1-(bromomethyl)-4-nitrobenzene (65.7 g, 304 mmol) and K₂CO₃ (83.9 g, 608 mmol) in DMF (700 mL) was stirred at room temperature for 1 h. EtOAc was added and water was used to wash for three times. The organic layer was dried over anhydrous Na₂SO₄ and concentrated under vacuum to afford 73.8 g (78% yield) of the title compound as a yellow solid. ¹H NMR (300 MHz, DMSO-d₆, ppm) δ 8.34-8.24 (m, 2H), 7.81-7.70 (m, 2H), 7.62 (dd, J=7.9, 1.6 Hz, 1H), 7.36 (ddd, J=8.3, 7.3, 1.6 Hz, 1H), 7.20 (dd, J=8.3, 1.5 Hz, 1H), 6.94 (td, J=7.6, 1.4 Hz, 1H), 5.39 (s, 2H).

### Step 3: 4-((2-Bromophenoxy)methyl)aniline

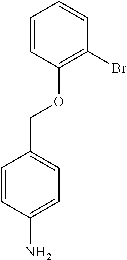 [SMILES: Nc1ccc(COc2ccccc2Br)cc1]

Under nitrogen, to a solution of 1-bromo-2-((4-nitrobenzyl)oxy)benzene (43.0 g, 139.5 mmol) and K₂CO₃ (115 g, 837 mmol) in acetonitrile (800 mL) and water (400 mL) was added Na₂S₂O₄ (242 g, 1395 mmol) in portions at 0° C. The mixture was stirred at room temperature for 6 h. EtOAc was used to extract the product once. The organic layer was dried over anhydrous Na₂SO₄ and concentrated under vacuum to afford 35 g (crude) of the title compound as a yellow solid. LCMS (ESI) [M+H]⁺=278.

### Step 4: tert-Butyl (S)-(1-((4-((2-bromophenoxy)methyl)phenyl)amino)-6-(dimethylamino)-1-oxohexan-2-yl)carbamate

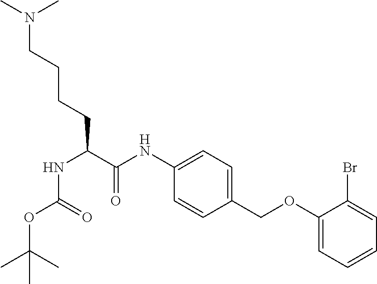 [SMILES: CN(C)CCCC[C@H](NC(=O)OC(C)(C)C)C(=O)Nc1ccc(COc2ccccc2Br)cc1]

Under nitrogen, to a solution of N²-(tert-butoxycarbonyl)-N⁶,N⁶-dimethyl-L-lysine (13.3 g, 48.4 mmol) and NMM (10.3 g, 96.9 mmol) in tetrahydrofuran (200 mL) was added iso-butyl chloroformate (7.91 g, 58.1 mmol) dropwise at −25° C. The reaction was stirred at −25° C. for 0.5 h. Then a solution of 4-((2-bromophenoxy)methyl)aniline (16.1 g, crude) in tetrahydrofuran (120 mL) was added at −25° C. The reaction was stirred at room temperature for 4 h. The solvent was concentrated under vacuum. DCM was added and washed with water. The organic layer was dried over anhydrous Na₂SO₄ and concentrated under vacuum. The residue was purified by flash chromatography on silica gel (solvent gradient: 0-9% MeOH/DCM) to afford 6.70 g (25% yield) of the title compound as a white solid. LCMS (ESI) [M+H]⁺=534.

### Step 5: (S)-2-Amino-N-(4-((2-bromophenoxy)methyl)phenyl)-6-(dimethylamino)hexanamide (2,2,2-trifluoroacetic acid Salt)

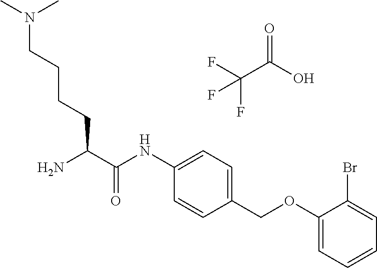 [SMILES: CN(C)CCCC[C@H](N)C(=O)Nc1ccc(COc2ccccc2Br)cc1 ; O=C(O)C(F)(F)F]

A solution of tert-butyl (S)-(1-((4-((2-bromophenoxy)methyl)phenyl)amino)-6-(dimethylamino)-1-oxohexan-2-yl)carbamate (4.00 g, 7.48 mmol) in 5% TFA/HFIP (50 mL) was stirred at room temperature for 3 h. The solvent was concentrated under vacuum and used in next step directly. LCMS (ESI) [M+H]⁺=434.

### Step 6: Ethyl (S)-1-((1-((4-((2-bromophenoxy)methyl)phenyl)amino)-6-(dimethylamino)-1-oxohexan-2-yl)carbamoyl)cyclobutane-1-carboxylate

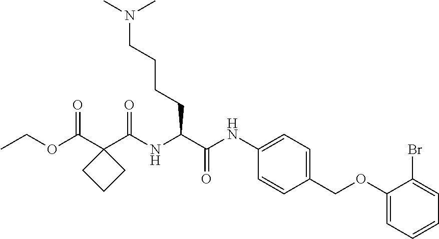 [SMILES: CCOC(=O)C1(C(=O)N[C@@H](CCCCN(C)C)C(=O)Nc2ccc(COc3ccccc3Br)cc2)CCC1]

To a solution of (S)-2-amino-N-(4-((2-bromophenoxy)methyl)phenyl)-6-(dimethylamino)hexanamide (2,2,2-trifluoroacetic acid salt) (crude from step 5), 1-(ethoxycarbonyl)cyclobutane-1-carboxylic acid (1.55 g, 8.98 mmol) and DIPEA (9.65 g, 74.8 mmol) in DMF (20 mL) was added HATU (3.41 g, 8.98 mmol) at 0° C. The mixture was stirred at room temperature for 0.5 h. The crude was purified by pre-packed C18 column (solvent gradient: 0-100% MeOH in water (0.05% NH₄HCO₃)) to afford 2.70 g (61% yield) of the title compound as a red solid. LCMS (ESI) [M+H]⁺=588. ¹H NMR (300 MHz, DMSO-d₆, ppm) δ 9.87 (s, 1H), 7.72 (d, J=7.9 Hz, 1H), 7.54-7.42 (m, 3H), 7.30 (d, J=8.6 Hz, 2H), 7.21 (ddd, J=8.8, 7.3, 1.6 Hz, 1H), 7.07 (dd, J=8.4, 1.5 Hz, 1H), 6.77 (td, J=7.6, 1.4 Hz, 1H), 5.02 (s, 2H), 4.30 (q, J=8.0 Hz, 1H), 4.00 (q, J=7.1 Hz, 2H), 2.35-2.21 (m, 2H), 2.03 (t, J=6.8 Hz, 2H), 1.96 (s, 6H), 1.80-1.41 (m, 4H), 1.30-1.14 (m, 6H), 1.06 (t, J=7.1 Hz, 3H).

### Step 7: Ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate

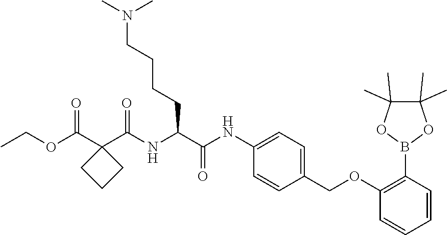 [SMILES: CCOC(=O)C1(C(=O)N[C@@H](CCCCN(C)C)C(=O)Nc2ccc(COc3ccccc3B3OC(C)(C)C(C)(C)O3)cc2)CCC1]

Under nitrogen, a solution of ethyl (S)-1-((1-((4-((2-bromophenoxy)methyl)phenyl)amino)-6-(dimethylamino)-1-oxohexan-2-yl)carbamoyl)cyclobutane-1-carboxylate (500 mg, 0.852 mmol), B₂Pin₂ (649 mg, 2.55 mmol), Pd(dppf)Cl₂ (124 mg, 0.170 mmol) and KOAc (250 mg, 2.55 mmol) in 1,4-dioxane (5 mL) was stirred at 80° C. for 2 h. The reaction was diluted with DCM and washed with water. The organic layer was dried over anhydrous sodium sulfate and concentrated under vacuum. The residue was purified by flash chromatography on silica gel (gradient: 0%-20% MeOH/DCM (contain 0.3% 7 M NH₃/MeOH)) to yield 390 mg (72% yield) of the title compound as a yellow solid. LC-MS: (ESI, m/z): [M+H]⁺=636.

### Example 2

### Synthesis of a Portion of an E3LB

### Intermediate 2: (2S,4R)—N—((S)-1-(4-Cyanophenyl)ethyl)-4-hydroxy-1-((R)-3-methyl-2-(3-(2-oxoethoxy)isoxazol-5-yl)butanoyl)pyrrolidine-2-carboxamide

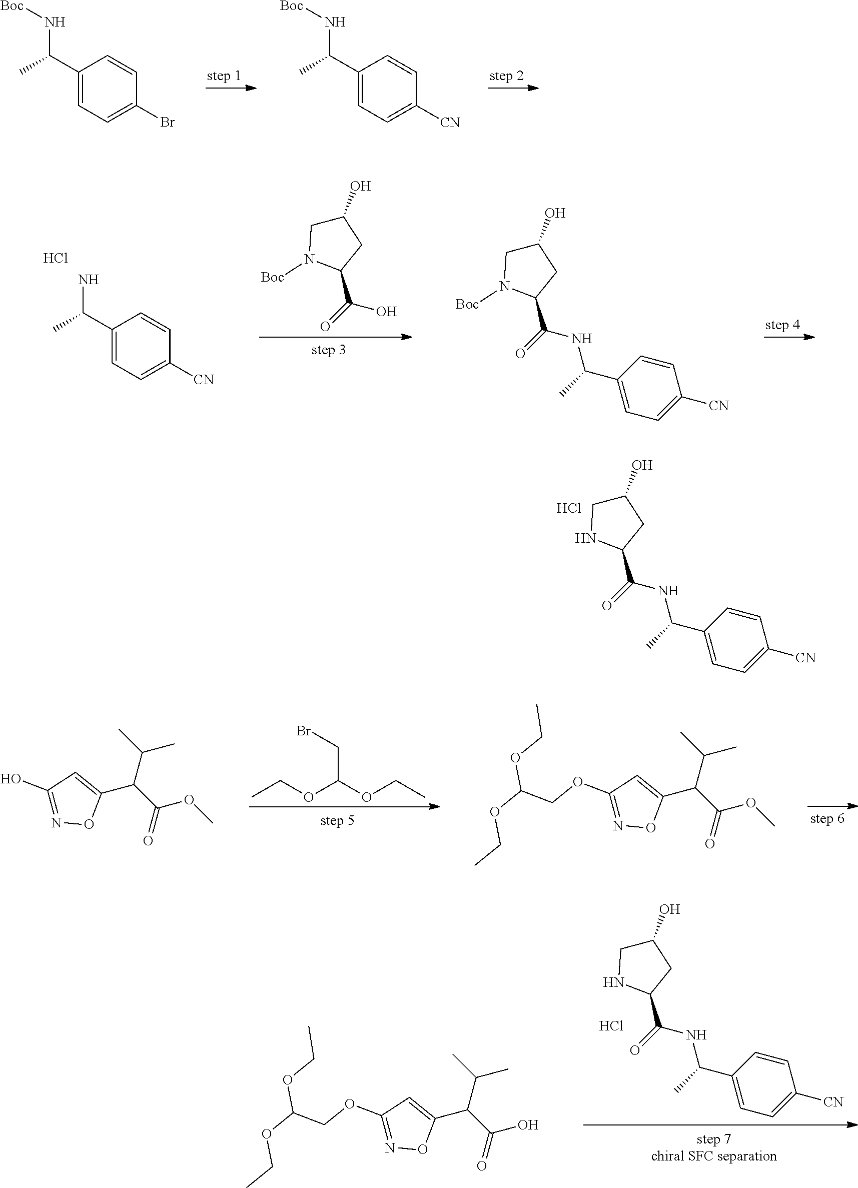

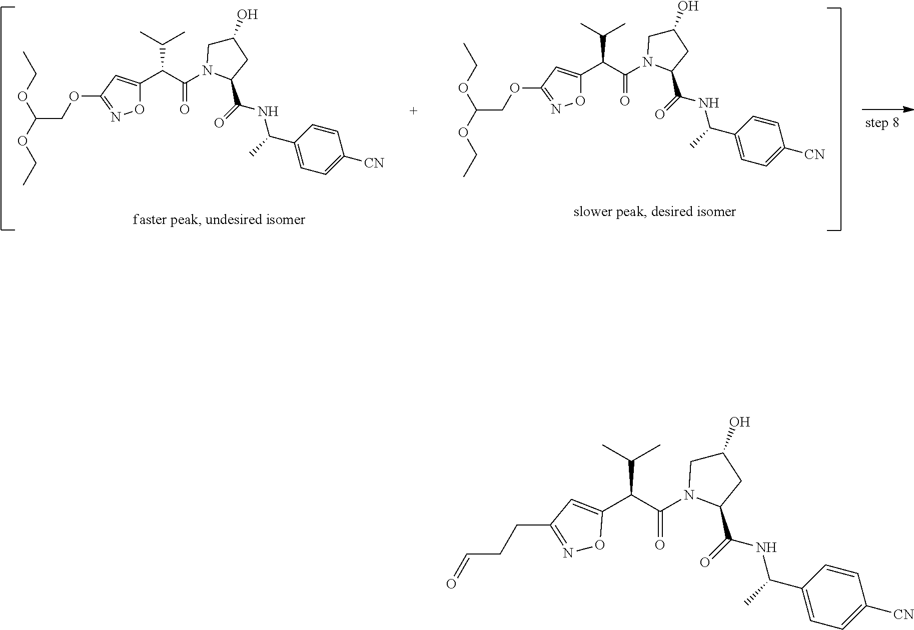

### Step 1: tert-Butyl (S)-(1-(4-cyanophenyl)ethyl)carbamate

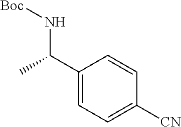 [SMILES: C[C@H](NC(=O)OC(C)(C)C)c1ccc(C#N)cc1]

Under nitrogen, a solution of tert-butyl (S)-(1-(4-bromophenyl)ethyl)carbamate (5.00 g, 16.7 mmol), cyanazine (2.32 g, 20.1 mmol) and Pd(PPh₃)₄ (184 mg, 0.159 mmol) in DMF (50 mL) was stirred at 80° C. for 30 min. Then the reaction mixture was poured into water and the solids were collected by filtration and washed with water. The solids were purified by flash chromatography on silica gel (solvent gradient: 0%-30% EtOAc/petroleum ether) to yield 2.80 g (68% yield) of the title compound as a colorless oil. LCMS (ESI): [M+H]⁺=247.

### Step 2: (S)-4-(1-Aminoethyl)benzonitrile (hydrogen chloride Salt)

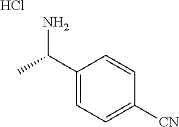

Under nitrogen, a solution of tert-butyl (S)-(1-(4-cyanophenyl)ethyl)carbamate (2.80 g, 11.4 mmol) in CH₂Cl₂ (10 mL) and 4 M HCl/dioxane (20 mL) was stirred at room temperature for 1 h. The resulting mixture was concentrated under vacuum to yield 2.8 g (crude) of the title compound as a white solid. LC-MS: (ESI, m/z): [M+H]⁺=147.

### Step 3: tert-Butyl (2S,4R)-2-(((S)-1-(4-cyanophenyl)ethyl)carbamoyl)-4-hydroxypyrrolidine-1-carboxylate

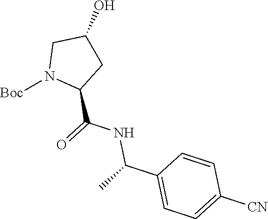 [SMILES: C[C@H](NC(=O)[C@@H]1C[C@@H](O)CN1C(=O)OC(C)(C)C)c1ccc(C#N)cc1]

Under nitrogen, a solution of (S)-4-(1-aminoethyl)benzonitrile (2.35 g, 16.1 mmol), (2S,4R)-1-(tert-butoxycarbonyl)-4-hydroxypyrrolidine-2-carboxylic acid (3.71 g, 16.1 mmol), HATU (7.34 g, 19.3 mmol) and DIPEA (20.7 g, 161 mmol) in DMF (20 mL) was stirred at room temperature for 30 min. The reaction was purified by pre-packed C18 column (solvent gradient: 0-60% ACN in water (0.05% NH₄HCO₃)) to yield 4.60 g (50% yield) of the title compound as a white solid. LC-MS: (ESI, m/z): [M+H]⁺=360.

### Step 4: (2S,4R)—N—((S)-1-(4-cyanophenyl)ethyl)-4-hydroxypyrrolidine-2-carboxamide (hydrogen chloride Salt)

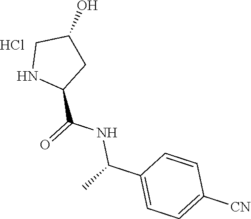 [SMILES: Cl ; C[C@H](NC(=O)[C@@H]1C[C@@H](O)CN1)c1ccc(C#N)cc1]

Under nitrogen, a solution of tert-butyl (2S,4R)-2-(((S)-1-(4-cyanophenyl)ethyl)carbamoyl)-4-hydroxypyrrolidine-1-carboxylate (3.50 g, 9.7 mmol) in CH₂Cl₂ (35 mL) and 4 M HCl/dioxane (70 mL) was stirred at room temperature for 30 mins. The resulting mixture was concentrated under vacuum to yield 3.1 g (crude) of the title compound as a white solid. LC-MS: (ESI, m/z): [M+H]⁺=260.

### Step 5: Methyl 2-(3-(2,2-diethoxyethoxy)isoxazol-5-yl)-3-methylbutanoate

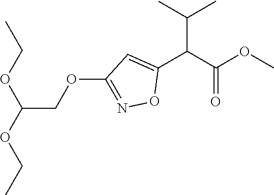 [SMILES: CCOC(COc1cc(C(C(=O)OC)C(C)C)on1)OCC]

A solution of methyl 2-(3-hydroxyisoxazol-5-yl)-3-methylbutanoate (10.0 g, 50.2 mmol), 2-bromo-1,1-diethoxyethane (11.3 mL, 75.1 mmol) and K₂CO₃ (13.9 g, 101 mmol) in DMF (150 mL) was stirred at 70° C. overnight. The reaction mixture was diluted with EtOAc and washed with water. The organic layer was washed with brine, dried over anhydrous sodium sulfate and concentrated under reduced pressure. The residue was purified by pre-packed C18 column (solvent gradient: 0-100% ACN in water (0.05% NH₄HCO₃)) to yield 12.5 g (79% yield) of the title compound as a yellow oil. LC-MS: (ESI, m/z): [M+H]⁺=316.

### Step 6: 2-(3-(2,2-Diethoxyethoxy)isoxazol-5-yl)-3-methylbutanoic acid

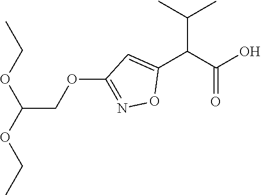 [SMILES: CCOC(COc1cc(C(C(=O)O)C(C)C)on1)OCC]

A solution of methyl 2-(3-(2,2-diethoxyethoxy)isoxazol-5-yl)-3-methylbutanoate (12.5 g, 39.6 mmol) and LiOH (4.75 g, 198 mmol) in tetrahydrofuran (100 mL) and water (100 mL) was stirred at room temperature for 4 h. The pH was adjusted to 4-5 with 1 M hydrogen chloride and extracted with ethyl acetate. The combined organic layers were dried over anhydrous sodium sulfate and concentrated under vacuum to yield 13 g (crude) of the title compound as a white oil. LC-MS: (ESI, m/z): [M+Na]⁺=324.

### Step 7: (2S,4R)—N—((S)-1-(4-Cyanophenyl)ethyl)-1-((R)-2-(3-(2,2-diethoxyethoxy)isoxazol-5-yl)-3-methylbutanoyl)-4-hydroxypyrrolidine-2-carboxamide

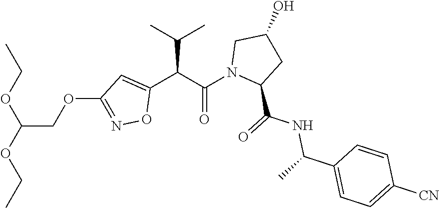 [SMILES: CCOC(COc1cc([C@H](C(=O)N2C[C@H](O)C[C@H]2C(=O)N[C@@H](C)c2ccc(C#N)cc2)C(C)C)on1)OCC]

A solution of (2S,4R)—N—((S)-1-(4-cyanophenyl)ethyl)-4-hydroxypyrrolidine-2-carboxamide (hydrogen chloride salt) (3.00 g, crude from step 4), 2-(3-(2,2-diethoxyethoxy)isoxazol-5-yl)-3-methylbutanoic acid (3.00 g, 9.96 mmol), HATU (4.56 g, 12.0 mmol) and DIPEA (3.85 g, 29.8 mmol) in DMF (30 mL) was stirred at room temperature for 30 min. The crude was purified by pre-packed C18 column (solvent gradient: 0-100% ACN in water (0.05% NH₄HCO₃)) to afford 4.3 g of mixture of two isomers as a yellow solid. The mixture was separated by Prep Chiral SFC with the following conditions: Column: CHIRALPAK IG-3, 3.0*50 mm, 3 m; Mobile Phase B: IPA (0.1% DEA); Flow rate: 2 mL/min; Gradient: isocratic 10% B; Wavelength: 220 nm to yield 2.14 g (faster peak, undesired isomer) and 2.6 g (slower peak, desired isomer) as yellow solids.

For faster peak (undesired isomer): LC-MS: (ESI, m/z): [M+Na]⁺=565. ¹H NMR (300 MHz, DMSO-d₆, ppm) δ 8.35 (8.95) (d, J=7.6 Hz, 1H), 7.85-7.70 (m, 2H), 7.52-7.30 (m, 2H), 6.08 (6.16) (s, 1H), 5.10 (d, J=3.6 Hz, 1H), 5.06-4.83 (m, 1H), 4.79 (td, J=5.2, 1.7 Hz, 1H), 4.55-4.29 (m, 1H), 4.27-4.19 (m, 1H), 4.12-3.99 (m, 2H), 3.73 (d, J=8.6 Hz, 1H), 3.67-3.38 (m, 6H), 2.34-2.15 (m, 1H), 2.09-1.81 (m, 1H), 1.78-1.60 (m, 1H), 1.40-1.25 (m, 3H), 1.19-1.05 (m, 6H), 1.05-0.90 (m, 3H), 0.85-0.70 (m, 3H).

For slower peak (desired isomer) LC-MS: (ESI, m/z): [M+Na]⁺=565. ¹H NMR (300 MHz, DMSO-d₆, ppm) δ 8.46 (8.85) (d, J=7.4 Hz, 1H), 7.83-7.72 (m, 2H), 7.45 (d, J=8.5 Hz, 2H), 6.13 (5.92) (m, 1H), 5.09 (d, J=3.3 Hz, 1H), 4.98-4.82 (m, 1H), 4.79 (td, J=5.2, 2.0 Hz, 1H), 4.39-4.28 (m, 1H), 4.24 (s, 1H), 4.07 (d, J=5.2 Hz, 2H), 3.80-3.59 (m, 4H), 3.56-3.39 (m, 3H), 2.29-2.13 (m, 1H), 2.05-1.65 (m, 2H), 1.45-1.25 (m, 3H), 1.20-1.00 (m, 6H), 0.93 (t, J=6.9 Hz, 3H), 0.79 (dd, J=9.4, 6.7 Hz, 3H).

### Step 8: (2S,4R)—N—((S)-1-(4-Cyanophenyl)ethyl)-4-hydroxy-1-((R)-3-methyl-2-(3-(2-oxoethoxy)isoxazol-5-yl)butanoyl)pyrrolidine-2-carboxamide

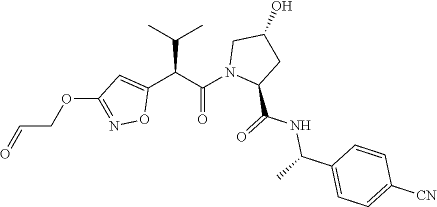 [SMILES: CC(C)[C@@H](C(=O)N1C[C@H](O)C[C@H]1C(=O)N[C@@H](C)c1ccc(C#N)cc1)c1cc(OCC=O)no1]

A solution of (2S,4R)—N—((S)-1-(4-cyanophenyl)ethyl)-1-((R)-2-(3-(2,2-diethoxyethoxy)isoxazol-5-yl)-3-methylbutanoyl)-4-hydroxypyrrolidine-2-carboxamide (300 mg, 0.550 mmol) in tetrahydrofuran (3 mL) and 1 M H₂SO₄ (3 mL) was stirred at 50° C. for 2 h. The solution was cooled and pH was adjusted to 8 by saturated sodium bicarbonate solution. The product was extracted with ethyl acetate. The combined organic layer was washed with brine, dried over sodium sulfate and concentrated in vacuum to yield 250 mg (crude) of the title compound as a white solid. LC-MS: (ESI, m/z): [M+H]⁺=469.

### Example 3

### Synthesis of a Portion of an E3LB

### Intermediate 3: (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide

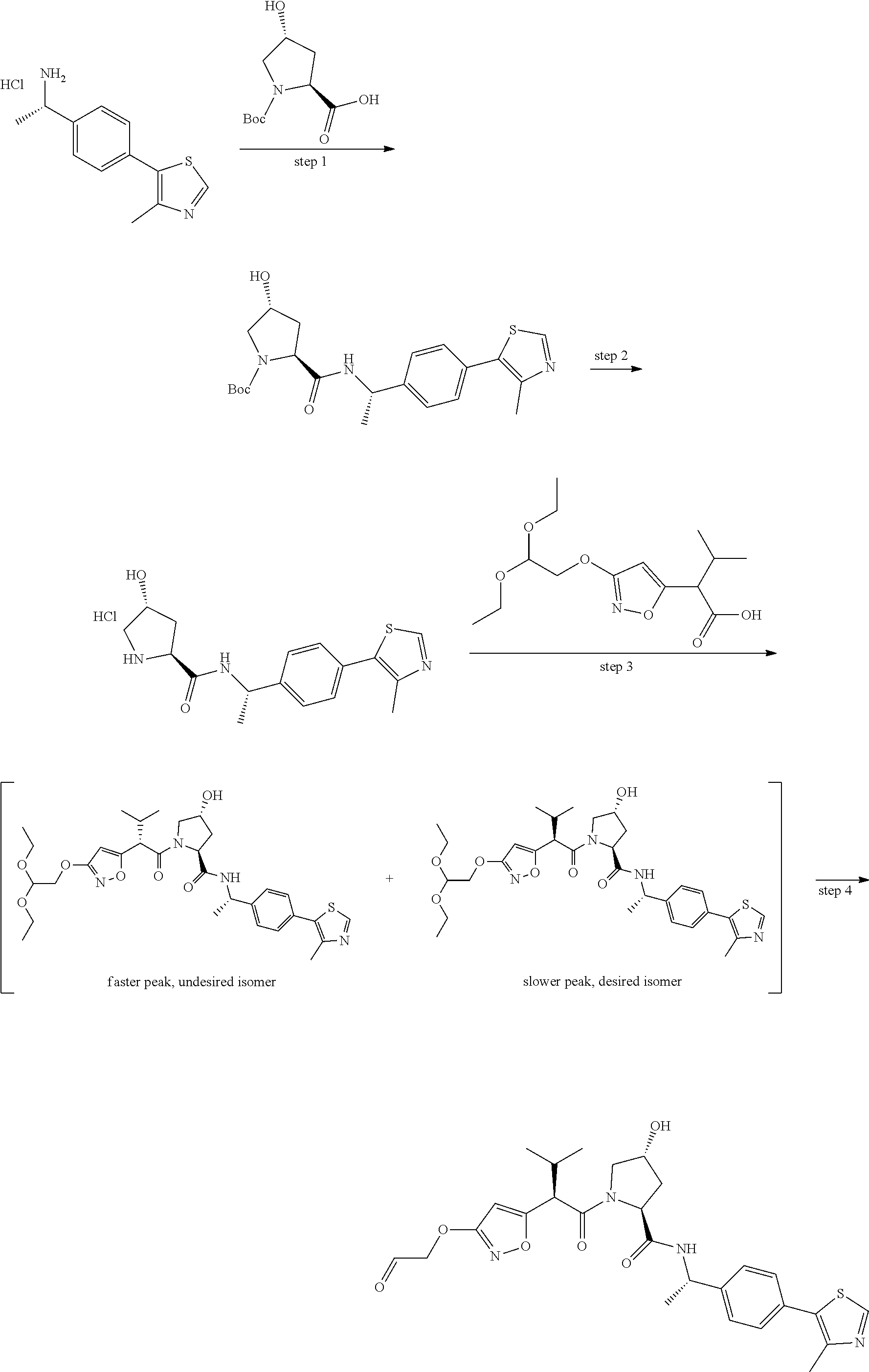

### Step 1: tert-Butyl (2S,4R)-4-hydroxy-2-(((S)-1-(4-(4-methylthiazol-5-yl)phenyl)ethyl)carbamoyl)pyrrolidine-1-carboxylate

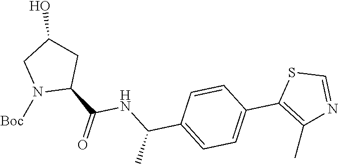 [SMILES: Cc1ncsc1-c1ccc([C@H](C)NC(=O)[C@@H]2C[C@@H](O)CN2C(=O)OC(C)(C)C)cc1]

A solution of(S)-1-(4-(4-methylthiazol-5-yl)phenyl)ethan-1-amine (hydrochloride salt) (10.0 g, 39.3 mmol), (2S,4R)-1-(tert-butoxycarbonyl)-4-hydroxypyrrolidine-2-carboxylic acid (9.07 g, 39.2 mmol), HATU (17.9 g, 47.0 mmol) and DIPEA (25.3 g, 195 mmol) in DMF (100 mL) was stirred at room temperature for 1 h. The reaction was diluted with EtOAc and washed with water. The organic layer was dried over anhydrous Na₂SO₄ and concentrated under reduced pressure. The residue was purified by pre-packed C18 column (solvent gradient: 0-100% ACN in water (0.05% NH₄HCO₃)) to yield 14.2 g (83% yield) of the title compound as an off-white solid. LC-MS: (ESI, m/z): [M+H]⁺=432.

### Step 2: (2S,4R)-4-Hydroxy-N—((S)-1-(4-(4-methylthiazol-5-yl)phenyl)ethyl)pyrrolidine-2-carboxamide (hydrochloride Salt)

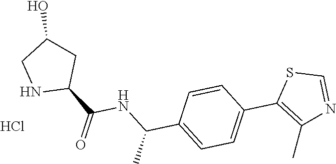 [SMILES: Cl ; Cc1ncsc1-c1ccc([C@H](C)NC(=O)[C@@H]2C[C@@H](O)CN2)cc1]

A solution of tert-butyl (2S,4R)-4-hydroxy-2-(((S)-1-(4-(4-methylthiazol-5-yl)phenyl)ethyl)carbamoyl)pyrrolidine-1-carboxylate (6.89 g, 13.5 mmol) in 4 M HCl/dioxane (50 mL) and dichloromethane (100 mL) was stirred at room temperature for 1 hour. The solvent was concentrated under vacuum to yield 5.8 g (crude) of the title compound as a white solid. LC-MS: (ESI, m/z): [M+H]⁺=332.

### Step 3: (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide

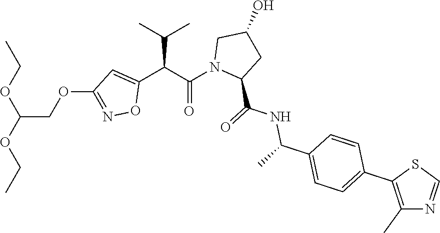 [SMILES: CCOC(COc1cc([C@H](C(=O)N2C[C@H](O)C[C@H]2C(=O)N[C@@H](C)c2ccc(-c3scnc3C)cc2)C(C)C)on1)OCC]

A solution of 2-(3-(2,2-diethoxyethoxy)isoxazol-5-yl)-3-methylbutanoic acid (4.80 g, 15.9 mmol), (2S,4R)-4-hydroxy-N—((S)-1-(4-(4-methylthiazol-5-yl)phenyl)ethyl)pyrrolidine-2-carboxamide (5.78 g, 15.7 mmol), HATU (7.29 g, 19.2 mmol) and DIPEA (6.19 g, 48.0 mmol) in DMF (60 mL) was stirred at room temperature for 30 min. The crude was purified by pre-packed C18 column (solvent gradient: 0-100% ACN in water (0.05% NH₄HCO₃)) to afford 7.8 g of the mixture of two isomers as a yellow solid. The mixture was separated by Prep chiral SFC with the following conditions: Column: CHIRAL ART Amylose-SA, 5*25 cm, 5 m; Mobile Phase A: CO₂, Mobile Phase B: MeOH; Flow rate: 200 mL/min; Gradient: isocratic 40% B; Column Temperature (° C.): 35; Back Pressure (bar): 100; Wavelength: 220 nm; RT1(min): 2.61; RT2(min): 3.63; Sample Solvent: MeOH (0.1% 2M NH₃-MeOH) to yield 2.57 g faster peak (undesired isomer) and 3.00 g slower peak (desired isomer) as yellow solids.

For faster peak (undesired isomer): LC-MS: (ESI, m/z): [M+H]⁺=615; ¹H NMR (300 MHz, DMSO-d₆, ppm) δ 9.02 (s, 1H), 8.31 (d, J=7.8 Hz, 1H), 7.58-7.29 (m, 4H), 6.16 (6.23) (s, 1H), 5.09 (d, J=3.6 Hz, 1H), 5.05-4.71 (m, 2H), 4.52-4.38 (m, 1H), 4.25 (s, 1H), 4.19-4.04 (m, 2H), 3.83-3.39 (m, 7H), 2.44 (d, J=3.4 Hz, 3H), 2.32-1.74 (m, 3H), 1.54-1.30 (m, 3H), 1.23-1.09 (m, 6H), 1.08-0.73 (m, 6H).

For slower peak (desired isomer): LC-MS: (ESI, m/z): [M+H]⁺=615; ¹H NMR (300 MHz, DMSO-d₆, ppm) δ 8.97 (d, J=2.6 Hz, 1H), 8.40 (8.80) (d, J=7.7 Hz, 1H), 7.50-7.26 (m, 4H), 6.12 (5.94) (s, 1H), 5.08 (d, J=3.3 Hz, 1H), 4.95-4.80 (m, 1H), 4.79 (t, J=5.2 Hz, 1H), 4.36 (t, J=7.8 Hz, 1H), 4.26 (s, 1H), 4.08 (d, J=5.2 Hz, 2H), 3.73-3.36 (m, 7H), 2.44 (d, J=1.5 Hz, 3H), 2.29-1.96 (m, 2H), 1.80-1.65 (m, 1H), 1.50-1.30 (m, 3H), 1.20-1.05 (m, 6H), 1.05-0.90 (m, 3H), 0.85-0.70 (m, 3H).

### Step 4: (2S,4R)-4-Hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide

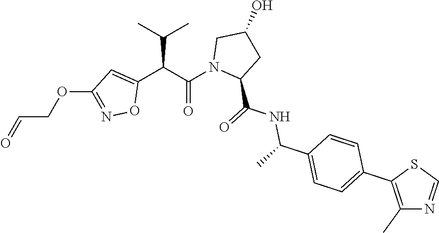 [SMILES: Cc1ncsc1-c1ccc([C@H](C)NC(=O)[C@@H]2C[C@@H](O)CN2C(=O)[C@@H](c2cc(OCC=O)no2)C(C)C)cc1]

A solution of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (300 mg, 0.490 mmol) in tetrahydrofuran (3 mL) and 1 M H₂SO₄ (3 mL) was stirred at 50° C. for 2 h. The solution was cooled and the pH was adjusted to 8 by saturated sodium bicarbonate solution. The product was extracted with ethyl acetate. The combined organic layer was washed with brine, dried over anhydrous sodium sulfate and concentrated under vacuum to yield 260 mg (crude) of the title compound as a white solid. LC-MS: (ESI, m/z): [M+H]⁺=541.

### Example 4

### Syntheses of a Portion of a Hydrolysable Linker

### Intermediate 4: 1-(5-Aminopentyl)-1H-pyrrole-2,5-dione (2,2,2-trifluoroacetic acid)

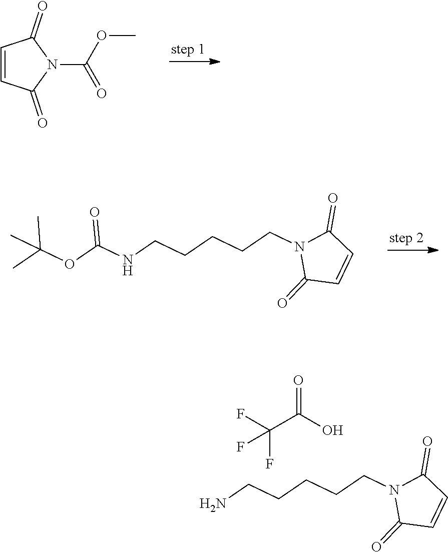

### Step 1: tert-Butyl (5-(2,5-dioxo-2,5-dihydro-1H-pyrrol-1-yl)pentyl)carbamate

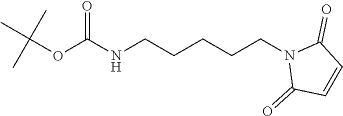 [SMILES: CC(C)(C)OC(=O)NCCCCCN1C(=O)C=CC1=O]

Under nitrogen, to a solution of tert-butyl (5-aminopentyl)carbamate (2.60 g, 12.8 mmol) in dioxane (20 mL) and saturated NaHCO₃ (64 mL) was added methyl 2,5-dioxo-2,5-dihydro-1H-pyrrole-1-carboxylate (2.00 g, 12.9 mmol) at 0° C. The solution was stirred at 0° C. for 40 min. Then the reaction mixture was stirred at room temperature for 1 hour. The reaction was extracted with ethyl acetate. The organic layer was dried over anhydrous sodium sulfate and concentrated under vacuum. The residue was purified by flash chromatography on silica gel (gradient: 0%-30% EtOAc/petroleum ether) to yield 2.00 g (54% yield) of the title compound as a brown oil. LC-MS: (ESI, m/z): [M+H]⁺=283.

### Step 2: 1-(5-Aminopentyl)-1H-pyrrole-2,5-dione (2,2,2-trifluoroacetic acid)

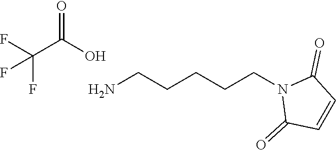

Under nitrogen, a solution of tert-butyl (5-(2,5-dioxo-2,5-dihydro-1H-pyrrol-1-yl)pentyl)carbamate (200 mg, 0.709 mmol) in TFA (0.5 mL) and dichloromethane (2 mL) was stirred at room temperature for 30 min. The resulting mixture was concentrated under vacuum to yield 270 mg (crude) of the title compound as a yellow oil. LC-MS: (ESI, m/z): [M+H]⁺=183.

### Example 5

### Synthesis of a Portion of a Hydrolysable Linker

### Intermediate 5: Ethyl (S)-1-((6-(((allyloxy)carbonyl)amino)-1-((4-(hydroxymethyl)phenyl)amino)-1-oxohexan-2-yl)carbamoyl)cyclobutane-1-carboxylate

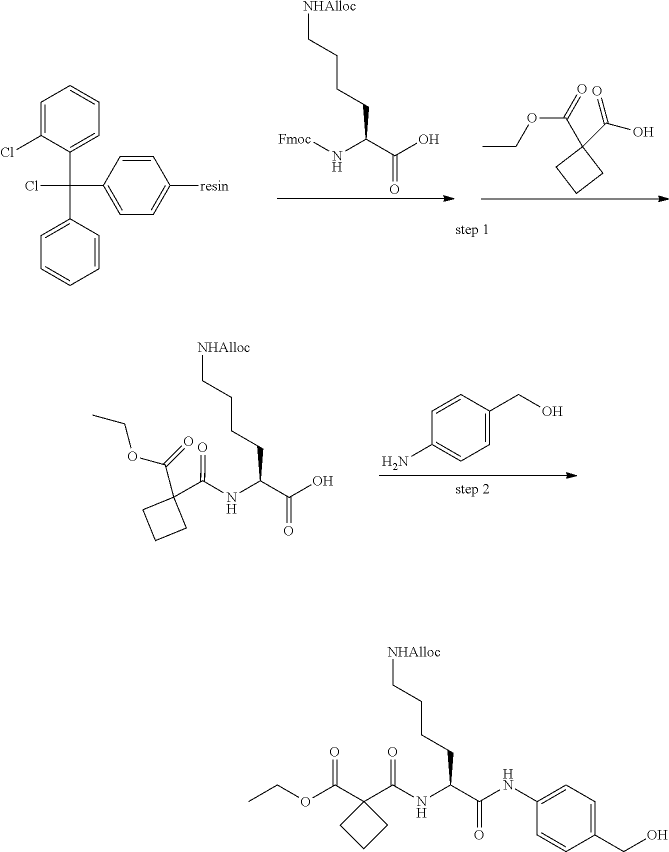

### Step 1: N⁶-((allyloxy)carbonyl)-N²-(1-(ethoxycarbonyl)cyclobutane-1-carbonyl)-L-lysine

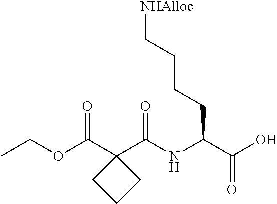

A solution of N²-(((9H-fluoren-9-yl)methoxy)carbonyl)-N⁶-((allyloxy)carbonyl)-L-lysine (900 mg, 1.98 mmol) and DIPEA (1 g, 7.75 mmol) in DCM (10 mL) and DMF (10 mL) was added to 2-chlorotrityl chloride resin (2 g). The mixture was agitated at room temperature for 1 h. 5 mL MeOH was added and stirred at room temperature for 0.5 h. DMF was used to wash the resin for three times. A solution of piperidine (4 mL) and DMF (16 mL) was added and agitated at room temperature for 0.5 h. Then DMF was used to wash for four times. Then, to a solution of 1-ethoxycarbonylcyclobutanecarboxylic acid (514 mg, 2.98 mmol) and DIPEA (771 mg, 5.97 mmol) in DMF (15 mL) was added HATU (1.1 g, 2.89 mmol) and HOBt (454 mg, 2.98 mmol) at 0° C. and stirred at room temperature for 0.5 h. The solution was added into the above resin and stirred at room temperature for 1 h. DMF was used to wash the resin for three times followed by DCM. The resin was agitated in a solution of HFIP (4 mL) and DCM (16 mL) for 1 h. The resin was filtered and filtrate was concentrated to yield 900 mg (crude) of the title compound as a yellow oil. LCMS (ESI, m/z) [M+H]⁺=385.

### Step 2: Ethyl (S)-1-((6-(((allyloxy)carbonyl)amino)-1-((4-(hydroxymethyl)phenyl)amino)-1-oxohexan-2-yl)carbamoyl)cyclobutane-1-carboxylate

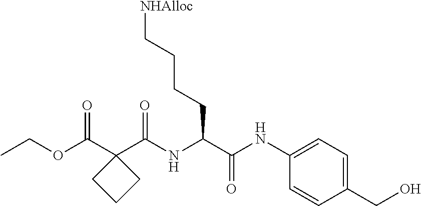

A solution of N⁶-((allyloxy)carbonyl)-N²-(1-(ethoxycarbonyl)cyclobutane-1-carbonyl)-L-lysine (4.2 g, 10.96 mmol), (4-aminophenyl)methanol (1.48 g, 11.9 mmol), EEDQ (5.41 g, 21.8 mmol) in DCM (40 mL) and MeOH (20 mL) was stirred at 30° C. for 1 h. Solvent was evaporated and crude was purified by flash chromatography on silica gel (solvent gradient: 0-10% MeOH in DCM) to yield 2.6 g (49% yield) of the title compound as a yellow oil. LCMS (ESI, m/z) [M+H]⁺=490.

### Example 6

### Synthesis of a Portions of a Linker L2

### Intermediate 6: tert-Butyl 4-((1r,3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate

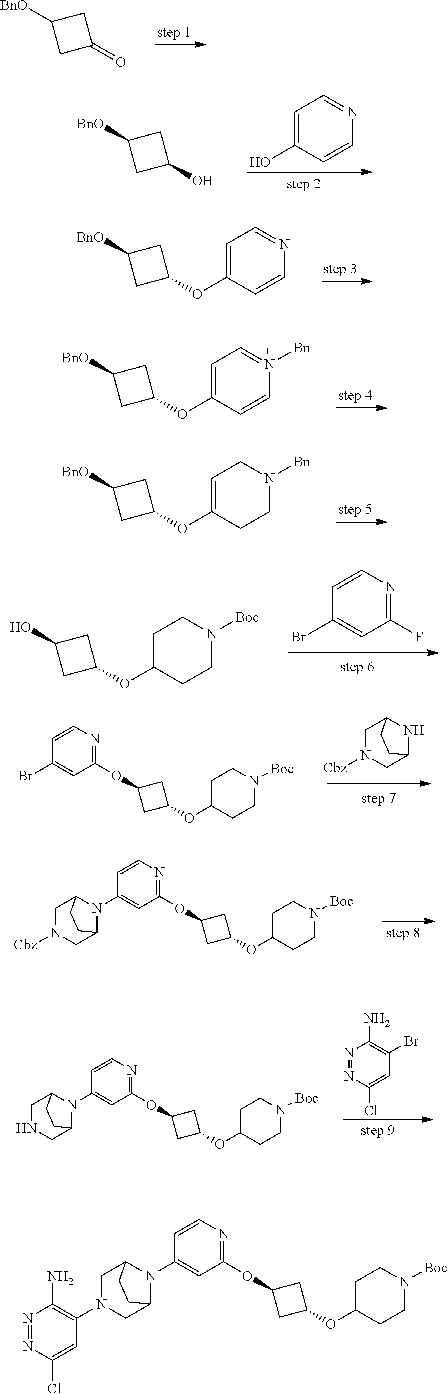

### Step 1: (1s,3s)-3-(Benzyloxy)cyclobutan-1-ol

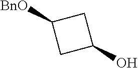 [SMILES: O[C@H]1C[C@@H](OCc2ccccc2)C1]

Under nitrogen, to a solution of 3-(benzyloxy)cyclobutan-1-one (10.0 g, 56.8 mmol) in methanol (100 mL) was added NaBH₄ (2.15 g, 56.9 mmol) at 0° C. The resulting solution was stirred at 0° C. for 1 h. The reaction was quenched with water and extracted with EtOAc. The organic layers were combined, dried over anhydrous sodium sulfate and concentrated under vacuum. The residue was purified by flash chromatography on silica gel (solvent gradient: 0-50% ethyl acetate/petroleum ether) to yield 9.85 g (97.4% yield) of the title compound as a colorless oil. LC-MS: (ESI, m/z): [M+H]⁺=179.

### Step 2: 4-((1r,3r)-3-(benzyloxy)cyclobutoxy)pyridine

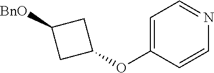 [SMILES: c1ccc(CO[C@H]2C[C@H](Oc3ccncc3)C2)cc1]

Under nitrogen, to a solution of (1s,3s)-3-(benzyloxy)cyclobutan-1-ol (11.0 g, 61.7 mmol), pyridin-4-ol (8.80 g, 92.5 mmol) and PPh₃ (19.4 g, 74.2 mmol) in tetrahydrofuran (110 mL) was added DIAD (14.9 g, 74.1 mmol) at 0° C. The reaction was stirred at 50° C. for 2 h. The organic layer was concentrated under vacuum. The resulting solution was diluted with water and extracted with dichloromethane. The organic layers were combined, dried over anhydrous sodium sulfate and concentrated under vacuum. The residue was purified by flash chromatography on silica gel (solvent gradient: 0-50% ethyl acetate/petroleum ether) to yield 15.0 g (crude) of the title compound as a yellow oil. LC-MS: (ESI, m/z): [M+H]⁺=256.

### Step 3: 1-Benzyl-4-((1r,3r)-3-(benzyloxy)cyclobutoxy)pyridin-1-ium

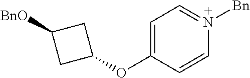 [SMILES: c1ccc(CO[C@H]2C[C@H](Oc3cc[n+](Cc4ccccc4)cc3)C2)cc1]

Under nitrogen, a solution of 4-((1r,3r)-3-(Benzyloxy)cyclobutoxy)pyridine (7.00 g, crude) and BnBr (4.69 g, 27.4 mmol) in toluene (70 mL) was stirred at 80° C. overnight. The solution was concentrated under vacuum. Petroleum ether was added and the solution was stirred until there are white solids precipitated. After filtration, the solid was collected to afford 7.86 g of the title compound as a white solid. LC-MS: (ESI, m/z): [M]⁺=346.

### Step 4: 1-Benzyl-4-((1r,3r)-3-(benzyloxy)cyclobutoxy)-1,2,3,6-tetrahydropyridine

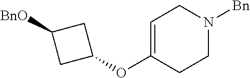 [SMILES: C1=C(O[C@H]2C[C@H](OCc3ccccc3)C2)CCN(Cc2ccccc2)C1]

Under nitrogen, to a solution of 1-benzyl-4-((1r,3r)-3-(benzyloxy)cyclobutoxy)pyridin-1-ium (7.50 g, 21.7 mmol) in ethanol (75 mL) was added NaBH₄ (4.90 g, 129 mmol) at 0° C. The resulting solution was stirred at room temperature for 1 h. The resulting solution was diluted with ethyl acetate and washed with water. The organic layer was dried over anhydrous sodium sulfate and concentrated under vacuum. The residue was purified by flash chromatography on silica gel (gradient: 0-100% ethyl acetate/petroleum ether) to yield 4.13 g (55.1% yield) of the title compound as a yellow oil. LC-MS: (ESI, m/z): [M+H]⁺=350.

### Step 5: tert-Butyl 4-((1r,3r)-3-hydroxycyclobutoxy)piperidine-1-carboxylate

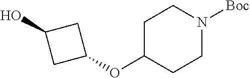 [SMILES: CC(C)(C)OC(=O)N1CCC(O[C@H]2C[C@H](O)C2)CC1]

Under hydrogen (3 atm), a solution of 1-benzyl-4-((1r,3r)-3-(benzyloxy)cyclobutoxy)-1,2,3,6-tetrahydropyridine (4.00 g, 11.4 mmol), Boc₂O (7.49 g, 34.3 mmol), 20% Pb(OH)₂/C (800 mg) and 10% Pd/C (800 mg) in methanol (400 mL) was stirred at room temperature for 48 h. The reaction mixture was filtered, the filtrate was concentrated under vacuum. The residue was purified by flash chromatography on silica gel (solvent gradient: 0-100% ethyl acetate/petroleum ether) to yield 900 mg (28.9% yield) of the title compound as a white solid. LC-MS: (ESI, m/z): [M+H]⁺=272.

### Step 6: tert-Butyl 4-((1r,3r)-3-((4-bromopyridin-2-yl)oxy)cyclobutoxy)piperidine-1-carboxylate

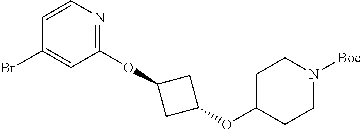 [SMILES: CC(C)(C)OC(=O)N1CCC(O[C@H]2C[C@H](Oc3cc(Br)ccn3)C2)CC1]

Under nitrogen, a solution of tert-butyl 4-((1r,3r)-3-hydroxycyclobutoxy)piperidine-1-carboxylate (3.27 g, 12.0 mmol), 4-bromo-2-fluoro-pyridine (3.92 g, 22.4 mmol) and Cs₂CO₃ (7.81 g, 24.0 mmol) in ACN (150 mL) was stirred at 90° C. for 12 h. The solids were filtered and the filtrate was concentrated under vacuum. The residue was purified by flash chromatography on silica gel (solvent gradient: 0-30% ethyl acetate/petroleum ether) to yield 4.50 g (87% yield) of the title compound as a white solid. LC-MS: (ESI, m/z): [M+H]⁺=427.

### Step 7: Benzyl 8-(2-((1r,3r)-3-((1-(tert-butoxycarbonyl)piperidin-4-yl)oxy)cyclobutoxy)pyridin-4-yl)-3,8-diazabicyclo[3.2.1]octane-3-carboxylate

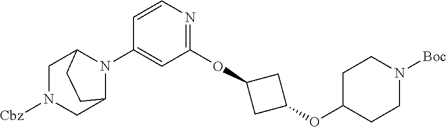 [SMILES: CC(C)(C)OC(=O)N1CCC(O[C@H]2C[C@H](Oc3cc(N4C5CCC4CN(C(=O)OCc4ccccc4)C5)ccn3)C2)CC1]

Under nitrogen, a solution of tert-butyl 4-((1r,3r)-3-((4-bromopyridin-2-yl)oxy)cyclobutoxy)piperidine-1-carboxylate (4.50 g, 10.5 mmol), benzyl 3,8-diazabicyclo[3.2.1]octane-3-carboxylate (3.23 g, 13.1 mmol), Cs₂CO₃ (16.7 g, 51.6 mmol) and Ruphos Pd G2 (1.73 g, 2.22 mmol) in toluene (300 mL) was stirred at 90° C. for 12 h. The solution was concentrated under vacuum. The residue was purified by flash chromatography on silica gel (solvent gradient: 0-30% ethyl acetate/petroleum ether) to yield 4.70 g (75.2% yield) of the title compound as a white solid. LC-MS: (ESI, m/z): [M+H]⁺=593.

### Step 8: tert-Butyl 4-((1r,3r)-3-((4-(3,8-diazabicyclo[3.2.1]octan-8-yl)pyridin-2-yl)oxy)cyclobutoxy)piperidine-1-carboxylate

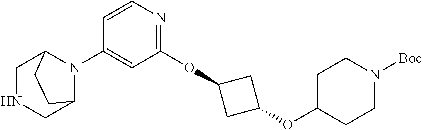 [SMILES: CC(C)(C)OC(=O)N1CCC(O[C@H]2C[C@H](Oc3cc(N4C5CCC4CNC5)ccn3)C2)CC1]

Under hydrogen (3 atm), a solution of benzyl 8-(2-((1r,3r)-3-((1-(tert-butoxycarbonyl)piperidin-4-yl)oxy)cyclobutoxy)pyridin-4-yl)-3,8-diazabicyclo[3.2.1]octane-3-carboxylate (260 mg, 0.438 mmol), 20% Pb(OH)₂/C (61.6 mg) in ethanol (40 mL) and THF (40 ml) was stirred at 60° C. for 3 h. After filtration, the filtrate was collected and concentrated under vacuum to afford 220 mg (crude) of the title compound as a black solid. LC-MS: (ESI, m/z): [M+H]⁺=459.

### Step 9: tert-Butyl 4-((1r,3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate

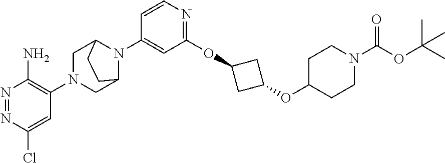 [SMILES: CC(C)(C)OC(=O)N1CCC(O[C@H]2C[C@H](Oc3cc(N4C5CCC4CN(c4cc(Cl)nnc4N)C5)ccn3)C2)CC1]

Under nitrogen, a solution of 4-bromo-6-chloro-pyridazin-3-amine (0.60 g, 2.98 mmol), tert-butyl 4-((1r,3r)-3-((4-(3,8-diazabicyclo[3.2.1]octan-8-yl)pyridin-2-yl)oxy)cyclobutoxy)piperidine-1-carboxylate (1.10 g, 2.40 mmol) and DIPEA (3.10 g, 24.0 mmol) in DMSO (15 mL) was stirred at 130° C. for 18 h. The reaction mixture was diluted with ethyl acetate and washed with water. The organic layer was dried over anhydrous sodium sulfate and concentrated under vacuum. The residue was purified by pre-packed C18 column (solvent gradient: 0-100% ACN in water (0.05% NH₃·H₂O)) to yield 1.10 g (78% yield) of the title compound as a white solid. LC-MS: (ESI, m/z): [M+H]⁺=586. ¹H NMR (300 MHz, DMSO-d₆, ppm) δ 7.76 (d, J=6.0 Hz, 1H), 6.90 (s, 1H), 6.50 (dd, J=6.1, 2.1 Hz, 1H), 6.12 (d, J=2.0 Hz, 1H), 5.85 (s, 2H), 5.26-5.12 (m, 1H), 4.45 (s, 2H), 4.31 (p, J=6.0 Hz, 1H), 3.66 (dt, J=13.4, 4.7 Hz, 2H), 3.50-3.37 (m, 1H), 3.18 (d, J=11.5 Hz, 2H), 2.92 (dd, J=35.6, 11.5 Hz, 4H), 2.40-2.21 (m, 4H), 2.13 (d, J=7.2 Hz, 2H), 1.97-1.88 (m, 2H), 1.80-1.70 (m, 2H), 1.39 (s, 9H), 1.33-1.20 (m, 2H).

### Example 7

### Synthesis of Portions of a Linker L2 and a PB

### Intermediate 7: tert-Butyl (3R)-4-(2-((4-(3-(3-amino-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate

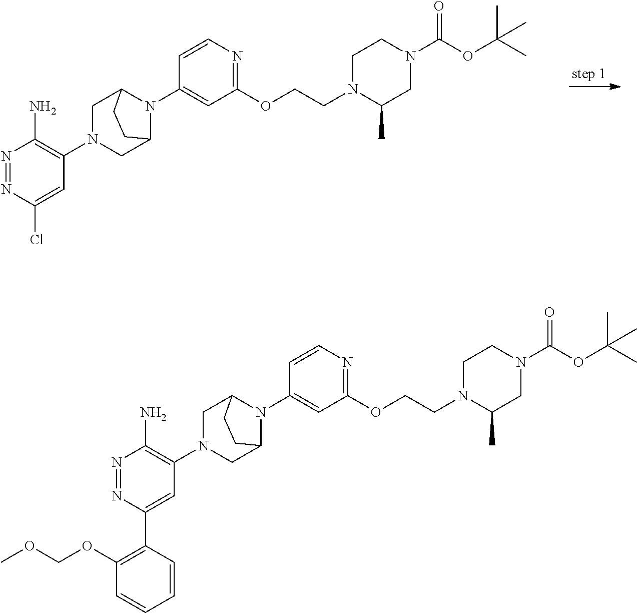

### Step 1: tert-Butyl (3R)-4-(2-((4-(3-(3-amino-6-(2-(methoxymethoxy)phenyl)pyridazin-4-yl)-3,8-diazabicyclo[3.2.1]octan-8-yl)pyridin-2-yl)oxy)ethyl)-3-methylpiperazine-1-carboxylate

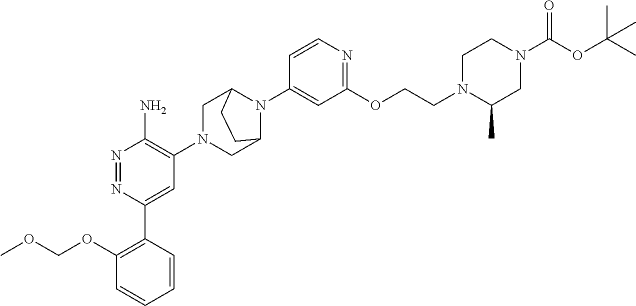 [SMILES: COCOc1ccccc1-c1cc(N2CC3CCC(C2)N3c2ccnc(OCCN3CCN(C(=O)OC(C)(C)C)C[C@H]3C)c2)c(N)nn1]

Under nitrogen, a solution of tert-butyl (3R)-4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (1.00 g, 1.79 mmol, provided by Genentech), (2-(methoxymethoxy)phenyl)boronic acid (391 mg, 2.15 mmol), Pd(PPh₃)₄ (413 mg, 0.358 mmol) and K₂CO₃ (741 mg, 5.37 mmol) in dioxane (10 mL) and water (2 mL) was stirred at 100° C. for 1 hour. The reaction was diluted with water and extracted with dichloromethane. The organic layers were combined, dried over anhydrous sodium sulfate and concentrated under vacuum. The residue was purified by flash chromatography on silica gel (solvent gradient: 0%-10% MeOH/DCM) to yield 670 mg (57% yield) of the title compound as a yellow solid. LC-MS: (ESI, m/z): [M+H]⁺=661. ¹H NMR (300 MHz, DMSO-d₆, ppm) δ 7.76 (d, J=5.9 Hz, 1H), 7.58 (dd, J=7.6, 1.8 Hz, 1H), 7.34 (ddd, J=9.0, 7.3, 1.8 Hz, 1H), 7.18-7.01 (m, 3H), 6.51 (dd, J=6.1, 2.0 Hz, 1H), 6.12 (d, J=2.0 Hz, 1H), 5.72 (s, 2H), 5.14 (s, 2H), 4.47 (s, 2H), 4.25 (t, J=6.1 Hz, 2H), 3.53 (d, J=12.8 Hz, 2H), 3.22 (s, 3H), 3.14-2.67 (m, 8H), 2.64-2.56 (m, 1H), 2.46-2.36 (m, 1H), 2.32-2.22 (m, 1H), 2.22-2.13 (m, 2H), 2.00-1.90 (m, 2H), 1.38 (s, 9H), 0.96 (d, J=6.2 Hz, 3H).

### Example 8

### Synthesis of Portions of a Hydrolysable Linker, a PB and a Linker L2

### Intermediate 8: 1-(((2S)-1-((4-((2-(6-Amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

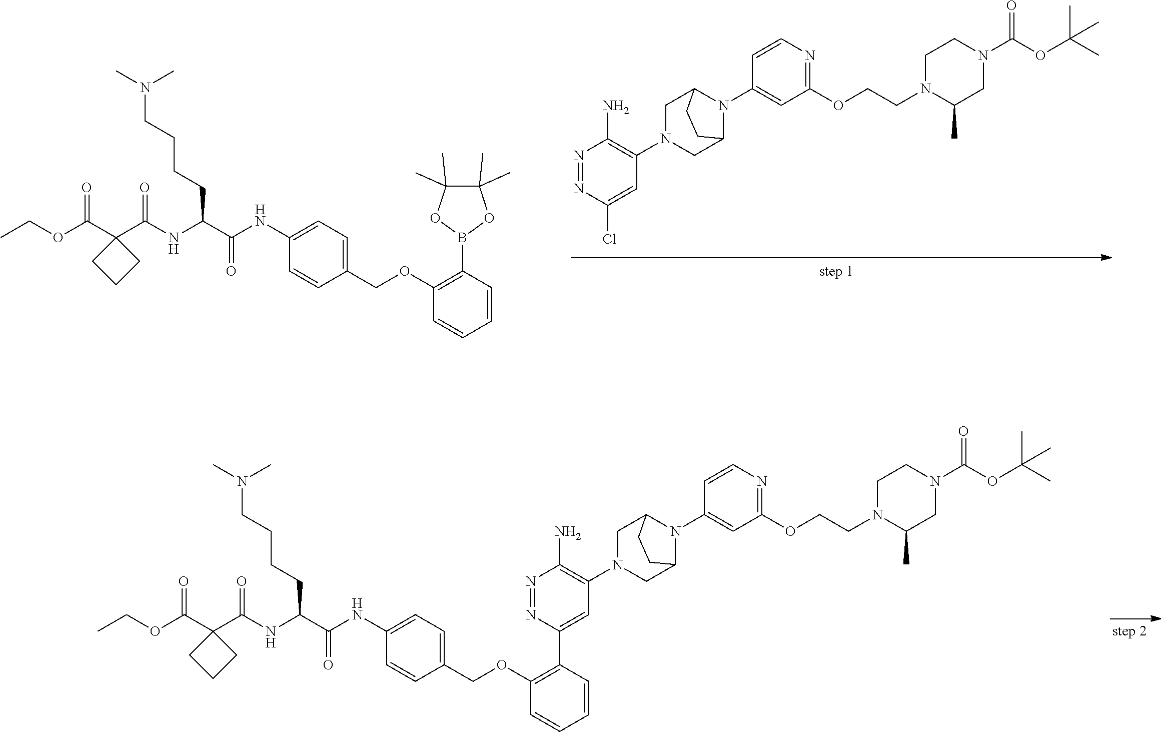 [SMILES: CCOC(=O)C1(C(=O)N[C@@H](CCCCN(C)C)C(=O)Nc2ccc(COc3ccccc3B3OC(C)(C)C(C)(C)O3)cc2)CCC1 ; C[C@@H]1CN(C(=O)OC(C)(C)C)CCN1CCOc1cc(N2C3CCC2CN(c2cc(Cl)nnc2N)C3)ccn1 ; CCOC(=O)C1(C(=O)N[C@@H](CCCCN(C)C)C(=O)Nc2ccc(COc3ccccc3-c3cc(N4CC5CCC(C4)N5c4ccnc(OCCN5CCN(C(=O)OC(C)(C)C)C[C@H]5C)c4)c(N)nn3)cc2)CCC1]

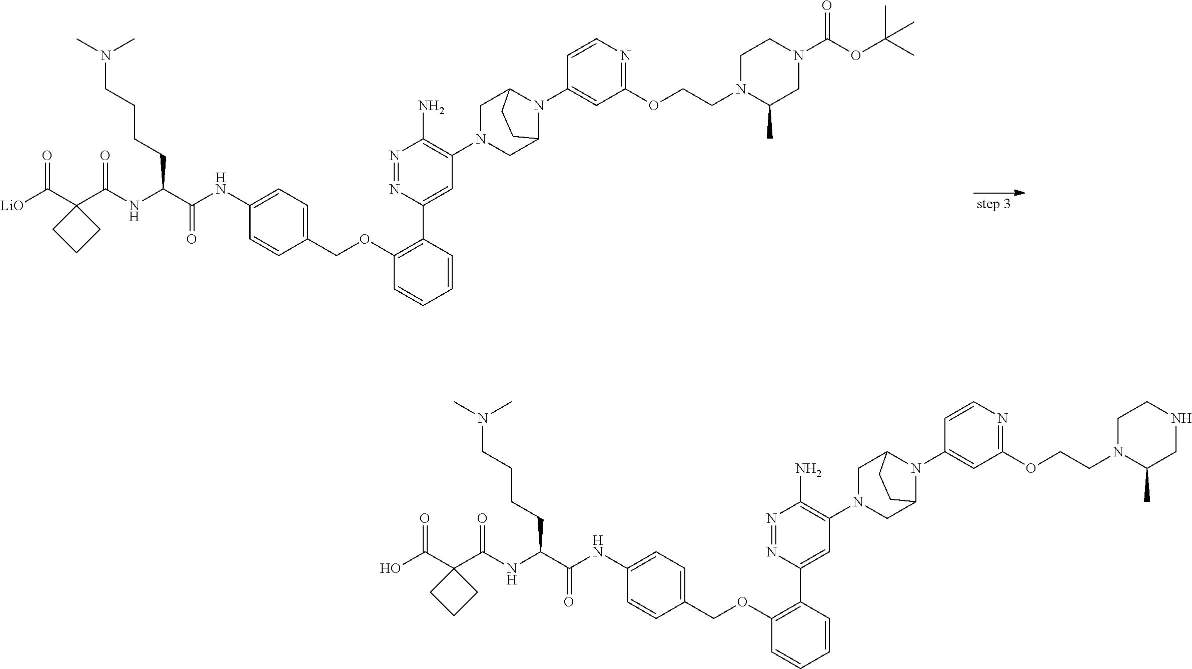

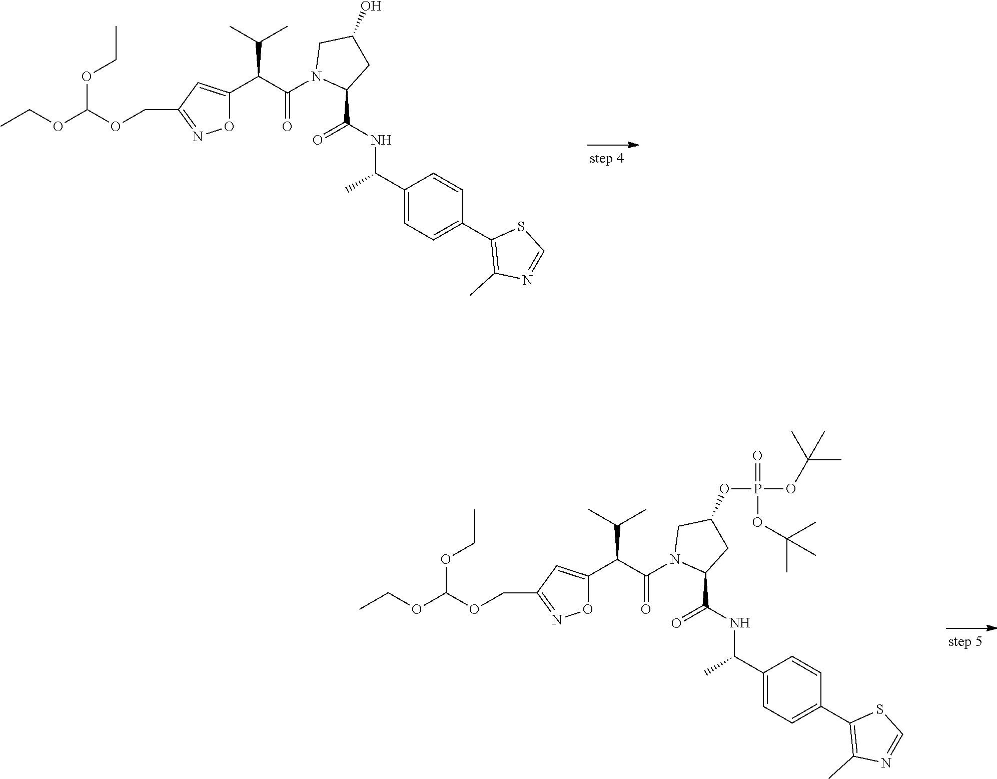 [SMILES: CCOC(OCC)OCc1cc([C@H](C(=O)N2C[C@H](O)C[C@H]2C(=O)N[C@@H](C)c2ccc(-c3scnc3C)cc2)C(C)C)on1 ; CCOC(OCC)OCc1cc([C@H](C(=O)N2C[C@H](OP(=O)(OC(C)(C)C)OC(C)(C)C)C[C@H]2C(=O)N[C@@H](C)c2ccc(-c3scnc3C)cc2)C(C)C)on1]

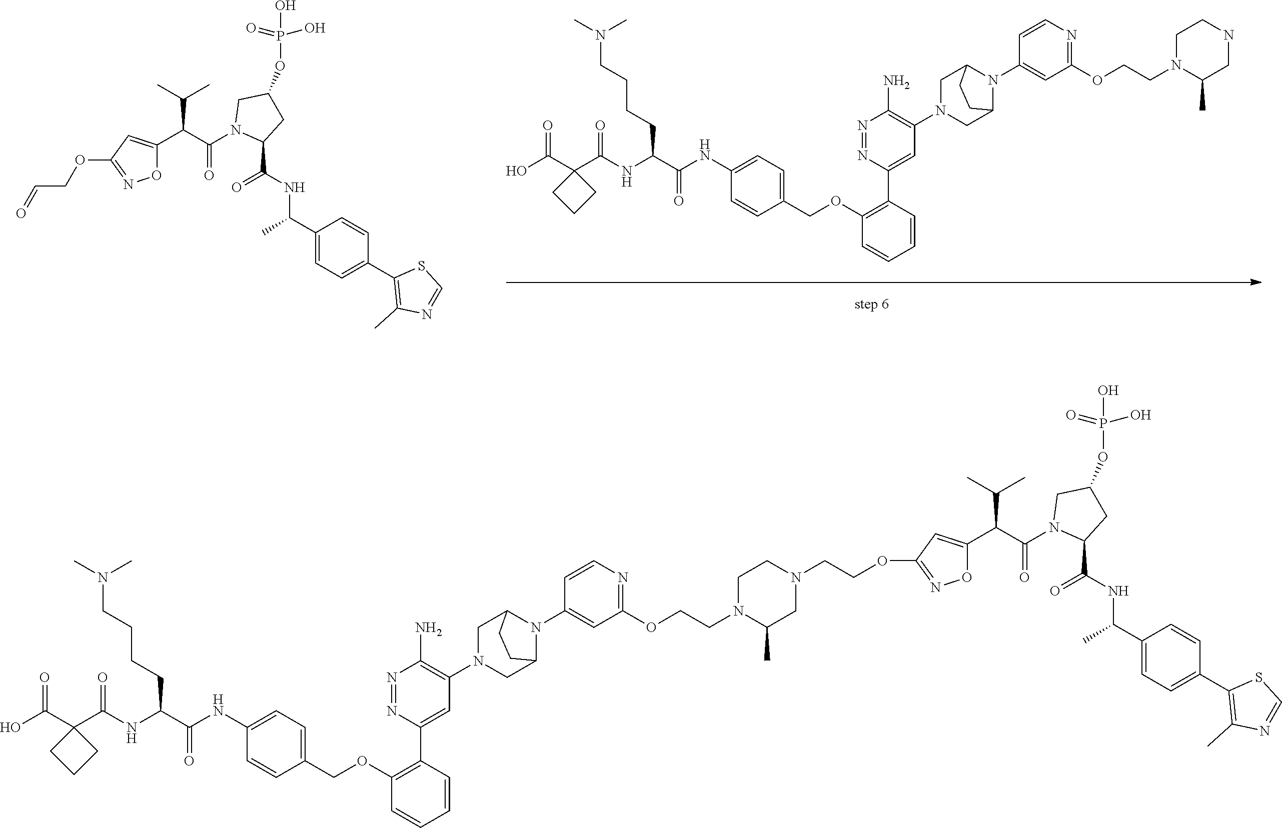 [SMILES: Cc1ncsc1-c1ccc([C@H](C)NC(=O)[C@@H]2C[C@@H](OP(=O)(O)O)CN2C(=O)[C@@H](c2cc(OCC=O)no2)C(C)C)cc1 ; C[C@@H]1CNCCN1CCOc1cc(N2C3CCC2CN(c2cc(-c4ccccc4OCc4ccc(NC(=O)[C@H](CCCCN(C)C)NC(=O)C5(C(=O)O)CCC5)cc4)nnc2N)C3)ccn1 ; Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)[C@H](C)C3)no2)C(C)C)cc1]

### Step 1: tert-Butyl (3R)-4-(2-((4-(3-(3-amino-6-(2-((4-((S′)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate

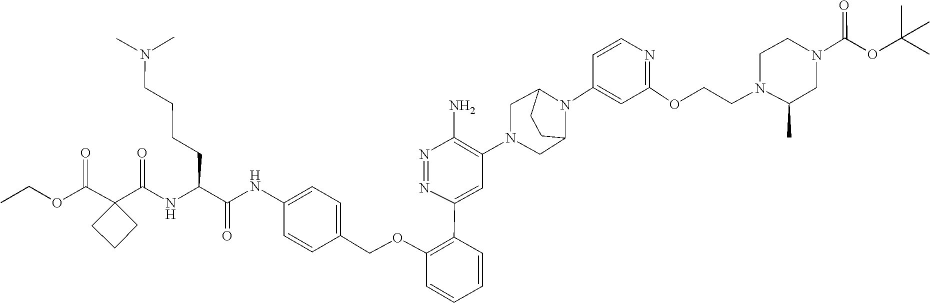 [SMILES: CCOC(=O)C1(C(=O)N[C@@H](CCCCN(C)C)C(=O)Nc2ccc(COc3ccccc3-c3cc(N4CC5CCC(C4)N5c4ccnc(OCCN5CCN(C(=O)OC(C)(C)C)C[C@H]5C)c4)c(N)nn3)cc2)CCC1]

Under nitrogen, a solution of ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate (316 mg, 0.570 mmol), tert-butyl (3R)-4-(2-((4-(3-(3-amino-6-chloropyridazin-4-yl)-3,8-diazabicyclo[3.2.1]octan-8-yl)pyridin-2-yl)oxy)ethyl)-3-methylpiperazine-1-carboxylate (538 mg, 0.850 mmol), K₃PO₄ (240 mg, 1.13 mmol) and Ad₂nBuPPdG₂ (37.8 mg, 0.0600 mmol) in 1,4-dioxane (4 mL) and water (1 mL) was stirred at 95° C. for 3 h. Water was added and EtOAc was used to extract for three times. The organic solvent was combined and concentrated under vacuum. The residue was purified by pre-packed C18 column (solvent gradient: 0-100% ACN in water (0.05% NH₄HCO₃)) to afford 230 mg (39% yield) of the title compound as a red solid. LCMS (ESI) [M+H]⁺=1032.

### Step 2: Lithium 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate

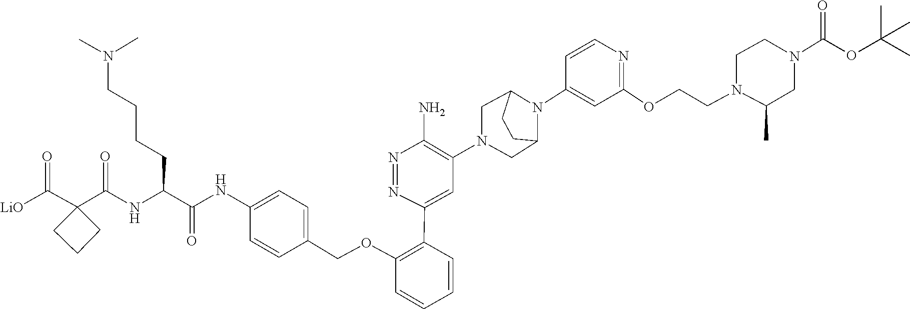

A solution of tert-butyl (3R)-4-(2-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazine-1-carboxylate (210 mg, 0.200 mmol) and LiOH·H₂O (25.6 mg, 0.610 mmol) in tetrahydrofuran (3 mL) and water (1 mL) was stirred at room temperature for 1 hour. The solvent was concentrated under vacuum to afford 254 mg (crude) of the title compound as a yellow solid.

### Step 3: 1-(((2S)-1-((4-((2-(6-Amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

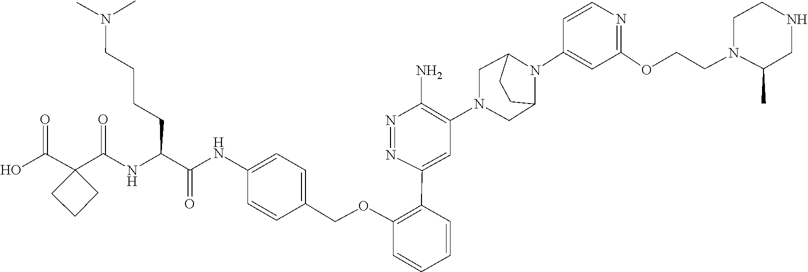 [SMILES: C[C@@H]1CNCCN1CCOc1cc(N2C3CCC2CN(c2cc(-c4ccccc4OCc4ccc(NC(=O)[C@H](CCCCN(C)C)NC(=O)C5(C(=O)O)CCC5)cc4)nnc2N)C3)ccn1]

A solution of lithium 1-(((2S)-1-((4-((2-(6-amino-5-(8-(2-(2-((R)-4-(tert-butoxycarbonyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate (254 mg, 0.250 mmol) in 5% TFA/HFIP (20 mL) was stirred at room temperature for 3 h. The solvent was concentrated under vacuum. The residue was purified by pre-packed C18 column (solvent gradient: 0-100% MeOH in water (0.05% NH₄HCO₃)) to yield 102 mg (44% yield) of the title compound as a red solid. LCMS (ESI, m/z) [M+H]⁺=905.

### Step 4: Di-tert-butyl ((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate

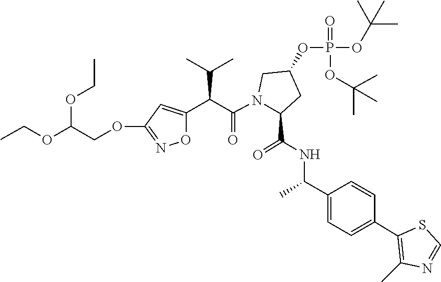 [SMILES: CCOC(COc1cc([C@H](C(=O)N2C[C@H](OP(=O)(OC(C)(C)C)OC(C)(C)C)C[C@H]2C(=O)N[C@@H](C)c2ccc(-c3scnc3C)cc2)C(C)C)on1)OCC]

Under nitrogen, to a solution of (2S,4R)-1-((R)-2-(3-(2,2-diethoxyethoxy)isoxazol-5-yl)-3-methylbutanoyl)-4-hydroxy-N—((S)-1-(4-(4-methylthiazol-5-yl)phenyl)ethyl)pyrrolidine-2-carboxamide (500 mg, 0.81 mmol) and 1H-tetrazole (5.4 mL, 0.45 M in ACN) in THF (5 mL) was added di-tert-butyl diisopropylphosphoramidite (451 mg, 1.63 mmol) dropwise at 0° C. The reaction was stirred at room temperature overnight. Then tBuOOH (0.4 mL, 5 M in decane) was added at −30° C. and stirred at −30° C. for 30 min. Then the mixture was stirred at room temperature for 4 hours. The reaction was quenched with aqueous NaHSO₃ and extracted with EtOAc. The combined organic layer was washed with water and brine. The organic layer was dried over anhydrous sodium sulfate and concentrated under vacuum. The residue was purified by pre-packed C18 column (solvent gradient: 0-70% ACN in water (0.05% NH₄HCO₃)) to yield 340 mg (51%) of the title compound as a yellow oil. LC-MS: (ESI, m/z): [M+H]⁺=807.

### Step 5: (3R,5S)-1-((R)-3-Methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate

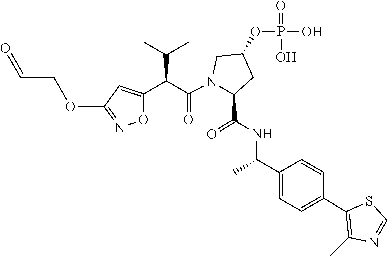 [SMILES: Cc1ncsc1-c1ccc([C@H](C)NC(=O)[C@@H]2C[C@@H](OP(=O)(O)O)CN2C(=O)[C@@H](c2cc(OCC=O)no2)C(C)C)cc1]

Under nitrogen, a solution of di-tert-butyl ((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate (150 mg, 0.185 mmol) in HCOOH (0.5 mL) and water (0.5 mL) was stirred at 60° C. for 1 h. The solution was concentrated under vacuum to afford 121 mg (crude) of the title compound as a solid. LC-MS: (ESI, m/z): [M+H]⁺=695.

### Step 6: 1-(((2S)-1-((4-((2-(6-Amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

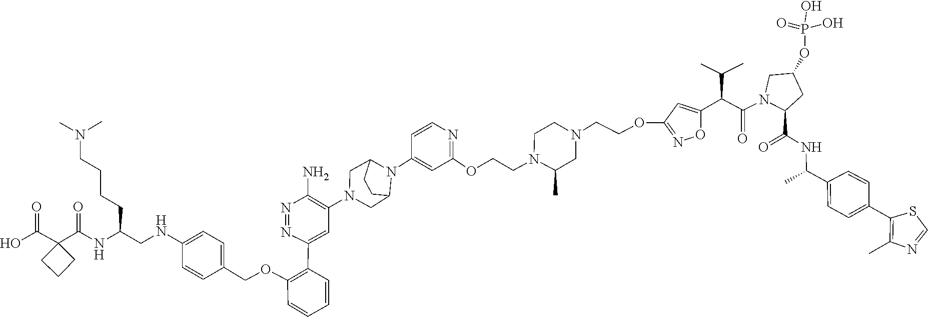 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC[C@H](CCCCN(C)C)NC(=O)C8(C(=O)O)CCC8)cc7)nnc5N)C6)ccn4)[C@H](C)C3)no2)C(C)C)cc1]

A solution of 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (100 mg, 0.110 mmol), (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate (121 mg, 0.190 mmol) and NaOAc (13.6 mg, 0.170 mmol) in methyl alcohol (1.2 mL) and dichloromethane (0.4 mL) was stirred at room temperature for 1 h. Then NaBH₃CN (34.7 mg, 0.550 mmol) was added and stirred at room temperature for 0.5 h. Water was added to quench the reaction. The solvent was concentrated under vacuum. The product was purified by Prep-HPLC (Column: YMC-Actus Triart C18 ExRS, 30*150 mm, 5 m; Mobile Phase A: Water (10 mmol/L NH₄HCO₃), Mobile Phase B: ACN; Flow rate: 60 mL/min; Gradient: 36% B to 60% B in 9 min, 60% B; Wavelength: 254/220 nm; R_{T} (min): 7.63) to afford 30.0 mg (18% yield) of the title compound as a white solid. LCMS (ESI, m/z): [M+H]⁺=1509.

### Example 9

### Synthesis of a Portion of a Hydrolysable Linker

### Intermediate 9: Ethyl (S)-1-((6-(((allyloxy)carbonyl)amino)-1-((4-(hydroxymethyl)phenyl)amino)-1-oxohexan-2-yl)carbamoyl)cyclobutane-1-carboxylate

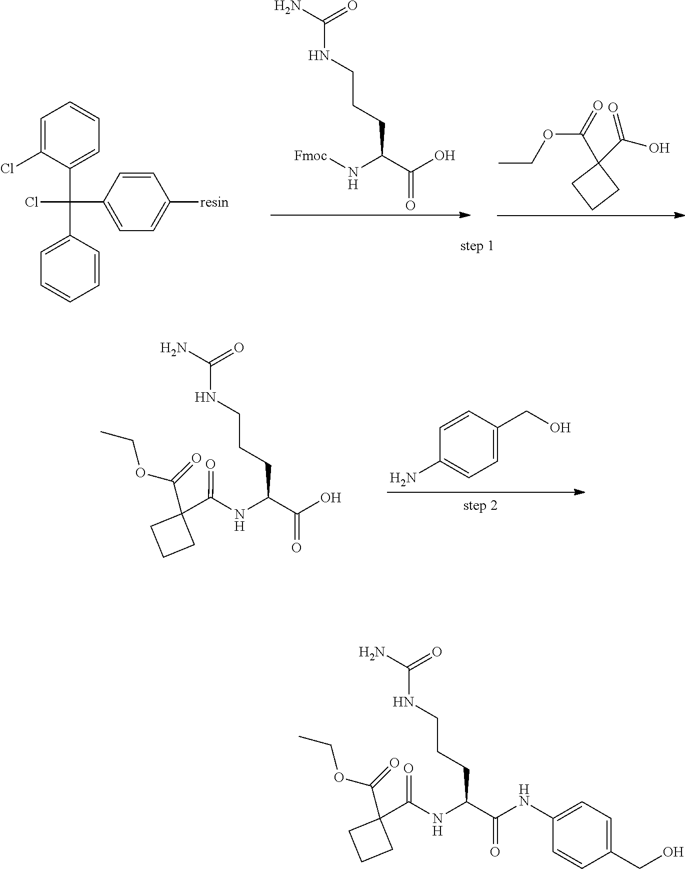

### Step 1: (S)-2-(1-(Ethoxycarbonyl)cyclobutane-1-carboxamido)-5-ureidopentanoic acid

A solution of Fmoc-CIT-OH (4.00 g, 10.0 mmol) and DIPEA (5.20 g, 40.3 mmol) in DCM (40 mL) and DMF (40 mL) was added to 2-chlorotrityl chloride resin (10.0 g). The mixture was agitated for 1 h. 8 mL MeOH was added and the resulting solution was stirred at room temperature for 0.5 h. DMF was used to wash the resin for three times. A solution of piperidine (15 mL) and DMF (60 mL) was added and agitated at room temperature for 0.5 h. Then DMF was used to wash for four times. Then, to a solution of 1-ethoxycarbonylcyclobutanecarboxylic acid (2.60 g, 15.1 mmol) and DIPEA (3.90 g, 30.2 mmol) in DMF (60 mL) was added HATU (5.70 g, 15.0 mmol) and HOBt (2.30 g, 15.1 mmol) at 0° C. and the reaction was stirred at room temperature for 0.5 h. The resulting solution was added into the above resin and stirred at room temperature for 1 h. DMF was used to wash the resin for three times followed by DCM. The resin was agitated in a solution of HFIP (15 mL) and DCM (60 mL) for 1 h. The resin was filtered and filtrate was concentrated to yield 3.7 g (crude) of the title compound as a yellow oil. LCMS (ESI, m/z) [M+H]⁺=329.

### Step 2: Ethyl (S)-1-((1-((4-(hydroxymethyl)phenyl)amino)-1-oxo-5-ureidopentan-2-yl)carbamoyl)cyclobutane-1-carboxylate

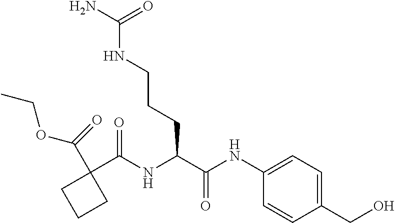 [SMILES: CCOC(=O)C1(C(=O)N[C@@H](CCCNC(N)=O)C(=O)Nc2ccc(CO)cc2)CCC1]

A solution of (S)-2-(1-(ethoxycarbonyl)cyclobutane-1-carboxamido)-5-ureidopentanoic acid (3.20 g, 9.73 mmol), (4-aminophenyl)methanol (1.32 g, 10.6 mmol), EEDQ (4.81 g, 19.4 mmol) in DCM (20 mL) and MeOH (10 mL) was stirred at 30° C. for 2 h. Solvent was evaporated and the crude was purified by flash chromatography on silica gel (solvent gradient: 0-20% MeOH/DCM) to yield 1.75 g of the title compound as a brown oil. LCMS (ESI) [M+H]⁺=434.

### Example 10

### Synthesis of a Portion of a Hydrolysable Linker

### Intermediate 10: 1-(((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

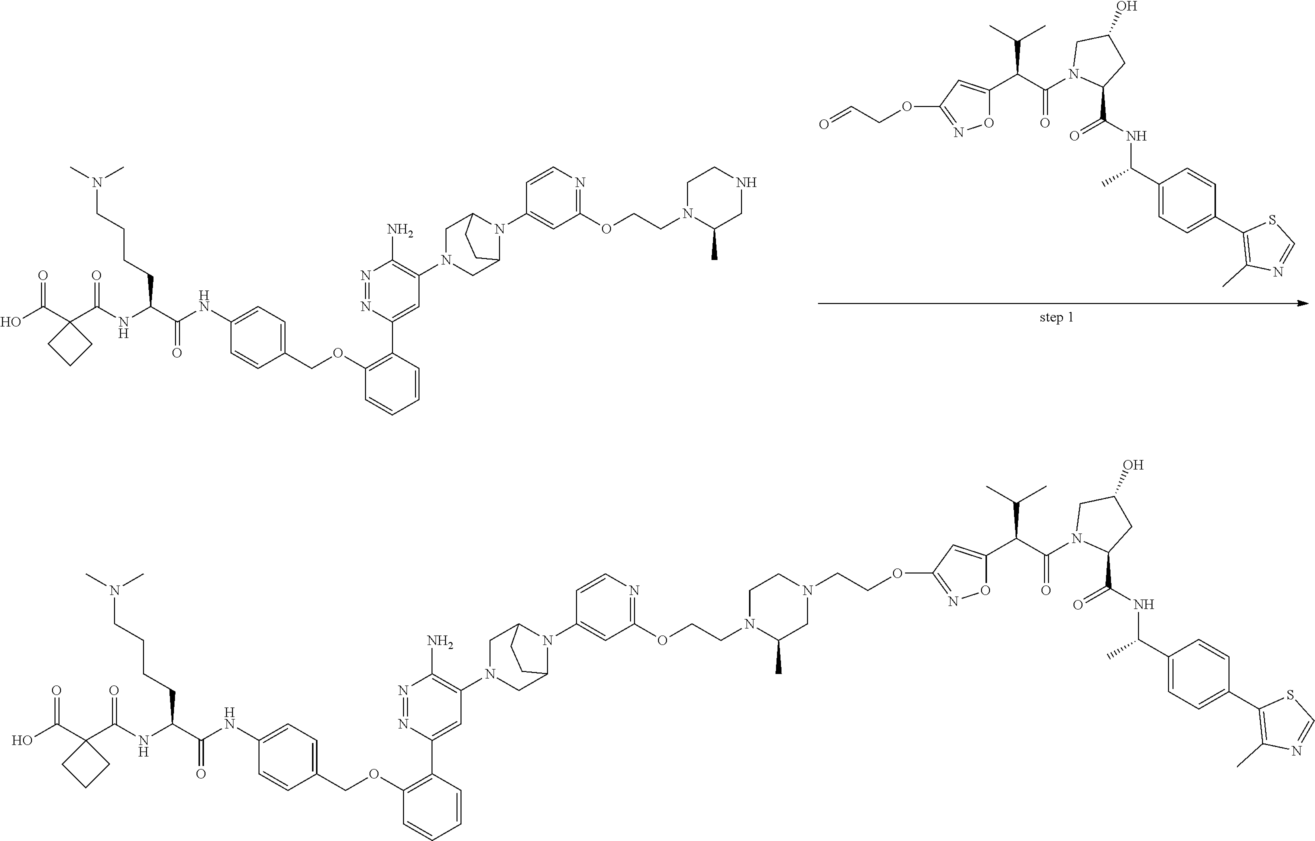 [SMILES: C[C@@H]1CNCCN1CCOc1cc(N2C3CCC2CN(c2cc(-c4ccccc4OCc4ccc(NC(=O)[C@H](CCCCN(C)C)NC(=O)C5(C(=O)O)CCC5)cc4)nnc2N)C3)ccn1 ; Cc1ncsc1-c1ccc([C@H](C)NC(=O)[C@@H]2C[C@@H](O)CN2C(=O)[C@@H](c2cc(OCC=O)no2)C(C)C)cc1 ; Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)[C@H](C)C3)no2)C(C)C)cc1]

### Step 1: 1-(((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

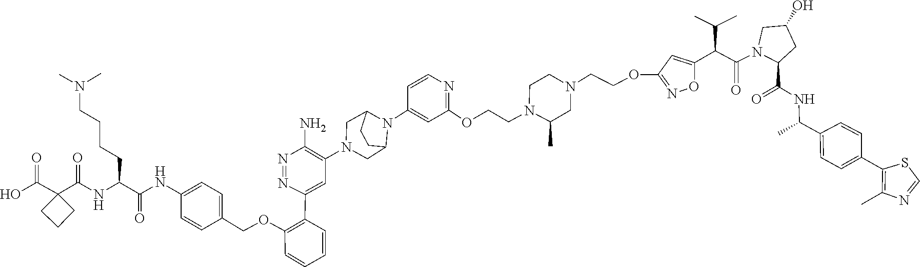 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)[C@H](C)C3)no2)C(C)C)cc1]

A solution of 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (102 mg, 0.110 mmol), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (61.0 mg, 0.110 mmol) and CH₃COOH (13.6 mg, 0.230 mmol) in methyl alcohol (1.2 mL) and dichloromethane (0.4 mL) was stirred at room temperature for 1 hour. Then NaBH₃CN (21.3 mg, 0.340 mmol) was added and stirred at room temperature for 0.5 h. Water was added to quench the reaction. The solvent was concentrated under vacuum. The residue was purified by pre-packed C18 column (solvent gradient: 0-100% MeOH in water (0.05% NH₄HCO₃)) to afford 80.0 mg (49% yield) of the title compound as a yellow solid. LCMS (ESI, m/z) [M+H]⁺=1429.

### Example 11

### Synthesis of a Portion of a Hydrolysable Linker

### Intermediate 11: Ethyl (S)-1-((6-(dimethylamino)-1-((4-(hydroxymethyl)phenyl)amino)-1-oxohexan-2-yl)carbamoyl)cyclobutane-1-carboxylate

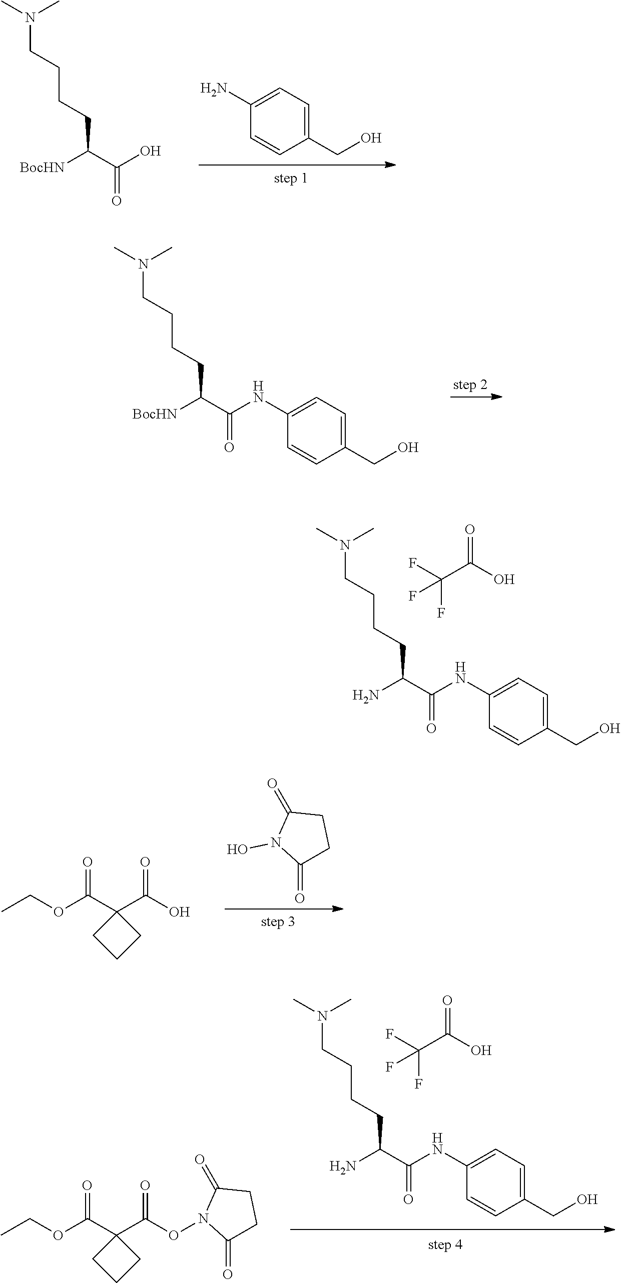

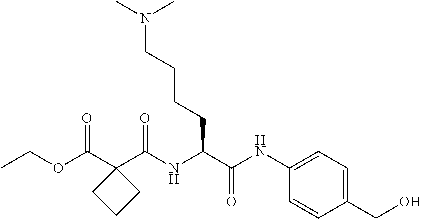 [SMILES: CCOC(=O)C1(C(=O)N[C@@H](CCCCN(C)C)C(=O)Nc2ccc(CO)cc2)CCC1]

### Step 1: tert-Butyl (S)-(6-(dimethylamino)-1-((4-(hydroxymethyl)phenyl)amino)-1-oxohexan-2-yl)carbamate

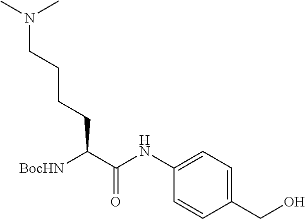 [SMILES: CN(C)CCCC[C@H](NC(=O)OC(C)(C)C)C(=O)Nc1ccc(CO)cc1]

Under nitrogen, a solution of N²-(tert-butoxycarbonyl)-N⁶,N⁶-dimethyl-L-lysine (5.00 g, 18.2 mmol), (4-aminophenyl)methanol (2.46 g, 20.1 mmol) and ethyl 2-ethoxyquinoline-1(2H)-carboxylate (9.01 g, 36.5 mmol) in MeOH (50 mL) and DCM (20 mL) was stirred at 30° C. overnight. The solvent was concentrated under vacuum. The residue was purified by flash chromatography on silica gel (solvent gradient: 0-20% EtOAc/petroleum ether) to yield 2.32 g (33% yield) of the title compound as a white solid. LCMS (ESI): [M+H]⁺=380.

### Step 2: (S)-2-Amino-6-(dimethylamino)-N-(4-(hydroxymethyl)phenyl)hexanamide (2,2,2-trifluoroacetic acid Salt)

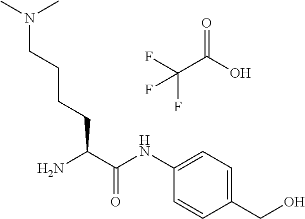 [SMILES: CN(C)CCCC[C@H](N)C(=O)Nc1ccc(CO)cc1 ; O=C(O)C(F)(F)F]

A solution of tert-butyl (S)-(6-(dimethylamino)-1-((4-(hydroxymethyl)phenyl)amino)-1-oxohexan-2-yl)carbamate (2.20 g, 5.78 mmol) in CH₂Cl₂ (15 mL) and TFA (5 mL) was stirred at room temperature for 1 h. The solvent was concentrated under vacuum and the crude was used in next step directly. LC-MS: (ESI, m/z): [M+H]⁺=280.

### Step 3: 1-(2,5-Dioxopyrrolidin-1-yl) 1-ethyl cyclobutane-1,1-dicarboxylate

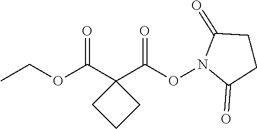 [SMILES: CCOC(=O)C1(C(=O)ON2C(=O)CCC2=O)CCC1]

A solution of 1-(ethoxycarbonyl)cyclobutane-1-carboxylic acid (3.00 g, 17.4 mmol), 1-hydroxypyrrolidine-2,5-dione (2.21 g, 19.2 mmol) and DCC (3.95 g, 19.2 mmol) in THF (50 mL) was stirred at room temperature for 5 h. The reaction mixture was filtered, the filtrate was concentrated under vacuum to yield 5.19 g (crude) of the title compound as a yellow oil. LC-MS: (ESI, m/z): [M+H]⁺=270.

### Step 4: Ethyl (S)-1-((6-(dimethylamino)-1-((4-(hydroxymethyl)phenyl)amino)-1-oxohexan-2-yl)carbamoyl)cyclobutane-1-carboxylate

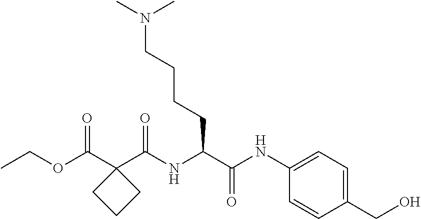 [SMILES: CCOC(=O)C1(C(=O)N[C@@H](CCCCN(C)C)C(=O)Nc2ccc(CO)cc2)CCC1]

A solution of (S)-2-amino-6-(dimethylamino)-N-(4-(hydroxymethyl)phenyl)hexanamide (2.9 g, 10.4 mmol) and NaHCO₃ (2.27 g, 83.2 mmol) in H₂O (30 mL) was stirred at room temperature for 30 min. Then a solution of 1-(2,5-dioxopyrrolidin-1-yl) 1-ethyl cyclobutane-1,1-dicarboxylate (1.51 g, 15.5 mmol) in DME (30 mL) was added and stirred at room temperature overnight. The reaction mixture was filtered, the filtrate was concentrated under vacuum. The residue was purified by pre-packed C18 column (solvent gradient: 0-100% MeOH in water (0.05% NH₄HCO₃)) to yield 1.26 g (49% yield) of the title compound as a colorless oil. LC-MS: (ESI, m/z): [M+H]⁺=434. ¹H NMR (300 MHz, DMSO-d₆, ppm) δ 9.77 (s, 1H), 7.73 (d, J=7.9 Hz, 1H), 7.42 (d, J=8.2 Hz, 2H), 7.12 (d, J=8.2 Hz, 2H), 4.99 (s, 1H), 4.36-4.22 (m, 3H), 4.05-3.97 (m, 2H), 2.34-2.19 (m, 4H), 2.07 (t, J=6.9 Hz, 2H), 1.99 (s, 6H), 1.83-1.51 (m, 4H), 1.37-1.16 (m, 4H), 1.07 (d, J=7.0 Hz, 3H).

### Example 12

### Synthesis of a Portion of a Hydrolysable Linker

### Intermediate 12: tert-Butyl (S)-(5-(3-(tert-butoxy)-2-(2,5-dioxo-2,5-dihydro-1H-pyrrol-1-yl)propanamido)pentyl)carbamate

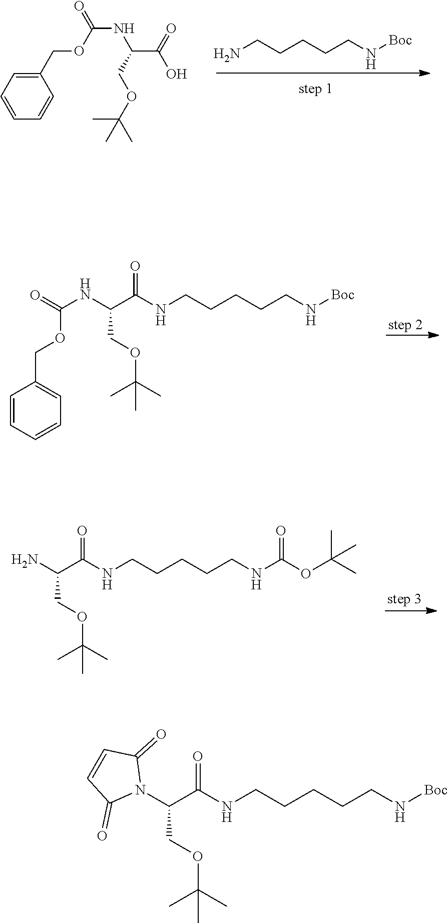 [SMILES: CC(C)(C)OC[C@H](NC(=O)OCc1ccccc1)C(=O)O ; CC(C)(C)OC(=O)NCCCCCN ; CC(C)(C)OC[C@H](NC(=O)OCc1ccccc1)C(=O)NCCCCCNC(=O)OC(C)(C)C ; CC(C)(C)OC[C@H](N)C(=O)NCCCCCNC(=O)OC(C)(C)C ; CC(C)(C)OC[C@@H](C(=O)NCCCCCNC(=O)OC(C)(C)C)N1C(=O)C=CC1=O]

### Step 1: Benzyl (S)-(2,2,16,16-tetramethyl-4,12-dioxo-3,15-dioxa-5,11-diazaheptadecan-13-yl)carbamate

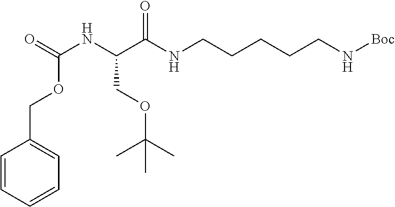 [SMILES: CC(C)(C)OC[C@H](NC(=O)OCc1ccccc1)C(=O)NCCCCCNC(=O)OC(C)(C)C]

Under nitrogen, a solution of N-((benzyloxy)carbonyl)-O-(tert-butyl)-L-serine (1.00 g, 3.39 mmol), tert-butyl (5-aminopentyl)carbamate (684 mg, 3.39 mmol), HATU (1.67 g, 4.40 mmol) and DIPEA (1.31 g, 10.1 mmol) in DMF (10 mL) was stirred at room temperature for 30 min. The reaction was purified by pre-packed C18 column (solvent gradient: 0-60% ACN in water (0.05% NH₄HCO₃)) to yield 980 mg (60% yield) of the title compound as a white solid. LC-MS: (ESI, m/z): [M+H]⁺=480.

### Step 2: tert-Butyl (S)-(5-(2-amino-3-(tert-butoxy)propanamido)pentyl)carbamate

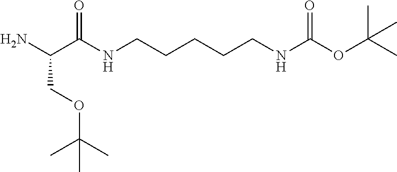 [SMILES: CC(C)(C)OC[C@H](N)C(=O)NCCCCCNC(=O)OC(C)(C)C]

Under hydrogen (3 atm), a solution of benzyl (S)-(2,2,16,16-tetramethyl-4,12-dioxo-3,15-dioxa-5,11-diazaheptadecan-13-yl)carbamate (980 mg, 2.046 mmol) and 10% Pd/C (196 mg) in MeOH (200 mL) was stirred at room temperature for 1 hour. The solids were filtered through a Celite pad and the filtrate was concentrated under reduced pressure to yield 750 mg (crude) of the title compound as a yellow oil. LC-MS: (ESI, m/z): [M+H]⁺=346.

### Step 3: tert-Butyl (S)-(5-(3-(tert-butoxy)-2-(2,5-dioxo-2,5-dihydro-1H-pyrrol-1-yl)propanamido)pentyl)carbamate

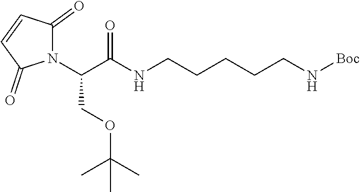 [SMILES: CC(C)(C)OC[C@@H](C(=O)NCCCCCNC(=O)OC(C)(C)C)N1C(=O)C=CC1=O]

Under nitrogen, to a solution of tert-butyl (S)-(5-(2-amino-3-(tert-butoxy)propanamido)pentyl)carbamate (740 mg, 2.14 mmol) and NaHCO₃ (540 mg, 6.43 mmol) in dioxane (5 mL) and H₂O (16 mL) was added methyl 2,5-dioxo-2,5-dihydro-1H-pyrrole-1-carboxylate (332 mg, 2.14 mmol) at 0° C. The solution was stirred at 0° C. for 40 min. Then the reaction mixture was stirred at room temperature for 0.5 h. Ethyl acetate was used to extract for three times. The organic layers were combined, dried over anhydrous sodium sulfate and concentrated under vacuum. The residue was purified by flash chromatography on silica gel (gradient: 0%-60% EtOAc/petroleum ether) to yield 540 mg (59% yield) of the title compound as a colorless oil. LC-MS: (ESI, m/z): [M+H]⁺=426.

### Example 13

### Synthesis of Hy-B-CIDE-6

### N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-(2-(2,5-dioxo-2,5-dihydro-1H-pyrrol-1-yl)acetamido)pentyl)cyclobutane-1,1-dicarboxamide (2,2,2-trifluoroacetic acid Salt)

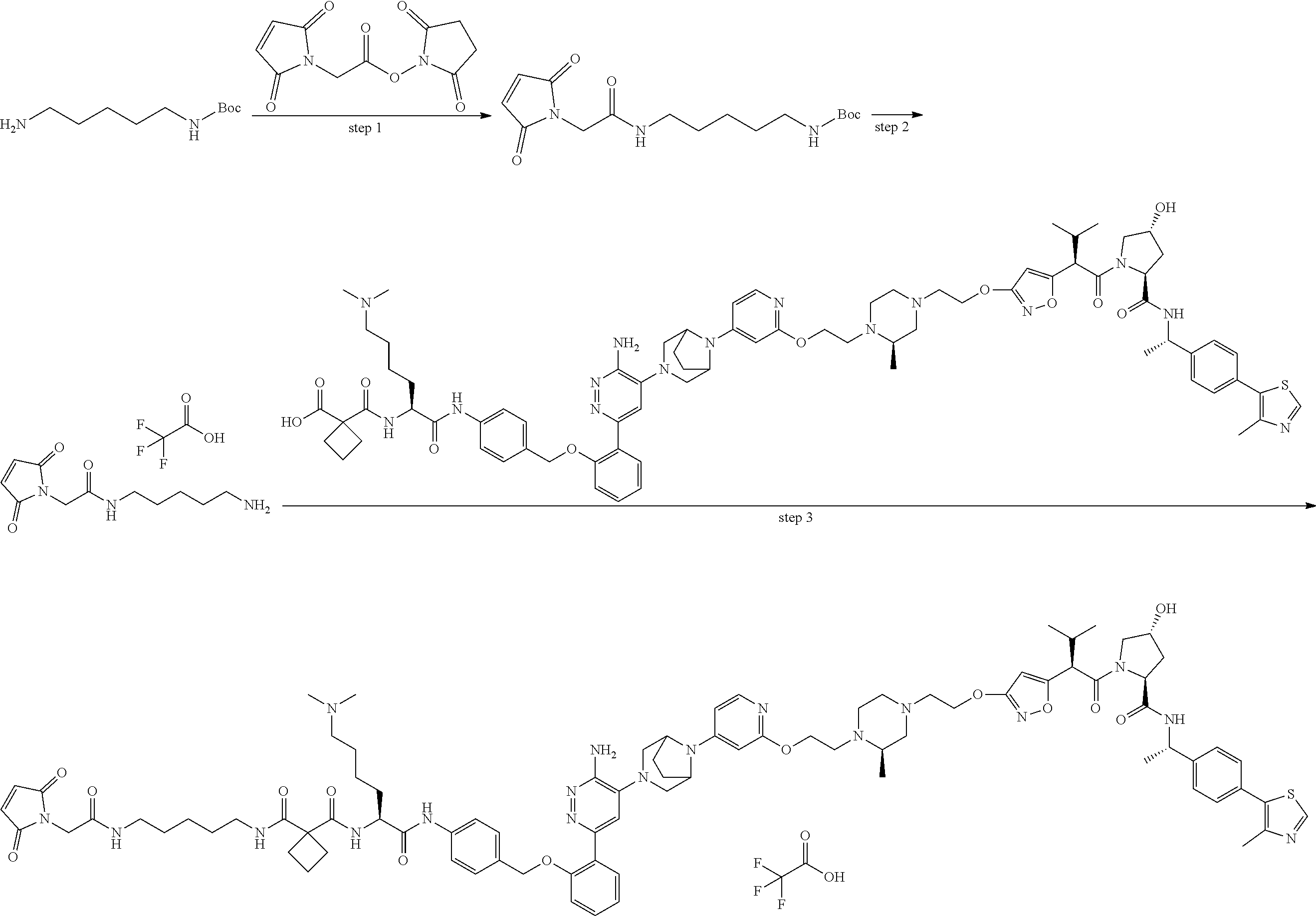

### Step 1: tert-butyl (5-(2-(2,5-dioxo-2,5-dihydro-1H-pyrrol-1-yl)acetamido)pentyl)carbamate

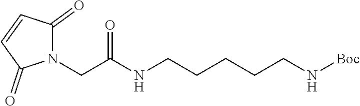 [SMILES: CC(C)(C)OC(=O)NCCCCCNC(=O)CN1C(=O)C=CC1=O]

A solution of tert-butyl (5-aminopentyl)carbamate (500 mg, 2.47 mmol) and 2,5-dioxopyrrolidin-1-yl 2-(2,5-dioxo-2,5-dihydro-1H-pyrrol-1-yl)acetate (1.25 g, 4.94 mmol) and NMM (476 mg, 4.45 mmol) in DMF (3 mL) was stirred at room temperature for 1 hour. The residue was purified by pre-packed C18 column (solvent gradient: 0-100% ACN in water (0.05% TFA)) and then purified again by flash chromatography on silica gel (gradient: 0%-10% MeOH/DCM) to yield 343 mg (41% yield) of the title compound as a red oil. LCMS (ESI) [M+H]⁺=340.

### Step 2: N-(5-aminopentyl)-2-(2,5-dioxo-2,5-dihydro-1H-pyrrol-1-yl)acetamide (2,2,2-trifluoroacetic acid Salt)

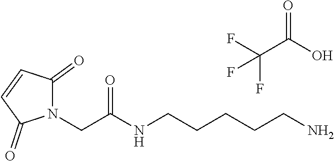

A solution of tert-butyl (5-(2-(2,5-dioxo-2,5-dihydro-1H-pyrrol-1-yl)acetamido)pentyl)carbamate (30.0 mg, 0.0900 mmol) in dichloromethane (2 mL) and 2,2,2-trifluoroacetic acid (0.5 mL) was stirred at room temperature for 1 hours. The solvent was concentrated under vacuum to afford 32.0 mg (crude) of the title compound as a yellow oil. LCMS (ESI) [M+H]⁺=240.

### Step 3: N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-(2-(2,5-dioxo-2,5-dihydro-1H-pyrrol-1-yl)acetamido)pentyl)cyclobutane-1,1-dicarboxamide (2,2,2-trifluoroacetic acid Salt)

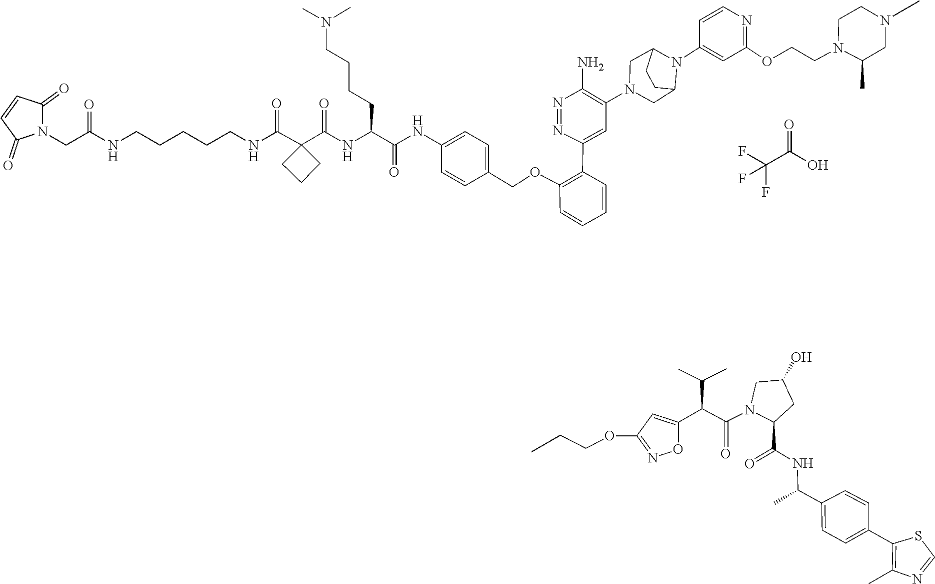

To a solution of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (60.0 mg, 0.0400 mmol), N-(5-aminopentyl)-2-(2,5-dioxo-2,5-dihydro-1H-pyrrol-1-yl)acetamide (2,2,2-trifluoroacetic acid salt) (30.0 mg, crude) and DIPEA (54.2 mg, 0.420 mmol) in DMF (0.5 mL) was added HATU (19.2 mg, 0.0500 mmol) at room temperature. The reaction was stirred at room temperature for 10 minutes. The crude was purified by Prep-HPLC (Column: Xselect CSH F-Phenyl OBD column, 19*250 mm, 5 m; Mobile Phase A: Water (0.05% TFA), Mobile Phase B: ACN; Flow rate: 25 mL/min; Gradient: 20% B to 27% B in 11 min, 27% B; Wavelength: 254 nm; RT1(min): 10.4) to afford 31.5 mg (45% yield) of the title compound as a yellow solid. LCMS (ESI) [M+H]⁺=1650. ¹H NMR (300 MHz, DMSO-d6) δ 10.20 (s, 1H), 9.51 (s, 1H), 8.99 (s, 1H), 8.38 (d, J=7.6 Hz, 1H), 8.11 (t, J=5.4 Hz, 1H), 7.95-7.80 (m, 3H), 7.67 (d, J=8.3 Hz, 2H), 7.58 (td, J=5.1, 2.4 Hz, 2H), 7.51-7.25 (m, 8H), 7.23-7.03 (m, 5H), 6.69 (d, J=6.5 Hz, 1H), 6.32 (s, 1H), 6.11 (s, 1H), 5.10 (s, 2H), 4.91 (t, J=7.3 Hz, 1H), 4.55 (s, 2H), 4.47 (s, 2H), 4.43-4.20 (m, 5H), 3.99 (s, 2H), 3.50-3.30 (m, 7H), 3.30-3.15 (m, 4H), 3.15-3.05 (m, 6H), 3.05-2.95 (m, 5H), 2.75 (d, J=4.4 Hz, 6H), 2.50-2.35 (m, 8H), 2.23-1.60 (m, 13H), 1.53-1.04 (m, 15H), 1.05-0.90 (m, 3H), 0.85-0.70 (m, 3H).

### Example 14

### Synthesis of Hy-B-CIDE-1

### N-((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((S)-2-(2,5-dioxo-2,5-dihydro-1H-pyrrol-1-yl)-3-hydroxypropanamido)pentyl)cyclobutane-1,1-dicarboxamide (2,2,2-trifluoroacetic acid salt)

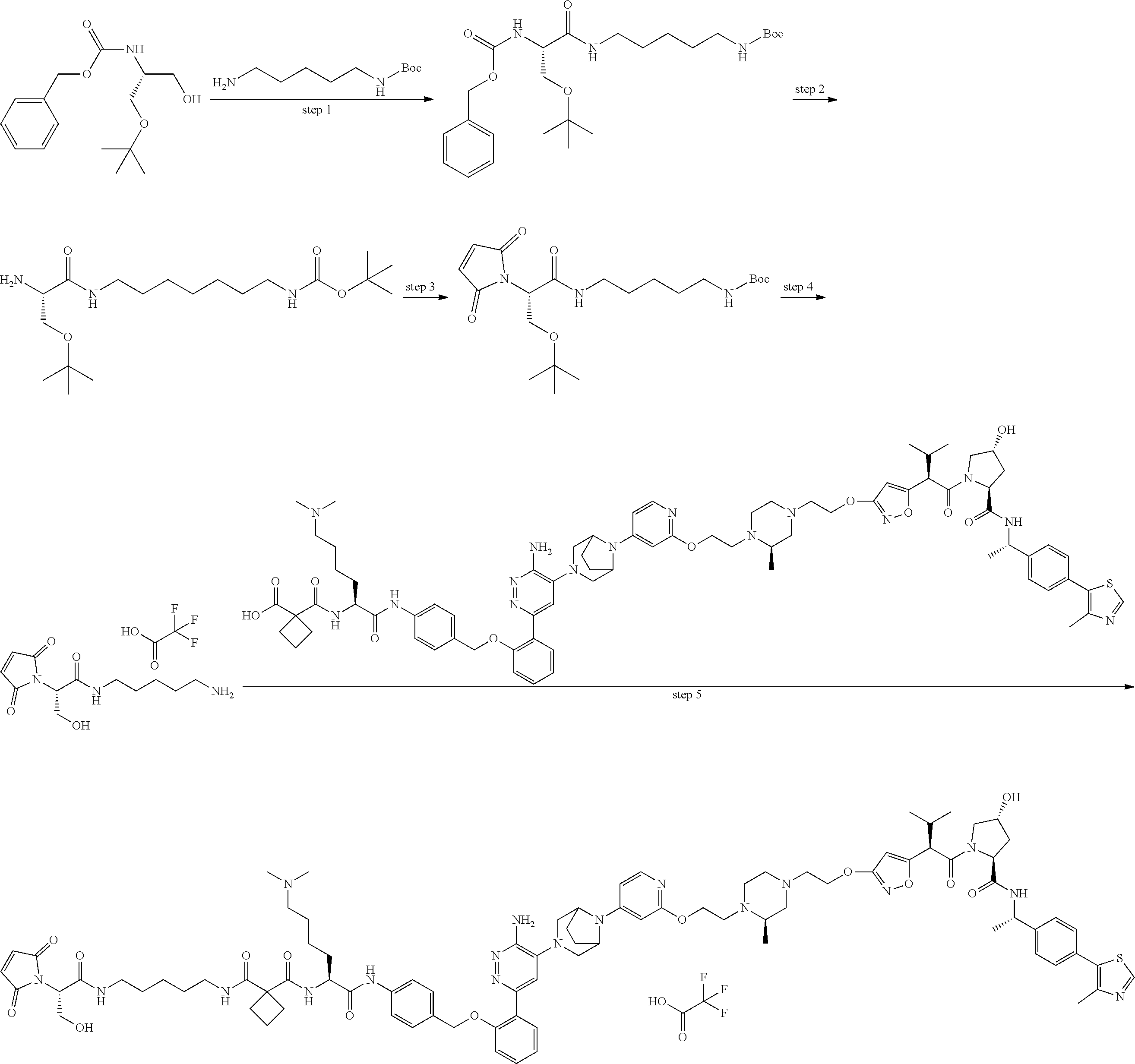

### Step 1: benzyl (S)-(2,2,16,16-tetramethyl-4,12-dioxo-3,15-dioxa-5,11-diazaheptadecan-13-yl)carbamate

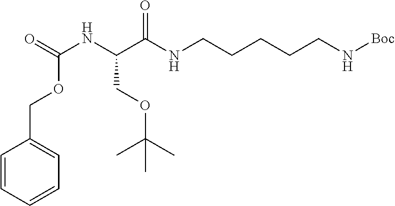 [SMILES: CC(C)(C)OC[C@H](NC(=O)OCc1ccccc1)C(=O)NCCCCCNC(=O)OC(C)(C)C]

Under nitrogen, a solution of N-((benzyloxy)carbonyl)-O-(tert-butyl)-L-serine (1.0 g, 3.390 mmol), tert-butyl (5-aminopentyl)carbamate (684 mg, 3.39 mmol), HATU (1.67 g, 4.40 mmol) and DIPEA (1.31 g, 10.1 mmol) in DMF (10 mL) was stirred at room temperature for 30 min. The reaction was purified by pre-packed C18 column (solvent gradient: 0-60% ACN in water (0.05% NH₄HCO₃)) to yield 980 mg (60% yield) of the title compound as a white solid. LC-MS: (ESI, m/z): [M+H]⁺=480.

### Step 2: tert-butyl (S)-(5-(2-amino-3-(tert-butoxy)propanamido)pentyl)carbamate

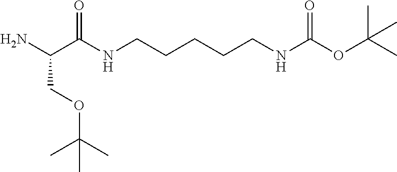 [SMILES: CC(C)(C)OC[C@H](N)C(=O)NCCCCCNC(=O)OC(C)(C)C]

Under hydrogen (1 atm), a solution of benzyl (S)-(2,2,16,16-tetramethyl-4,12-dioxo-3,15-dioxa-5,11-diazaheptadecan-13-yl)carbamate (980 mg, 2.046 mmol) and Pd/C (196 mg) in MeOH (200 mL) was stirred at room temperature for 1 hour. The solids were filtered through a Celite pad and the filtrate was concentrated under reduced pressure to yield 750 mg (crude) of the title compound as a yellow oil. LC-MS: (ESI, m/z): [M+H]⁺=346.

### Step 3: tert-Butyl (S)-(5-(3-(tert-butoxy)-2-(2,5-dioxo-2,5-dihydro-1H-pyrrol-1-yl)propanamido)pentyl)carbamate

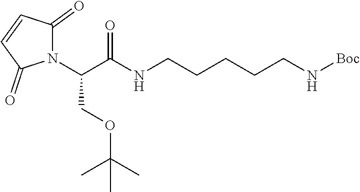 [SMILES: CC(C)(C)OC[C@@H](C(=O)NCCCCCNC(=O)OC(C)(C)C)N1C(=O)C=CC1=O]

Under nitrogen, to a solution of tert-butyl (S)-(5-(2-amino-3-(tert-butoxy)propanamido)pentyl)carbamate (740 mg, 2.145 mmol) and NaHCO₃ (540 mg, 6.43 mmol) in dioxane (5 mL) and H₂O (16 mL) was added methyl 2,5-dioxo-2,5-dihydro-1H-pyrrole-1-carboxylate (332 mg, 2.14 mmol) at 0° C. The solution was stirred at 0° C. for 40 min. Then the reaction mixture was stirred at room temperature for 0.5 hours. The reaction was extracted with ethyl acetate. The organic layers were combined, dried over anhydrous sodium sulfate and concentrated under vacuum. The residue was purified by flash chromatography on silica gel (gradient: 0%-60% EtOAc/petroleum ether) to yield 540 mg (59% yield) of the title compound as a colorless oil. LC-MS: (ESI, m/z): [M+H]⁺=426

### Step 4: (S)—N-(5-aminopentyl)-2-(2,5-dioxo-2,5-dihydro-1H-pyrrol-1-yl)-3-hydroxypropanamide (2,2,2-trifluoroacetic acid Salt)

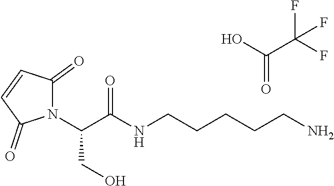

Under nitrogen, a solution of tert-butyl (S)-(5-(3-(tert-butoxy)-2-(2,5-dioxo-2,5-dihydro-1H-pyrrol-1-yl)propanamido)pentyl)carbamate (15.0 mg, 0.035 mmol) in TFA (0.5 mL) was stirred at room temperature for 1 hour. The resulting mixture was concentrated under vacuum to yield 20.0 mg (crude) of the title compound as a colorless oil. LC-MS: (ESI, m/z): [M+H]⁺=270.

### Step 5: N-((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((S)-2-(2,5-dioxo-2,5-dihydro-1H-pyrrol-1-yl)-3-hydroxypropanamido)pentyl)cyclobutane-1,1-dicarboxamide (2,2,2-trifluoroacetic acid Salt)

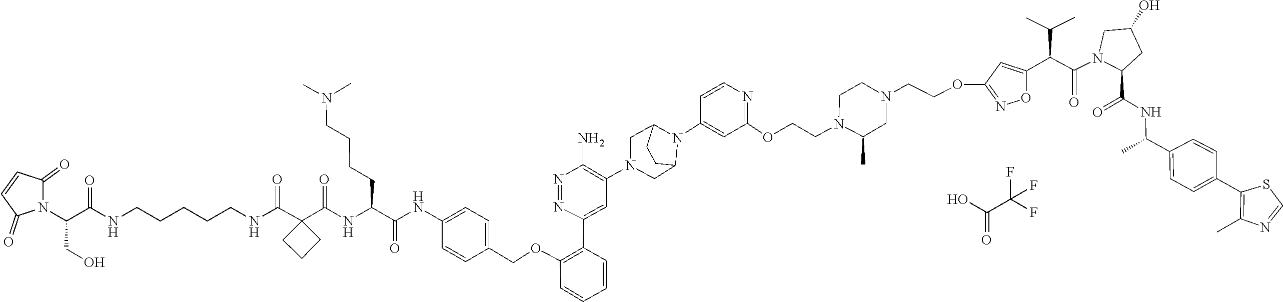 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCCCCNC(=O)[C@H](CO)N9C(=O)C=CC9=O)CCC8)cc7)nnc5N)C6)ccn4)[C@H](C)C3)no2)C(C)C)cc1 ; O=C(O)C(F)(F)F]

Under nitrogen, to a solution of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (30.0 mg, 0.021 mmol), (S)—N-(5-aminopentyl)-2-(2,5-dioxo-2,5-dihydro-1H-pyrrol-1-yl)-3-hydroxypropanamide (2,2,2-trifluoroacetic acid salt) (20.0 mg, crude), DIPEA (27.1 mg, 0.210 mmol) in DMF (0.9 mL) was added HATU (10.4 mg, 0.027 mmol) at room temperature. The resulting solution was stirred at room temperature for 10 min. The resulting solution was purified by Prep-HPLC (XBridge Shield RP18 OBD Column, 19×250 mm 10 m; Mobile Phase A: Water (0.05% TFA), Mobile Phase B: ACN; Flow rate: 25 mL/min; Gradient: 20% B to 35% B in 10 min; 254/220 nm; R_{T1}: 8.26) to yield 22.6 mg (64% yield) of HY—B-CIDE-1 as a white solid. LC-MS: (ESI, m/z): [M+H]⁺=1680. ¹H NMR (300 MHz, DMSO-d₆) δ 10.21 (s, 1H), 9.45 (br, 1H), 9.00 (d, J=1.7 Hz, 1H), 8.40 (d, J=7.8 Hz, 1H), 7.98-7.84 (m, 4H), 7.67 (d, J=8.3 Hz, 2H), 7.58 (d, J=7.4 Hz, 2H), 7.52-7.28 (m, 8H), 7.19-6.91 (m, 5H), 6.69 (s, 1H), 6.31 (s, 1H), 6.13 (s, 1H), 5.11 (s, 2H), 4.95-4.80 (m, 1H), 4.62-4.52 (m, 2H), 4.52-4.45 (m, 3H), 4.42-4.32 (m, 4H), 4.32-4.25 (m, 2H), 3.95-3.86 (m, 9H), 3.50-3.40 (m, 3H), 3.40-3.28 (m, 4H), 3.15-3.05 (m, 5H), 3.05-2.95 (m, 5H), 2.75 (d, J=4.7 Hz, 6H), 2.45-2.35 (m, 8H), 2.25-2.10 (m, 1H), 2.05-1.86 (m, 5H), 1.84-1.52 (m, 7H), 1.50-1.30 (m, 8H), 1.30-1.10 (m, 6H), 0.97 (d, J=6.4 Hz, 3H), 0.88-0.76 (m, 3H).

### Example 15

### Synthesis of Hy-B-CIDE-2

### N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((S)-2-(2,5-dioxo-2,5-dihydro-1H-pyrrol-1-yl)-3-methoxypropanamido)pentyl)cyclobutane-1,1-dicarboxamide (2,2,2-trifluoroacetic acid Salt)

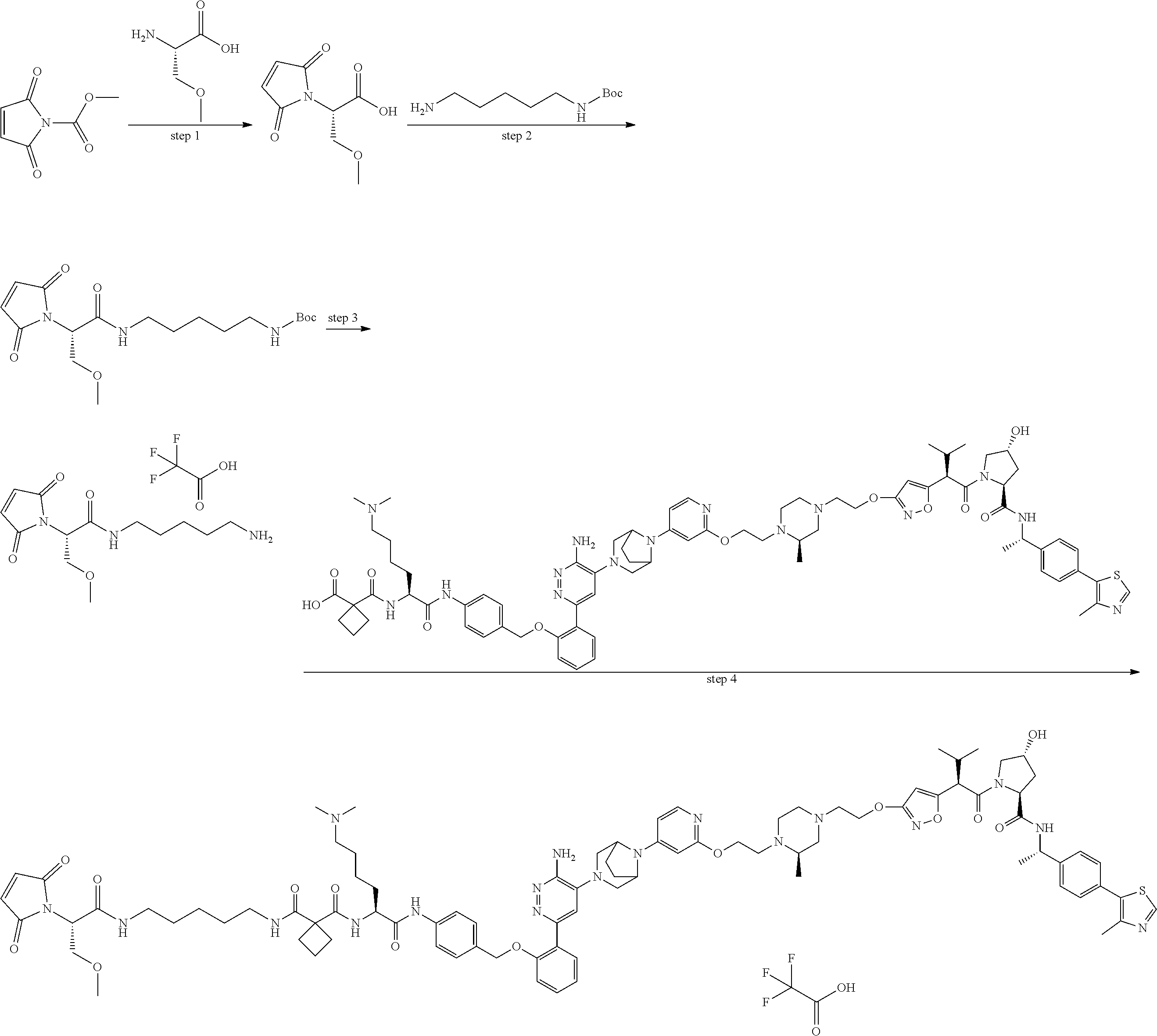

### Step 1: (S)-2-(2,5-dioxo-2,5-dihydro-1H-pyrrol-1-yl)-3-methoxypropanoic acid

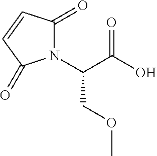

To a solution of methyl 2,5-dioxo-2,5-dihydro-1H-pyrrole-1-carboxylate (500 mg, 3.22 mmol) and NaHCO₃ (810 mg, 9.64 mmol) in water (10 mL) was added methyl O-methyl-L-serine (384 mg, 3.22 mmol) at room temperature. The reaction was stirred at room temperature for 2 h. The pH was adjusted to 5 with KHSO₄. The product was extracted with dichloromethane for four times. The combined organic layer was washed with brine and dried over anhydrous sodium sulfate. The solvent was concentrated under vacuum to yield 600 mg (93% yield) of the title compound as a white oil. LC-MS: (ESI, m/z): M⁻=198.

### Step 2: tert-Butyl (S)-(5-(2-(2,5-dioxo-2,5-dihydro-1H-pyrrol-1-yl)-3-methoxypropanamido)pentyl)carbamate

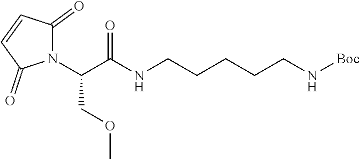 [SMILES: COC[C@@H](C(=O)NCCCCCNC(=O)OC(C)(C)C)N1C(=O)C=CC1=O]

A solution of tert-butyl (5-aminopentyl)carbamate (0.62 mL, 2.97 mmol), (S)-2-(2,5-dioxo-2,5-dihydro-1H-pyrrol-1-yl)-3-methoxypropanoic acid (600 mg, 3.01 mmol), HATU (1.50 g, 3.94 mmol) and DIPEA (1.20 g, 9.3 mmol) in DMF (10 mL) was stirred at room temperature for 1 h. The reaction was diluted with EtOAc and washed with water. The organic layers were combined, dried under vacuum and concentrated under vacuum. The crude was purified by flash chromatography on silica gel (gradient: 0%-100% ethyl acetate/petroleum ether) to yield 150 mg (13% yield) of the title compound as a yellow oil. LC-MS: (ESI, m/z): [M+H]⁺=384.

### Step 3: (S)—N-(5-aminopentyl)-2-(2,5-dioxo-2,5-dihydro-1H-pyrrol-1-yl)-3-methoxypropanamide (2,2,2-trifluoroacetic acid Salt)

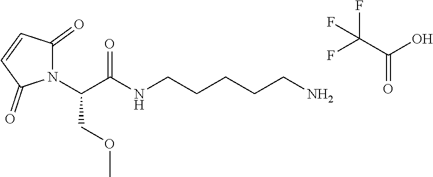

A solution of tert-butyl (S)-(5-(2-(2,5-dioxo-2,5-dihydro-1H-pyrrol-1-yl)-3-methoxypropanamido)pentyl)carbamate (25.0 mg, 0.0700 mmol) in dichloromethane (3 mL) and 2,2,2-trifluoroacetic acid (1 mL) was stirred at room temperature for 1 hour. The solvent was concentrated under vacuum to yield 18 mg (crude) of the title compound as a yellow oil. LC-MS: (ESI, m/z): [M+H]⁺=284.

### Step 4: N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((S)-2-(2,5-dioxo-2,5-dihydro-1H-pyrrol-1-yl)-3-methoxypropanamido)pentyl)cyclobutane-1,1-dicarboxamide (2,2,2-trifluoroacetic acid Salt)

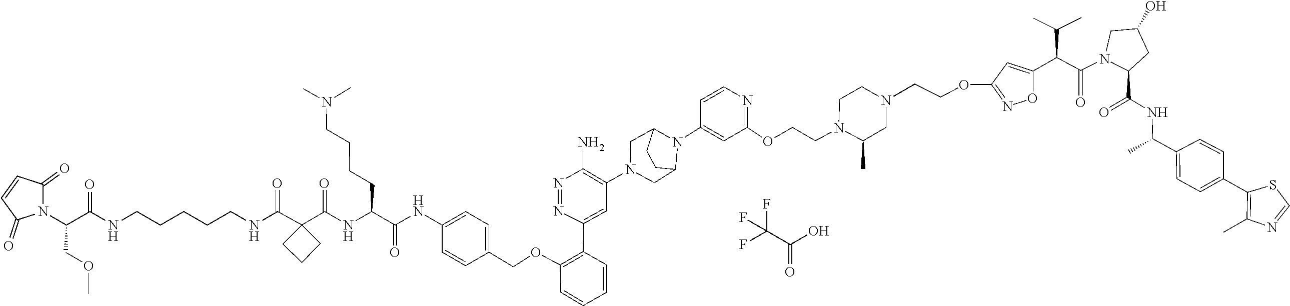 [SMILES: COC[C@@H](C(=O)NCCCCCNC(=O)C1(C(=O)N[C@@H](CCCCN(C)C)C(=O)Nc2ccc(COc3ccccc3-c3cc(N4CC5CCC(C4)N5c4ccnc(OCCN5CCN(CCOc6cc([C@H](C(=O)N7C[C@H](O)C[C@H]7C(=O)N[C@@H](C)c7ccc(-c8scnc8C)cc7)C(C)C)on6)C[C@H]5C)c4)c(N)nn3)cc2)CCC1)N1C(=O)C=CC1=O ; O=C(O)C(F)(F)F]

To a solution of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (60.0 mg, 0.0400 mmol), (S)—N-(5-aminopentyl)-2-(2,5-dioxo-2,5-dihydro-1H-pyrrol-1-yl)-3-methoxypropanamide (2,2,2-trifluoroacetic acid salt) (18.0 mg, crude) and DIPEA (54.2 mg, 0.420 mmol) in DMF (2 mL) was added HATU (19.2 mg, 0.0500 mmol) at room temperature. The reaction was stirred at room temperature for 10 min. The residue was purified by Prep-HPLC (XBridge Shield RP18 OBD Column, 30×150 mm Sum; Mobile Phase A: Water (10 mmol/L NH₄HCO₃), Mobile Phase B: ACN; Flow rate: 60 mL/min; Gradient: 6% B to 21% B in 12 min; 254 nm; R_{T1}:14.45) to yield 34 mg (44% yield) of Hy-B-CIDE-2 as a white solid. LC-MS: (ESI, m/z): [M+H]⁺=1694. ¹H NMR (300 MHz, DMSO-d6, ppm) δ 10.22 (s, 1H), 9.52 (s, 1H), 8.99 (d, J=1.5 Hz, 1H), 8.40 (d, J=7.7 Hz, 1H), 8.03 (t, J=5.7 Hz, 1H), 7.89 (d, J=6.6 Hz, 3H), 7.68 (d, J=8.3 Hz, 2H), 7.57 (t, J=7.4 Hz, 2H), 7.50-7.28 (m, 8H), 7.18-7.10 (m, 2H), 7.10-6.90 (m, 3H), 6.66 (s, 1H), 6.28 (s, 1H), 6.12 (s, 1H), 5.10 (s, 3H), 4.91 (t, J=7.2 Hz, 1H), 4.71-4.66 (m, 1H), 4.60-4.45 (m, 4H), 4.40-4.27 (m, 5H), 3.89-3.79 (m, 5H), 3.70-3.65 (m, 4H), 3.50-3.40 (m, 3H), 3.17 (s, 4H), 3.13-3.08 (m, 4H), 3.06-2.96 (m, 6H), 2.75 (d, J=4.5 Hz, 8H), 2.47-2.43 (m, 5H), 2.42-2.35 (m, 4H), 2.25-2.15 (m, 1H), 2.10-1.55 (m, 10H), 1.52-1.27 (m, 9H), 1.27-1.10 (m, 6H), 0.97 (dd, J=6.5, 3.7 Hz, 3H), 0.88-0.75 (m, 3H).

### Example 16

### Synthesis of Hy-B-CIDE-5

### N-((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(2-((2-(2,5-dioxo-2,5-dihydro-1H-pyrrol-1-yl)ethyl)(methyl)amino)ethyl)cyclobutane-1,1-dicarboxamide (2,2,2-trifluoroacetic acid Salt)

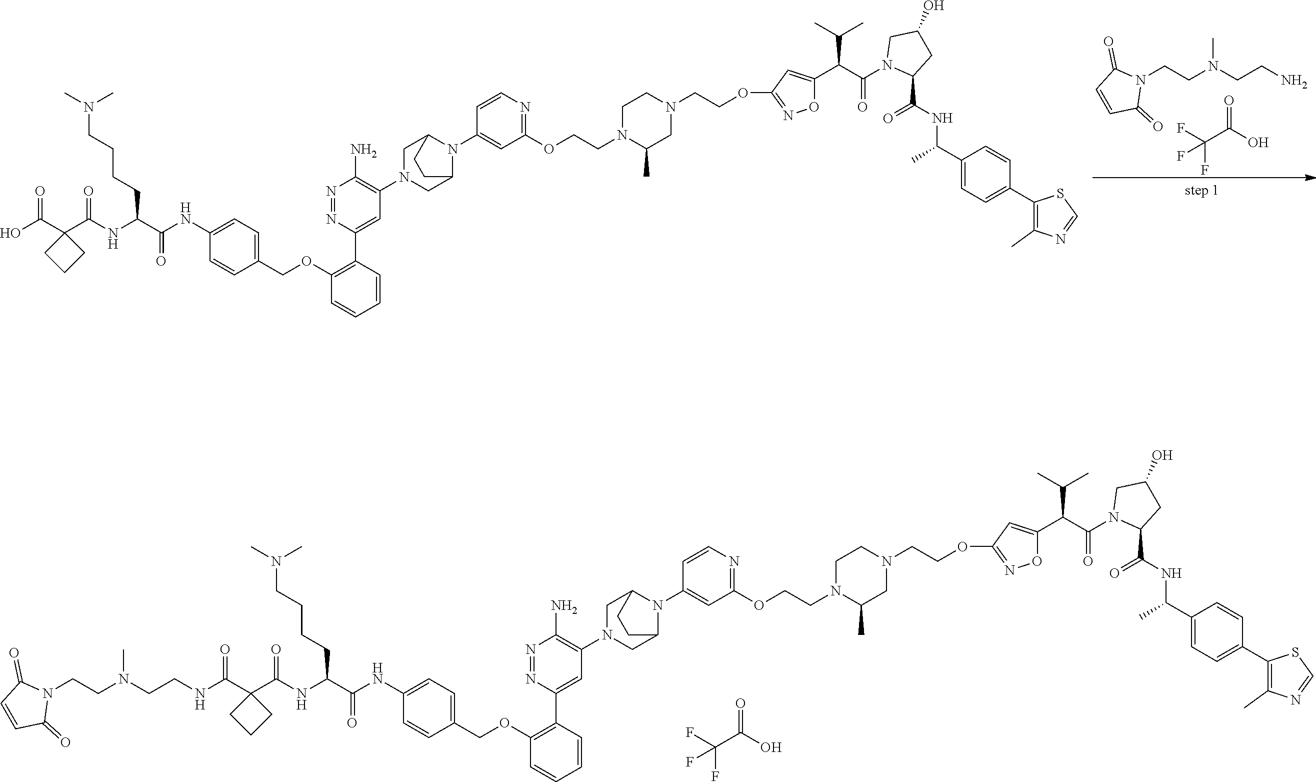

### Step 1: N-((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(2-((2-(2,5-dioxo-2,5-dihydro-1H-pyrrol-1-yl)ethyl)(methyl)amino)ethyl)cyclobutane-1,1-dicarboxamide (2,2,2-trifluoroacetic acid Salt)

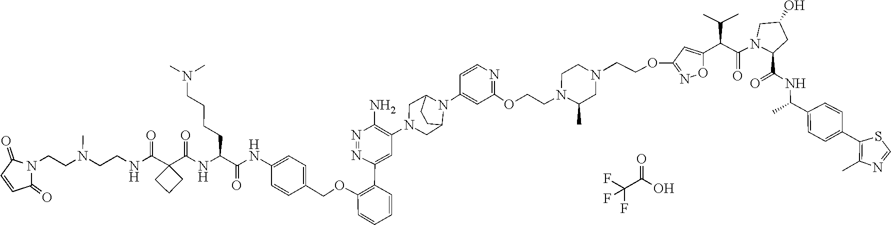 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)[C@H](C)C3)no2)C(C)C)cc1 ; O=C(O)C(F)(F)F]

To a solution of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (60 mg, 0.0420 mmol), 1-(2-((2-aminoethyl)(methyl)amino)ethyl)-1H-pyrrole-2,5-dione (2,2,2-trifluoroacetic acid salt) (12.4 mg, crude) and DIPEA (54.2 mg, 0.420 mmol) in DMF (1 mL) was added HATU (19.2 mg, 0.0505 mmol) at room temperature. The reaction was stirred at room temperature for 10 minutes. The reaction solution was purified by Prep-HPLC (Column: Xselect CSH C18 OBD Column 30*150 mm 5 m; Mobile Phase A: Water (0.05% TFA), Mobile Phase B: ACN; Flow rate: 60 mL/min; Gradient: 5% B to 25% B in 12 min, 25% B; Wavelength: 254/220 nm; R_{T1}(min): 12 min) to yield 34.3 mg (51% yield) of Hy-B-CIDE-5 as a white solid. LC-MS: (ESI, m/z): [M+H]⁺=1608. ¹H NMR (300 MHz, DMSO-d₆) δ 10.21 (s, 1H), 9.59 (br, 1H), 8.99 (d, J=1.6 Hz, 1H), 8.38 (d, J=7.6 Hz, 1H), 8.11 (t, J=5.9 Hz, 1H), 7.86 (dd, J=18.6, 7.0 Hz, 2H), 7.68-7.51 (m, 4H), 7.49-7.26 (m, 9H), 7.14 (t, J=7.5 Hz, 1H), 7.07 (s, 3H), 6.67 (d, J=6.5 Hz, 1H), 6.29 (s, 1H), 6.11 (s, 1H), 5.10 (s, 2H), 4.91 (t, J=7.3 Hz, 1H), 4.60-4.40 (m, 5H), 4.38-4.31 (m, 3H), 4.29 (s, 1H), 3.50-3.05 (m, 25H), 2.85 (s, 3H), 2.75 (s, 6H), 2.46 (s, 9H), 2.05-1.87 (m, 5H), 1.86-1.55 (m, 8H), 1.50-1.30 (m, 5H), 1.25-1.15 (m, 4H), 0.97 (d, J=6.6, 3H), 0.89-0.70 (m, 3H).

### Example 17

### Synthesis of Hy-B-CIDE-8

### N-((2S)-1-((4-((2-(6-Amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((2-(2,5-dioxo-2,5-dihydro-1H-pyrrol-1-yl)ethyl)(methyl)amino)pentyl)cyclobutane-1,1-dicarboxamide (2,2,2-trifluoroacetic acid Salt)

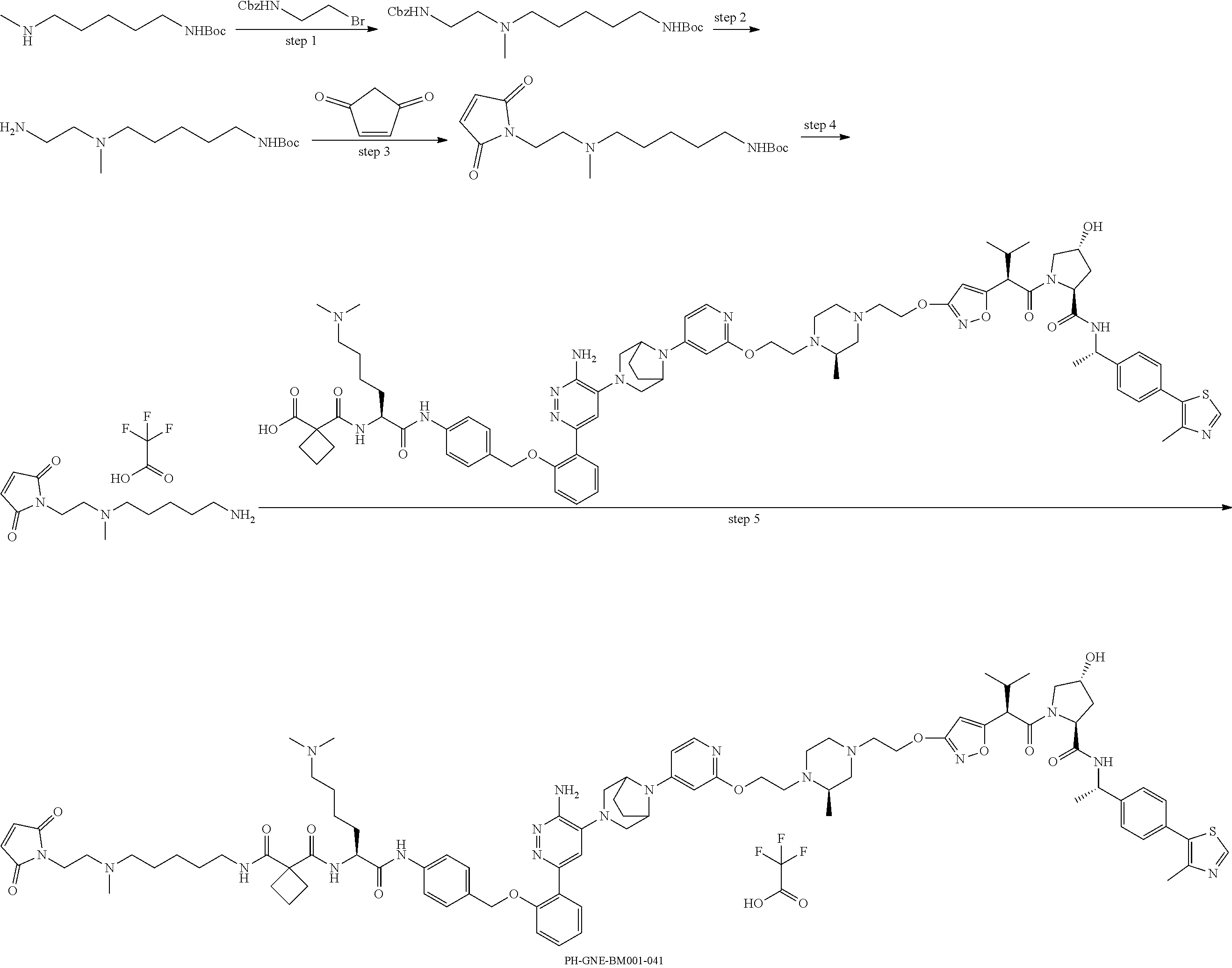

### Step 1: tert-Butyl(5-((2-(((benzyloxy)carbonyl)amino)ethyl)(methyl)amino)pentyl)carbamate

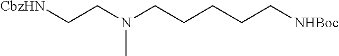 [SMILES: CN(CCCCCNC(=O)OC(C)(C)C)CCNC(=O)OCc1ccccc1]

Under nitrogen, a solution of tert-butyl (5-(methylamino)pentyl)carbamate (1.00 g, 4.62 mmol), benzyl N-(2-bromoethyl)carbamate (1.80 g, 6.97 mmol) and DIPEA (1.80 g, 13.9 mmol) in acetonitrile (10 mL) was stirred at 50° C. for 1 h. The solvent was concentrated under vacuum and water was added. The resulting solution was extracted with DCM and the organic layers were combined. The organic layer was dried over anhydrous sodium sulfate and concentrated under vacuum. The residue was purified by flash chromatography on silica gel (solvent gradient: 0-5% MeOH/DCM) to afford 1.30 g (7100 yield) of the title compound as a colorless oil. LCMS (ESI, m/z): [M+H]⁺=394.

### Step 2: tert-Butyl (5-((2-aminoethyl)(methyl)amino)pentyl)carbamate

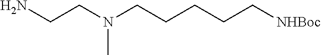 [SMILES: CN(CCN)CCCCCNC(=O)OC(C)(C)C]

Under hydrogen (3 atm), a solution of methylamine (2 M in THF) (7.5 mL, 15.2 mmol) and 100% Pd/C (240 mg) in MeOH (50 mL) was stirred at room temperature for 30 min (to remove trace formaldehyde in MeOH). Then tert-butyl (5-((2-(((benzyloxy)carbonyl)amino)ethyl)(methyl)amino)pentyl)carbamate (1.20 g, 3.05 mmol) was added and the reaction was stirred at room temperature for 2 h. After filtration, the filtrate was concentrated under reduced pressure to afford 810 mg (crude) of the title compound as a yellow oil. LCMS (ESI, m/z): [M+H]⁺=260.

### Step 3: tert-Butyl (5-((2-(2,5-dioxo-2,5-dihydro-1H-pyrrol-1-yl)ethyl)(methyl)amino)pentyl)carbamate

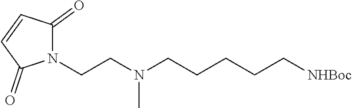 [SMILES: CN(CCCCCNC(=O)OC(C)(C)C)CCN1C(=O)C=CC1=O]

Under nitrogen, a solution of tert-butyl (5-((2-aminoethyl)(methyl)amino)pentyl)carbamate (800 mg, 3.08 mmol) and maleicanhydride (300 mg, 3.05 mmol) in CH₃COOH (10 mL) was stirred at room temperature for 1 h. The solution was concentrated under vacuum and the residue was trituated with DCM/hexane (1:4) for 3 times, Toluene (10 mL) was added followed by triethylamine (1.56 g, 15.4 mmol) and 4 Å molecular sieve (2.86 g). The reaction was stirred at 110° C. for 1 h. After filtration, the filtrate was concentrated under reduced pressure. The residue was purified by flash chromatography on silica gel (solvent gradient: 0-100% EtOAc/petroleum ether) to afford 825 mg of the title compound as a yellow oil. LCMS (ESI, m/z): [M+H]⁺=340.

### Step 4: 1-(2-((5-Aminopentyl)(methyl)amino)ethyl)-1H-pyrrole-2,5-dione (2,2,2-trifluoroacetic acid)

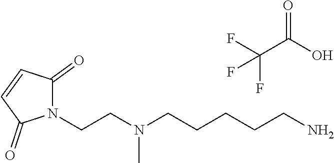

Under nitrogen, a solution of tert-butyl (5-((2-(2,5-dioxo-2,5-dihydro-1H-pyrrol-1-yl)ethyl)(methyl)amino)pentyl)carbamate (13.0 mg, 0.0400 mmol) in DCM (1 mL) and 2,2,2-trifluoroacetic acid (0.3 mL) was stirred at room temperature for 30 min. The solvent was evaporated to afford 30 mg (crude) of the title compound as a yellow oil. LCMS (ESI, m/z): [M+H]⁺=240.

### Step 5: N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((2-(2,5-dioxo-2,5-dihydro-1H-pyrrol-1-yl)ethyl)(methyl)amino)pentyl)cyclobutane-1,1-dicarboxamide (2,2,2-trifluoroacetic acid Salt)

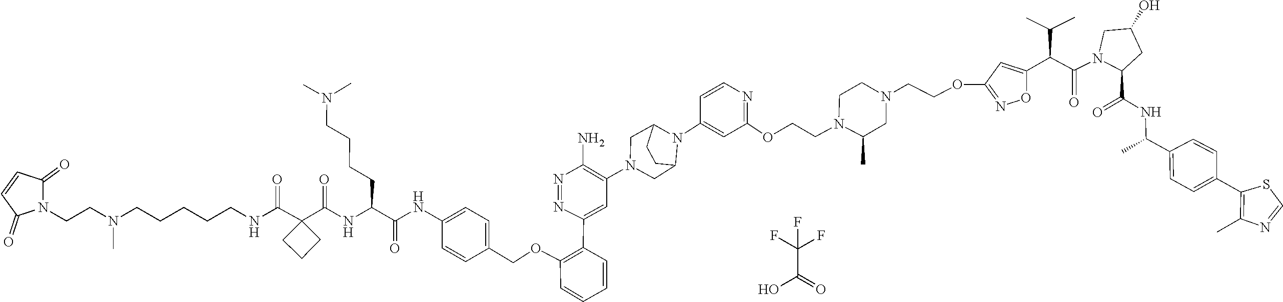 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCCCCN(C)CCN9C(=O)C=CC9=O)CCC8)cc7)nnc5N)C6)ccn4)[C@H](C)C3)no2)C(C)C)cc1 ; O=C(O)C(F)(F)F]

Under nitrogen, to a solution of 1-(2-((5-aminopentyl)(methyl)amino)ethyl)-1H-pyrrole-2,5-dione (2,2,2-trifluoroacetic acid salt) (8.0 mg, crude), 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (40.0 mg, 0.0300 mmol, Intermediate 10) and DIPEA (38.7 mg, 0.300 mmol) in N,N-dimethylformamide (1 mL) was added HATU (14.0 mg, 0.0400 mmol) at room temperature. The reaction was stirred at room temperature for 10 min. The crude was purified by Prep-HPLC with the following conditions: Column: Xselect CSH C18 OBD Column 30*150 mm 5 m, n; Mobile Phase A: Water (0.05% TFA), Mobile Phase B: ACN; Flow rate: 60 mL/min; Gradient: 19% B to 34% B in 11 min, 34% B; Wave Length: 254/220 nm; RT1(min): 10; to afford 7.0 mg of Hy-B-CIDE-8 as a white solid. LCMS (ESI, m/z): [M+H]⁺=1649. ¹H NMR (300 MHz, DMSO-d₆, ppm) δ 10.13 (s, 1H), 9.57 (s, 1H), 8.99 (s, 1H), 8.41 (d, J=7.7 Hz, 1H), 8.00-7.80 (m, 3H), 7.55 (d, J=8.3 Hz, 2H), 7.50-7.39 (m, 2H), 7.39-7.11 (m, 8H), 7.08-6.97 (m, 2H), 6.96 (s, 2H), 6.56 (d, J=6.5 Hz, 1H), 6.18 (s, 1H), 6.00 (s, 1H), 4.98 (s, 2H), 4.79 (t, J=7.2 Hz, 1H), 4.50-4.12 (m, 10H), 3.60-3.51 (m, 5H), 3.37 (q, J=11.1, 10.2 Hz, 10H), 3.06-2.83 (m, 11H), 2.72-2.57 (m, 9H), 2.45-2.35 (m, 7H), 2.31-2.10 (m, 2H), 1.99-1.85 (m, 3H), 1.79 (s, 2H), 1.72-1.39 (m, 9H), 1.34 (d, J=7.0 Hz, 3H), 1.25 (d, J=7.0 Hz, 3H), 1.20-0.90 (m, 7H), 0.85 (dd, J=6.6, 3.7 Hz, 3H), 0.69 (dd, J=10.5, 6.6 Hz, 3H).

### Example 18

### Synthesis of Hy-B-PCIDE-2

### (3R,5S)-1-((2R)-2-(3-(2-(4-((1r,3R)-3-((4-(3-(3-Amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-(((S)-1-(4-cyanophenyl)ethyl)carbamoyl)pyrrolidin-3-yl dihydrogen phosphate (2,2,2-trifluoroacetic acid Salt)

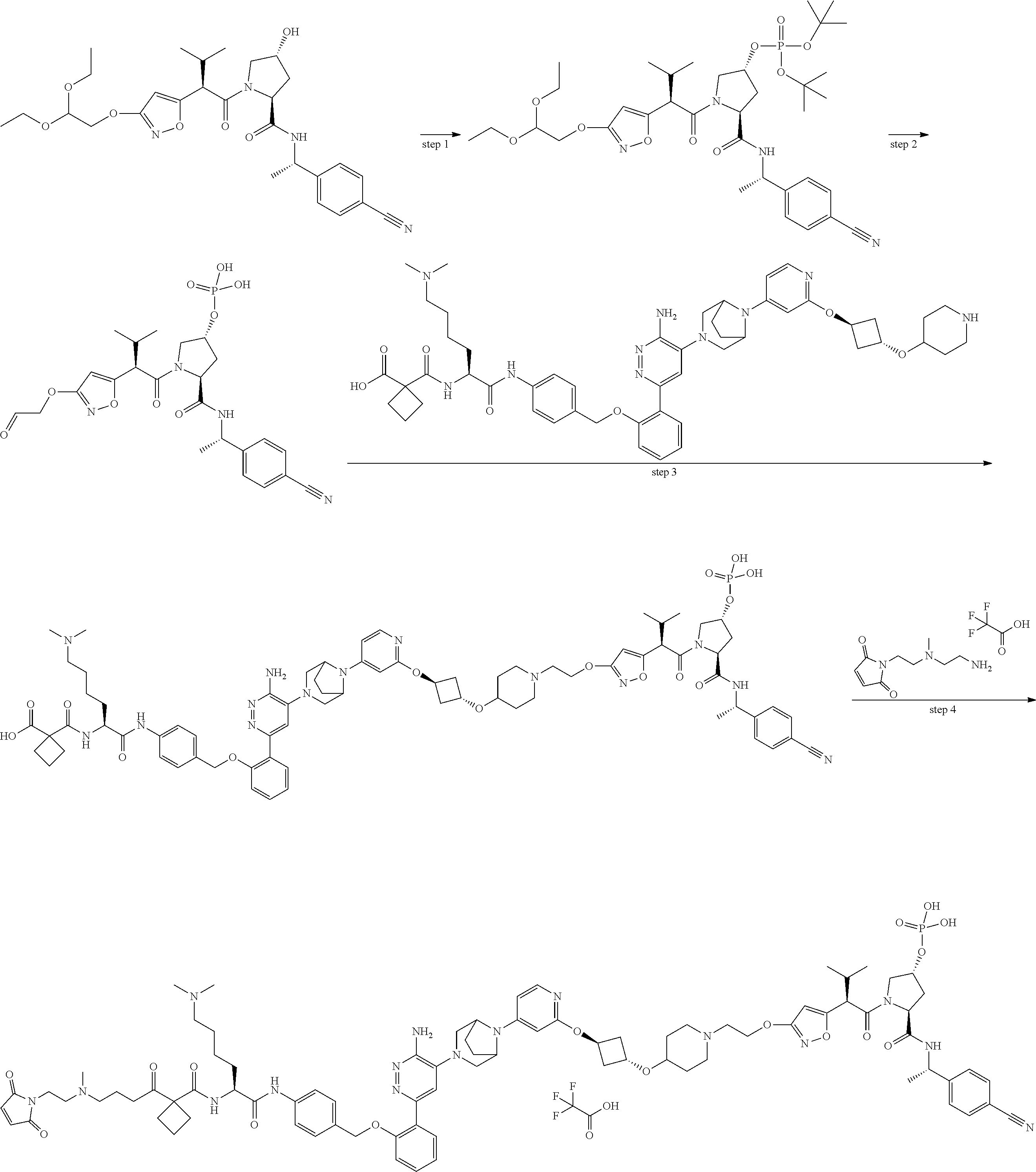

### Step 1: di-tert-butyl ((3R,5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl) phosphate

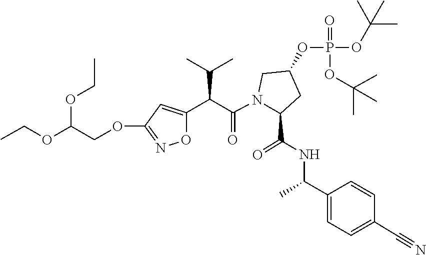 [SMILES: CCOC(COc1cc([C@H](C(=O)N2C[C@H](OP(=O)(OC(C)(C)C)OC(C)(C)C)C[C@H]2C(=O)N[C@@H](C)c2ccc(C#N)cc2)C(C)C)on1)OCC]

Under nitrogen, to a solution of (2S,4R)—N—((S)-1-(4-cyanophenyl)ethyl)-1-((R)-2-(3-(2,2-diethoxyethoxy)isoxazol-5-yl)-3-methylbutanoyl)-4-hydroxypyrrolidine-2-carboxamide (500 mg, 0.921 mmol) and 1H-tetrazole (6.1 mL, 0.45 M in ACN) in THF (15 mL) was added di-tert-butyl diisopropylphosphoramidite (511 mg, 1.84 mmol) at 0° C. The resulting solution was stirred at room temperature overnight. Then t-BuOOH (5M in decane) (0.74 mL, 3.69 mmol) was added at −30° C. and stirred at room temperature for 5 h. The reaction was quenched with NaHSO₃. The resulting solution was extracted with EtOAc and the organic layers were combined. The organic layer was dried over anhydrous sodium sulfate and concentrated under vacuum. The residue was purified by flash chromatography on silica gel (gradient: 0%-87% ethyl acetate/petroleum ether) to yield 420 mg (62% yield) of the title compound as a colorless oil. LC-MS: (ESI, m/z): [M+H]⁺=735.

### Step 2: (3R,5S)-5-(((S)-1-(4-Cyanophenyl)ethyl)carbamoyl)-1-((R)-3-methyl-2-(3-(2-oxoethoxy)isoxazol-5-yl)butanoyl)pyrrolidin-3-yl dihydrogen phosphate

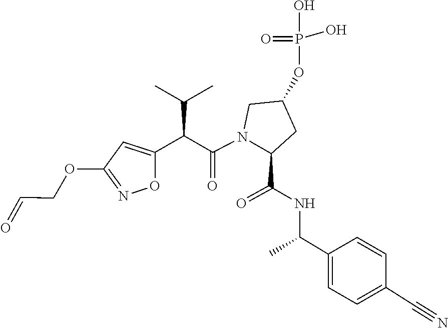 [SMILES: CC(C)[C@@H](C(=O)N1C[C@H](OP(=O)(O)O)C[C@H]1C(=O)N[C@@H](C)c1ccc(C#N)cc1)c1cc(OCC=O)no1]

Under nitrogen, a solution of di-tert-butyl ((3R,5S)-5-(((S)-1-(4-cyanophenyl)ethyl)carbamoyl)-1-((R)-2-(3-(2,2-diethoxyethoxy)isoxazol-5-yl)-3-methylbutanoyl)pyrrolidin-3-yl) phosphate (70.0 mg, 0.100 mmol) in HCOOH (0.5 mL) and water (0.5 mL) was stirred at 60° C. for 1 h. The solution was concentrated under vacuum to afford 50 mg (crude) of the title compound as a solid. LC-MS: (ESI, m/z): [M+H]⁺=549.

### Step 3: 1-(((2S)-1-((4-((2-(6-Amino-5-(8-(2-(3-((1-(2-((5-((R)-1-((2S,4R)-2-(((S)-1-(4-cyanophenyl)ethyl)carbamoyl)-4-(phosphonooxy)pyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

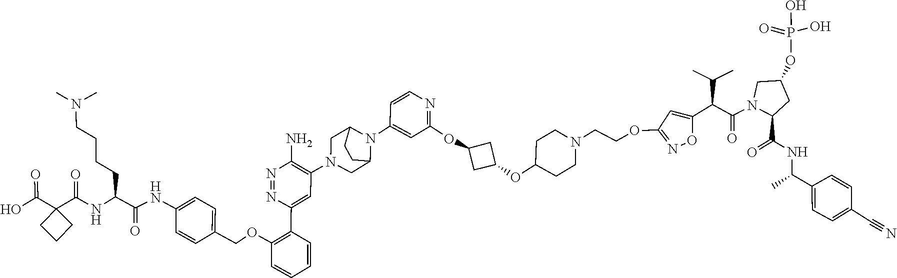 [SMILES: CC(C)[C@@H](C(=O)N1C[C@H](OP(=O)(O)O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)C3)CC2)no1]

Under nitrogen, a solution of (3R,5S)-5-(((S)-1-(4-cyanophenyl)ethyl)carbamoyl)-1-((R)-3-methyl-2-(3-(2-oxoethoxy)isoxazol-5-yl)butanoyl)pyrrolidin-3-yl dihydrogen phosphate (62.8 mg, 0.115 mmol), 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (56.0 mg, 0.0602 mmol), CH₃COONa (14.9 mg, 0.182 mmol) in methanol (1.5 mL) and dichloromethane (0.5 ml) was stirred at room temperature for 1 hour. Then NaBH₃CN (5.6 mg, 0.0891 mmol) was added and stirred at room temperature for 3 hours. The reaction was quenched by water. The reaction mixture was concentrated under vacuum. The residue was purified by pre-packed C18 column (solvent gradient: 0-100% ACN in water (0.05% NH₄HCO₃)) to yield 60 mg (65% yield) of the title compound as a white solid. LC-MS: (ESI, m/z): [M+H]⁺=1464.

### Step 4: (3R,5S)-1-((2R)-2-(3-(2-(4-((1r,3R)-3-((4-(3-(3-Amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidin-1-yl)ethoxy)isoxazol-5-yl)-3-methylbutanoyl)-5-((( S)-1-(4-cyanophenyl)ethyl)carbamoyl)pyrrolidin-3-yl dihydrogen phosphate (2,2,2-trifluoroacetic acid Salt)

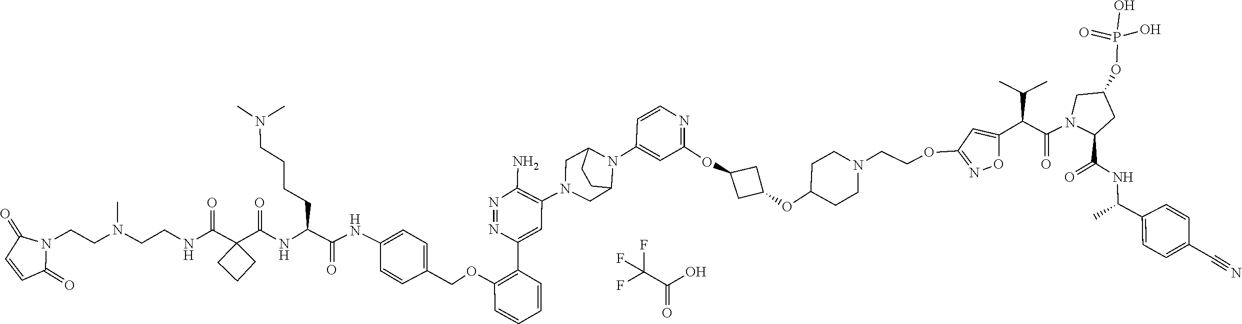 [SMILES: CC(C)[C@@H](C(=O)N1C[C@H](OP(=O)(O)O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)no1 ; O=C(O)C(F)(F)F]

To a solution of 1-(((2S)-1-((4-((2-(6-amino-5-(8-(2-(3-((1-(2-((5-((R)-1-((2S,4R)-2-(((S)-1-(4-cyanophenyl)ethyl)carbamoyl)-4-(phosphonooxy)pyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (60.0 mg, 0.0410 mmol), 1-(2-((2-aminoethyl)(methyl)amino)ethyl)-1H-pyrrole-2,5-dione (2,2,2-trifluoroacetic acid salt) (10.5 mg, crude) and DIPEA (54.2 mg, 0.420 mmol) in DMF (1 mL) was added HATU (18.7 mg, 0.0492 mmol) at room temperature. The reaction was stirred at room temperature for 10 minutes. The crude was purified by Prep-HPLC (Column: Xselect CSH C18 OBD Column 30*150 mm 5 m, n; Mobile Phase A: Water (0.05% TFA), Mobile Phase B: ACN; Flow rate: 60 mL/min; Gradient: 14% B to 23% B in 12 min, 23% B; Wave Length: 254/220 nm; R_{T1}(min): 11.75 min) to yield 20.3 mg (30% yield) of Hy-B-PCIDE-2 as a white solid.

LC-MS: (ESI, m/z): [M+H]⁺=1643. ¹H NMR (300 MHz, DMSO-d₆) δ 10.24 (s, 1H), 9.67 (s, 1H), 8.56 (d, J=7.3 Hz, 1H), 8.12 (s, 1H), 7.88-7.73 (m, 4H), 7.68-7.49 (m, 4H), 7.49-7.25 (m, 6H), 7.20-6.80 (m, 4H), 6.61 (s, 1H), 6.21-5.95 (m, 2H), 5.30-5.00 (m, 3H), 4.90 (t, J=6.9 Hz, 1H), 4.76 (s, 1H), 4.52-4.27 (m, 7H), 3.76-3.67 (m, 6H), 3.55-3.40 (m, 9H), 3.30-3.20 (m, 5H), 3.15-2.95 (m, 5H), 2.81 (s, 3H), 2.73 (s, 6H), 2.45-2.30 (m, 9H), 2.01-1.54 (m, 15H), 1.49-1.20 (m, 5H), 0.94 (d, J=6.4 Hz, 3H), 0.79 (d, J=7.0 Hz, 3H).

### Example 19

### Synthesis of Hy-B-CIDE-7

### N-((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(2-((2-(2,5-dioxo-2,5-dihydro-1H-pyrrol-1-yl)ethyl)(methyl)amino)ethyl)cyclobutane-1,1-dicarboxamide (2,2,2-trifluoroacetic acid Salt)

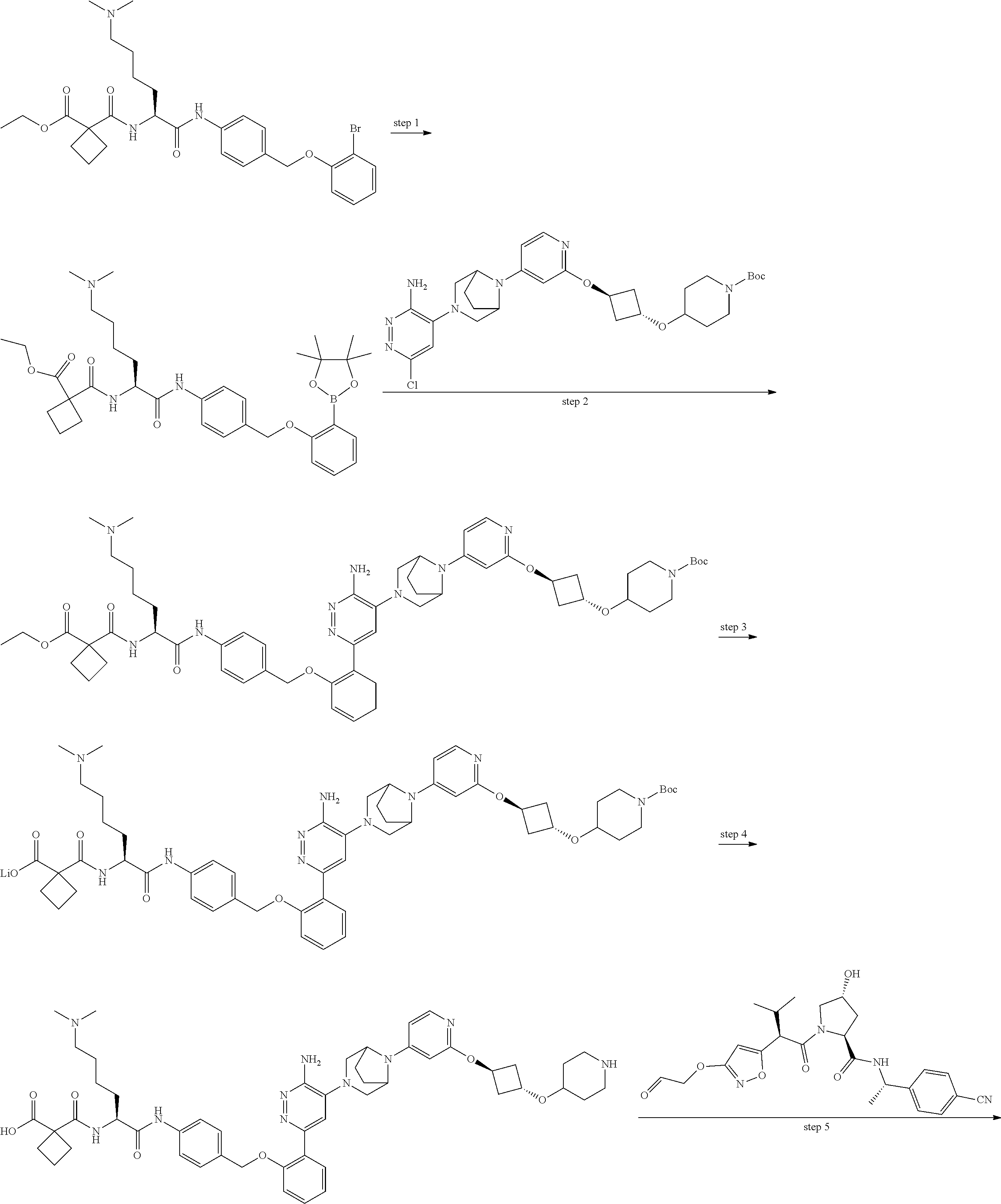

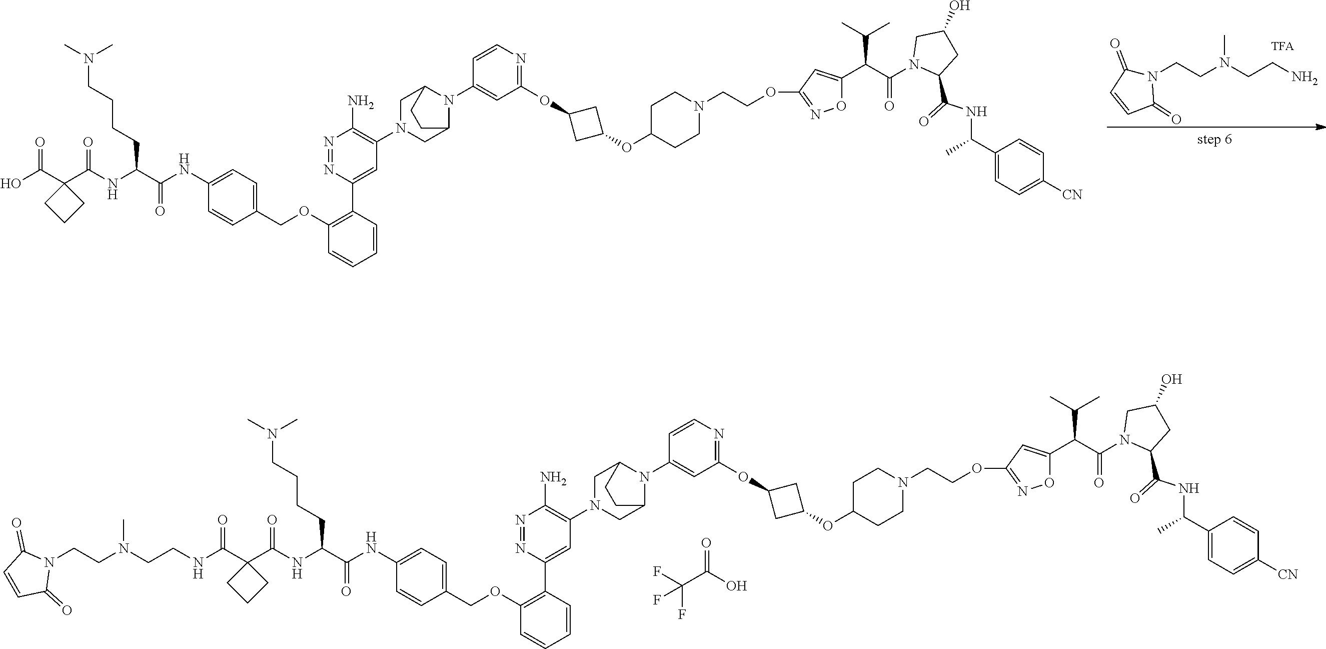 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)C3)CC2)no1 ; CN(CCN)CCN1C(=O)C=CC1=O ; CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)no1 ; O=C(O)C(F)(F)F]

### Step 1: Ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate

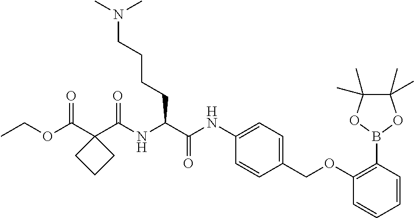 [SMILES: CCOC(=O)C1(C(=O)N[C@@H](CCCCN(C)C)C(=O)Nc2ccc(COc3ccccc3B3OC(C)(C)C(C)(C)O3)cc2)CCC1]

Under nitrogen, a solution of ethyl (S)-1-(1-((4-((2-bromophenoxy)methyl)phenyl)amino)-6-(dimethylamino)-1-oxohexan-2-yl)carbamoyl)cyclobutane-1-carboxylate (500 mg, 0.852 mmol), B₂Pin₂ (649 mg, 2.55 mmol), Pd(dppf)Cl₂ (124 mg, 0.170 mmol) and KOAc (250 mg, 2.55 mmol) in 1,4-dioxane (5 mL) was stirred at 80° C. for 2 hours. The reaction was diluted with DCM and washed with water. The organic layer was dried over anhydrous sodium sulfate and concentrated under vacuum. The residue was purified by flash chromatography on silica gel (gradient: 0%-20% MeOH/DCM (contain 0.3% 7 M NH₃/MeOH)) to yield 390 mg (72% yield) of the title compound as a yellow solid. LC-MS: (ESI, m/z): [M+H]⁺=636.

### Step 2: tert-Butyl 4-((1r,3r)-3-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidine-1-carboxylate

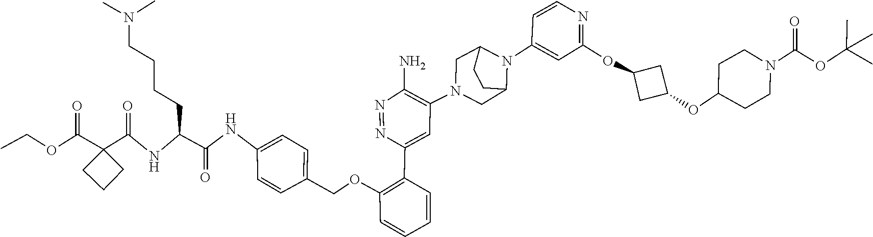 [SMILES: CCOC(=O)C1(C(=O)N[C@@H](CCCCN(C)C)C(=O)Nc2ccc(COc3ccccc3-c3cc(N4CC5CCC(C4)N5c4ccnc(O[C@H]5C[C@H](OC6CCN(C(=O)OC(C)(C)C)CC6)C5)c4)c(N)nn3)cc2)CCC1]

Under nitrogen, a solution of ethyl (5)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate (390 mg, 0.614 mmol), tert-butyl 4-((1r,3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate (395 mg, 0.676 mmol), Ad₂nBuPPdG2 (41.0 mg, 0.061 mmol) and K₂CO₃ (260 mg, 1.22 mmol) in dioxane (5.0 mL) and H₂O (1.2 mL) was stirred at 95° C. for 2 hours. The resulting solution diluted with water and extracted with EtOAc. Organic layer was concentrated under vacuum. The residue was purified by pre-packed C18 column (solvent gradient: 0-100% MeOH in water (0.1% NH₄HCO₃)) to yield 310 mg (47% yield) of the title compound as a white solid. LC-MS: (ESI, m/z): [M+H]⁺=1060.

### Step 3: lithium 1-(((2S)-1-((4-((2-(6-amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate

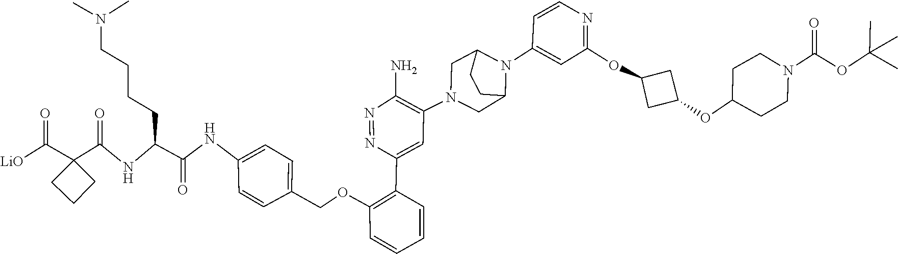

A solution of tert-butyl 4-((1r,3r)-3-((4-(3-(3-amino-6-(2-((4-((S)-6-(dimethylamino)-2-(1-(ethoxycarbonyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)cyclobutoxy)piperidine-1-carboxylate (270 mg, 0.255 mmol) and LiOH·H₂O (32.1 mg, 0.765 mmol) in THF (2 mL) and H₂O (2 mL) was stirred at room temperature for 1 h. The resulting mixture was concentrated under vacuum. The crude was used in next step directly. LC-MS: (ESI, m/z): [M+H]⁺=1032.

### Step 4: 1-(((2S)-1-((4-((2-(6-Amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

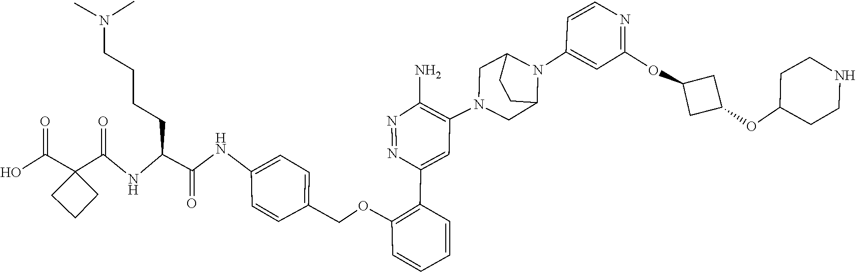 [SMILES: CN(C)CCCC[C@H](NC(=O)C1(C(=O)O)CCC1)C(=O)Nc1ccc(COc2ccccc2-c2cc(N3CC4CCC(C3)N4c3ccnc(O[C@H]4C[C@H](OC5CCNCC5)C4)c3)c(N)nn2)cc1]

A solution of lithium 1-(((2S)-1-((4-((2-(6-amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylate (crude from last step) in TFA (0.75 mL) and HFIP (14 mL) was stirred at room temperature for 30 min. The resulting mixture was concentrated under vacuum. The obtained crude product was purified by pre-packed C18 column (solvent gradient: 0-100% MeOH in water (0.1% NH₄HCO₃)) to yield 170 mg (71% yield) of the title compound as a yellow solid. LC-MS: (ESI, m/z): [M+H]⁺=932.

### Step 5: 1-(((2S)-1-((4-((2-(6-Amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

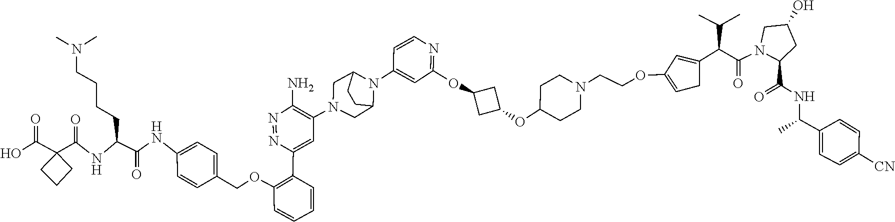 [SMILES: CC(C)[C@@H](C(=O)N1C[C@H](O)C[C@H]1C(=O)N[C@@H](C)c1ccc(C#N)cc1)C1=CC(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)C3)CC2)=CC1]

Under nitrogen, a solution of 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (170.0 mg, 0.183 mmol), (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (111 mg, 0.237 mmol), HOAc (21.9 mg, 0.365 mmol) in DCM (1.5 mL) and MeOH (0.5 mL) was stirred at room temperature for 1 hour. Then NaBH₃CN (17.3 mg, 0.274 mmol) was added at 0° C. and stirred at room temperature temperature for 30 min. The reaction was quenched with water. The resulting solution was concentrated under vacuum. The obtained crude product was purified by pre-packed C18 column (gradient: 0˜100% MeOH in water (0.05% NH₄HCO₃)) to yield 180 mg (71% yield) of the title compound as a white solid. LC-MS: (ESI, m/z): [M+H]⁺=1384.

### Step 5: N-((2S)-1-((4-((2-(6-Amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(2-((2-(2,5-dioxo-2,5-dihydro-1H-pyrrol-1-yl)ethyl)(methyl)amino)ethyl)cyclobutane-1,1-dicarboxamide (2,2,2-trifluoroacetic acid Salt)

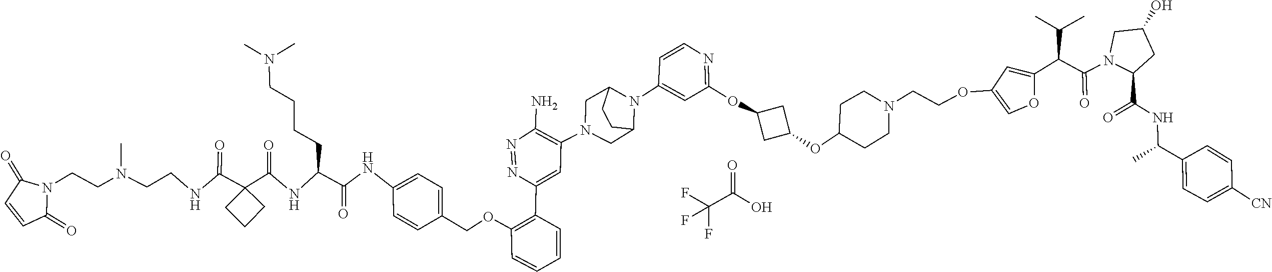 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)co1 ; O=C(O)C(F)(F)F]

To a solution of 1-(((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (2,2,2-trifluoroacetic acid salt) (60.0 mg, crude), 1-(2-((2-aminoethyl)(methyl)amino)ethyl)-1H-pyrrole-2,5-dione (12.8 mg, 0.065 mmol), DIPEA (56.0 mg, 0.434 mmol) in DMF (1 mL) was added HATU (19.8 mg, 0.052 mmol) at room temperature. The reaction was stirred at room temperature for 30 min. The resulting solution was purified by Prep-HPLC (Xselect CSH C18 OBD Column 30×150 mm 5 m; Mobile Phase A: Water (0.05% TFA), Mobile Phase B: ACN; Flow rate: 60 mL/min; Gradient: 8% B to 32% B in 8 min; 254/220 nm; R_{T1}: 8 min) to yield 29.5 mg (43% yield) of Hy-B-CIDE-7 as a white solid. LC-MS: (ESI, m/z): [M+H]⁺=1563. ¹H NMR (300 MHz, DMSO-d₆) δ 10.22 (s, 1H), 9.69 (br, 2H), 8.46 (d, J=7.3 Hz, 1H), 8.12 (s, 1H), 7.90-7.70 (m, 4H), 7.70-7.50 (m, 4H), 7.51-7.27 (m, 6H), 7.19-6.96 (m, 5H), 6.65 (s, 1H), 6.17-5.98 (m, 2H), 5.22 (s, 1H), 5.10 (s, 2H), 4.90 (d, J=7.2 Hz, 1H), 4.51 (s, 4H), 4.45-4.19 (m, 5H), 3.76-3.65 (m, 7H), 3.43 (s, 5H), 3.30-3.20 (m, 5H), 3.14-2.97 (m, 6H), 2.84 (s, 3H), 2.75 (s, 7H), 2.42-2.30 (m, 7H), 2.27-2.24 (m, 1H), 2.07-1.52 (m, 17H), 1.50-1.20 (m, 5H), 0.96 (t, J=6.3 Hz, 3H), 0.82 (dd, J=9.1, 6.6 Hz, 3H).

### Example 20

### Synthesis of Hy-B-CIDE-9

### 6-(8-Chloro-4-(2-(dimethylamino)ethyl)-5,6-dihydro-4H-[1,4]oxazepino[5,6,7-de]quinazolin-9-yl)-4-methyl-5-(trifluoromethyl)pyridin-2-amine (2,2,2-trifluoroacetic acid Salt)

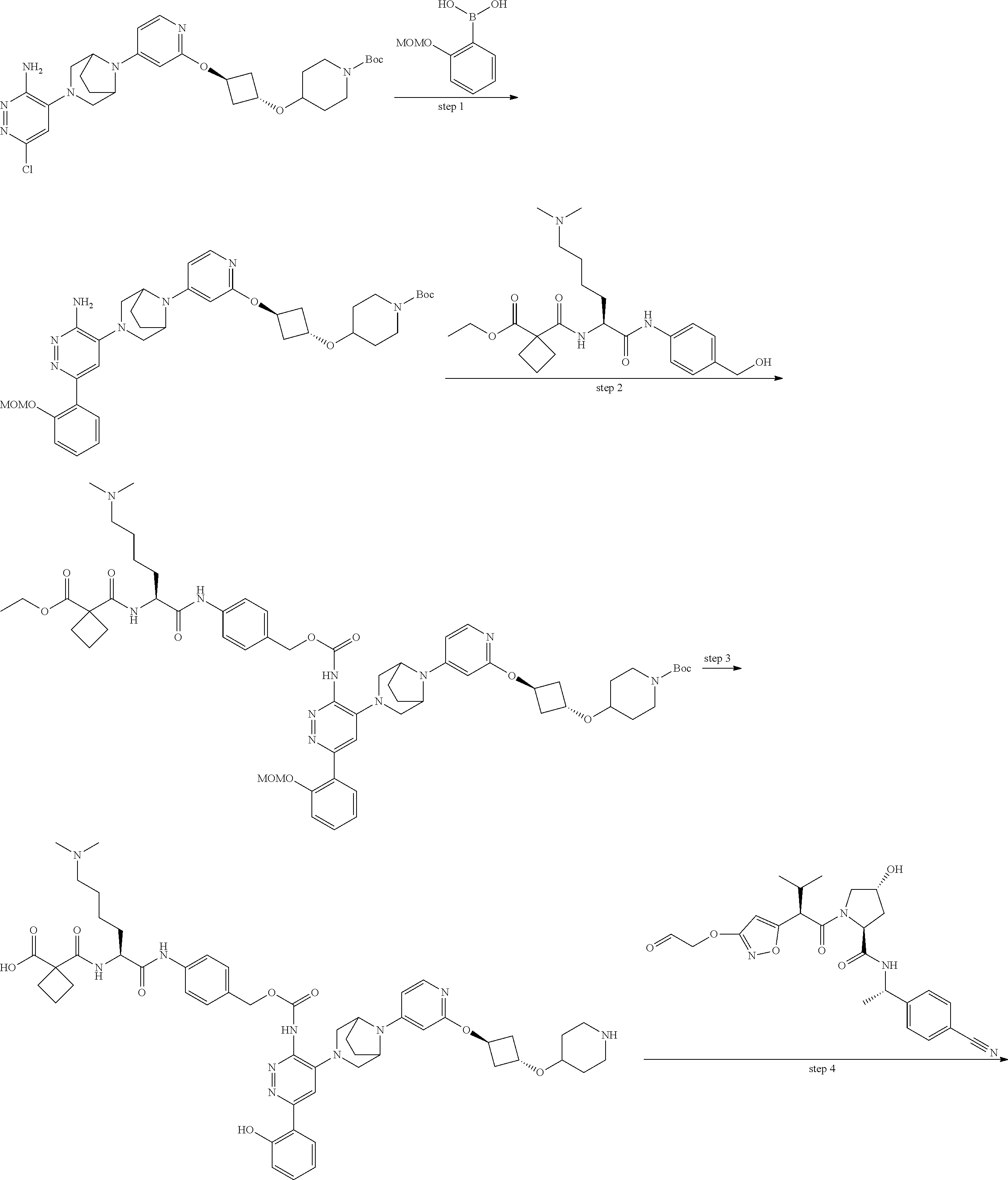

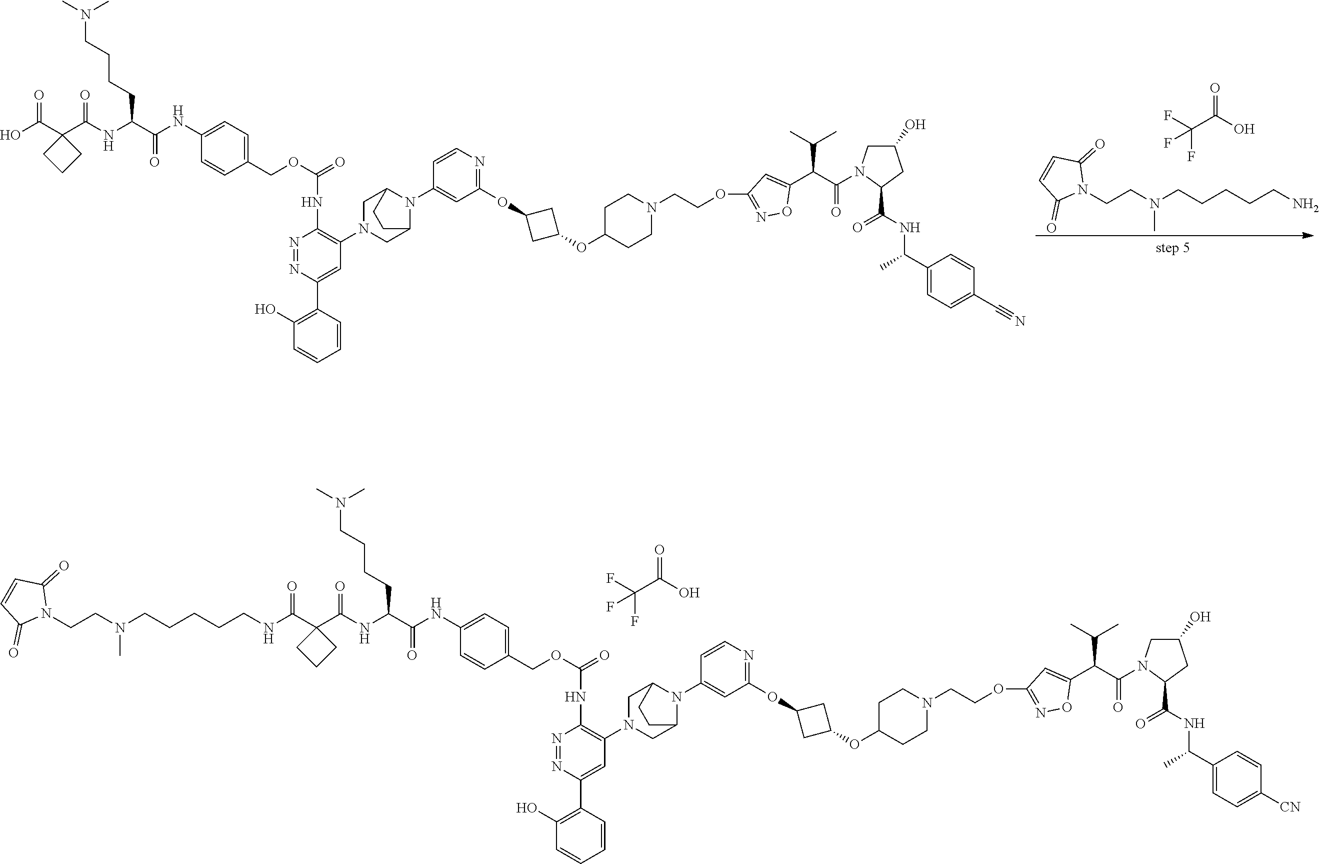

### Step 1: tert-Butyl 4-((1r,3r)-3-((4-(3-(3-amino-6-(2-(methoxymethoxy)phenyl)pyridazin-4-yl)-3,8-diazabicyclo[3.2.1]octan-8-yl)pyridin-2-yl)oxy)cyclobutoxy)piperidine-1-carboxylate

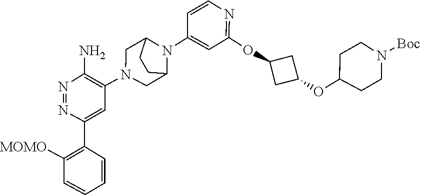

Under nitrogen, a solution of tert-butyl 4-((1r,3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate (1.50 g, 2.56 mmol), 2-(methoxymethoxy)phenylboronic acid (559 mg, 3.07 mmol), Pd(PPh₃)₄ (591 mg, 0.512 mmol) and K₂CO₃ (1.06 g, 7.68 mmol) in dioxane (12.5 mL) and H₂O (2.5 mL) was stirred at 100° C. for 1 h. The reaction mixture was diluted with water and extracted with DCM. The combined organic layer was dried over anhydrous Na₂SO₄ and concentrated under reduced pressure. The obtained crude product was purified flash chromatography on silica gel column (solvent gradient: 0-10% MeOH/DCM) to afford 1.33 g (75% yield) of the title compound as a yellow solid. LCMS (ESI, m/z): [M+H]⁺=688.

### Step 2: tert-Butyl 4-((1r,3r)-3-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-(methoxymethoxy)phenyl)pyridazin-4-yl)-3,8-diazabicyclo[3.2.1]octan-8-yl)pyridin-2-yl)oxy)cyclobutoxy)piperidine-1-carboxylate

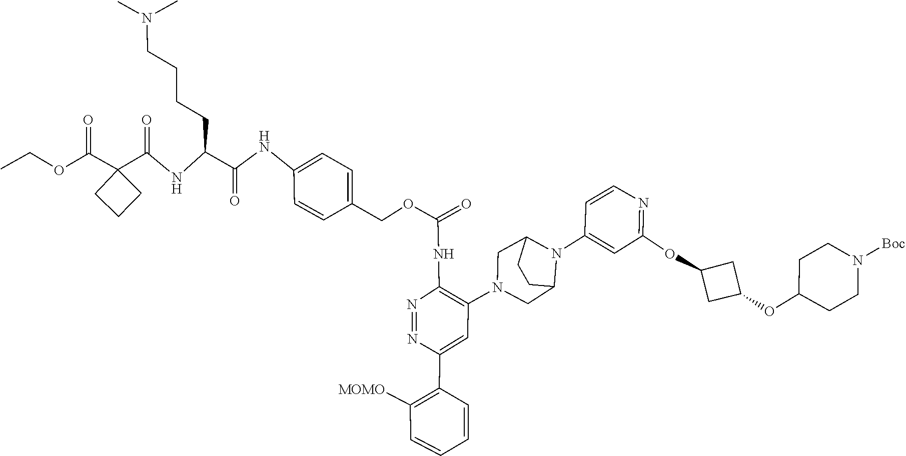

Under nitrogen, to a solution of tert-butyl 4-((1r,3r)-3-((4-(3-(3-amino-6-(2-(methoxymethoxy)phenyl)pyridazin-4-yl)-3,8-diazabicyclo[3.2.1]octan-8-yl)pyridin-2-yl)oxy)cyclobutoxy)piperidine-1-carboxylate (400 mg, 0.582 mmol), ethyl (S)-1-((6-(dimethylamino)-1-((4-(hydroxymethyl)phenyl)amino)-1-oxohexan-2-yl)carbamoyl)cyclobutane-1-carboxylate (882 mg, 2.04 mmol) and DIPEA (1.19 g, 9.20 mmol) in THF (10 mL) was added triphosgene (313 mg, 1.05 mmol) at 0° C. The reaction was stirred at room temperature for 0.5 h. Solvent was evaporated and residue was purified by pre-packed C18 column (solvent gradient: 0-100% MeOH in water (0.05% NH₄HCO₃)) to yield 160 mg (23%) of the title compound as a yellow solid. LC-MS: (ESI, m/z): [M+H]⁺=1148.

### Step 3: 1-(((2S)-6-(dimethylamino)-1-((4-((((6-(2-hydroxyphenyl)-4-(8-(2-((1r,3r)-3-(piperidin-4-yloxy)cyclobutoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid

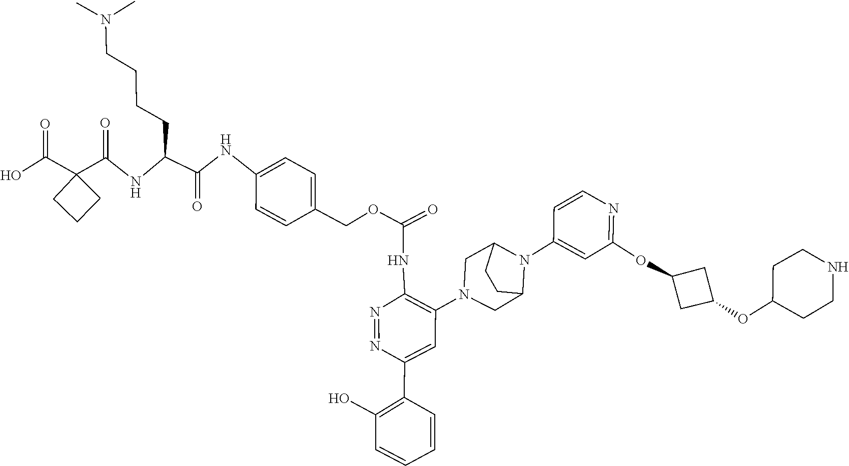 [SMILES: CN(C)CCCC[C@H](NC(=O)C1(C(=O)O)CCC1)C(=O)Nc1ccc(COC(=O)Nc2nnc(-c3ccccc3O)cc2N2CC3CCC(C2)N3c2ccnc(O[C@H]3C[C@H](OC4CCNCC4)C3)c2)cc1]

A solution of tert-butyl 4-((1r,3r)-3-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-(methoxymethoxy)phenyl)pyridazin-4-yl)-3,8-diazabicyclo[3.2.1]octan-8-yl)pyridin-2-yl)oxy)cyclobutoxy)piperidine-1-carboxylate (60.0 mg, 0.0520 mmol) and LiOH·H₂O (6.58 mg, 0.156 mmol) in THF (0.5 ml) and H₂O (0.5 ml) was stirred at room temperature for 1 h. The resulting mixture was concentrated under vacuum. Then it was dissolved in a solution of concentrated HCl (0.33 ml) and H₂O (0.67 ml). The reaction was stirred at room temperature for 20 min. The resulting mixture was basified to pH=8 with saturated NaHCO₃ solution at 0° C. and purified by pre-packed C18 column (solvent gradient: 0-100% MeOH in water (0.05% NH₄HCO₃)) to yield 35 mg of the title compound as a yellow solid. LCMS (ESI, m/z): [M+H]⁺=976.

### Step 4: 1-(((2S)-1-((4-((((4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid

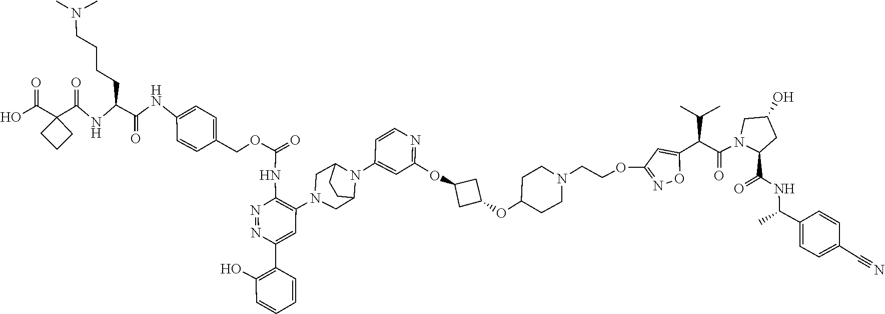 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7O)nnc5NC(=O)OCc5ccc(NC(=O)[C@H](CCCCN(C)C)NC(=O)C7(C(=O)O)CCC7)cc5)C6)ccn4)C3)CC2)no1]

Under nitrogen, a solution of 1-(((2S)-6-(dimethylamino)-1-((4-((((6-(2-hydroxyphenyl)-4-(8-(2-((1r,3r)-3-(piperidin-4-yloxy)cyclobutoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid (30.0 mg, 0.0310 mmol), (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (28.8 mg, 0.0620 mmol) and HOAc (3.69 mg, 0.0620 mmol) in DCM (0.75 mL) and MeOH (0.25 mL) was stirred at room temperature for 1 h. Then NaBH₃CN (2.90 mg, 0.0460 mmol) was added and stirred at room temperature for 0.5 h. The reaction was quenched with water at 0° C. The resulting mixture was concentrated under vacuum and purified by pre-packed C18 column (solvent gradient: 0-100% MeOH in water (0.05% NH₄HCO₃)) to yield 27 mg of the title compound as a white solid. LCMS (ESI, m/z): [M+H]⁺=1428.

### Step 5: 4-((S)-6-(Dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl (4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-hydroxyphenyl)pyridazin-3-yl)carbamate (2,2,2-trifluoroacetic acid Salt)

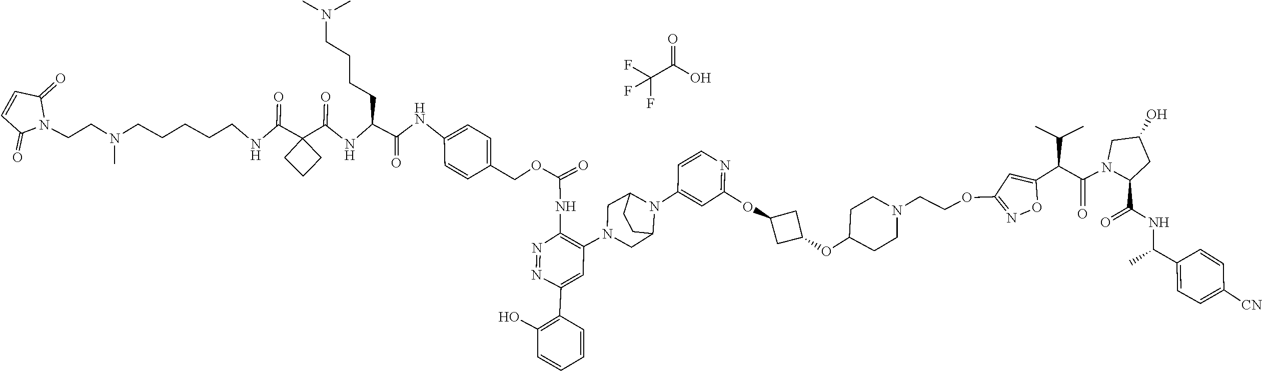 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7O)nnc5NC(=O)OCc5ccc(NC(=O)[C@H](CCCCN(C)C)NC(=O)C7(C(=O)NCCCCCN(C)CCN8C(=O)C=CC8=O)CCC7)cc5)C6)ccn4)C3)CC2)no1 ; O=C(O)C(F)(F)F]

To a solution of 1-(((2S)-1-((4-((((4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid (22.0 mg, 0.0150 mmol), 1-(2-((5-aminopentyl)(methyl)amino)ethyl)-1H-pyrrole-2,5-dione (2,2,2-trifluoroacetic acid salt) (5.5 mg, crude) and DIPEA (19.9 mg, 0.154 mmol) in DMF (1 mL) was added HATU (7.62 mg, 0.0200 mmol) at room temperature. The reaction was stirred at room temperature for 10 min. The resulting mixture was purified by Prep-HPLC with the following conditions: Column: XBridge Shield RP18 OBD Column, 30*150 mm, 5 m; Mobile Phase A: Water (0.05% TFA), Mobile Phase B: ACN; Flow rate: 60 mL/min; Gradient: 2% B to 32% B in 10 min, 32% B; Wave Length: 220/254 nm; R_{T}(min): 11.93 to yield 13.8 mg of Hy-B-CIDE-9 as a white solid.

LCMS (ESI, m/z): [M+H]⁺=1649. ¹H NMR (300 MHz, DMSO-d₆, ppm) δ 10.17 (s, 1H), 10.03 (s, 1H), 9.70 (br, 1H), 9.47 (br, 2H), 8.47 (d, J=7.3 Hz, 1H), 7.99-7.83 (m, 4H), 7.78 (m, J=9.1, 7.3 Hz, 2H), 7.64 (d, J=8.2 Hz, 3H), 7.45 (m, J=8.5, 2.4 Hz, 2H), 7.37-7.25 (m, 3H), 7.08 (d, J=3.9 Hz, 2H), 6.98-6.90 (m, 2H), 6.72 (s, 1H), 6.22 (s, 1H), 6.14 (s, 1H), 5.20 (s, 1H), 5.11 (s, 2H), 4.89 (t, J=7.0 Hz, 1H), 4.69 (s, 2H), 4.50 (s, 2H), 4.44-4.38 (m, 1H), 4.31 (t, J=11.3 Hz, 3H), 3.75-3.69 (m, 7H), 3.42-3.39 (m, 2H), 3.38-3.33 (m, 2H), 3.33-3.27 (m, 3H), 3.27-3.12 (m, 7H), 3.12-3.00 (m, 4H), 3.00 (s, 3H), 2.74 (s, 6H), 2.46-2.38 (m, 7H), 2.37-2.09 (m, 1H), 2.05-2.00 (m, 2H), 1.95-1.85 (m, 5H), 1.85-1.75 (m, 5H), 1.75-1.59 (m, 5H), 1.43 (t, J=6.6 Hz, 3H), 1.33 (d, J=7.0 Hz, 4H), 1.30-1.20 (m, 3H), 0.94 (d, J=6.5 Hz, 3H), 0.78 (d, J=6.8 Hz, 3H).

### Example 21

### Synthesis of Hy-B-CIDE-10

### 4-((S)-6-(Dimethylamino)-2-(1-((5-((S)-2-(2,5-dioxo-2,5-dihydro-1H-pyrrol-1-yl)-3-hydroxypropanamido)pentyl)carbamoyl)cyclobutane-1-carboxamido)hexanamido)benzyl (4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-hydroxyphenyl)pyridazin-3-yl)carbamate

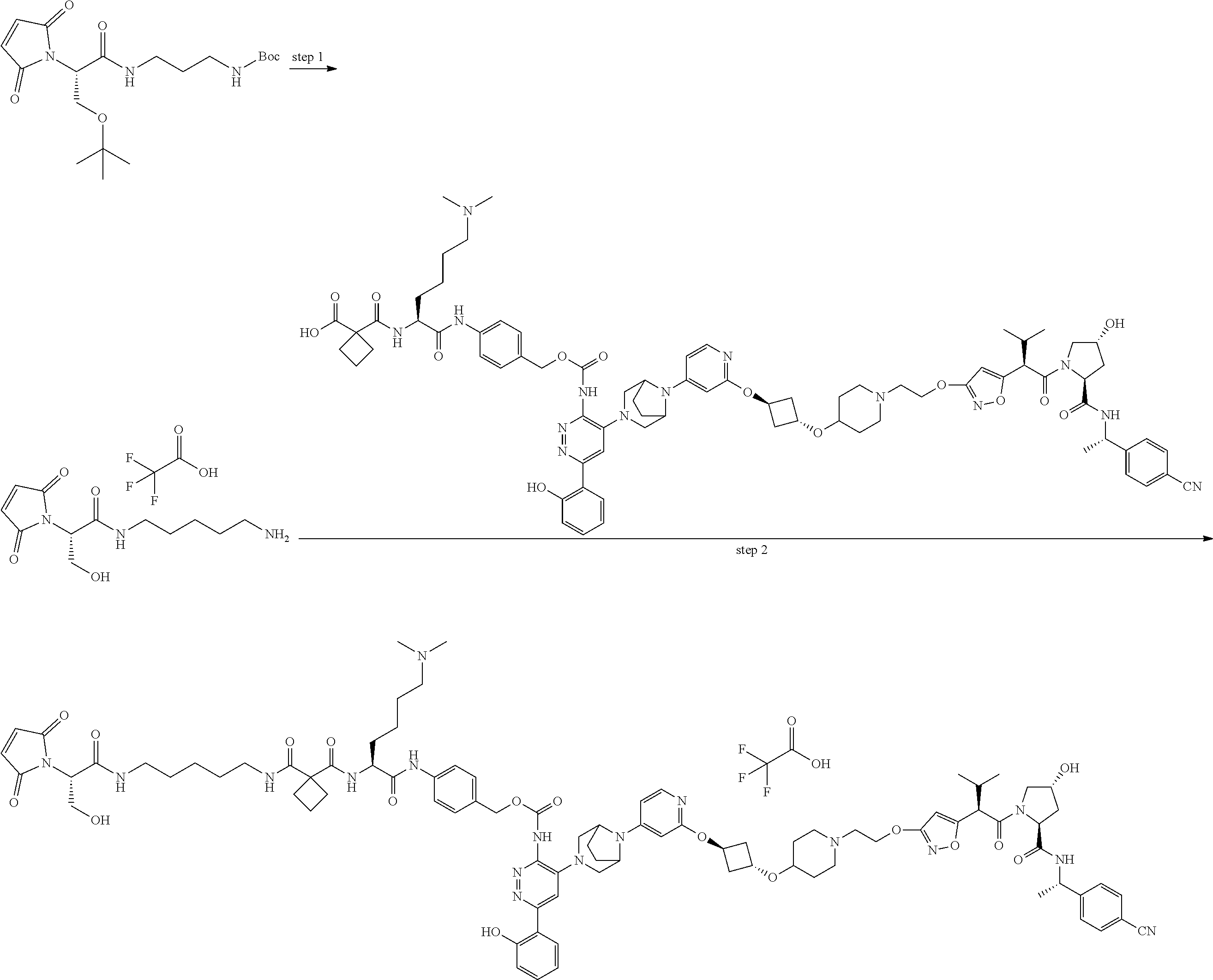

### Step 1: (S)-N-(5-aminopentyl)-2-(2,5-dioxo-2,5-dihydro-1H-pyrrol-1-yl)-3-hydroxypropanamide (2,2,2-trifluoroacetic acid)

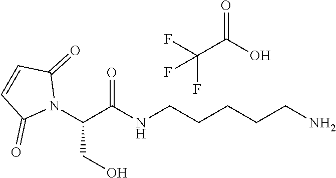

Under nitrogen a solution of tert-butyl (S)-(5-(3-(tert-butoxy)-2-(2,5-dioxo-2,5-dihydro-1H-pyrrol-1-yl)pentyl)carbamate (8.8 mg, 0.0094 mmol) in TFA (0.3 mL) was stirred at room temperature for 1.5 h. The solvent was concentrated under vacuum to afford 10 mg (crude) of the title compound as a colorless oil. LCMS (ESI, m/z): [M+H]⁺=270.

### Step 2: 4-((S)-6-(Dimethylamino)-2-(1-((5-((S)-2-(2,5-dioxo-2,5-dihydro-1H-pyrrol-1-yl)-3-hydroxypropanamido)pentyl)carbamoyl)cyclobutane-1-carboxamido)hexanamido)benzyl (4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-hydroxyphenyl)pyridazin-3-yl)carbamate (2,2,2-trifluoroacetic acid Salt)

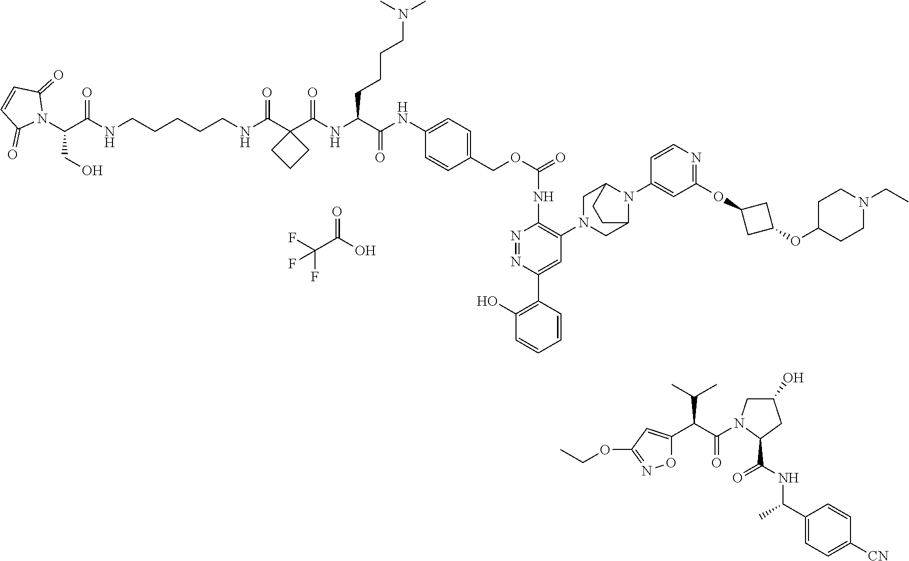

To a solution of 1-(((2S)-1-((4-((((4-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid (27.0 mg, 0.0190 mmol), (S)—N-(5-aminopentyl)-2-(2,5-dioxo-2,5-dihydro-1H-pyrrol-1-yl)-3-hydroxypropanamide (2,2,2-trifluoroacetic acid) (8.8 mg, crude) and DIPEA (24.4 mg, 0.189 mmol) in DMF (0.9 mL) was added HATU (9.3 mg, 0.025 mmol) at room temperature. The reaction was stirred at room temperature for 10 min. The crude was purified by Prep-HPLC with the following conditions: Column: Xselect CSH C18 OBD Column 30*150 mm 5 m, n; Mobile Phase A: Water (0.05% TFA), Mobile Phase B: ACN; Flow rate: 60 mL/min; Gradient: 15% B to 27% B in 12 min, 27% B; Wave Length: 254/220 nm; R_{T} (min): 12; to yield 9.9 mg of Hy-B-CIDE-10 as a white solid.

LCMS (ESI, m/z): [M+H]⁺=1679. ¹H NMR (300 MHz, DMSO-d₆, ppm) δ 10.16 (s, 1H), 10.04 (s, 1H), 9.60 (br, 1H), 9.35 (br, 1H), 8.47 (d, J=7.4 Hz, 1H), 8.06-7.84 (m, 4H), 7.79 (t, J=8.3 Hz, 2H), 7.69-7.58 (m, 3H), 7.45 (d, J=8.4, 2H), 7.40-7.34 (m, 3H), 7.04 (s, 2H), 7.00-6.88 (m, 2H), 6.72 (s, 1H), 6.21 (s, 1H), 6.14 (s, 1H), 5.19 (s, 1H), 5.11 (s, 2H), 4.89 (t, J=7.1 Hz, 1H), 4.68 (d, J=7.1 Hz, 2H), 4.55-4.23 (m, 7H), 3.97-3.82 (m, 2H), 3.67 (d, J=9.5 Hz, 3H), 3.62-3.52 (m, 8H), 3.17-2.95 (m, 10H), 2.73 (d, J=4.1 Hz, 6H), 2.45-2.30 (m, 8H), 2.19-1.97 (m, 4H), 1.95-1.85 (m, 5H), 1.82-1.53 (m, 8H), 1.45-1.30 (m, 8H), 1.25-1.15 (m, 4H), 0.95 (t, J=6.4 Hz, 3H), 0.90-0.70 (m, 3H).

### Example 22

### Synthesis of Hy-B-CIDE-11

### N-((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((2-(2,5-dioxo-2,5-dihydro-1H-pyrrol-1-yl)ethyl)(methyl)amino)pentyl)cyclobutane-1,1-dicarboxamide (2,2,2-trifluoroacetic acid Salt)

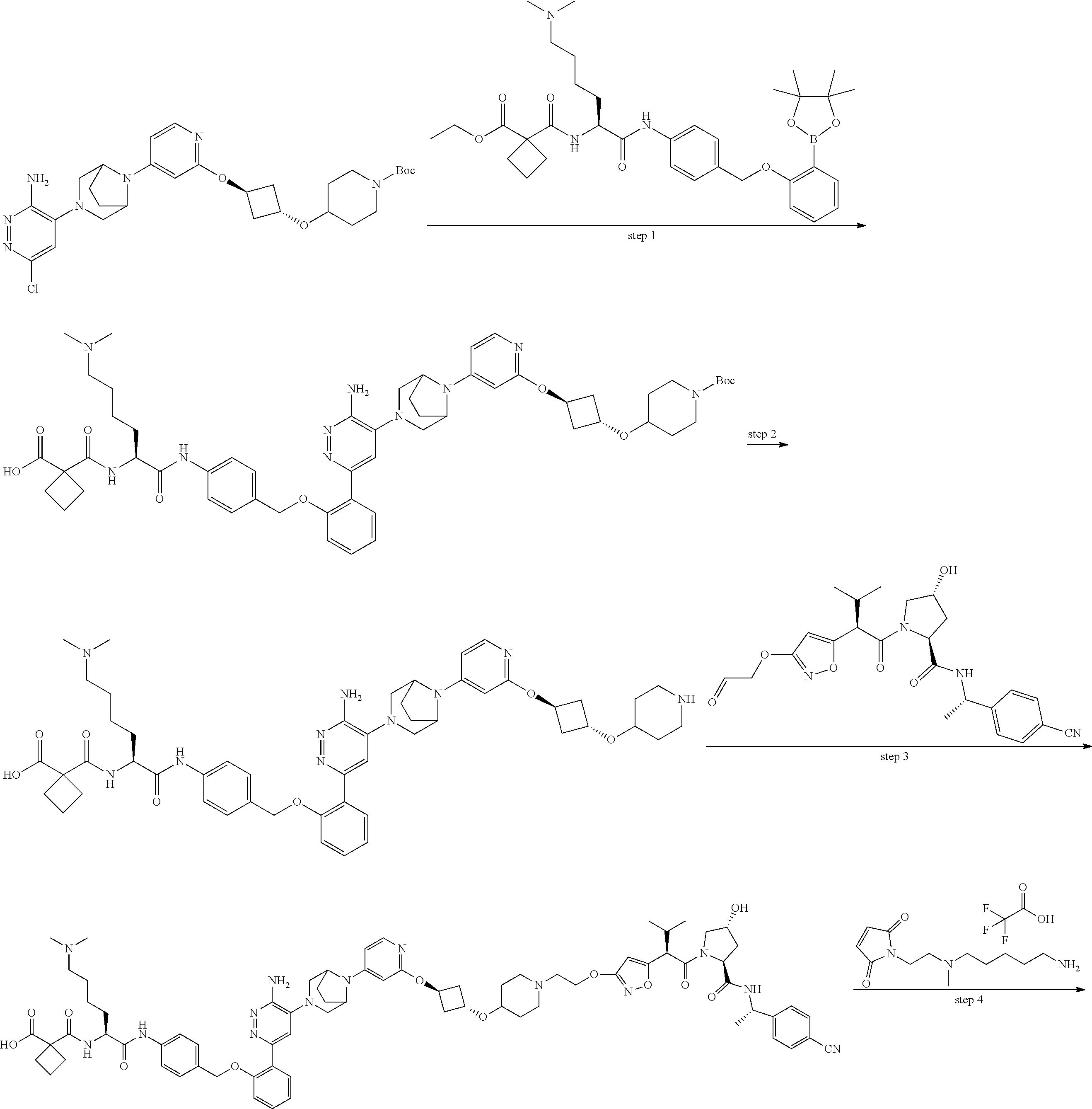

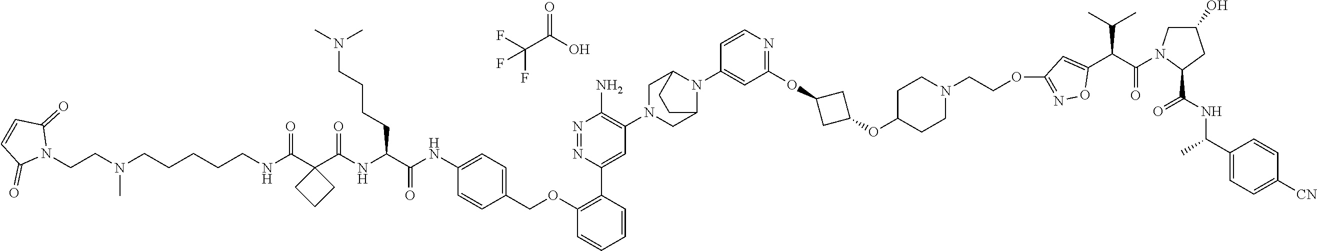 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCCCCN(C)CCN9C(=O)C=CC9=O)CCC8)cc7)nnc5N)C6)ccn4)C3)CC2)no1 ; O=C(O)C(F)(F)F]

### Step 1: 1-(((2S)-1-((4-((2-(6-Amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

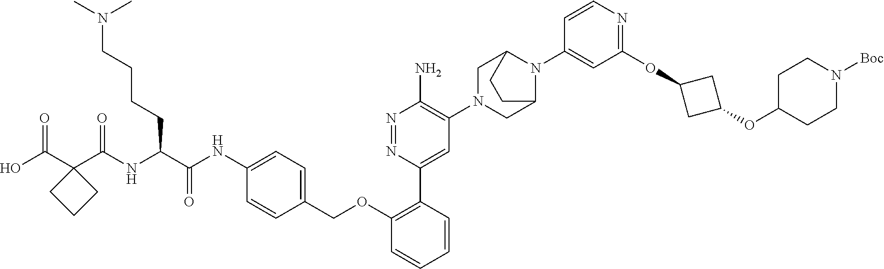 [SMILES: CN(C)CCCC[C@H](NC(=O)C1(C(=O)O)CCC1)C(=O)Nc1ccc(COc2ccccc2-c2cc(N3CC4CCC(C3)N4c3ccnc(O[C@H]4C[C@H](OC5CCN(C(=O)OC(C)(C)C)CC5)C4)c3)c(N)nn2)cc1]

Under nitrogen, a solution of tert-butyl 4-((1r,3r)-3-((4-(3-(3-amino-6-chloropyridazin-4-yl)-3,8-diazabicyclo[3.2.1]octan-8-yl)pyridin-2-yl)oxy)cyclobutoxy)piperidine-1-carboxylate (550 mg, 0.900 mmol), ethyl (S)-1-((6-(dimethylamino)-1-oxo-1-((4-((2-(4,4,5,5-tetramethyl-1,3,2-dioxaborolan-2-yl)phenoxy)methyl)phenyl)amino)hexan-2-yl)carbamoyl)cyclobutane-1-carboxylate (600 mg, 0.900 mmol), Pd(dppf)Cl₂ (138 mg, 0.200 mmol) and Na₂CO₃ (300 mg, 2.800 mmol) in 1,4-dioxane (10 mL) and water (2 mL) was stirred at 110° C. for 1 h. The reaction was cooled to room temperature and LiOH·H₂O (160 mg, 3.800 mmol) was added. The reaction was stirred at room temperature for 1 h. The resulting mixture was purified by pre-packed C18 column (solvent gradient: 0-70% ACN in water (0.5% NH₄HCO₃)) to afford 383 mg (39% yield) of the title compound as a brown solid. LCMS (ESI, m/z): [M+H]⁺=1032.

### Step 2: 1-(((2S)-1-((4-((2-(6-Amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (2,2,2-trifluoroacetic acid Salt)

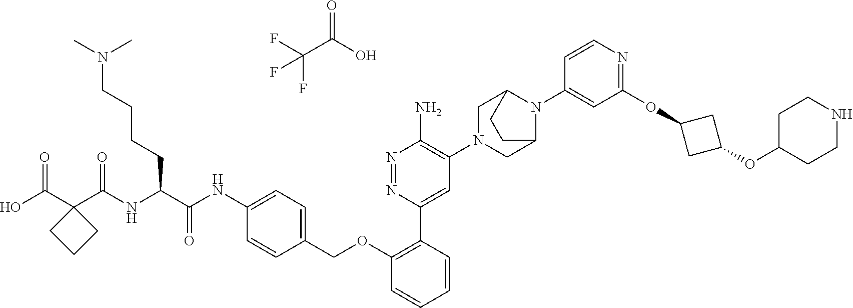 [SMILES: CN(C)CCCC[C@H](NC(=O)C1(C(=O)O)CCC1)C(=O)Nc1ccc(COc2ccccc2-c2cc(N3CC4CCC(C3)N4c3ccnc(O[C@H]4C[C@H](OC5CCNCC5)C4)c3)c(N)nn2)cc1 ; O=C(O)C(F)(F)F]

A solution of 1-(((2S)-1-((4-((2-(6-amino-5-(8-(2-((1r,3r)-3-((1-(tert-butoxycarbonyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (383.0 mg, 0.3700 mmol) in DCM (1 mL) and 2,2,2-trifluoroacetic acid (1 mL) was stirred at room temperature for 30 min. The solvent was evaporated under vacuum and the residue was purified by pre-packed C18 column (solvent gradient: 0-100% ACN in water (0.05% TFA)) to afford 290 mg of the title compound as a white solid. LCMS (ESI, m/z): [M+H]⁺=931.

### Step 3: 1-(((2S)-1-((4-((2-(6-Amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

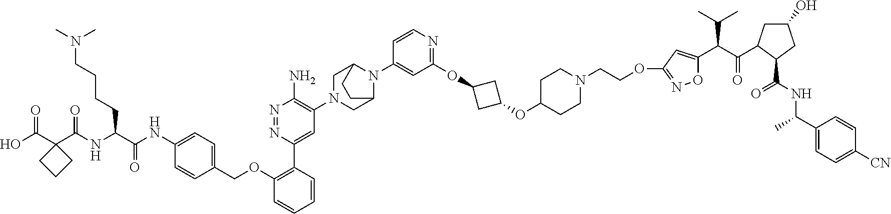 [SMILES: CC(C)[C@@H](C(=O)C1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)C3)CC2)no1]

A solution of 1-(((2S)-1-((4-((2-(6-amino-5-(8-(2-((1r,3r)-3-(piperidin-4-yloxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (2,2,2-trifluoroacetic acid salt) (260 mg, 0.248 mmol), (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (195 mg, 0.420 mmol) and NaOAc (228 mg, 2.79 mmol) in MeOH (1 mL) and DCM (3 mL) was stirred at room temperature for 1 h. Then NaBH₃CN (52.0 mg, 0.830 mmol) was added and stirred at room temperature for 1 h. The crude was purified by pre-packed C18 column (solvent gradient: 0-100% MeOH in water (0.05% NH₄HCO₃)) to afford 210 mg of the title compound as a yellow solid. LCMS (ESI, m/z): [M+H]⁺=1383.

### Step 4: N-((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((2-(2,5-dioxo-2,5-dihydro-1H-pyrrol-1-yl)ethyl)(methyl)amino)pentyl)cyclobutane-1,1-dicarboxamide (2,2,2-trifluoroacetic acid Salt)

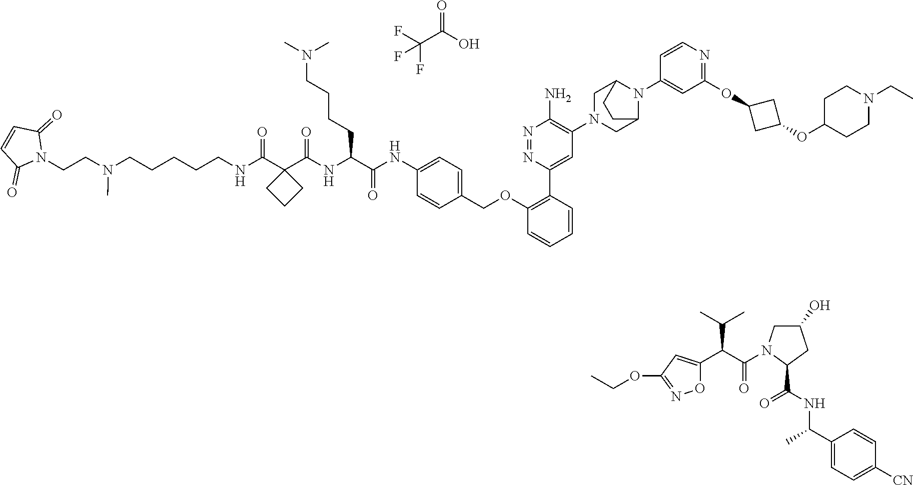

To a solution of 1-(((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (50.0 mg, 0.0400 mmol), 1-(2-((5-aminopentyl)(methyl)amino)ethyl)-1H-pyrrole-2,5-dione (2,2,2-trifluoroacetic acid salt) (30.0 mg, crude) and DIPEA (46.6 mg, 0.360 mmol) in DMF (1 mL) was added HATU (18.0 mg, 0.0500 mmol) at room temperature. The reaction was stirred at room temperature for 10 min. The crude was purified by Prep-HPLC with the following conditions: Column: Xselect CSH C18 OBD Column 30*150 mm 5 m, n; Mobile Phase A: Water (0.05% TFA), Mobile Phase B: ACN; Flow rate: 60 mL/min; Gradient: 8% B to 26% B in 12 min, 26% B; Wave Length: 254/220 nm; R_{T} (min): 12; to afford 16.7 mg of Hy-B-CIDE-11 as a white solid. LCMS (ESI, m/z): [M+H]⁺=1604. ¹H NMR (300 MHz, DMSO-d₆, ppm) δ 10.12 (s, 1H), 9.53 (s, 2H), 8.36 (d, J=7.4 Hz, 1H), 8.00-7.77 (m, 3H), 7.74 (d, J=8.1 Hz, 2H), 7.55 (d, J=8.3 Hz, 2H), 7.51-7.39 (m, 2H), 7.45-7.29 (m, 5H), 7.21 (d, J=8.4 Hz, 1H), 7.02 (t, J=7.6 Hz, 1H), 6.96 (s, 2H), 6.87 (s, 1H), 6.51 (s, 1H), 6.04 (d, J=9.9 Hz, 2H), 5.09 (s, 1H), 4.97 (s, 2H), 4.80 (t, J=7.0 Hz, 1H), 4.69-4.49 (m, 1H), 4.39 (s, 4H), 4.37-4.30 (m, 1H), 4.27-4.03 (m, 3H), 3.70-3.52 (m, 6H), 3.40-3.30 (m, 5H), 3.03-2.78 (m, 11H), 2.82-2.70 (m, 9H), 2.45-2.30 (m, 8H), 2.22-2.15 (m, 1H), 1.97-1.84 (m, 4H), 1.77 (s, 4H), 1.69-1.54 (m, 5H), 1.54-1.40 (m, 5H), 1.39-1.28 (m, 3H), 1.25-1.14 (m, 8H), 0.84 (t, J=6.3 Hz, 3H), 0.69 (dd, J=9.2, 6.6 Hz, 3H).

### Example 23

### Synthesis of Hy-B-CIDE-12

### N-((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((S)-2-(2,5-dioxo-2,5-dihydro-1H-pyrrol-1-yl)-3-hydroxypropanamido)pentyl)cyclobutane-1,1-dicarboxamide (2,2,2-trifluoroacetic acid salt)

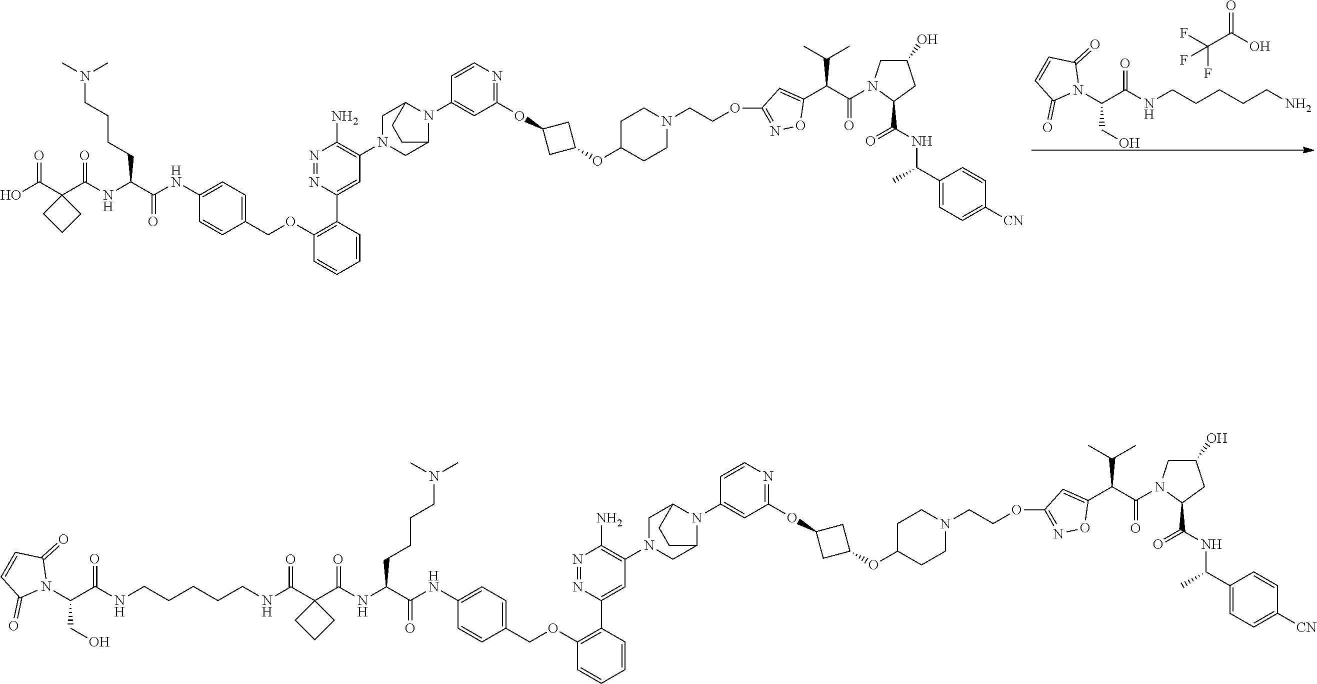

### Step 1: N-((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)-N-(5-((S)-2-(2,5-dioxo-2,5-dihydro-1H-pyrrol-1-yl)-3-hydroxypropanamido)pentyl)cyclobutane-1,1-dicarboxamide (2,2,2-trifluoroacetic acid Salt)

Under nitrogen, to a solution of 1-(((2S)-1-((4-((2-(6-amino-5-(8-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (40.0 mg, 0.0300 mmol), (S)—N-(5-aminopentyl)-2-(2,5-dioxo-2,5-dihydro-1H-pyrrol-1-yl)-3-hydroxypropanamide (2,2,2-trifluoroacetic acid salt) (30.0 mg, crude) and DIPEA (37.0 mg, 0.290 mmol) in N,N-dimethylformamide (1 mL) was added HATU (14.0 mg, 0.0400 mmol) at room temperature. The reaction was stirred at room temperature for 10 min. The product was purified by Prep-HPLC with the following conditions: Column: Xselect CSH C18 OBD Column 30*150 mm 5 m, n; Mobile Phase A: Water (0.05% TFA), Mobile Phase B: ACN; Flow rate: 60 mL/min; Gradient: 12% B to 27% B in 12 min, 27% B; Wave Length: 254/220 nm; R_{T}(min): 12; to afford 7.2 mg of Hy-B-CIDE-12 as a white solid.

LCMS (ESI, m/z): [M+H]⁺=1634. ¹H NMR (300 MHz, DMSO-d₆, ppm) δ 10.23 (s, 1H), 9.59 (d, J=47.0 Hz, 2H), 8.49 (d, J=7.3 Hz, 1H), 8.00-7.75 (m, 6H), 7.68 (d, J=8.2 Hz, 2H), 7.57 (t, J=8.6 Hz, 2H), 7.52-7.28 (m, 6H), 7.14 (t, J=7.6 Hz, 1H), 7.05 (s, 2H), 6.98 (s, 1H), 6.64 (s, 1H), 6.17 (d, J=8.2 Hz, 2H), 5.25-5.05 (m, 4H), 4.92 (t, J=6.8 Hz, 2H), 4.58-4.25 (m, 9H), 4.01-3.85 (m, 3H), 3.74-3.66 (m, 3H), 3.17-2.96 (m, 11H), 2.80-2.70 (m, 7H), 2.45-2.30 (m, 8H), 2.29-2.18 (m, 2H), 2.16-1.52 (m, 18H), 1.49-1.13 (m, 12H), 0.97 (d, J=6.4 Hz, 3H), 0.82 (dd, J=9.3, 6.6 Hz, 3H).

### Example 24

### Synthesis of Hy-B-CIDE-13

### 4—((S)-6-(dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl(4-(5-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-2-oxa-5,8-diazaspiro[3.5]nonan-8-yl)-6-(2-hydroxyphenyl)pyridazin-3-yl)carbamate (2,2,2-trifluoroacetic acid Salt)

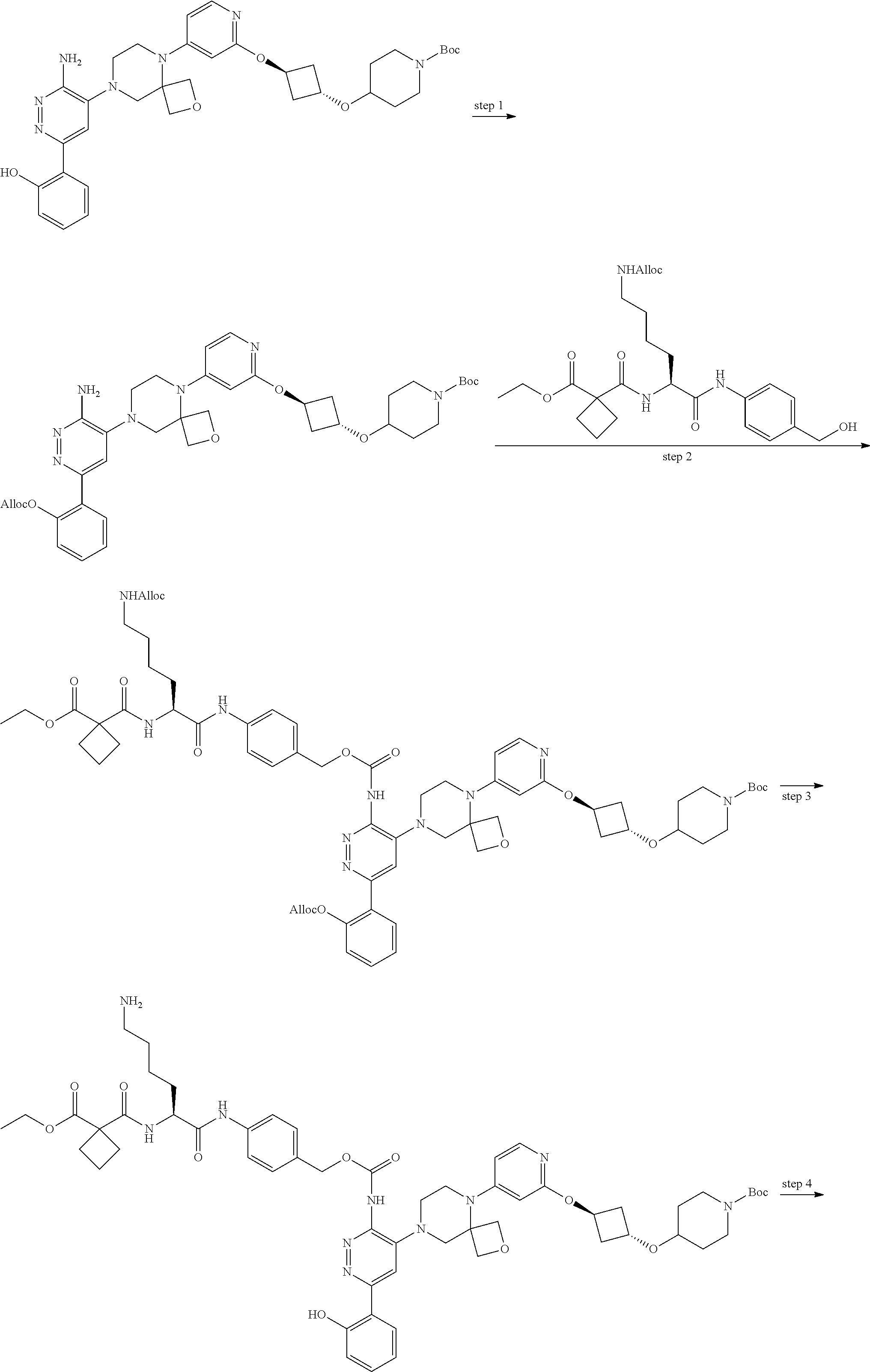

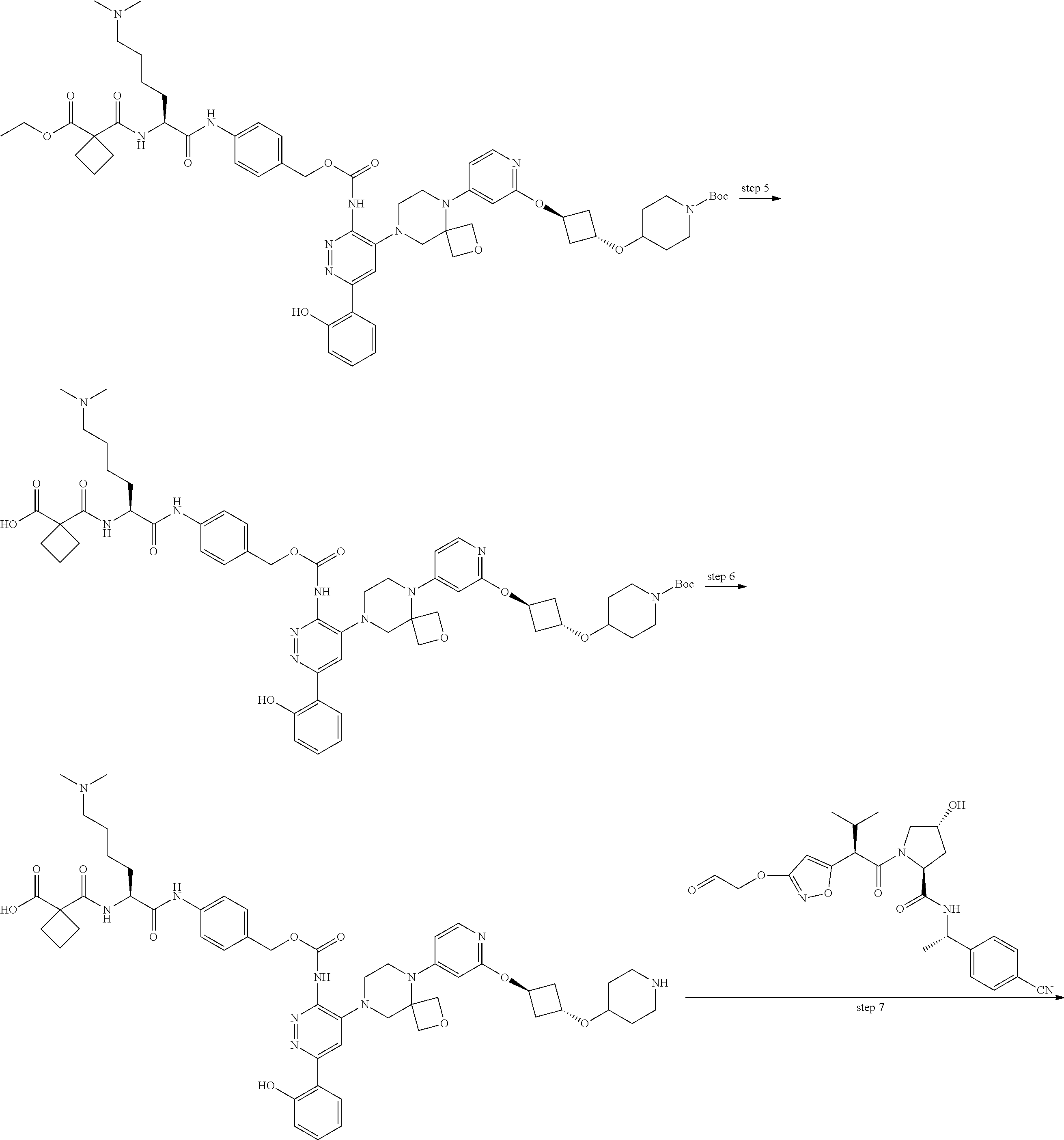

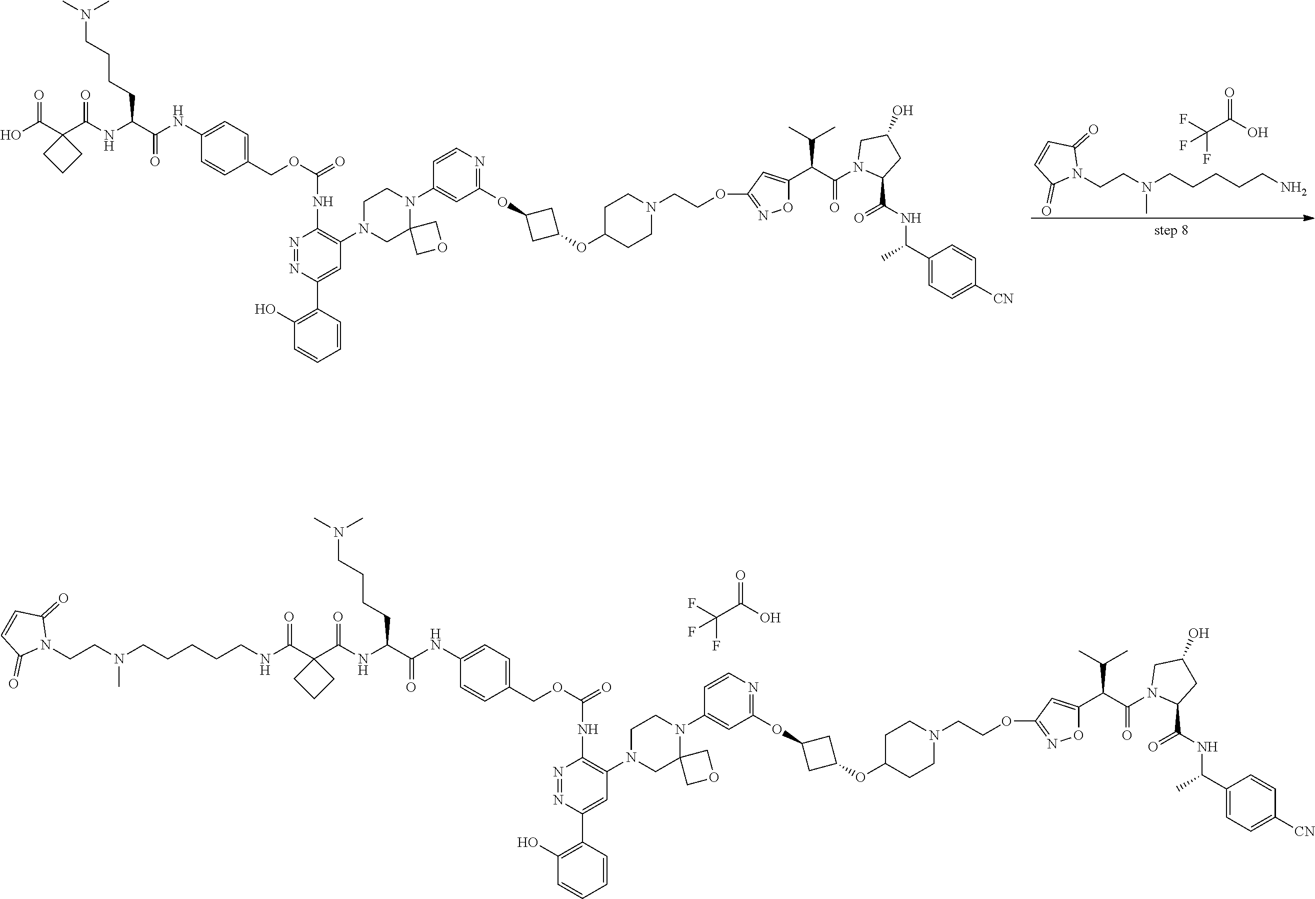

### Step 1: tert-Butyl 4-((1r,3r)-3-((4-(8-(6-(2-(((allyloxy)carbonyl)oxy)phenyl)-3 aminopyridazin-4-yl)-2-oxa-5,8-diazaspiro[3.5]nonan-5-yl)pyridin-2-yl)oxy)cyclobutoxy)piperidine-1-carboxylate

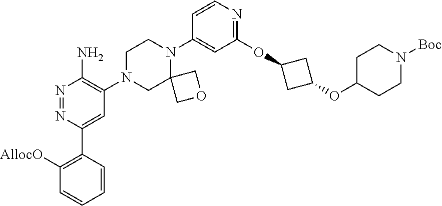

Under nitrogen, to a solution of tert-butyl 4-((1r,3r)-3-((4-(8-(3-amino-6-(2-hydroxyphenyl)pyridazin-4-yl)-2-oxa-5,8-diazaspiro[3.5]nonan-5-yl)pyridin-2-yl)oxy)cyclobutoxy)piperidine-1-carboxylate (480 mg, 7.28 mmol) and DPIEA (847 mg, 65.6 mmol) in DCM (6 mL) was added AllocCl (306 mg, 25.5 mmol) at 0° C. The resulting solution was stirred at room temperature for 30 min. The crude was purified by flash chromatography on silica gel (solvent gradient: 0-5% MeOH/DCM) to afford 505 mg (86% yield) of the title compound as a yellow solid. LC-MS: (ESI, m/z): [M+H]⁺=744.

### Step 2: tert-Butyl 4-((1r,3r)-3-((4-(8-(3-((((4-((S)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-2-oxa-5,8-diazaspiro[3.5]nonan-5-yl)pyridin-2-yl)oxy)cyclobutoxy)piperidine-1-carboxylate

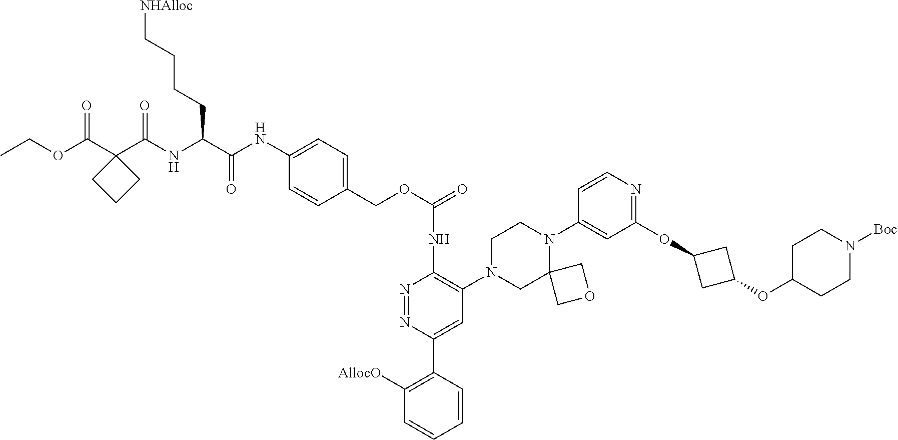

Under nitrogen, to a solution of tert-butyl 4-((1r,3r)-3-((4-(8-(6-(2-(((allyloxy)carbonyl)oxy)phenyl)-3-aminopyridazin-4-yl)-2-oxa-5,8-diazaspiro[3.5]nonan-5-yl)pyridin-2-yl)oxy)cyclobutoxy)piperidine-1-carboxylate (460 mg, 0.619 mmol), ethyl (S)-1-((6-(((allyloxy)carbonyl)amino)-1-((4-(hydroxymethyl)phenyl)amino)-1-oxohexan-2-yl)carbamoyl)cyclobutane-1-carboxylate (908 mg, 1.86 mmol) and DIPEA (480 mg, 3.72 mmol) in THF (6 mL) was added triphosgene (208 mg, 0.433 mmol) at 0° C. The reaction was stirred at room temperature for 0.5 h. Solvent was evaporated and residue was purified by pre-packed C18 column (solvent gradient: 0-100% ACN in water (0.05% NH₄HCO₃)) to yield 230 mg (29%) of the title compound as a yellow solid. LC-MS: (ESI, m/z): [M+H]⁺=1260.

### Step 3: tert-Butyl 4-((1r,3r)-3-((4-(8-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-2-oxa-5,8-diazaspiro[3.5]nonan-5-yl)pyridin-2-yl)oxy)cyclobutoxy)piperidine-1-carboxylate

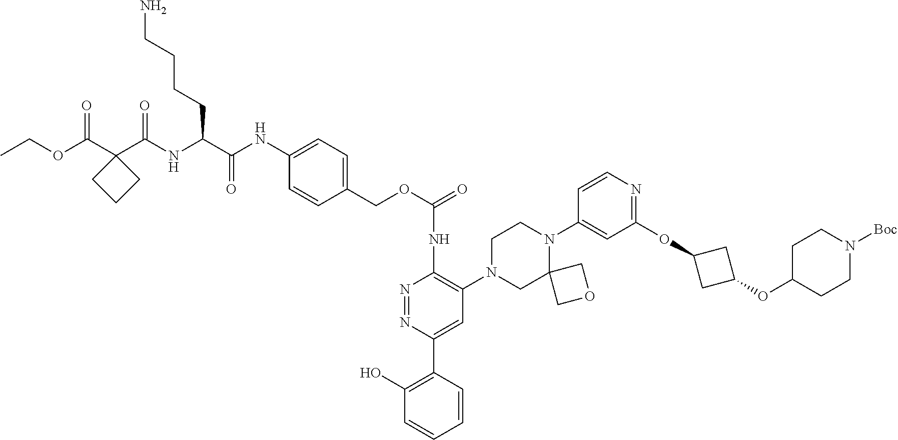 [SMILES: CCOC(=O)C1(C(=O)N[C@@H](CCCCN)C(=O)Nc2ccc(COC(=O)Nc3nnc(-c4ccccc4O)cc3N3CCN(c4ccnc(O[C@H]5C[C@H](OC6CCN(C(=O)OC(C)(C)C)CC6)C5)c4)C4(COC4)C3)cc2)CCC1]

Under nitrogen, a solution of tert-butyl 4-((1r,3r)-3-((4-(8-(3-((((4-((S)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-2-oxa-5,8-diazaspiro[3.5]nonan-5-yl)pyridin-2-yl)oxy)cyclobutoxy)piperidine-1-carboxylate (230 mg, 0.183 mmol), pyrrolidine (131 mg, 1.83 mmol) and Pd(PPh₃)₄ (22.0 mg, 0.01830 mmol) in DCM (4 mL) was stirred at room temperature for 1 h. The solvent was evaporated and crude was used in the next step directly.

### Step 4: tert-Butyl 4-((1r,3r)-3-((4-(8-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-2-oxa-5,8-diazaspiro[3.5]nonan-5-yl)pyridin-2-yl)oxy)cyclobutoxy)piperidine-1-carboxylate

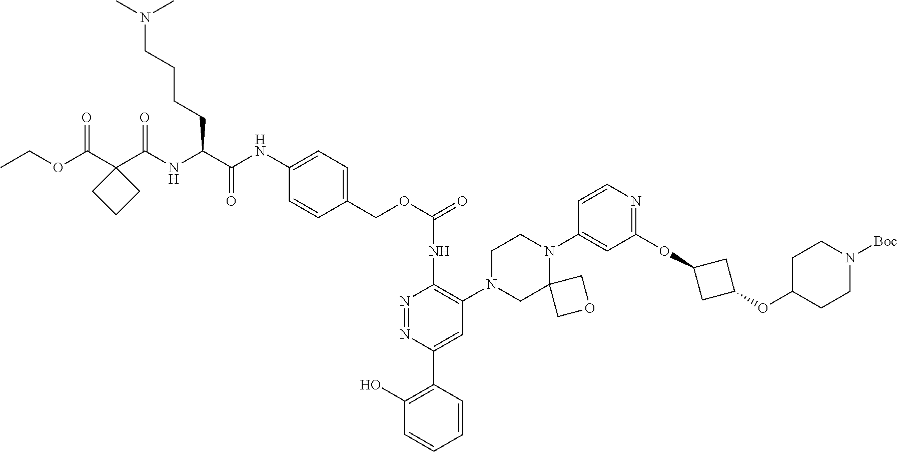 [SMILES: CCOC(=O)C1(C(=O)N[C@@H](CCCCN(C)C)C(=O)Nc2ccc(COC(=O)Nc3nnc(-c4ccccc4O)cc3N3CCN(c4ccnc(O[C@H]5C[C@H](OC6CCN(C(=O)OC(C)(C)C)CC6)C5)c4)C4(COC4)C3)cc2)CCC1]

Under nitrogen, a solution of tert-butyl 4-((1r,3r)-3-((4-(8-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-2-oxa-5,8-diazaspiro[3.5]nonan-5-yl)pyridin-2-yl)oxy)cyclobutoxy)piperidine-1-carboxylate (230 mg, crude) and 37% aqueous CH₂O (230 mg) in MeOH (4 mL) was stirred at room temperature for 1 h. Then NaBH₃CN (63.5 mg, 1.06 mmol) was added and the solution was stirred at room temperature for 15 min. The reaction was quenched with water and the resulting residue was purified by pre-packed C18 column (solvent gradient: 0-90% MeOH in water (0.05% NH₄HCO₃)) to afford 140 mg (55% yield) of the title compound as a yellow solid. LC-MS: (ESI, m/z): [M+H]⁺=1120.

### Step 5: 1-(((S)-1-((4-((((4-(5-(2-((1r,3r)-3-((1-(tert-Butoxycarbonyl)piperidin-4-yl)oxy)cyclobutoxy)pyridin-4-yl)-2-oxa-5,8-diazaspiro[3.5]nonan-8-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid

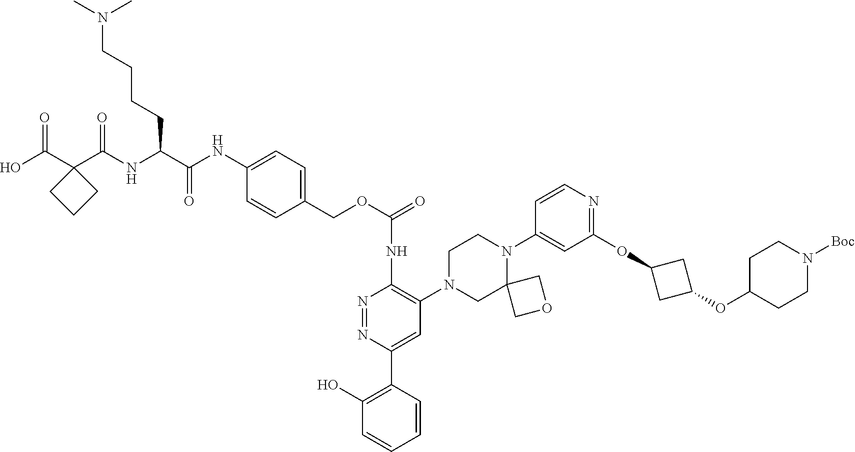 [SMILES: CN(C)CCCC[C@H](NC(=O)C1(C(=O)O)CCC1)C(=O)Nc1ccc(COC(=O)Nc2nnc(-c3ccccc3O)cc2N2CCN(c3ccnc(O[C@H]4C[C@H](OC5CCN(C(=O)OC(C)(C)C)CC5)C4)c3)C3(COC3)C2)cc1]

Under nitrogen, a solution of tert-butyl 4-((1r,3r)-3-((4-(8-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-2-oxa-5,8-diazaspiro[3.5]nonan-5-yl)pyridin-2-yl)oxy)cyclobutoxy)piperidine-1-carboxylate (140 mg, 0.125 mmol) and LiOH·H₂O (15.9 mg, 0.375 mmol) in THF (2 mL) and H₂O (2 mL) was stirred at room temperature for 1 h. The resulting mixture was concentrated under vacuum to afford 160 mg (crude) of the title compound as a yellow solid. LC-MS: (ESI, m/z): [M+H]⁺=1092.

### Step 6: 1-(((S)-6-(Dimethylamino)-1-((4-((((6-(2-hydroxyphenyl)-4-(5-(2-((1r,3r)-3-(piperidin-4-yloxy)cyclobutoxy)pyridin-4-yl)-2-oxa-5,8-diazaspiro[3.5]nonan-8-yl)pyridazin-3-yl)carbamoyl)oxy)methyl)phenyl)amino)-1-oxohexan-2-yl)carbamoyl)cyclobutane-1-carboxylic acid

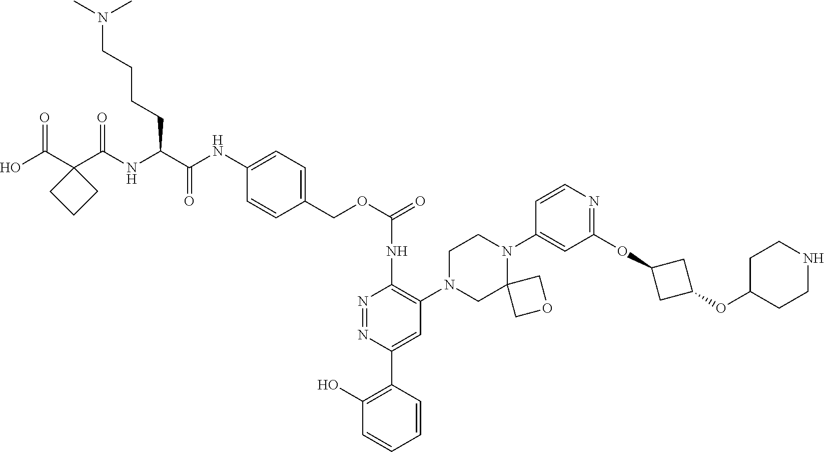 [SMILES: CN(C)CCCC[C@H](NC(=O)C1(C(=O)O)CCC1)C(=O)Nc1ccc(COC(=O)Nc2nnc(-c3ccccc3O)cc2N2CCN(c3ccnc(O[C@H]4C[C@H](OC5CCNCC5)C4)c3)C3(COC3)C2)cc1]

Under nitrogen, a solution of 1-(((S)-1-((4-((((4-(5-(2-((1r,3r)-3-((1-(tert-butoxycarbonyl)piperidin-4-yl)oxy)cyclobutoxy)pyridin-4-yl)-2-oxa-5,8-diazaspiro[3.5]nonan-8-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid (160 mg, crude) in HFIP (4.8 mL) and 2,2,2-trifluoroacetic acid (0.25 mL) was stirred at room temperature for 15 min. The solvent was evaporated under vacuum and purified by pre-packed C18 column (solvent gradient: 0-70% ACN in water (0.05% NH₄HCO₃)) to afford 74.0 mg (51% yield) of the title compound as a white solid. LC-MS: (ESI, m/z): [M+H]⁺=992.

### Step 7: 1-(((S)-1-((4-((((4-(5-(2-((1R,3r)-3-((1-(2-((5-((R)-1-((2S,4R)-2-(((S)-1-(4-Cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-2-oxa-5,8-diazaspiro[3.5]nonan-8-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid

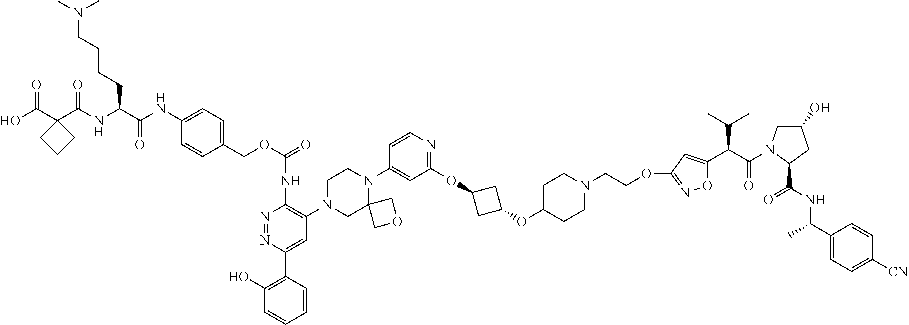 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5CCN(c6cc(-c7ccccc7O)nnc6NC(=O)OCc6ccc(NC(=O)[C@H](CCCCN(C)C)NC(=O)C7(C(=O)O)CCC7)cc6)CC56COC6)ccn4)C3)CC2)no1]

Under nitrogen, a solution of 1-(((S)-6-(dimethylamino)-1-((4-((((6-(2-hydroxyphenyl)-4-(5-(2-((1r,3r)-3-(piperidin-4-yloxy)cyclobutoxy)pyridin-4-yl)-2-oxa-5,8-diazaspiro[3.5]nonan-8-yl)pyridazin-3-yl)carbamoyl)oxy)methyl)phenyl)amino)-1-oxohexan-2-yl)carbamoyl)cyclobutane-1-carboxylic acid (30.0 mg, 0.0300 mmol), (2S,4R)—N—((S)-1-(4-cyanophenyl)ethyl)-4-hydroxy-1-((R)-3-methyl-2-(3-(2-oxoethoxy)isoxazol-5-yl)butanoyl)pyrrolidine-2-carboxamide (28.5 mg, 0.061 mmol) and CH₃COOH (3.60 mg, 0.0610 mmol) in MeOH (0.25 mL) and DCM (0.75 mL) was stirred at room temperature for 1 h. Then NaBH₃CN (3.00 mg, 0.0450 mmol) was added and stirred at room temperature for 0.5 h. The crude was purified by pre-packed C18 column (solvent gradient: 0-80% MeOH in water (0.05% NH₄HCO₃)) to afford 18.0 mg (41% yield) of the title compound as a white solid. LC-MS: (ESI, m/z): [M+H]⁺=1444.

### Step 8: 4-((S)-6-(Dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl (4-(5-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-2-oxa-5,8-diazaspiro[3.5]nonan-8-yl)-6-(2-hydroxyphenyl)pyridazin-3-yl)carbamate (2,2,2-trifluoroacetic acid Salt)

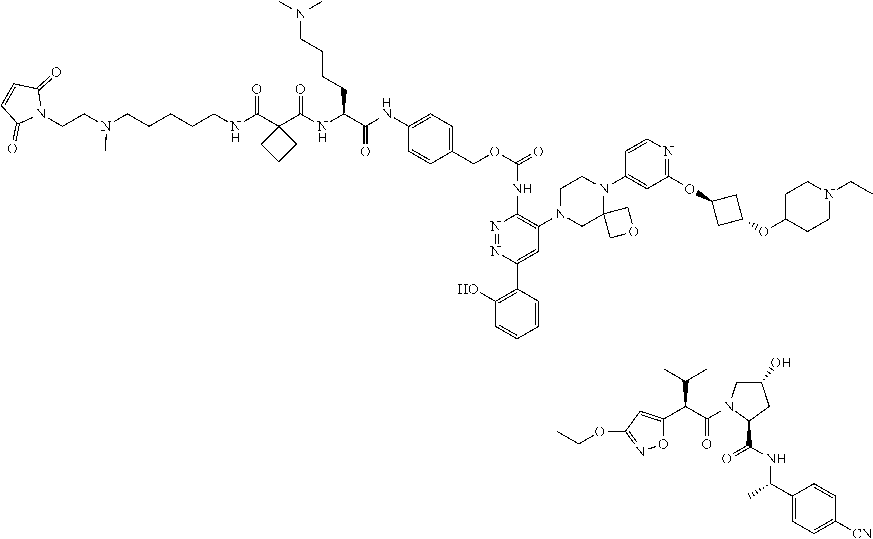

Under nitrogen, to a solution of 1-(((S)-1-((4-((((4-(5-(2-((1R,3r)-3-((1-(2-((5-((R)-1-((2S,4R)-2-(((S)-1-(4-cyanophenyl)ethyl)carbamoyl)-4-hydroxypyrrolidin-1-yl)-3-methyl-1-oxobutan-2-yl)isoxazol-3-yl)oxy)ethyl)piperidin-4-yl)oxy)cyclobutoxy)pyridin-4-yl)-2-oxa-5,8-diazaspiro[3.5]nonan-8-yl)-6-(2-hydroxyphenyl)pyridazin-3-yl)carbamoyl)oxy)methyl)phenyl)amino)-6-(dimethylamino)-1-oxohexan-2-yl)carbamoyl)cyclobutane-1-carboxylic acid (18.0 mg, 0.0125 mmol), 1-(2-((5-aminopentyl)(methyl)amino)ethyl)-1H-pyrrole-2,5-dione (2,2,2-trifluoroacetic acid salt) (13.0 mg, crude) and DIPEA (25.8 mg, 0.200 mmol) in DMF (1 mL) was added HATU (9.50 mg, 0.025 mmol) at room temperature. The reaction was stirred at room temperature for 10 min. The crude was purified by Prep-HPLC with following conditions: Column: Xselect CSH C18 OBD Column 30*150 mm 5 m, n; Mobile Phase A: Water (0.05% TFA), Mobile Phase B: ACN; Flow rate: 60 mL/min; Gradient: 14% B to 28% B in 10 min, 28% B; Wave Length: 254/220 nm; R_{T}(min): 10 to afford 14.9 mg (71.7% yield) of Hy-B-CIDE-13 as a white solid. LC-MS: (ESI, m/z): [M+H]⁺=1665. ¹H NMR (300 MHz, DMSO-d₆, ppm) δ 10.17 (d, J=17.2 Hz, 2H), 9.55 (br, 1H), 9.40 (br, 1H), 8.49 (d, J=7.3 Hz, 1H), 8.03 (d, J=7.9 Hz, 1H), 7.95-7.82 (m, 3H), 7.79 (d, J=8.3 Hz, 2H), 7.67 (d, J=8.4 Hz, 2H), 7.47 (m, J=8.4, 2.7 Hz, 2H), 7.38 (t, J=8.5 Hz, 3H), 7.10 (s, 2H), 7.02-6.92 (m, 2H), 6.42-6.34 (m, 1H), 6.16 (s, 1H), 5.75 (s, 1H), 5.20 (s, 1H), 5.14 (s, 2H), 4.91 (t, J=7.2 Hz, 1H), 4.63 (d, J=4.4 Hz, 3H), 4.51 (s, 2H), 4.42 (s, 1H), 4.33 (t, J=8.2 Hz, 2H), 3.81-3.66 (m, 10H), 3.21-2.94 (m, 13H), 2.82-2.74 (m, 9H), 2.45-2.20 (m, 12H), 2.18-2.00 (m, 3H), 1.92-1.70 (m, 7H), 1.70-1.50 (m, 5H), 1.47 (q, J=7.1 Hz, 3H), 1.39-1.21 (m, 8H), 0.96 (d, J=6.5 Hz, 3H), 0.80 (d, J=6.8 Hz, 3H).

### Example 25

### Synthesis of Hy-B-CIDE-14

### 4-((S)-6-(Dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamate (2,2,2-trifluoroacetic acid salt)

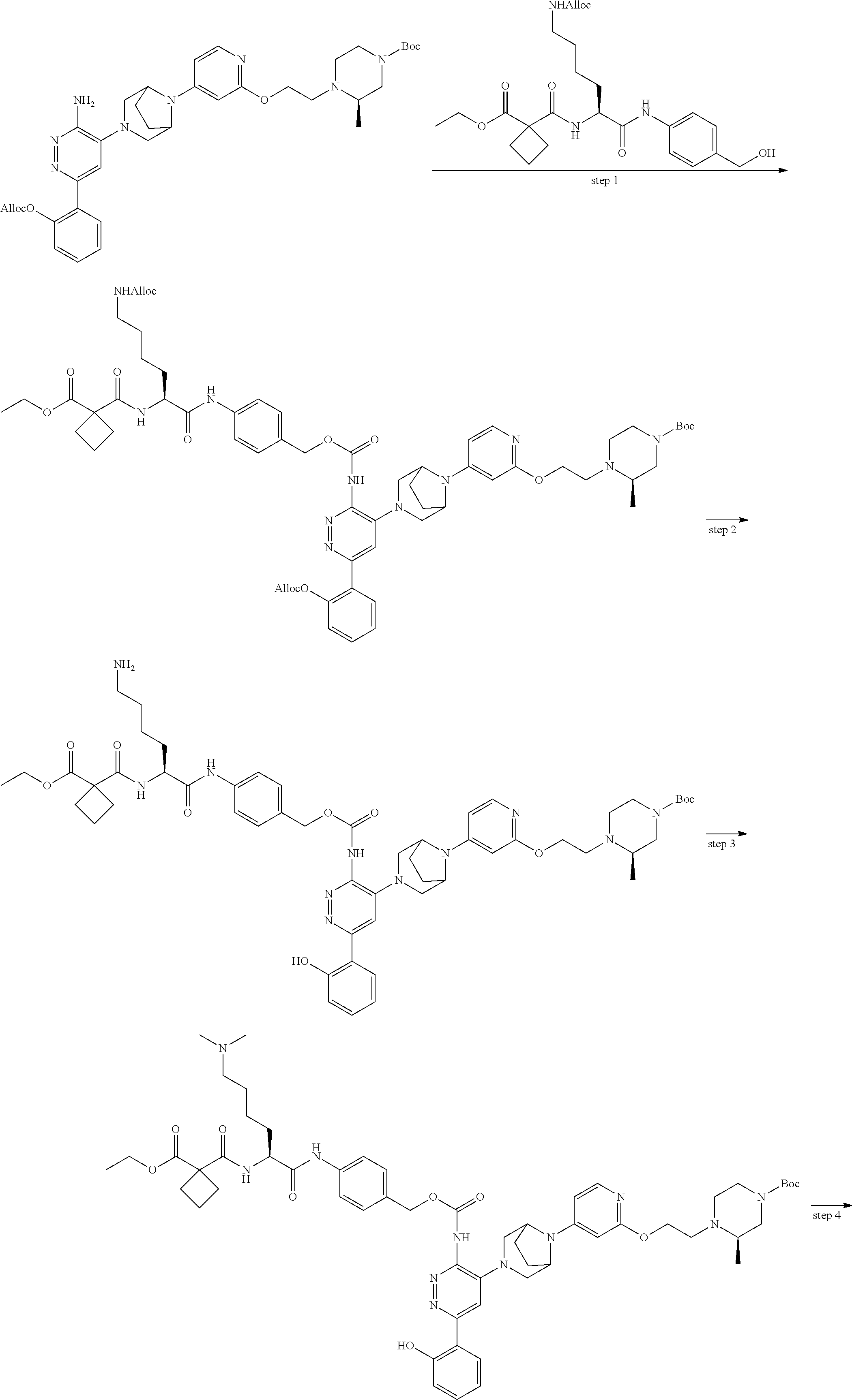

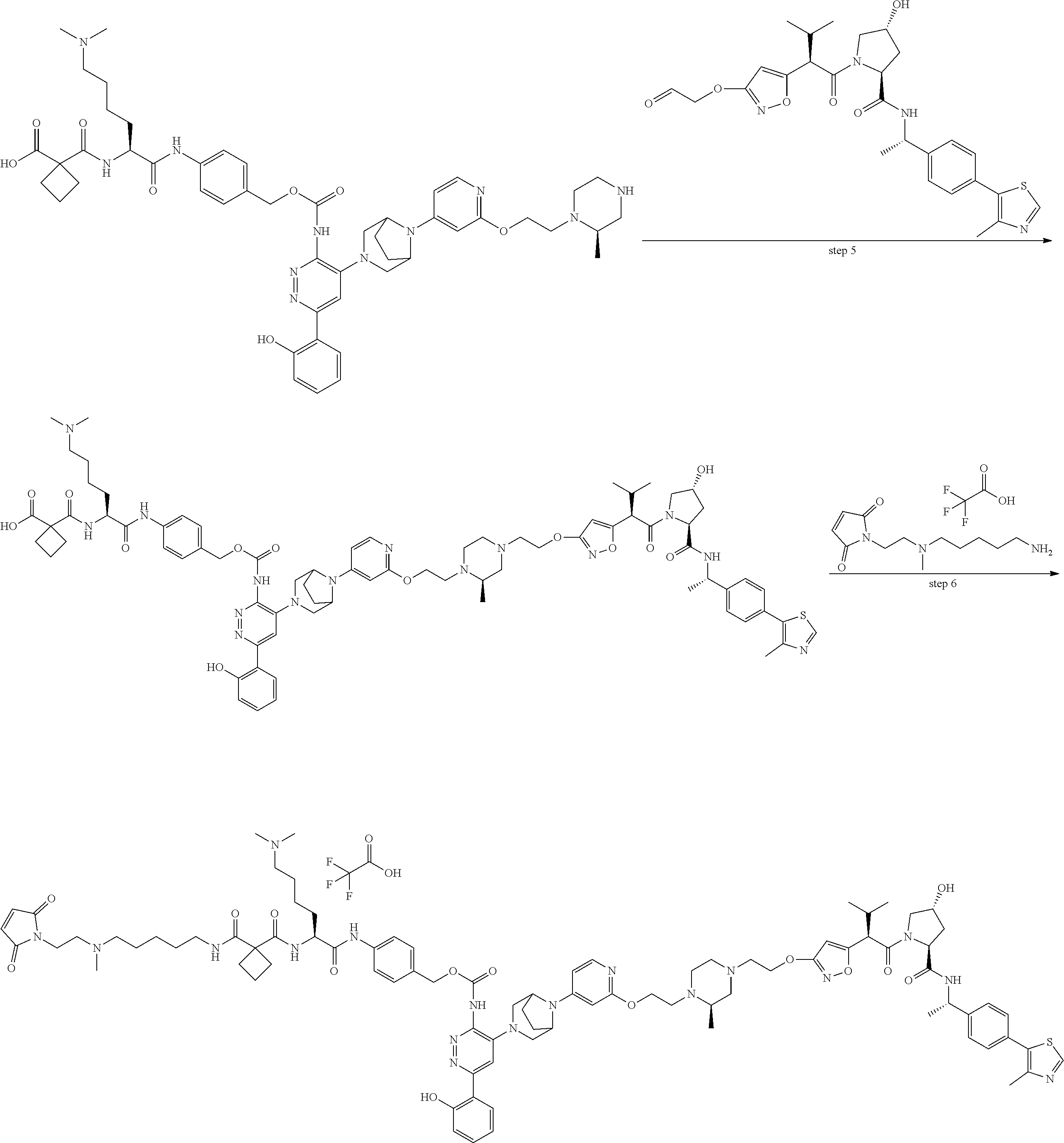

### Step 1: tert-Butyl (3R)-4-(2-((4-(3-(3-((((4-((S′)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-3,8-diazabicyclo[3.2.1]octan-8-yl)pyridin-2-yl)oxy)ethyl)-3-methylpiperazine-1-carboxylate

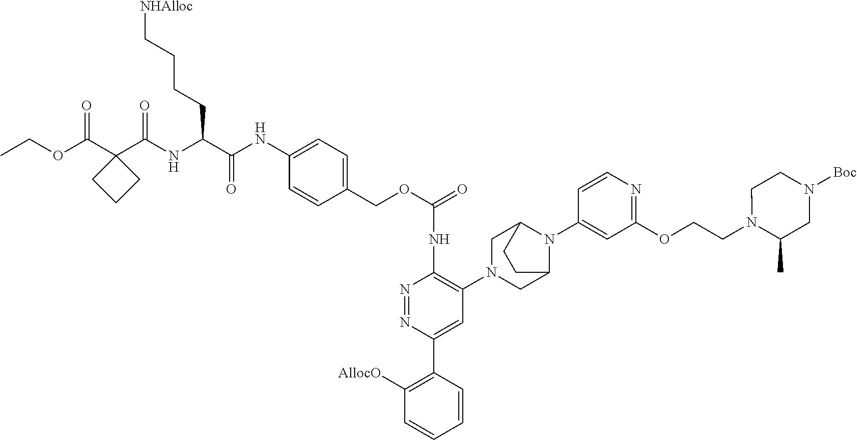

Under nitrogen, to a solution of tert-butyl (3R)-4-(2-((4-(3-(6-(2-(((allyloxy)carbonyl)oxy)phenyl)-3-aminopyridazin-4-yl)-3,8-diazabicyclo[3.2.1]octan-8-yl)pyridin-2-yl)oxy)ethyl)-3-methylpiperazine-1-carboxylate (500 mg, 0.714 mmol), ethyl (S)-1-((6-(((allyloxy)carbonyl)amino)-1-((4-(hydroxymethyl)phenyl)amino)-1-oxohexan-2-yl)carbamoyl)cyclobutane-1-carboxylate (1.05 g, 2.15 mmol) and DIPEA (554 mg, 4.29 mmol) in DCM (15 mL) was added a solution of triphosgene (109 mg, 0.367 mmol) in DCM (1 mL) at 0° C. The reaction was stirred at room temperature for 2 h. The solvent was concentrated under vacuum. The residue was purified by pre-packed C18 column (solvent gradient: 0-100% CH₃CN in water (0.05% NH₄HCO₃)) to yield 480 mg (55% yield) of the title compound as a white solid. LC-MS: (ESI, m/z): [M+H]⁺=1216.

### Step 2: tert-Butyl (3R)-4-(2-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate

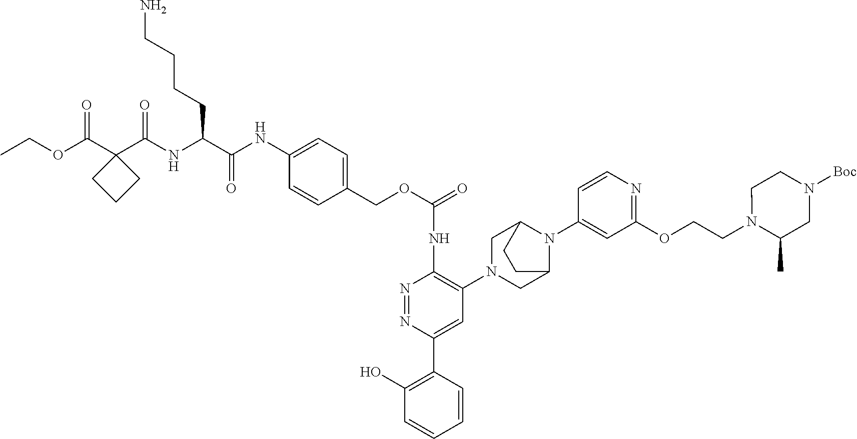 [SMILES: CCOC(=O)C1(C(=O)N[C@@H](CCCCN)C(=O)Nc2ccc(COC(=O)Nc3nnc(-c4ccccc4O)cc3N3CC4CCC(C3)N4c3ccnc(OCCN4CCN(C(=O)OC(C)(C)C)C[C@H]4C)c3)cc2)CCC1]

Under nitrogen, to a solution of tert-butyl (3R)-4-(2-((4-(3-(3-((((4-((S)-6-(((allyloxy)carbonyl)amino)-2-(1-(ethoxycarbonyl)cyclobutane-1-carboxamido)hexanamido)benzyl)oxy)carbonyl)amino)-6-(2-(((allyloxy)carbonyl)oxy)phenyl)pyridazin-4-yl)-3,8-diazabicyclo[3.2.1]octan-8-yl)pyridin-2-yl)oxy)ethyl)-3-methylpiperazine-1-carboxylate (400 mg, 0.329 mmol), pyrrolidine (234 mg, 3.29 mmol) in DCM (5 mL) was added Pd(PPh₃)₄ (76.0 mg, 0.0658 mmol) at room temperature. The reaction was stirred at room temperature for 1 h. The reaction mixture was diluted with DCM (100 mL) and washed with water. The organic layer was dried over anhydrous sodium sulfate and concentrated under vacuum to afford 390 mg (crude) of the title compound as a yellow solid. LC-MS: (ESI, m/z): [M+H]⁺=1048.

### Step 3: tert-Butyl (3R)-4-(2-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate

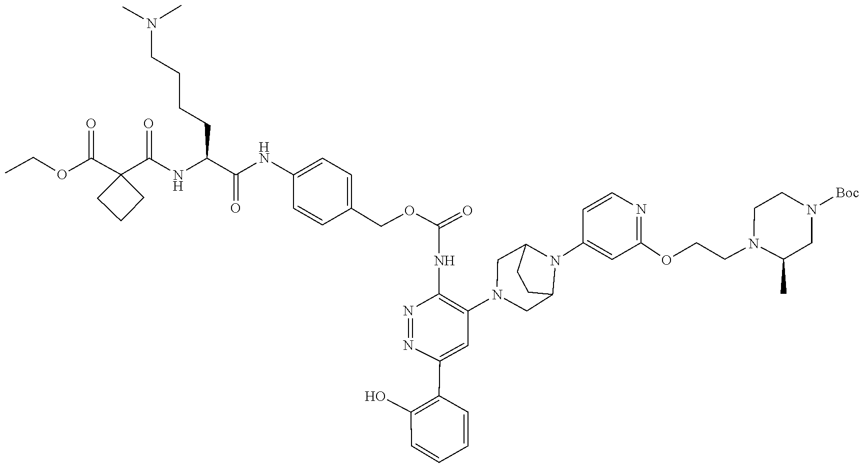 [SMILES: CCOC(=O)C1(C(=O)N[C@@H](CCCCN(C)C)C(=O)Nc2ccc(COC(=O)Nc3nnc(-c4ccccc4O)cc3N3CC4CCC(C3)N4c3ccnc(OCCN4CCN(C(=O)OC(C)(C)C)C[C@H]4C)c3)cc2)CCC1]

A solution of tert-butyl (3R)-4-(2-((4-(3-(3-((((4-((S)-6-amino-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (465 mg, 0.440 mmol) and 37% aqueous CH₂O (465 mg, 5.73 mmol) in CH₃OH (5 mL) was stirred at room temperature for 1 h. Then NaBH₃CN (140 mg, 2.22 mmol) was added and stirred at room temperature for 1 hour. The reaction was quenched by water (0.2 mL). The residue was purified by pre-packed C18 column (solvent gradient: 0-100% MeOH in water (0.05% NH₄HCO₃)) to yield 260 mg (54% yield) of the title compound as a white solid. LC-MS: (ESI, m/z): [M]⁺=1077.

### Step 4: 1-(((2S)-6-(Dimethylamino)-1-((4-((((6-(2-hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid

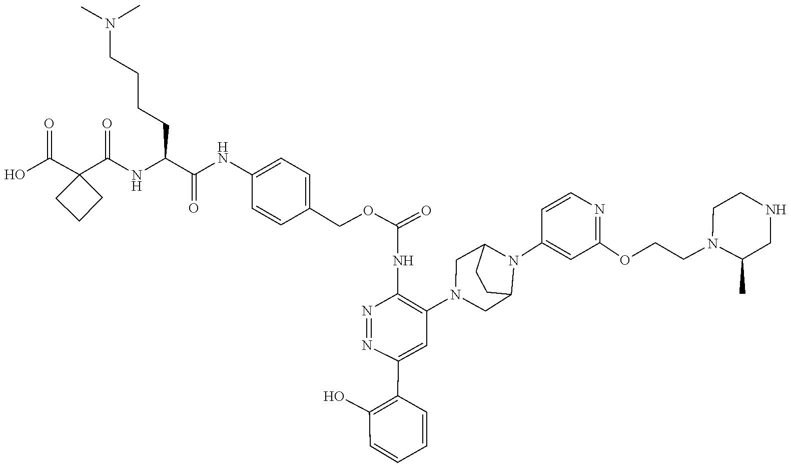 [SMILES: C[C@@H]1CNCCN1CCOc1cc(N2C3CCC2CN(c2cc(-c4ccccc4O)nnc2NC(=O)OCc2ccc(NC(=O)[C@H](CCCCN(C)C)NC(=O)C4(C(=O)O)CCC4)cc2)C3)ccn1]

A solution of tert-butyl (3R)-4-(2-((4-(3-(3-((((4-((S)-6-(dimethylamino)-2-(1-(ethoxycarbonyl)cyclobutane-1-carboxamido)hexanamido)benzyl)oxy)carbonyl)amino)-6-(2-hydroxyphenyl)pyridazin-4-yl)-3,8-diazabicyclo[3.2.1]octan-8-yl)pyridin-2-yl)oxy)ethyl)-3-methylpiperazine-1-carboxylate (75.0 mg, 0.0697 mmol) and LiOH·H₂O (8.4 mg, 0.210 mmol) in THF (5 mL) and water (5 mL) was stirred at room temperature for 1 h. The solvent was concentrated under vacuum. Then 5% TFA in HFIP (3 mL) was added and the solution was stirred at room temperature for 1 h. The solvent was concentrated under vacuum. The residue was purified by pre-packed C18 column (solvent gradient: 0-100% MeOH in water (0.05% NH₄HCO₃)) to yield 55.0 mg (83% yield) of the title compound as a white solid. LC-MS: (ESI, m/z): [M]⁺=949.

### Step 5: 1-(((2S)-6-(Dimethylamino)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-1-oxohexan-2-yl)carbamoyl)cyclobutane-1-carboxylic acid

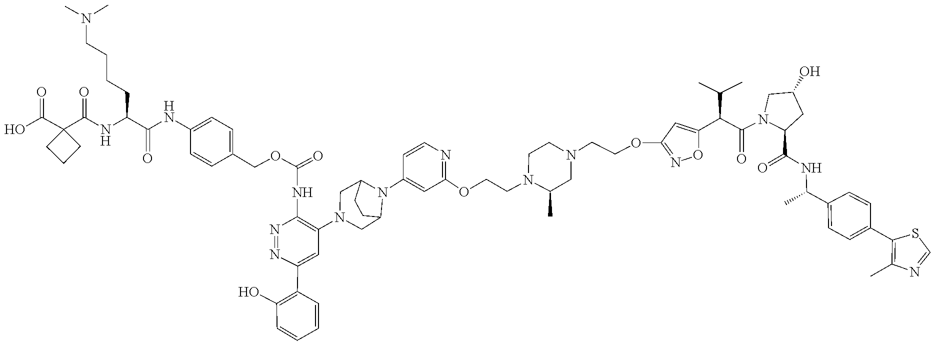 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)O)CCC7)cc5)C6)ccn4)[C@H](C)C3)no2)C(C)C)cc1]

Under nitrogen, a solution of 1-(((2S)-6-(dimethylamino)-1-((4-((((6-(2-hydroxyphenyl)-4-(8-(2-(2-((R)-2-methylpiperazin-1-yl)ethoxy)pyridin-4-yl)-3,8-diazabicyclo[3.2.1]octan-3-yl)pyridazin-3-yl)carbamoyl)oxy)methyl)phenyl)amino)-1-oxohexan-2-yl)carbamoyl)cyclobutane-1-carboxylic acid (20.0 g, 0.0211 mmol), (2S,4R)-4-hydroxy-1-((R)-3-methyl-2-(3-(2-oxoethoxy)isoxazol-5-yl)butanoyl)-N—((S)-1-(4-(4-methylthiazol-5-yl)phenyl)ethyl)pyrrolidine-2-carboxamide (19.4 mg, 0.0359 mmol) and NaOAc (4.0 mg, 0.0488 mmol) in methyl alcohol (1.5 mL) was stirred at room temperature for 1 hour. Then NaBH₃CN (5.2 mg, 0.0827 mol) was added and stirred at room temperature for 3 h. The reaction solution was concentrated under vacuum. The residue was purified by pre-packed C18 column (solvent gradient: 0-100% CH₃OH in water (0.05% NH₄HCO₃)) to yield 18 mg (58% yield) of the title compound as a white solid. LC-MS: (ESI, m/z): [M+H]⁺=1473.

### Step 6: 4-((S)-6-(Dimethylamino)-2-(1-((5-((2-(2,5-dioxo-2,5-dihydro-1H-pyrrol-1-yl)ethyl)(methyl)amino)pentyl)carbamoyl)cyclobutane-1-carboxamido)hexanamido)benzyl (4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamate (2,2,2-trifluoroacetic acid salt)

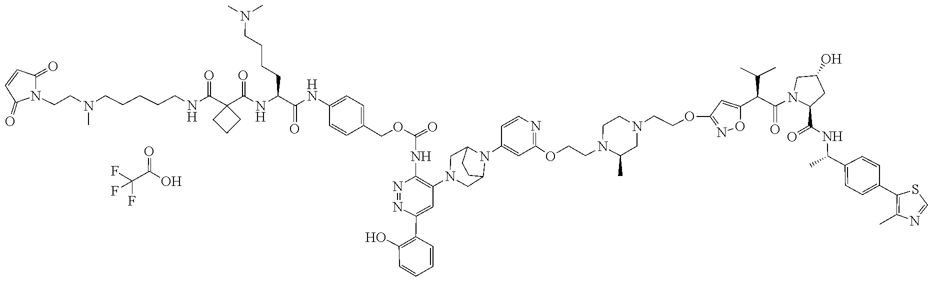 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7O)nnc5NC(=O)OCc5ccc(NC(=O)[C@H](CCCCN(C)C)NC(=O)C7(C(=O)NCCCCCN(C)CCN8C(=O)C=CC8=O)CCC7)cc5)C6)ccn4)[C@H](C)C3)no2)C(C)C)cc1 ; O=C(O)C(F)(F)F]

To a solution of 1-(((2S)-6-(dimethylamino)-1-((4-((((4-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)-6-(2-hydroxyphenyl)pyridazin-3-yl)carbamoyl)oxy)methyl)phenyl)amino)-1-oxohexan-2-yl)carbamoyl)cyclobutane-1-carboxylic acid (40.0 mg, 0.0272 mmol), 1-(2-((5-aminopentyl)(methyl)amino)ethyl)-1H-pyrrole-2,5-dione (2,2,2-trifluoroacetic acid salt) (9.70 mg, crude) and DIPEA (35.1 mg, 0.272 mmol) in DMF (1 mL) was added HATU (13.5 mg, 0.0355 mmol) at room temperature. The reaction was stirred at room temperature for 10 min. The crude was purified by Prep-HPLC with following conditions: Column: XSelect CSH Fluoro Phenyl, 30*150 mm, 5 m; Mobile Phase A: Water (0.05% TFA), Mobile Phase B: ACN; Flow rate: 60 mL/min; Gradient: 15% B to 20% B in 10 min; Wave Length: 254/220 nm; R_{T}(min): 10.5 to yield 13.0 mg (28.3% yield) of Hy-B-CIDE-14 as a yellow solid. LC-MS: (ESI, m/z): [M+H]⁺=1808. ¹H NMR (300 MHz, DMSO-d₆, ppm) δ 10.09 (s, 1H), 9.97 (s, 1H), 9.50 (s, 2H), 8.88 (d, J=1.3 Hz, 1H), 8.30 (d, J=7.7 Hz, 1H), 8.00-7.80 (m, 4H), 7.58-7.50 (m, 3H), 7.39-7.16 (m, 7H), 6.96 (s, 2H), 6.84 (ddd, J=11.0, 7.0, 2.3 Hz, 2H), 6.65 (d, J=6.6 Hz, 1H), 6.29 (s, 1H), 6.00 (s, 1H), 5.01 (s, 2H), 4.95-4.80 (m, 1H), 4.59 (s, 2H), 4.37 (s, 2H), 4.35-4.21 (m, 4H), 4.21-4.15 (m, 2H), 3.59-3.48 (m, 6H), 3.40-3.30 (m, 4H), 3.27-2.97 (m, 14H), 2.89 (s, 4H), 2.70-2.60 (m, 9H), 2.45-2.34 (m, 4H), 2.30-2.19 (m, 2H), 2.17-2.03 (m, 2H), 2.05-1.83 (m, 5H), 1.83-1.66 (m, 5H), 1.66-1.49 (m, 4H), 1.34 (d, J=7.1 Hz, 3H), 1.25 (d, J=7.0 Hz, 3H), 1.20-1.10 (m, 6H), 0.85 (d, J=6.6 Hz, 3H), 0.69 (dd, J=10.7, 6.6 Hz, 3H).

### Example 26

### Synthesis of Hy-B-CIDE-3

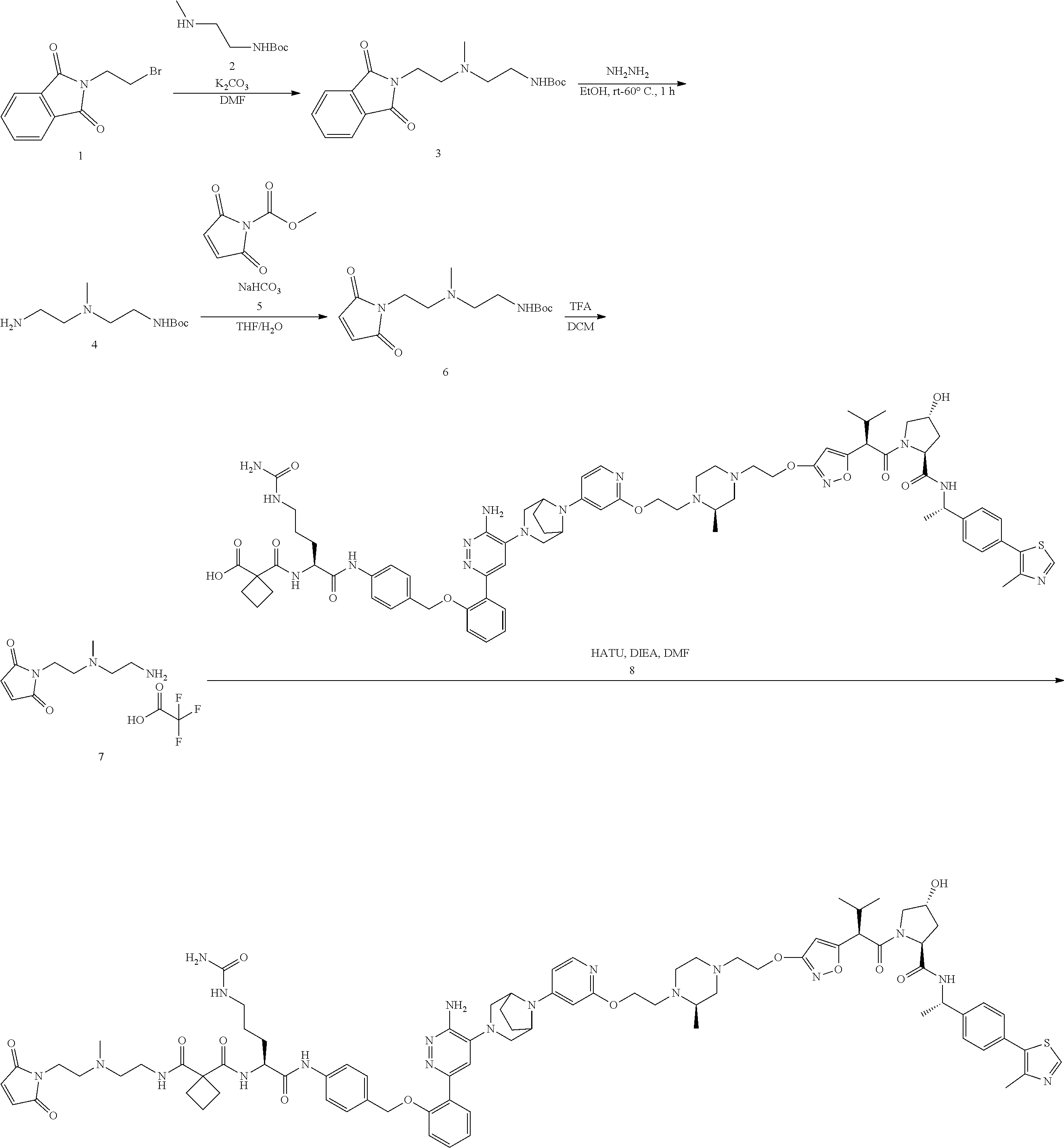

### Step 1: tert-butyl (2-((2-(1,3-dioxoisoindolin-2-yl)ethyl)(methyl)amino)ethyl)carbamate

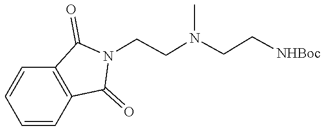 [SMILES: CN(CCNC(=O)OC(C)(C)C)CCN1C(=O)c2ccccc2C1=O]

A mixture of potassium carbonate (10.9 g, 78.7 mmol), 2-(2-bromoethyl)isoindoline-1,3-dione (10.0 g, 39.4 mmol) and tert-butyl (2-(methylamino)ethyl)carbamate (8.23 g, 47.2 mmol) in N,N-dimethylformamide (200 mL) was stirred at 90° C. for 16 h under N₂ atmosphere. The reaction was cooled to 25° C. and diluted with water (30 mL) and extracted with ethyl acetate (50 mL×3). The organics were washed with brine (20 mL×2), dried over sodium sulfate, filtered and concentrated to dryness. The residue was purified by flash chromatography (silica gel, 100-200 mesh, 0-15% methanol in dichloromethane) to give the title compound (4.5 g, 32.9% yield) as a white solid.

¹H NMR (400 MHz, CDCl₃): δ (ppm) 7.92-7.82 (m, 2H), 7.73 (m, 2H), 4.32-4.28 (m, 1H), 4.01-3.98 (m, 1H), 3.84-3.74 (m, 1H), 3.50 (s, 1H), 3.37-3.09 (m, 3H), 2.94-2.78 (m, 1H), 2.69-2.65 (m, 1H), 2.55-2.50 (m, 1H), 2.31-2.35 (m, 1H), 1.49-1.32 (m, 10H).

### Step 2: tert-butyl (2-((2-aminoethyl)(methyl)amino)ethyl)carbamate

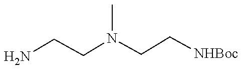

To a stirred solution of tert-butyl (2-((2-(1,3-dioxoisoindolin-2-yl)ethyl)(methyl)amino)ethyl)carbamate (4.00 g, 11.5 mmol) in acetonitrile (60 mL) was added hydrazine monohydrate (22.4 mL, 230 mmol) dropwise. The resulted mixture was stirred at 70° C. for 16 h under N₂ atmosphere. The reaction mixture was poured into ice water (15.0 mL), and extracted with ethyl acetate (20.0 mL×3). The combined organic layers were washed with brine (20.0 mL×2) and dried with anhydrous sodium sulfate, concentrated to give crude product, which was purified by prep-TLC (5% methanol in dichloromethane) to give the title compound (1.50 g, 60% yield) as a colorless oil.

¹H NMR (400 MHz, CDCl₃) δ (ppm) 4.13-4.10 (m, 2H), 3.43-3.33 (m, 2H), 3.30-3.25 (m, 2H), 2.96-2.92 (m, 5H), 1.41 (s, 9H).

### Step 3: tert-butyl (2-((2-(2,5-dioxo-2,5-dihydro-1H-pyrrol-1-yl)ethyl)(methyl)amino)ethyl)carbamate

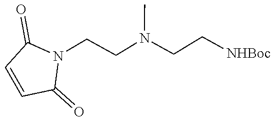 [SMILES: CN(CCNC(=O)OC(C)(C)C)CCN1C(=O)C=CC1=O]

To a solution of tert-butyl (2-((2-aminoethyl)(methyl)amino)ethyl)carbamate (100 mg, 0.46 mmol) in tetrahydrofuran (2.00 mL) was added methyl 2,5-dioxo-2,5-dihydro-1H-pyrrole-1-carboxylate (107 mg, 0.69 mmol) and a solution of sodium bicarbonate (116 mg, 1.38 mmol) in water (1.00 mL). The reaction mixture was stirred at 0° C. for 2 h. The reaction mixture was filtrated and the organic layer was concentrated to dryness in vacuum. The resulting solution was added water (20.0 mL) and extracted with ethyl acetate (3×20 mL). The combined organic layers dried with anhydrous sodium sulfate and concentrated to dryness. The reside was purified by (silica gel, 100-200 mesh, 0-10% methanol in dichloromethane) to afford the title compound (73 mg, 53.4% yield) as a colorless oil.

¹H NMR (400 MHz, CDCl₃) δ (ppm) 6.71 (s, 2H), 3.65-3.62 (m, 2H), 3.16-3.13 (m, 2H), 2.62-2.41 (m, 4H), 2.25 (s, 3H), 1.45 (s, 10H).

### Step 4: 1-(2-((2-aminoethyl)(methyl)amino)ethyl)-1H-pyrrole-2,5-dione 2,2,2-trifluoroacetate

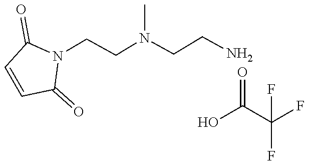

To a mixture of tert-butyl (2-((2-(2,5-dioxo-2,5-dihydro-1H-pyrrol-1-yl)ethyl)(methyl) amino)ethyl)carbamate (73.0 mg, 0.25 mmol) in dichloromethane (2.0 mL) was added trifluoroacetic acid (2.31 mL, 29.95 mmol). The reaction mixture was stirred at 20° C. for 5 h. The re action was concentrated to afford the title compound (40 mg, 82.6%) as a yellow oil.

### Step 5: N-((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-1-oxo-5-ureidopentan-2-yl)-N-(2-((2-(2,5-dioxo-2,5-dihydro-1H-pyrrol-1-yl)ethyl)(methyl)amino)ethyl)cyclobutane-1,1-dicarboxamide

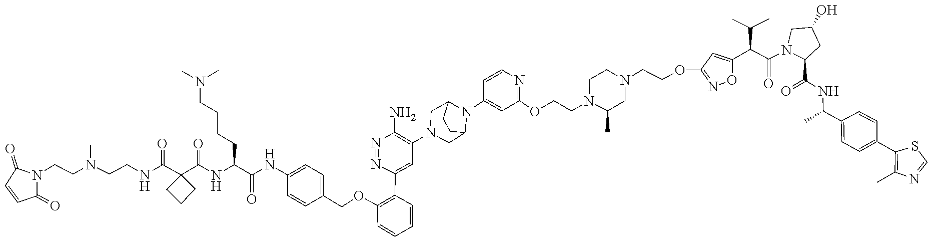 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)[C@H](C)C3)no2)C(C)C)cc1]

To a mixture of 1-(((2S)-1-((4-((2-(6-amino-5-(8-(2-(2-((R)-4-(2-((5-((R)-1-((2S,4R)-4-hydroxy-2-(((S)-1-(4-(4-methylthiazol-5-yl)phenyl)ethyl)carbamoyl)pyrrolidin-1-yl)-3-methyl-1-oxobutan-2-yl)isoxazol-3-yl)oxy)ethyl)-2-methylpiperazin-1-yl)ethoxy)pyridin-4-yl)-3,8-di azabicyclo[3.2.1]octan-3-yl)pyridazin-3-yl)phenoxy)methyl)phenyl)amino)-1-oxo-5-ureidopentan-2-yl)carbamoyl)cyclobutanecarboxylic acid (40.0 mg, 0.03 mmol) and 1-(2-((2-aminoeth yl)(methyl)amino)ethyl)-1H-pyrrole-2,5-dione 2,2,2-trifluoroacetate (10.5 mg, 0.03 mmol) in N,N-dimethylformamide (4.00 mL) was added to N,N-Diisopropylethylamine (0.01 mL, 0.08 mmol), 2-(7-azabenzotriazol-1-yl)-N,N,N′,N′-tetramethyluronium hexafluorophosphate (12.8 mg, 0.03 mmol). The reaction mixture was stirred at 25° C. for 3 h. The mixture was concentrated by oil pump. The residue was purified by prep-HPLC (Boston Green ODS 150*30 mm*5 um, water (0.075% trifluoroacetic acid)-acetonitrile 20%-50%) to afford the title compound (12.0 mg, 26.7% yield) as a white solid.

¹H NMR (400 MHz, DMSO-d₆) δ 10.19 (s, 1H), 9.02-8.86 (m, 1H), 8.39 (d, J=8.0 Hz, 1H), 7.94 (d, J=6.8 Hz, 1H), 7.87-7.78 (m, 2H), 7.65 (d, J=8.0 Hz, 2H), 7.51-7.41 (m, 5H), 7.36 (d, J=6.8 Hz, 5H), 7.29-7.09 (m, 1H), 6.97 (s, 2H), 6.76 (s, 1H), 6.42 (s, 1H), 6.15-5.96 (m, 2H), 5.07 (s, 2H), 4.93-4.86 (m, 1H), 4.71-4.60 (m, 2H), 4.52-4.24 (m, 9H), 3.66 (s, 4H), 3.33 (d, J=7.2 Hz, 5H), 3.09-2.96 (m, 8H), 2.45 (s, 3H), 2.42-2.34 (m, 4H), 2.29-2.14 (m, 2H), 2.04 (d, J=11.2 Hz, 3H), 1.91 (s, 2H), 1.83-1.55 (m, 5H), 1.51-1.30 (m, 9H), 1.19 (d, J=5.8 Hz, 5H), 0.96 (d, J=6.4 Hz, 3H), 0.86-0.75 (m, 3H).

LCMS (ESI) m/z: 1609.8 [M+H]⁺.

### Example 27

### Synthesis of Hy-B-PCIDE-1

### (3R,5S)-1-((2R)-2-(3-(2-((3R)-4-(2-((4-(3-(3-amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

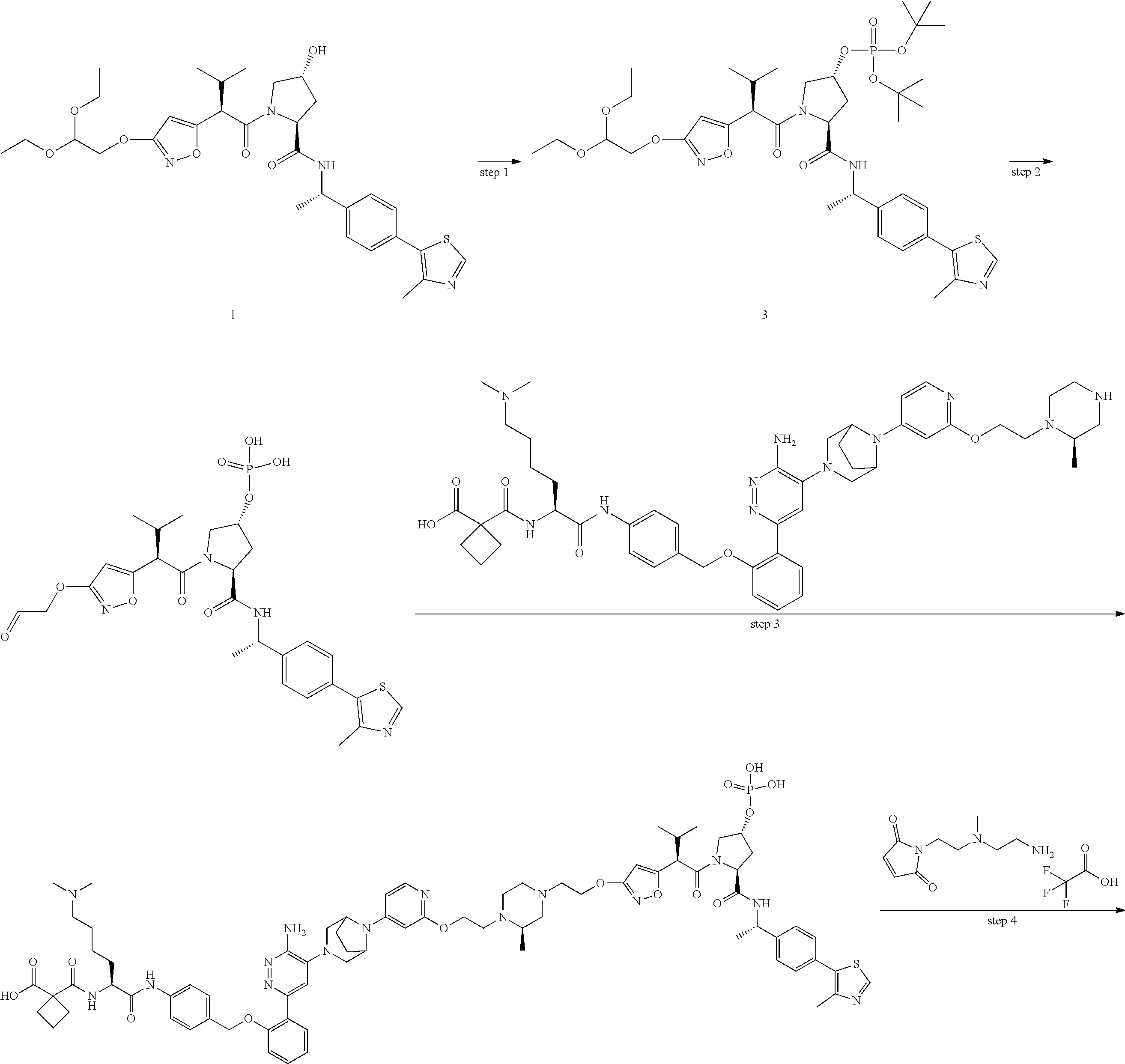

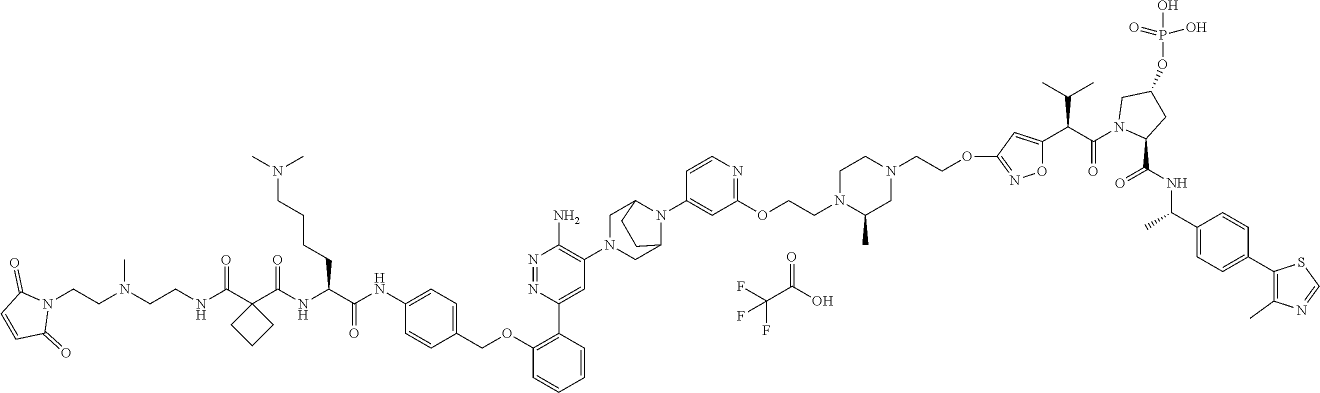 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)[C@H](C)C3)no2)C(C)C)cc1 ; O=C(O)C(F)(F)F]

### Step 1: di-tert-butyl ((3R,5S)-1-((R)-2-(3-(2,2-diethoxyethoxy) isoxazol-5-yl)-3-methylbutanoyl)-5-(((S)-1-(4-(4-methylthiazol-5-yl)phenyl)ethyl) carbamoyl) pyrrolidin-3-yl) phosphate

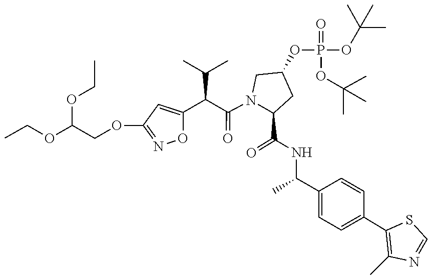 [SMILES: CCOC(COc1cc([C@H](C(=O)N2C[C@H](OP(=O)(OC(C)(C)C)OC(C)(C)C)C[C@H]2C(=O)N[C@@H](C)c2ccc(-c3scnc3C)cc2)C(C)C)on1)OCC]

Under nitrogen, to a solution of (2S,4R)-1-((R)-2-(3-(2,2-diethoxyethoxy) isoxazol-5-yl)-3-methylbutanoyl)-4-hydroxy-N—((S)-1-(4-(4-methylthiazol-5-yl)phenyl)ethyl) pyrrolidine-2-carboxamide (500 mg, 0.81 mmol) and 1H-tetrazole (5.4 mL, 0.45 M in ACN) in THF (5 mL) was added di-tert-butyl diisopropylphosphoramidite (451 mg, 1.63 mmol) dropwise at 0° C. The reaction was stirred at room temperature overnight. Then tBuOOH (0.4 mL, 5 M in decane) was added at −30° C. and stirred at −30° C. for 30 min. Then the mixture was stirred at room temperature for 4 hours. The reaction was quenched with aqueous NaHSO₃ and extracted with EtOAc. The combined organic layer was washed with water and brine. The organic layer was dried over anhydrous sodium sulfate and concentrated under vacuum. The residue was purified by pre-packed C18 column (solvent gradient: 0-70% ACN in water (0.05% NH₄HCO₃)) to yield 340 mg (51%) of the title compound as a yellow oil. LC-MS: (ESI, m/z): [M+H]⁺=807.

### Step 2: (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate

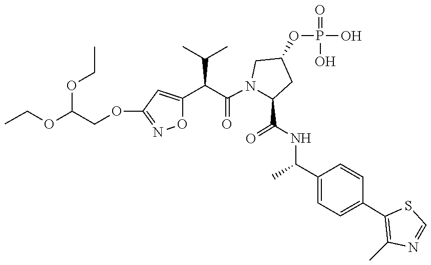 [SMILES: CCOC(COc1cc([C@H](C(=O)N2C[C@H](OP(=O)(O)O)C[C@H]2C(=O)N[C@@H](C)c2ccc(-c3scnc3C)cc2)C(C)C)on1)OCC]

Under nitrogen, a solution of di-tert-butyl ((3R,5S)-1-((R)-2-(3-(2,2-diethoxyethoxy)isoxazol-5-yl)-3-methylbutanoyl)-5-(((S)-1-(4-(4-methylthiazol-5-yl)phenyl)ethyl)carbamoyl)pyrrolidin-3-yl) phosphate (150 mg, 0.185 mmol) in HCOOH (0.5 mL) and water (0.5 mL) was stirred at 60° C. for 1 h. The solution was concentrated under vacuum to afford 121 mg (crude) of the title compound as a solid. LC-MS: (ESI, m/z): [M+H]+=695.

### Step 3: 1-(((2S)-1-((4-((2-(6-Amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid

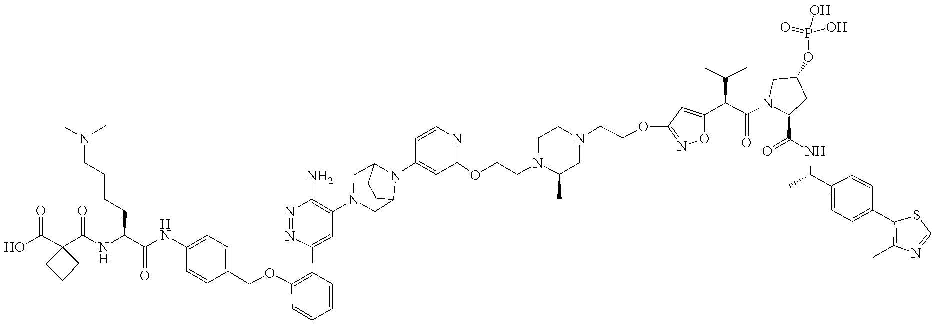 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)O)CCC8)cc7)nnc5N)C6)ccn4)[C@H](C)C3)no2)C(C)C)cc1]

A solution of 1-(((2S)-1-((4-((2-(6-amino-5-(8-(2-(2-((R)-2-methylpiperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (100 mg, 0.110 mmol), (3R,5S)-1-((R)-3-methyl-2-(3-(2-oxoethoxy)isoxazol-5-yl)butanoyl)-5-(((S)-1-(4-(4-methylthiazol-5-yl)phenyl)ethyl)carbamoyl)pyrrolidin-3-yl dihydrogen phosphate (121 mg, 0.190 mmol) and NaOAc (13.6 mg, 0.170 mmol) in methyl alcohol (1.2 mL) and dichloromethane (0.4 mL) was stirred at room temperature for 1 hours. Then NaBH₃CN (34.7 mg, 0.550 mmol) was added and stirred at room temperature for 0.5 hours. Water was added to quench the reaction. The solvent was concentrated under vacuum. The product was purified by Prep-HPLC (Column: YMC-Actus Triart C18 ExRS, 30*150 mm, 5 m; Mobile Phase A: Water (10 mmol/L NH₄HCO₃), Mobile Phase B: ACN; Flow rate: 60 mL/min; Gradient: 36% B to 60% B in 9 min, 60% B; Wavelength: 254/220 nm; RT1(min): 7.63) to afford 30.0 mg (18% yield) of the title compound as a white solid. LCMS (ESI) [M+H]⁺=1509.

### Step 4: (3R,5S)-1-((2R)-2-(3-(2-((3R)-4-(2-((4-(3-(3-amino-6-(2-((4-((S)-6-(dimethylamino)-2-(1-((2-((2-(2,5-dioxo-2,5-dihydro-1H-pyrrol-1-yl)ethyl)(methyl)amino)ethyl)carbamoyl)cyclobutane-1-carboxamido)hexanamido)benzyl)oxy)phenyl)pyridazin-4-yl)-3,8-diazabicyclo[3.2.1]octan-8-yl)pyridin-2-yl)oxy)ethyl)-3-methylpiperazin-1-yl)ethoxy)isoxazol-5-yl)-3-methylbutanoyl)-5-(((S)-1-(4-(4-methylthiazol-5-yl)phenyl)ethyl)carbamoyl)pyrrolidin-3-yl dihydrogen phosphate (2,2,2-trifluoroacetic acid salt)

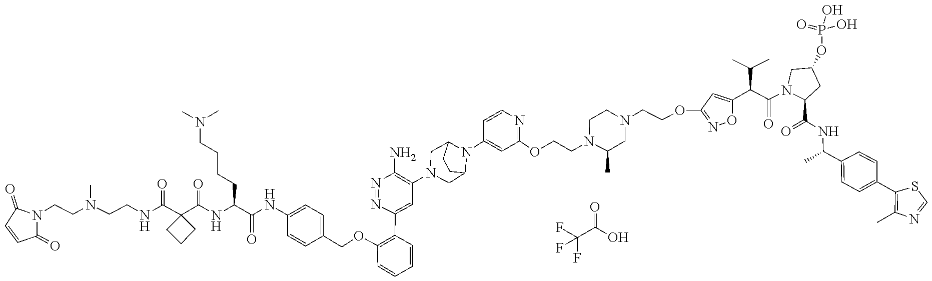 [SMILES: Cc1ncsc1-c1ccc([C@H](C)NC(=O)[C@@H]2C[C@@H](OP(=O)(O)O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)[C@H](C)C3)no2)C(C)C)cc1 ; O=C(O)C(F)(F)F]

To a solution of 1-(((2S)-1-((4-((2-(6-amino-5-(8-(2-(2-((R)-2-methyl-4-(2-((5-((R)-3-methyl-1-((2S,4R)-2-(((S)-1-(4-(4-methylthiazol-5-yl)phenyl)ethyl)carbamoyl)-4-(phosphonooxy)pyrrolidin-1-yl)-1-oxobutan-2-yl)isoxazol-3-yl)oxy)ethyl)piperazin-1-yl)ethoxy)pyridin-4-yl)-3,8-diazabicyclo[3.2.1]octan-3-yl)pyridazin-3-yl)phenoxy)methyl)phenyl)amino)-6-(dimethylamino)-1-oxohexan-2-yl)carbamoyl)cyclobutane-1-carboxylic acid (30.0 mg, 0.0200 mmol), 1-(2-((2-aminoethyl)(methyl)amino)ethyl)-1H-pyrrole-2,5-dione (2,2,2-trifluoroacetic acid salt) (12.3 mg, crude) and DIPEA (25.6 mg, 0.200 mmol) in DMF (1 mL) was added HATU (9.07 mg, 0.0200 mmol) at room temperature. The reaction was stirred at room temperature for 10 minutes. The crude was purified by Prep-HPLC (Column: Xselect CSH C18 OBD Column 30*150 mm 5 m; Mobile Phase A: Water (0.05% TFA), Mobile Phase B: ACN; Flow rate: 60 mL/min; Gradient: 12% B to 24% B in 10 min, 24% B; Wavelength: 254/220 nm; RT1(min): 10) to afford 8.70 mg (24% yield) of Hy-B-PCIDE-1 as a white solid. LCMS (ESI) [M+H]+=1688. ¹H NMR (300 MHz, DMSO-d6) δ 10.26 (s, 1H), 8.99 (s, 1H), 8.50 (d, J=7.5 Hz, 1H), 8.15 (t, 1H), 7.87 (d, J=6.5 Hz, 2H), 7.65 (d, J=8.3 Hz, 2H), 7.61-7.51 (m, 2H), 7.50-7.30 (m, 8H), 7.20-6.90 (m, 5H), 6.62 (s, 1H), 6.14 (s, 1H), 5.99 (s, 1H), 5.09 (s, 2H), 4.92 (t, J=7.2 Hz, 1H), 4.79 (s, 1H), 4.55-4.25 (m, 8H), 3.80-3.65 (m, 8H), 3.25-2.95 (m, 17H), 2.70 (s, 3H), 2.62 (s, 6H), 2.48-2.38 (m, 9H), 2.20-1.55 (m, 13H), 1.52-1.30 (m, 5H), 1.30-1.15 (m, 4H), 0.85 (d, J=6.4 Hz, 3H), 0.85-0.70 (m, 3H).

### Example 28

### Cell Assays to Determine DC50 and Dmax

HCC515 and H1944 cells were plated in 384 well plates at 4000 and 2500 cells/well, respectively. The next day Ab-CIDEs were added. Following 24 h of drug treatment the cells were fixed with 400 formaldehyde for 15 minutes. The plates were washed three time with PBS. The cells were incubated with IF blocking solution (100% FCS, 10% BSA, 0.1% Triton, 0.010% Azide, X-100 in PBS). After 1.5 h a 2× solution of primary antibody diluted in IF blocking buffer: BRM (Cell signaling Cat #11966, 1:2000) was added. The plates were incubated over night at 4° C. The following morning cells were washed three time with PBS. Cells were then incubated with secondary antibodies (rabbit-Alexa 488 A21206 (1:2000)) for 1 h at room temperature in the dark. Hoechst H3570 at 1:5000 was added to the wells and the plates were incubated for an additional 30 minutes. Plates were wash 3×PBS and image on Opera Phenix™ High Content Screening System. Using nuclear staining as a mask, nuclear BRM mean signal intensity was quantified.

Data are shown in the Table below. The data evidence the successful antibody targeting strategies disclosed herein and that Ab-CIDEs containing hydrolysable linkers are able to degrade target proteins at levels similar to Ab-CIDEs containing a non-hydrolysable linker.

| | | DC50 |
|---|---|---|
| | | μg/mL |
| | Ab | (NCI- |
| L1-D of the Antibody-conjugated CIDE | Target | H1944) |
| Non-hydrolysable | TfR2 | 0.112 |
| 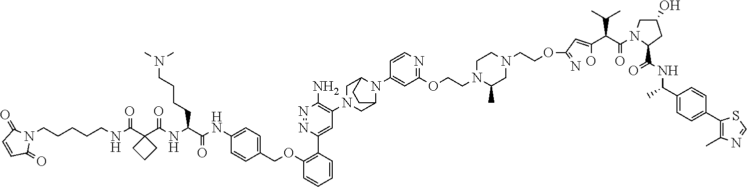 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCCCCN9C(=O)C=CC9=O)CCC8)cc7)nnc5N)C6)ccn4)[C@H](C)C3)no2)C(C)C)cc1] | | |
| Hy-B-CIDE-6 | TfR2 | 0.28 |
| Hy-B-CIDE-1 | TfR2 | 0.23 |
| Hy-B-CIDE-2 | TfR2 | 0.29 |
| Hy-B-CIDE-5 | TfR2 | 0.71 |
| Hy-B-CIDE-8 | TfR2 | 0.151 |
| Non-hydrolysable | TfR2 | 0.36 |
| 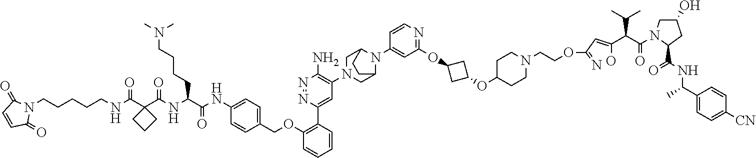 [SMILES: CC(C)[C@@H](C(=O)N1C[C@H](O)C[C@H]1C(=O)N[C@@H](C)c1ccc(C#N)cc1)/C(O)=C/C(=N)OCCN1CCC(O[C@H]2C[C@H](Oc3cc(N4C5CCC4CN(c4cc(-c6ccccc6OCc6ccc(NC(=O)[C@H](CCCCN(C)C)NC(=O)C7(C(=O)NCCCCCN8C(=O)C=CC8=O)CCC7)cc6)nnc4N)C5)ccn3)C2)CC1] | | |
| Hy-B-CIDE-7 | TfR2 | 0.32 |
| Hy-B-CIDE-7 | TfR2 | 0.14 |
| Hy-B-CIDE-11 | TfR2 | 37.28 |
| Hy-B-CIDE-12 | TfR2 | 20.59 |
| Hy-B-CIDE-9 | TfR2 | 3.87 |
| Hy-B-CIDE-10 | TfR2 | 7.46 |
| Hy-B-PCIDE-2 | TfR2 | ND |
| | DCmax | |
| | (NCI- | |
| L1-D of the Antibody-conjugated CIDE | H1944) | DAR |
| Non-hydrolysable | 90 | 6.14 |
| 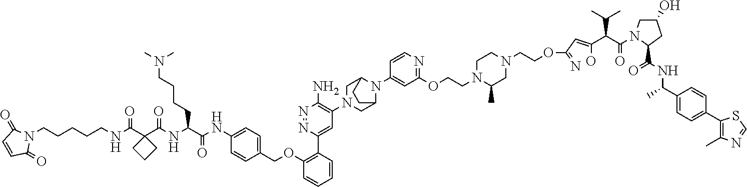 [SMILES: Cc1ncsc1-c1ccc([C@H](C)NC(=O)[C@@H]2C[C@@H](O)CN2C(=O)[C@@H](c2cc(OCCN3CCN(CCOc4cc(N5C6CCC5CN(c5cc(-c7ccccc7OCc7ccc(NC(=O)[C@H](CCCCN(C)C)NC(=O)C8(C(=O)NCCCCCN9C(=O)C=CC9=O)CCC8)cc7)nnc5N)C6)ccn4)[C@H](C)C3)no2)C(C)C)cc1] | | |
| Hy-B-CIDE-6 | 90 | 5.60 |
| Hy-B-CIDE-1 | 89 | 5.79 |
| Hy-B-CIDE-2 | 90 | 5.93 |
| Hy-B-CIDE-5 | 76 | 5.41 |
| Hy-B-CIDE-8 | 74 | 5.41 |
| Non-hydrolysable | 80 | 5.61 |
| 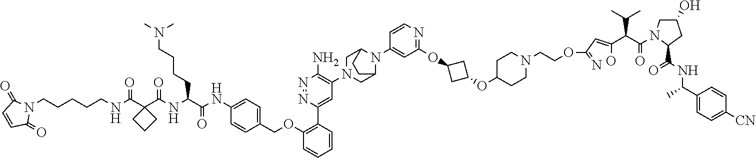 [SMILES: CC(C)[C@@H](C(=O)N1C[C@H](O)C[C@H]1C(=O)N[C@@H](C)c1ccc(C#N)cc1)/C(O)=C/C(=N)OCCN1CCC(O[C@H]2C[C@H](Oc3cc(N4C5CCC4CN(c4cc(-c6ccccc6OCc6ccc(NC(=O)[C@H](CCCCN(C)C)NC(=O)C7(C(=O)NCCCCCN8C(=O)C=CC8=O)CCC7)cc6)nnc4N)C5)ccn3)C2)CC1] | | |
| Hy-B-CIDE-7 | 62 | 5.61 |
| Hy-B-CIDE-7 | 75 | 13.75 |
| Hy-B-CIDE-11 | 53 | 5.51 |
| Hy-B-CIDE-12 | 60 | 5.77 |
| Hy-B-CIDE-9 | 82 | 5.80 |
| Hy-B-CIDE-10 | 80 | 5.48 |
| Hy-B-PCIDE-2 | 42 | 13.13 |

### Example 29

### Conjugation of Hydrolysable Linkers to Antibody to Achieve High DAR

Hydrolysable maleimide-containing linkers were shown to provide high DAR (values above 6). At this level of DAR, certain non-hydrolysable linkers become unstable, whereas the antibody-conjugated hydrolysable linkers were prepared, isolated and characterized. The hydrolysable linkers therefore are able to provide an advantage in obtaining high DAR, which results in high numbers of CTDEs that can be attached to a single antibody.

Data showing the level of DAiR obtainable with a hydrolysable linker-containing CIDE:

| L1-CIDE | DAR |
|---|---|
| 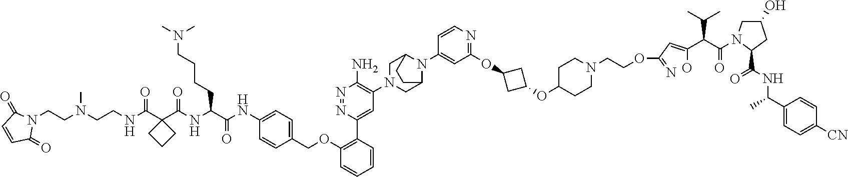 [SMILES: CC(C)[C@@H](C(=O)N1C[C@H](O)C[C@H]1C(=O)N[C@@H](C)c1ccc(C#N)cc1)/C(O)=C/C(=N)OCCN1CCC(O[C@H]2C[C@H](Oc3cc(N4C5CCC4CN(c4cc(-c6ccccc6OCc6ccc(NC(=O)[C@H](CCCCN(C)C)NC(=O)C7(C(=O)NCCN(C)CCN8C(=O)C=CC8=O)CCC7)cc6)nnc4N)C5)ccn3)C2)CC1] | 5.61 |
| 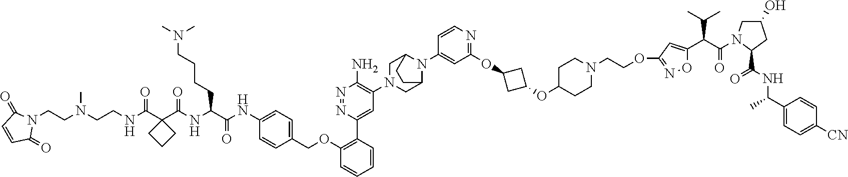 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)no1] | 13.75 |
| Hy-B-CIDE-7 | |

DAR levels for a hydrolysable linker containing CIDE conjugated to an antibody having advantageously high DAR compared to a non-hydrolysable linker containing CIDE:

| | DAR | |
|---|---|---|
| | 14 | |
| | syn- | Sta- |
| L1-CIDE | thesis | bility |
| 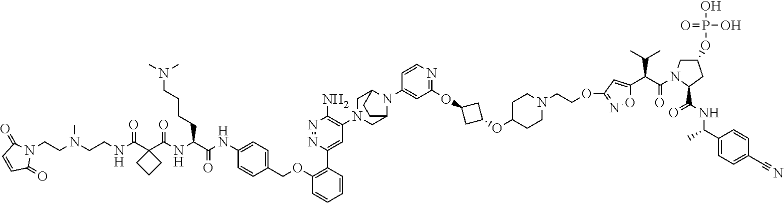 [SMILES: CC(C)[C@@H](C(=O)N1C[C@H](OP(=O)(O)O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)no1] | 13.13 | Stable |
| Hy-B-PCIDE-2 | | |
| 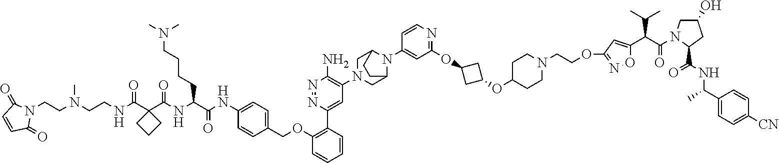 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCN(C)CCN9C(=O)C=CC9=O)CCC8)cc7)nnc5N)C6)ccn4)C3)CC2)no1] | 13.75 | Stable |
| Hy-B-CIDE-7 | | |
| Non-hydrolysable | High | Un- |
| 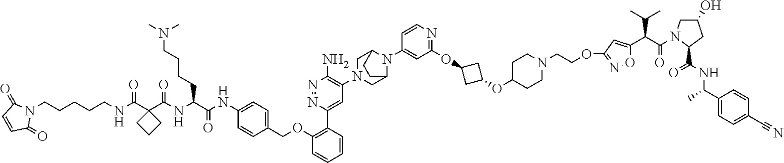 [SMILES: CC(C)[C@@H](C(=O)N1C[C@H](O)C[C@H]1C(=O)N[C@@H](C)c1ccc(C#N)cc1)c1cc(OCCN2CCC(O[C@H]3C[C@H](Oc4cc(N5C6CCC5CN(c5cc(-c7ccccc7OCc7ccc(NC(=O)[C@H](CCCCN(C)C)NC(=O)C8(C(=O)NCCCCCN9C(=O)C=CC9=O)CCC8)cc7)nnc5N)C6)ccn4)C3)CC2)no1] | DAR unat- tain- able | stable |

Efforts have been made to ensure accuracy with respect to numbers used (e.g. amounts, temperature, etc.) but some experimental errors and deviations should be accounted for.

One skilled in the art will recognize many methods and materials similar or equivalent to those described herein, which could be used in the practicing the subject matter described herein. The present disclosure is in no way limited to just the methods and materials described.

Unless defined otherwise, technical and scientific terms used herein have the same meaning as commonly understood by one of ordinary skill in the art to which this subject matter belongs, and are consistent with: Singleton et al (1994) Dictionary of Microbiology and Molecular Biology, 2nd Ed., J. Wiley & Sons, New York, NY; and Janeway, C., Travers, P., Walport, M., Shlomchik (2001) Immunobiology, 5th Ed., Garland Publishing, New York.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

As used herein, the term “about,” when referring to a value is meant to encompass variations of, in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Where a range of values is provided, it is understood that each intervening value, to the tenth of the unit of the lower limit, unless the context clearly dictates otherwise, between the upper and lower limit of the range and any other stated or intervening value in that stated range, is encompassed. The upper and lower limits of these small ranges which may independently be included in the smaller rangers is also encompassed, subject to any specifically excluded limit in the stated range. Where the stated range includes one or both of the limits, ranges excluding either or both of those included limits are also included.

Many modifications and other embodiments set forth herein will come to mind to one skilled in the art to which this subject matter pertains having the benefit of the teachings presented in the foregoing descriptions and the associated drawings. Therefore, it is to be understood that the subject matter is not to be limited to the specific embodiments disclosed and that modifications and other embodiments are intended to be included within the scope of the appended claims. Although specific terms are employed herein, they are used in a generic and descriptive sense only and not for purposes of limitation.

1. A compound having the structure:
L₁-D
wherein,
D is a CIDE;
L₁ is a linker-1 covalently bound to D and having a structure of Formula I: 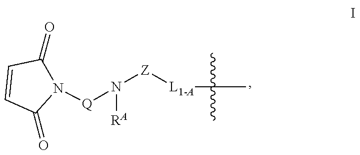
wherein,
Z is —(CH₂)ₚ— or —CH₂—(CH₂—O—CH₂)ₚ—CH₂—;
p is an integer from 1 to 24;
R^{A} is hydrogen, C₁₋₆ alkyl, or —(CH₂)ᵥ-aryl,
wherein, v is 0 or 1;
Q is selected from the group consisting of:
i) 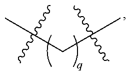
wherein q is 1, 2, 3 or 4; and
ii) 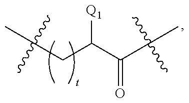
wherein t is 0, 1, 2, 3 or 4;
Q¹ is hydrogen, 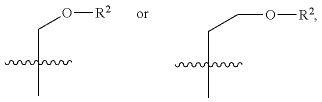
wherein R² is hydrogen, halo C₁₋₆ alkyl or C₁₋₆ alkyl; and
L_{1-A} is: 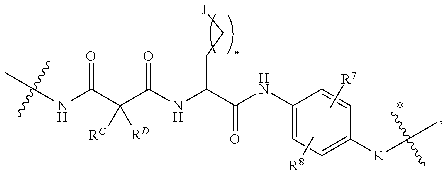
wherein 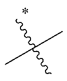
indicates the attachment point to D;
w is 0, 1, 2, 3, 4 or 5;
J is selected from the group consisting of —C₁₋₅ alkyl, —N(R^{x})(R^{y}), —C(O)NH₂, —NH—C(O)—NH₂, and —NH—C(NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

2. The compound of claim 1, wherein Q is: 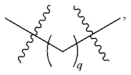
wherein q is 1, 2, 3 or 4.

3. (canceled)

4. The compound of claim 1, wherein Q is: 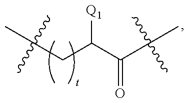
wherein t is 0, 1, 2, 3 or 4;
Q¹ is hydrogen, 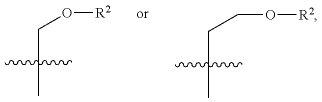
wherein R² is hydrogen, halo C₁₋₆ alkyl or C₁₋₆ alkyl.

5. The compound of claim 4, wherein R² is hydrogen or C₁₋₆ alkyl.

6-9. (canceled)

10. The compound of claim 4, wherein t is 0.

11. The compound of claim 1, wherein Z is —(CH₂)ₚ—, wherein p is 1, 2, 3, 4, 5 or 6.

12. (canceled)

13. (canceled)

14. The compound of claim 1, wherein Z is —CH₂—(CH₂—O—CH₂)ₚ—CH₂—, wherein p is 1, 2, 3, 4, 5 or 6.

15. (canceled)

16. (canceled)

17. The compound of claim 1, wherein R^{A} is hydrogen or C₁₋₆ alkyl.

18. (canceled)

19. The compound of claim 1, wherein R^{A} is phenyl or benzyl.

20. The compound of claim 1, wherein R⁷ and R⁸ are each hydrogen.

21. The compound of claim 1, wherein R^{C} and R^{D} together with the carbon to which each is attached form an optionally substituted C₃₋₆ cycloalkyl.

22. (canceled)

23. The compound of claim 1, wherein K is —CH(R)—, wherein R is hydrogen, C₁₋₃ alkyl, N(R^{x})(R^{y}) or C(O)—N(R^{x})(R^{y}), wherein R^{x} and R are each independently selected from hydrogen and C₁₋₃ alkyl, or R^{x} and R^{y} together with the nitrogen to which each is attached form an optionally substituted 5- to 7-member heterocyclyl.

24. (canceled)

25. (canceled)

26. An antibody-CIDE conjugate having the structure:
Ab-(L₁-D)ⱼ
wherein,
Ab is an antibody;
j is 1 to 16;
D is a CIDE;
L₁ is a linker-1 covalently bound to Ab and to D and having a structure of Formula I-A: 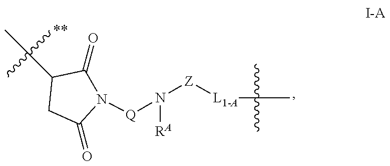
wherein,
Z is —(CH₂)ₚ— or —CH₂—(CH₂—O—CH₂)ₚ—CH₂—, wherein p is an integer from 1 to 24;
R^{A} is hydrogen, C₁₋₆ alkyl, or —(CH₂)ᵥ-aryl, wherein, v is 0 or 1;
Q is selected from the group consisting of:
i) 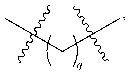
wherein q is 1, 2, 3 or 4; and
ii) 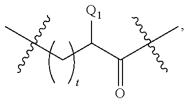
wherein t is 0, 1, 2, 3 or 4;
Q¹ is hydrogen, 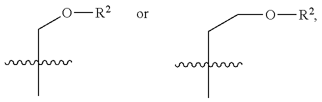
wherein R² is hydrogen, halo(C₁₋₆)alkyl or C₁₋₆ alkyl; and
L_{1-A} is: 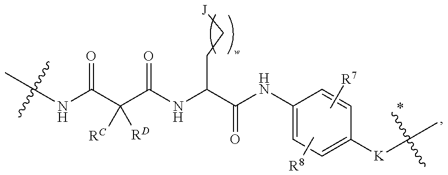
wherein 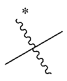
indicates the attachment point to D;
w is 0, 1, 2, 3, 4 or 5;
J is selected from the group consisting of —N(R^{x})(R^{y}), —C(O)NH₂, —NH—C(O)—NH₂, and —NH—C(═NH)(NH₂), wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

27-50. (canceled)

51. A pharmaceutical composition comprising the antibody-CIDE conjugate of claim 26 and one or more pharmaceutically acceptable excipients.

52. A method of treating a disease in a human in need thereof, comprising administering to said human an effective amount of the antibody-CIDE conjugate of claim 26.

53. The method of claim 52, wherein said disease is cancer.

54. The method of claim 53, wherein said cancer is BRM-dependent.

55. (canceled)

56. A method of reducing the level of a target protein in a subject comprising administering to said subject the antibody-CIDE conjugate of claim 26, wherein said PB portion binds said target protein, wherein ubiquitin ligase effects degradation of said bound target protein, wherein the level of said target protein is reduced.

57. An antibody-CIDE conjugate having the structure:
Ab-((L_{X})-D)ⱼ
wherein,
Ab is an antibody;
j is 1 to 16;
D is a CIDE;
L_{X} is selected from the group consisting of L₁ and L₁ₕ, wherein L₁ₕ is present in at least one instance of L_{X}:
L₁ is a linker-1 covalently bound to Ab and to D and having a structure of Formula I-A: 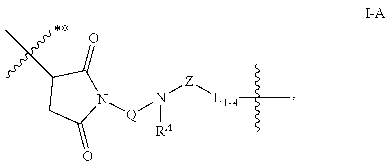
and,
L₁ₕ is a linker-1 covalently bound to D and having a structure of Formula I-B: 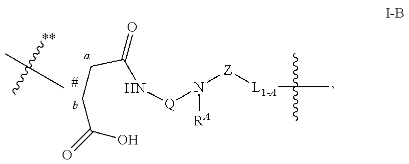
wherein, # indicates the point of attachment at position a or b;
in L₁ and L₁ₕ, 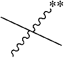
indicates the point of attachment to the antibody;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂)ᵥ-aryl, wherein, v is 0 or 1;
Q is selected from the group consisting of:
i) 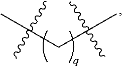
wherein q is 1, 2, 3 or 4; and
ii) 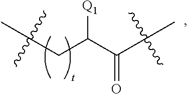
wherein t is 0, 1, 2, 3 or 4;
Q¹ is hydrogen, 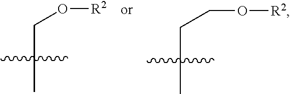
wherein R² is hydrogen, halo C₁₋₆ alkyl or C₁₋₆ alkyl; and
L_{1-A} is: 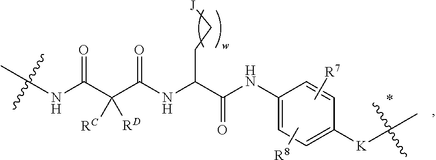
wherein 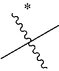
indicates the attachment point to D;
w is 0, 1, 2, 3, 4 or 5;
J is selected from the group consisting of —C₁₋₅ alkyl, —N(R^{x})(R^{y}), —C(O)NH₂, —NH—C(O)—NH₂, —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.

58. An antibody-conjugate having the structure:
Ab-(L_{Z})ⱼ
wherein,
Ab is an antibody;
j is 1 to 16;
L_{Z} is selected from the group consisting of L_{X1} and Lx-D, wherein L_{X1} is present in at least one instance of L_{Z}:
L_{X1} is covalently bound to Ab and has a structure of Formula I-G: 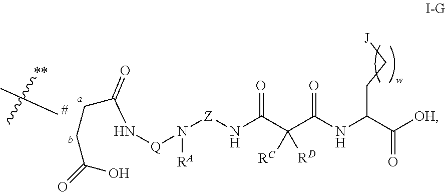
and,
Lx-D, wherein Lx is selected from the group consisting of L₁ and L₁ₕ:
L₁ is a linker-1 covalently bound to Ab and to D and having a structure of Formula I-A or Formula I-C: 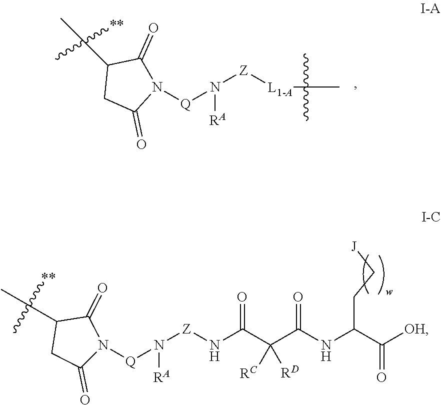
and,
L₁ₕ is a linker-1 covalently bound to D and having a structure of Formula I-B: 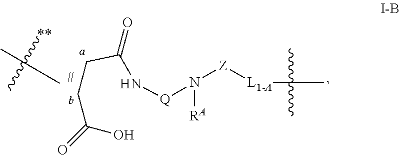
wherein, # indicates the point of attachment at position a or b; 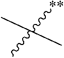
indicates the point of attachment to the antibody;
Z is —(CH₂)ₚ— or —CH₂—(CH₂—O—CH₂)ₚ—CH₂—, wherein p is 1, 2, 3, 4, 5 or 6;
R^{A} is hydrogen, C₁₋₆ alkyl, or —(CH₂)ᵥ-aryl, wherein, v is 0 or 1;
Q is selected from the group consisting of:
i) 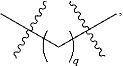
wherein q is 1, 2, 3 or 4; and
ii) 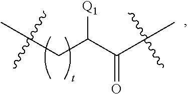
wherein t is 0, 1, 2, 3 or 4;
Q¹ is hydrogen, 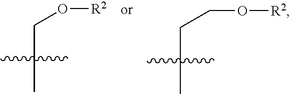
wherein R² is hydrogen, halo C₁₋₆ alkyl or C₁₋₆ alkyl; and
L_{1-A} is: 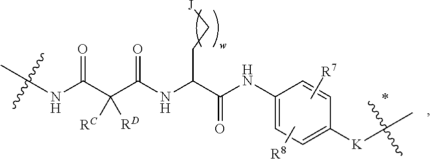
wherein 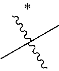
indicates the attachment point to D;
w is 0, 1, 2, 3, 4 or 5;
J is selected from the group consisting of —C₁₋₅ alkyl, —N(R^{x})(R^{y}), —C(O)NH₂, —NH—C(O)—NH₂, —NH—C(═NH)—NH₂, wherein, R^{x} and R^{y} are each independently selected from hydrogen and C₁₋₃ alkyl;
K is selected from the group consisting of C₁₋₃alkylene, —CH(R)—, —C(O)—, —C(O)—O—CH(R)—, —CH₂—O—C(O)—, —CH₂—O—C(O)—NH—CH₂—, and —CH₂—O—C(O)—R—[CH₂]ᵤ—O—, wherein R is hydrogen, C₁₋₃alkyl, N(R^{x})(R^{y}), —O—N(R^{x})(R^{y}) or C(O)—N(R^{x})(R^{y}), wherein u is 0, 1, 2, or 3, and wherein R^{x} and R^{y} are each independently selected from hydrogen and C₁₋₃alkyl, or R^{x} and R^{y} together with the nitrogen to which each is attached form an optionally substituted 5- to 7-member heterocyclyl;
R^{C} and R^{D} are each independently selected from hydrogen and C₁₋₃alkyl, or R^{C} and R^{D}, together with the carbon to which each is attached, form an optionally substituted C₃₋₆ cycloalkyl; and
R⁷ and R⁸ are each independently hydrogen, halo, C₁₋₅ alkyl, C₁₋₅ alkoxy or hydroxyl.
